SEQUENCE LISTING (24 sequences)

SEQ ID NO: 1 - protein, 667 aa, Camelia sativa
MAAATTTSSSSIPFSTKPSSSKSPLPISRFTLPFSLNPNKSSSSSRRRGIKSTSLSISAVLNTTANVSTTTPPSKPTKPEKKKFVSRFAPDQPRKGADILVEALERQGVETVFAYPGGASMEIHQALTRSSSIRNVLPRHEQGGVFAAEGYARSTGKPGICIATSGPGATNLVSGLADALLDSVPLVAITGQVPRRMIGTDAFQETPIVEVTRSITKHNYLVMDVEDIPRIVEEAFFLATSGRPGPVLVDVPKDIQQQLAIPNWEQSMRLPGYMSRMPKPPEDSHLEQIVRLVSESKKPVLYVGGGCLNSSEELGRFVELTGIPVASTLMGLGAYPCDDELSLHMLGMHGTVYANYSVEHSDLLLAFGVRFDDRVTGKLEAFASRAKIVHIDIDSAEIGKNKTPHVSVCGDVKLALQGMNKVLENRAEERKLDFGVWRSELNEQKQKFPLSFKTFGEAIPPQYAIQVLDELTDGKAIISTGVGQHQMWAAQFYKYKKPRQWLSSAGLGAMGFGLPAAIGASVANPDAIVVDIDGDGSFIMNVQELATIRVENLPVKILILNNQHLGMVMQWEDRFYKANRAHTYLGNPAAEDEIFPNMLQFASACGIPAARVTKKAELREAIQKMLDTPGPYLLDVICPHQEHVLPMIPSGGTFNDVITEGDGRTKY

SEQ ID NO: 2 - protein, 667 aa, Camelia sativa
MAAATTTSSSSIPFSAKPSSSKSPLPISRFTLPFSLNPNKSSSSSRRRGIKSTSLSISAVLNTTTNVSTTTPPSKQTKPEKKKFVSRFAPDQPRKGADILVEALERQGVETVFAYPGGASMEIHQALTRSSSIRNVLPRHEQGGVFAAEGYARSTGKPGICIATSGPGATNLVSGLADALLDSVPLVAITGQVPRRMIGTDAFQETPIVEVTRSITKHNYLVMDVEDIPRIVEEAFFLATSGRPGPVLVDVPKDIQQQLAIPNWEQSMRLPGYMSRMPKPPEDSHLEQIVRLVSESKKPVLYVGGGCLNSSEELGRFVELTGIPVASTLMGLGAYPCDDELSLHMLGMHGTVYANYSVEHSDLLLAFGVRFDDRVTGKLEAFASRAKIVHIDIDSAEIGKNKTPHVSVCGDVKLALQGMNKVLENRAEELKLDFGVWRSELNEQKQMFPLSFKTFGEAIPPQYAIQVLDELTDGRAIISTGVGQHQMWAAQFYKYKKPRQWLSSAGLGAMGFGLPAAIGASVANPDAIVVDIDGDGSFIMNVQELATIRVENLPVKILILNNQHPGMVIQWEARFYKANRAHTYLGNPAAEDEIFPNMLQFASACGIPAARVTKKAELREAIQKMLDTPGPYLLDVICPHQEHVLPMIPSGGTFNDVITEGDGRTKY

SEQ ID NO: 3 - protein, 666 aa, Camelia sativa
MAAATTPSSSSIPFSTKPSSSKSPLPISRFTLPFALNPTKSSSSRRRGIKSTSLSISAVLNTTTNVSTTTPPSKPTKPEKKKFVSRFAPDQPRKGADILVEALERQGVETVFAYPGGASMEIHQALTRSSSIRNVLPRHEQGGVFAAEGYARSTGKPGICIATSGPGATNLVSGLADALLDSVPLVAITGQVPRRMIGTDAFQETPIVEVTRSITKHNYLVMDVEDIPRIVEEAFFLATSGRPGPVLVDVPKDIQQQLAIPNWEQAMRLPGYMSRMPKPPEDSHLEQIVRLISESKKPVLYVGGGCLNSSEELGRFVELTGIPVASTLMGLGAYPCDDELSLHMLGMHGTVYANYSVEHSDLLLAFGVRFDDRVTGKLEAFASRAKIVHIDIDSAEIGKNKTPHVSVCGDVKLALQGMNKVLENRAEELKLDFGVWRSELNEQKQKFPLSFKTFGEAIPPQYAIQVLDELTDGRAIISTGVGQHQMWAAQFYKYKKPRQWLSSAGLGAMGFGLPAAIGASVANPDAIVVDIDGDGSFIMNVQELATIRVENLPVKILILNNQHLGMVMQWEDRFYKANRAHTYLGNPGAEDEIFPNMLQFASACGIPAARVTKKAELREAIQKMLDTPGPYLLDVICPHQEHVLPMIPSGGTFNDVITEGDGRTKY

SEQ ID NO: 4 - DNA, 2001 bp, Camelia sativa
  source 1..2001: mol_type = genomic DNA
atggcggcggcaacaacaacatcatcatcttcgatccccttctcaacaaaaccttcctcatccaaatcaccattaccaatctccaggttcaccctcccattctccctaaacccaaacaaatcctcctcctcctcccgccgccgcggtatcaaatcaacctccctctccatctccgccgttctcaacacaacagccaatgtctcaaccactactcctccatcaaaaccaaccaaacccgaaaaaaagaaattcgtctcccgatttgctccagatcagccacgcaaaggcgctgacatcctcgtcgaagctttagaacgccaaggcgtagaaacagtcttcgcttacccaggaggtgcatcaatggagattcaccaagccttaactcgttcctcctcgatccgtaacgtcctcccacgtcacgaacaaggaggcgtgttcgcagctgaaggatacgctagatccaccggtaaaccaggtatctgtatagctacctcaggtcctggagctacgaatctcgttagcggattagccgatgccttgttagatagtgttcctcttgtagcgatcacgggtcaagtccctcgtaggatgattggtactgatgcgtttcaagaaactcccatcgttgaggtgacgcgttccattactaaacataactatcttgtgatggatgttgaagatattcctaggattgttgaggaagctttctttttagctacttctggtagacctggacctgttttggttgatgttcctaaagatatacagcagcagcttgcgattcctaattgggaacagtctatgaggttacctggttatatgtctaggatgcctaaacctccggaagattctcatttggagcagattgttaggttggtttctgagtctaagaagcctgtgttgtatgttggtggtggttgtttgaattcgagtgaggagttgggtaggtttgtggaacttacgggtatacctgttgcgagtacgttgatgggtcttggtgcttacccttgtgatgacgagttgtcgttgcatatgcttggaatgcacgggaccgtgtacgcgaattactctgtggagcatagtgatttgttgttggcttttggggtgaggtttgatgatcgtgtcacgggtaagcttgaggcttttgctagtagggcgaagattgttcacattgatattgactcggctgagattgggaagaacaagactcctcatgtgtctgtgtgtggagatgttaagctggctttgcaagggatgaacaaggttcttgagaaccgagctgaggagcgtaagcttgattttggagtttggagaagtgagttgaacgaacagaaacaaaagttccctctgagcttcaagacgtttggggaagctattcctcctcagtatgcgattcaggtccttgatgagctgactgatgggaaagccattataagcactggtgtcggtcaacatcaaatgtgggcggcgcagttttacaagtacaaaaaaccgaggcagtggctatcatcagcaggccttggagctatgggttttggacttcccgctgctattggagcgtccgttgcgaaccctgatgcgattgttgtggatattgacggagatggaagcttcataatgaatgtgcaagagctggccactatcagggtagagaatctcccagtgaagatacttatactaaacaaccagcatcttggcatggttatgcaatgggaggatcggttctacaaagctaaccgagctcacacatatcttggtaacccggcagcggaggacgagatattcccgaacatgctgcagtttgcatcagcttgcggtattccagcggcgagggtgacaaagaaagcagagctccgagaagctatccagaaaatgctggataccccaggaccatacttgttggatgtgatttgtccgcaccaagaacatgtgttgccgatgatcccaagtggcggcactttcaatgatgtcataacggaaggagatggccggactaaatac

SEQ ID NO: 5 - DNA, 2001 bp, Camelia sativa
  source 1..2001: mol_type = genomic DNA
atggcggcggcaacaacaacatcatcatcttcgatccccttctcagcaaaaccttcctcatccaaatcaccattaccaatctccaggttcaccctcccattctccctaaacccaaacaaatcctcctcctcctcccgccgccgcggtatcaaatcaacctccctctccatctccgccgtcctcaacacaacaaccaatgtctcaaccaccactcctccatcaaaacaaaccaaacccgaaaaaaaaaaattcgtctcccgatttgctccagatcagccacgcaaaggcgctgacatcctcgtcgaagctttagaacgccaaggcgtagaaacagtcttcgcttacccaggaggtgcatcaatggagattcaccaagccttaactcgttcctcctcgatccgtaacgtccttccacgtcacgaacaaggtggcgtgttcgcagctgaaggctacgctagatccacaggtaaaccaggtatctgtatagcaacctcaggtcctggagctacaaatctcgttagcggattagccgatgccttgttagatagtgttcctcttgtagcgatcacgggtcaagtccctcgtaggatgattggtactgatgcgtttcaagaaactcccatcgttgaggtgacgcgttcgattactaaacataactatcttgtgatggatgttgaagatattcctaggattgttgaggaagctttctttttagctacctctggtagacctggacctgttttggttgatgttcctaaagatatacagcagcagctcgcgattcctaattgggaacagtctatgaggttacctggttatatgtctaggatgcctaaacctccggaggattctcatttggagcagattgttaggttggtttctgaatctaagaagcctgtgttgtatgttggtggtggttgtttgaattcgagtgaggagttgggtaggtttgttgaacttacgggtatccctgttgcgagtacgttgatgggtcttggtgcttatccttgtgatgacgagttgtcgttgcatatgcttggcatgcacgggactgtgtatgcgaattactctgtggagcatagtgatttgttgttggcttttggggtgaggtttgatgatcgtgtcacgggtaagcttgaggcttttgctagtagggcgaagattgttcacattgatattgactcggctgagattgggaagaataagactcctcatgtgtctgtgtgtggagatgttaagctggctttgcaagggatgaataaggttcttgagaaccgagctgaggagcttaagcttgattttggagtttggagaagtgagttgaacgaacagaaacaaatgttccctctgagcttcaagacttttggggaagctattcctcctcagtatgcgattcaggttcttgatgagctgactgatgggagagccattataagcactggtgtcgggcaacatcaaatgtgggcggcgcagttttacaagtacaaaaaaccaaggcagtggctatcatcagcaggccttggagctatgggttttggacttcccgctgctattggtgcgtccgttgcgaatcctgatgcgattgtagtggatattgacggagatggaagcttcataatgaatgtgcaagagctggccacaatccgggtggagaatctcccagtgaagatacttatactaaacaaccagcatcctggcatggttattcaatgggaggctcggttctacaaagctaaccgagctcacacatatcttggtaacccggcagcggaggacgagatattcccgaacatgctgcagtttgcatcagcttgcggtattccagcggcgagggtgacaaagaaagcagagctccgagaagctattcagaaaatgctggataccccaggaccatacttgttggatgtgatatgtccgcaccaagaacatgtgttgccgatgatcccaagtggcggcactttcaatgatgtcataacggaaggagatggccggactaaatac

SEQ ID NO: 6 - DNA, 1997 bp, Camelia sativa
  source 1..1997: mol_type = genomic DNA
atggcggcggcaacaacaccatcatcatcttcgatccccttctcaacaaaaccttcctcctccaaatcaccattaccaatctccaggttcaccctcccattcgcactaaaccccaccaaatcctcctcctcccgccgccgcggtatcaaatcaacctccctctccatctccgccgtgctcaacacaaccaccaatgtctcaaccacaactcctccatcaaaaccaaccaaacccgaaaaaaaaaattcgtctcccgattcgctccagatcagccacgcaaaggcgctgacatcctcgtcgaagctttagaacgtcaaggcgtagaaacagtcttcgcttacccaggaggtgcatcaatggagattcaccaagccttaactcgttcctcctcgatccgtaacgtccttccacgtcacgaacaaggaggcgtattcgcagctgaaggctacgctagatccacaggtaaaccaggtatctgtatagcaacctcaggtcctggagctacaaatctcgttagcggattagccgatgccttgttagatagtgttcctcttgtagcgatcacgggtcaagtccctcgtaggatgattggtactgatgcgtttcaagaaactcccatcgttgaggtgacgcgttcgattactaaacataactatcttgtgatggatgttgaagatattcctaggattgttgaggaagctttctttttagctacttctggtagacctggacctgttttggttgatgttcctaaagatatccagcagcagcttgcgattcctaattgggaacaggctatgaggttacctggttatatgtctaggatgcctaaacctccggaagattctcatttggagcagattgttaggttgatttctgaatctaagaagcctgtgttgtatgttggtggtggttgtttgaattcgagtgaggagttgggtaggtttgttgaacttactggtatccctgttgcgagtacgttgatgggtcttggtgcttatccttgtgatgatgagttgtcgttgcatatgcttggcatgcacgggaccgtgtatgctaattactctgtggagcatagtgatctgttgttggcttttggggtgaggtttgatgatcgtgtcacgggtaagcttgaggcttttgctagtagggcgaagattgttcacattgatattgactcggctgagattgggaagaacaagactcctcatgtgtctgtgtgtggagatgttaagttggctttgcaagggatgaacaaggttcttgagaaccgagctgaggagcttaagcttgatttcggggtttggagaagtgagttgaacgaacagaaacaaaagttccctctgagcttcaagacgtttggggaagctattcctccacagtatgcgattcaggtccttgatgagctgactgatgggagagccattataagcactggtgtcggtcagcatcaaatgtgggcggcgcagttttacaagtacaaaaaaccaaggcagtggctatcatcagcaggccttggagctatgggttttggacttcccgctgctattggagcgtccgttgcgaaccctgatgcgattgttgtggatattgacggagatggaagcttcataatgaatgtgcaagagctggccactatcagggtagagaatctcccagtgaagatacttatactaaacaaccagcatcttggcatggttatgcaatgggaggatcggttctacaaagctaaccgagctcacacatatcttggtaacccgggagcggaggacgagatattcccgaacatgctgcagtttgcatcagcttgcgggattccagcggcgagggtgacaaagaaagcagagctccgagaagctatccagaaaatgctggataccccaggaccatacttgttggatgtgatttgtccgcaccaagagcatgtgttgccgatgatcccaagtggcggcactttcaatgatgtcataacggaaggagatggccggacgaaatac

SEQ ID NO: 7 - protein, 667 aa, Camelia sativa
MAAATTPSSSSIPFSTKPSSSKSPLPISRFTLPFALNPTKSSSSSRRRGIKSTSLSISAVLNTTTNVSTTTPPSKPTKPRKKKFVSRFAPDQPRKGADILVEALERQGVETVFAYPGGASMEIHQALTRSSSIRNVLPRHEQGGVFAAEGYARSTGKPGICIATSGPGATNLVSGLADALLDSVPLVAITGQVSRRMIGTDAFQETPIVEVTRSITKHNYLVMDVEDIPRIVEEAFFLATSGRPGPVLVDVPKDIQQQLAIPNWEQAMRLPGYMSRMPKPPEDSHLEQIVRLISESKKPVLYVGGGCLNSSEELGRFVELTGIPVASTLMGLGAYPCDDELSLHMLGMHGTVYANYSVEHSDLLLAFGVRFDDRVTGKLEAFASRAKIVHIDIDSAEIGKNKTPHVSVCGDVKLALQGMNKVLENRAEELKLDFGVWRSELNEQKQKFPLSFKTFGEAIPPQYAIQVLDELTDGRAIISTGVGQHQMWAAQFYKYKKPRQWLSSAGLGAMGFGLPAAIGASVANPDAIVVDIDGDGSFIMNVQELATIRVENLPVKILILNNQHLGMVMQWEDRFYKANRAHTYLGNPGAEDEIFPNMLQFASACGIPAARVTKKAELREAIQKMLDTPGPYLLDVICPHQEHVLPMIPSGGTFNDVITEGDGRTKY

SEQ ID NO: 8 - protein, 667 aa, Camelia sativa
MAAATTTSSSSIPFSTKPSSSKSPLPISRFTLPFSLNPNKSSSSSRRRGIKSTSLSISAVLNTTANVSTTTPPSKPTKPEKKKFVSRFAPDQPRKGADILVEALERQGVETVFAYPGGASMEIHQALTRSSSIRNVLPRHEQGGVFAAEGYARSTGKPGICIATSGPGATNLVSGLADALLDSVPLVAITGQVSRRMIGTDAFQETPIVEVTRSITKHNYLVMDVEDIPRIVEEAFFLATSGRPGPVLVDVPKDIQQQLAIPNWEQSMRLPGYMSRMPKPPEDSHLEQIVRLISESKKPVLYVGGGCLNSSEELGRFVELTGIPVASTLMGLGAYPCDDELSLHMLGMHGTVYANYSVEHSDLLLAFGVRFDDRVTGKLEAFASRAKIVHIDIDSAEIGKNKTPHVSVCGDVKLALQGMNKVLENRAEERKLDFGVWRSELNEQKQKFPLSFKTFGEAIPPQYAIQVLDELTDGKAIISTGVGQHQMWAAQFYKYKKPRQWLSSAGLGAMGFGLPAAIGASVANPDAIVVDIDGDGSFIMNVQELATIRVENLPVKILILNNQHLGMVMQWEDRFYKANRAHTYLGNPAAEDEIFPNMLQFASACGIPAARVTKKAELREAIQKMLDTPGPYLLDVICPHQEHVLPMIPSGGTFNDVITEGDGRTKY

SEQ ID NO: 9 - DNA, 2004 bp, synthetic construct
  source 1..2004: mol_type = other DNA
atggcggcggcaacaacaccatcatcatcttcgatccccttctcaacaaaaccttcctcctccaaatcaccattaccaatctccaggttcaccctcccattcgcactaaaccccaccaaatcctcctcctcctcccgccgccgcggtatcaaatcaacctccctctccatctccgccgtgctcaacacaaccaccaatgtctcaaccacaactcctccatcaaaaccaaccaaaccccgaaaaaaaaaattcgtctcccgattcgctccagatcagccacgcaaaggcgctgacatcctcgtcgaagctttagaacgtcaaggcgtagaaacagtcttcgcttacccaggaggtgcatcaatggagattcaccaagccttaactcgttcctcctcgatccgtaacgtccttccacgtcacgaacaaggaggcgtattcgcagctgaaggctacgctagatccacaggtaaaccaggtatctgtatagctacctcaggtcctggagctacaaatctcgttagcggattagccgatgccttgttagatagtgttcctcttgtagcgatcactggtcaagtctctcgtaggatgattggtactgatgcgtttcaagaaactcccatcgttgaggtgacgcgttcgatcactaaacataactatcttgtgatggatgttgaagatattcctaggattgttgaggaagctttctttttagctacttctggtagacctggacctgttttggttgatgttcctaaagatatccagcagcagcttgcgattcctaattgggaacaggctatgaggttacctggttatatgtctaggatgcctaaacctccggaagattctcatttggagcagattgttaggttgatttctgaatctaagaagcctgtgttgtatgttggtggtggttgtttgaattcgagtgaggagttgggtaggtttgtggaacttactggtatccctgttgcgagtacgttgatgggtcttggtgcttatccttgtgatgatgagttgtcgttgcatatgcttggcatgcacgggaccgtgtatgctaattactctgtggagcatagtgatctgttgttggcttttggggtgaggtttgatgatcgtgtcacgggtaagcttgaggcttttgctagtagggcgaagattgttcacattgatattgactcggctgagattgggaagaacaagactcctcatgtgtctgtgtgtggagatgttaagttggctttgcaagggatgaacaaggttcttgagaaccgagctgaggagcttaagcttgatttcggggtttggagaagtgagttgaacgaacagaaacaaaagttccctctgagcttcaagacgtttggggaagctattcctccacagtatgcgattcaggtccttgatgagctgactgatgggagagccattataagcactggtgtcggtcagcatcaaatgtgggcggcgcagttttacaagtacaaaaaaccaaggcagtggctatcatcagcaggccttggagctatgggttttggacttcccgctgctattggagcgtccgttgcgaaccctgatgcgattgttgtggatattgacggagatggaagcttcataatgaatgtacaagagctggccactatcagggtagagaatctcccagtgaagatacttatactaaacaaccagcatcttggcatggttatgcaatgggaggatcggttctacaaagctaaccgagctcacacatatcttggtaacccgggagcggaggacgagatattcccgaacatgctgcagtttgcatcagcttgcgggattccagcggcgagggtgacaaagaaagcagagctccgagaagctatccagaaaatgctggataccccaggaccatacttgttggatgtgatttgtccgcaccaagagcatgtgttgccgatgatcccaagtggcggcactttcaatgatgtcataacggaaggagatggccggacgaaatactga

SEQ ID NO: 10 - DNA, 2001 bp, synthetic construct
  source 1..2001: mol_type = other DNA
atggcggcggcaacaacaacatcatcatcttcgatccccttctcaacaaaaccttcctcatccaaatcaccattaccaatctccaggttcaccctcccattctccctaaacccaaacaaatcctcctcctcctcccgccgccgcggtatcaaatcaacctccctctccatctccgccgttctcaacacaacagccaatgtctcaaccactactcctccatcaaaaccaaccaaacccgaaaaaaagaaattcgtctcccgatttgctccagatcagccacgcaaaggcgctgacatcctcgtcgaagctttagaacgccaaggcgtagaaacagtcttcgcttacccaggaggtgcatcaatggagattcaccaagccttaactcgttcctcctcgatccgtaacgtcctcccacgtcacgaacaaggaggcgtgttcgcagctgaaggctacgctagatccacaggtaaaccaggtatctgtatagctacctcaggtcctggagctacgaatctcgttagcggattagccgatgccttgttagatagtgttcctcttgtagcgatcacgggtcaagtctctcgtaggatgattggtactgatgcgtttcaagaaactcccatcgttgaggtgacgcgttccattactaaacataactatcttgtgatggatgttgaagatattcctaggattgttgaggaagctttctttttagctacttctggtagacctggacctgttttggttgatgttcctaaagatatacagcagcagcttgcgattcctaattgggaacagtctatgaggttacctggttatatgtctaggatgcctaaacctccggaagattctcatttggagcagattgttaggttgatttctgagtctaagaagcctgtgttgtatgttggtggtggttgtttgaattcgagtgaggagttgggtaggtttgtggaacttacgggtatacctgttgcgagtacgttgatgggtcttggtgcttacccttgtgatgacgagttgtcgttgcatatgcttggaatgcacgggaccgtgtacgcgaattactctgtggagcatagtgatttgttgttggcttttggggtgaggtttgatgatcgtgtcacgggtaagcttgaggcttttgctagtagggcgaagattgttcacattgatattgactcggctgagattgggaagaacaagactcctcatgtgtctgtgtgtggagatgttaagctggctttgcaagggatgaacaaggttcttgagaaccgagctgaggagcgtaagcttgattttggagtttggagaagtgagttgaacgaacagaaacaaaagttccctctgagcttcaagacgtttggggaagctattcctcctcagtatgcgattcaggtccttgatgagctgactgatgggaaagccattataagcactggtgtcggtcaacatcaaatgtgggcggcgcagttttacaagtacaaaaaaccgaggcagtggctatcatcagcaggccttggagctatgggttttggacttccggctgctattggagcgtccgttgcgaaccctgatgcgattgttgtggatattgacggagatggaagcttcataatgaatgtgcaagagctggccactatcagggtagagaatctcccagtgaagatacttatactaaacaaccagcatcttggcatggttatgcaatgggaggatcggttctacaaagctaaccgagctcacacatatcttggtaacccggcagcggaggacgagatattcccgaacatgctgcagtttgcatcagcttgcggtattccagcggcgagggtgacaaagaaagcagagctccgagaagctatccagaaaatgctggataccccaggaccatacttgttggatgtgatttgtccgcaccaagaacatgtgttgccgatgatcccaagtggcggcactttcaatgatgtcataacggaaggagatggccggactaaatac

SEQ ID NO: 11 - DNA, 2103 bp, Camelia sativa
  source 1..2103: mol_type = genomic DNA
ggagcccatctcactcatcatctcatcacactgttaaatctctctcttcctctctgtgaatctcatcttcaacctctctctctcgcgttttcgattcaacaatggctgacaagaagattaagatcggaatcaacggattcggaagaatcggtcgtttggtcgctagggttgttcttcagagggacgatgttgagctcgttgccgtcaacgaccccttcatcactactgagtacatgacctacatgttcaagtacgacagtgttcacggccaatggaagcacaatgaactcaagatcaaggatgacaagacccttctcttcggtgagaagccagtcactgttttcggcattaggaaccctgaggatatcccatgggccgaggctggagcagactacgttgttgagtctaccggtgtcttcactgacaaggacaaggctgctgctcacttgaagggtggtgccaagaaggttgtcatctctgcccccagcaaagacgctcccatgtttgttgttggtgtcaatgagcacgaatacaagtctgaccttgacattgtctccaacgctagctgcaccactaactgccttgctccccttgccaaggctacccagaagaccgttgatggtccatcaatgaaggactggagaggtggaagagctgcctcattcaacattattcccagcagcactggagctgccaaggctgtcggaaaggtgcttccagcccttaacggaaagttgactggaatgtctttccgtgttccaactgtggatgtctcagttgttgacctcaccgtcagactcgagaaggctgctacctacgatgaaatcaaaaaggctatcaaggaggaatctgaaggcaagctaaagggaatccttggatacaccgaagatgatgttgcaactgacttcgttggtgacaacaggtcgagcatctttgacgccaaggccggaattgcattgagcgacaagtttgtgaaattggtgtcatggtacgacaacgaatggggttacagttcccgtgtggtcgatttgatcgtccacatgtccaaggcctaaatctaagaagcagatgtcgaatgatggggagtggaaagtcatatgttcatccctagattggtctgaatttgtcggtttttctaataaaatttcttcgaacttaaaccttggtttttttttttgtgttatggtttttgctattctcattcatgtgaggtgatgggaatttgtagaccggtgttttaatgaagccttttttcgtttgcctaaaaaaaaaaaaaaaaaaactcgagtttttttttttttttttttttttgatgcaaagataagataaaactgatatactcacttcgggaagaagaggcgaaaaagtaacgagttattaaagggaaaatacaaaagtgtgaaatgatctgagtttgagataagagacgaagaagaagaatataataataaatcttttagtactagcgaaacagaccccttctttgatttataaaccaaacaagcaaaaaagaggaatctggctaatctcccttctatgccttgtttgcgatgttgttggagagccagtcaagtccctcatagagaccttctcctgaggtggcacatgtgctctgaatgtaccagtgtcgttggcggagggagtgaagcccaagcttgtcggttatctcagcagcgttcatggcgttgggaagatcttgcttgttagcaaaaacaagaagaacggcatccctcaattcatcctcattcagcatcctgtgaagctcatccctagcttcaacaacacggtcacgatcattactgtccacaacaaagatgagtccctgtgtgttctggaagtaatgtctccacaatggacggatcttgtcttgacccccgacatcccacacggtaaagctgatgttcttgtattcaacagtctcaacgttgaacccaatggtaggaatagtggtgacgatctctccaagtttgagcttgtacaagatggtcgtcttaccagcagcatcgagaccaaccatcagaatacgcatctctttcttcgcaaagagcctgctgaacaactttccaaaggacaaacccattttttccaagcaggaaaaacagc

SEQ ID NO: 12 - DNA, 20 bp, synthetic construct
  source 1..20: mol_type = other DNA
ctggcttcgtctttctcctg

SEQ ID NO: 13 - DNA, 21 bp, synthetic construct
  source 1..21: mol_type = other DNA
gttggagagacatgaacggag

SEQ ID NO: 14 - DNA, 18 bp, synthetic construct
  source 1..18: mol_type = other DNA
tgtaaaacgacggccagt

SEQ ID NO: 15 - DNA, 17 bp, synthetic construct
  source 1..17: mol_type = other DNA
caggaaacagctatgac

SEQ ID NO: 16 - DNA, 20 bp, synthetic construct
  source 1..20: mol_type = other DNA
atgttggtggtggttgtttg

SEQ ID NO: 17 - DNA, 20 bp, synthetic construct
  source 1..20: mol_type = other DNA
aagcaccaagacccatcaac

SEQ ID NO: 18 - DNA, 20 bp, synthetic construct
  source 1..20: mol_type = other DNA
gccatctccttccgttatga

SEQ ID NO: 19 - DNA, 20 bp, synthetic construct
  source 1..20: mol_type = other DNA
atctcgttagcggattagcc

SEQ ID NO: 20 - DNA, 21 bp, synthetic construct
  source 1..21: mol_type = other DNA
cctcaacgatgggagtttctt

SEQ ID NO: 21 - DNA, 24 bp, synthetic construct
  source 1..24: mol_type = other DNA
tgttcctcttgtagcgatcacggg

SEQ ID NO: 22 - DNA, 20 bp, synthetic construct
  source 1..20: mol_type = other DNA
agagccagtcaagtccctca

SEQ ID NO: 23 - DNA, 20 bp, synthetic construct
  source 1..20: mol_type = other DNA
gacaagcttgggcttcactc

SEQ ID NO: 24 - DNA, 24 bp, synthetic construct
  source 1..24: mol_type = other DNA
tccgccaacgacactggtacattc

(19) United States
(12) Patent Application Publication
(10) Pub. No.: US 2025/0146011 A1
(43) Pub. Date: May 8, 2025

(54) HERBICIDE-RESISTANT CAMELINA SATIVA PLANTS, AND VARIANT CAMELINA ACETOHYDROXYACID SYNTHASE POLYPEPTIDES

(71) Applicant: Smart Earth Camelina Corp., Saskatoon (CA)

(72) Inventors: Debbie Puttick, Saskatoon (CA); Christina Eynck, Saskatoon (CA); Jack Grushcow, Saskatoon (CA); David Csumrik, Saskatoon (CA)

(73) Assignee: Smart Earth Camelina Corp., Saskatoon, SK (CA)

(21) Appl. No.: 18/974,535

(22) Filed: Dec. 9, 2024

Related U.S. Application Data
(63) Continuation of application No. 17/420,649, filed on Jul. 2, 2021, now abandoned.
(60) Provisional application No. 62/787,638, filed on Jan. 2, 2019.

(51) Int. Cl. C12N 15/82; C12N 9/10

(52) U.S. Cl. CPC C12N 15/8274; C12N 9/1022; C12N 15/8278; C12Y 202/01006

(57) ABSTRACT

Provided are variants of the Camelina sativa acetohydroxyacid synthase (AHAS) enzyme that provide camelina plants with increased tolerance to Group 2 herbicides, such as for example thifensulfuron-methyl. Also provided are polynucleotides encoding the variant AHAS enzymes, and plants, plant parts, seeds and cells containing the variant polynucleotides and polypeptides. Uses of the plants and seeds are also disclosed, such as for producing progeny, for growing plants in a field, or for introgression of the herbicide resistance trait into another camelina variety.

### CROSS-REFERENCE TO RELATED APPLICATIONS

This application is the National Stage Application of PCT/CA2019/050192 filed Feb. 15, 2019, which claims priority to U.S. Provisional Patent Application No. 62/787,638 filed on Jan. 2, 2019, which is hereby incorporated by reference in its entirety.

### SEQUENCE LISTING

This application contains a Sequence Listing in computer readable form (File name: “18470-034002 Sequence Listing”; Date of Creation: Dec. 6, 2024; File size: 37 kilobytes), which is incorporated herein by reference in its entirety and forms part of the disclosure.

### FIELD

The disclosure relates to herbicide-resistant Camelina sativa, and more particularly to polynucleotide and polypeptide variants giving rise to herbicide resistance in Camelina sativa and the plant cells, plants, seeds and uses derived therefrom.

### BACKGROUND

Camelina (Camelina sativa [L.] Crantz), also known as “Gold of Pleasure” is an ancient oilseed crop originating from the steppe regions of Southwestern Asia and Southeastern Europe. Camelina sativa belongs to the family Brassicaceae (mustard family), and both spring and winter forms are in production. It is a low-input crop adapted to low fertility soils. Results from long-term experiments in Central Europe have shown that the seed yields of Camelina sativa are comparable to the yields of rapeseed oil.

Camelina oil was traditionally used as edible oil, with references dating back to 1800s in Denmark, Germany and Slovenia (Zubr, 2009; Lobe, 1845; Wacker, 1934; Rode, 2002). After the Great War, Europe became more interested in rapeseed oil because it was more suitable for making hydrogenated margarine. The specific nutritional qualities of camelina oil were therefore underestimated. Except for small areas in Southeastern Europe where it is still produced for human consumption and as dietary supplement, much of the cultivation of camelina in Europe ceased in the 1950s.

At the same time in Canada, field trials started to evaluate the potential of camelina in the short-season environment of the Canadian Prairies. More recently, camelina is grown commercially for its high-value oil, which is high in α-linolenic acid (20 to >35%), eicosenoic acid (11-19%) and tocopherols (Vitamin E), as well as naturally low in erucic acid (<4%), rendering camelina oil well-suited for a variety of food, feed and non-food applications. Cold-pressed camelina oil is mainly used as a sustainable replacement for fish oil in the aquaculture industry and is also used for cosmetics, as an industrial feedstock, and also for human consumption. Cold-pressed camelina oil has been approved by Health Canada since 2010 and more recently was approved as a feed ingredient for juvenile salmonids at up to 13% of the total feed ration. In the United States and Canada, there are several companies marketing camelina oil as a Low Risk Veterinary Health Product for horses, dogs, and cats. The co-product of the crushing process, camelina pressed cake or meal, has been approved for poultry at 12% of the total feed ration for broilers and at 10% of the ration for laying hens. Approvals for camelina meal as a feed ingredient for other livestock, such as dairy, are expected in the coming years.

Although camelina is best adapted to cool, semi-arid climatic zones, it is able to grow in most soil types except heavy clay and peat soil. It performs well on light soils because it tolerates drought conditions and it also shows cold tolerance both during germination and early season growth.

Due to the high oil content of camelina seeds and relatively low input requirements, there has been a renewed interest in camelina oil. Moreover, there is an increasing interest in camelina as animal feed and as a commercial crop to provide vegetable oils for biofuel production, without displacing food crops from rich soils. Camelina is particularly well suited given its ability to grow in most soil types.

As camelina has been a minor crop species, very little has been done in terms of its breeding and genetic improvement, aside from testing different accessions for agronomic traits and oil profiles. Indeed, the number of varieties available for commercial production is quite limited. In addition, there are few herbicides registered for use with camelina, and this has limited the adoption of camelina as an oilseed crop in North America. In particular, Camelina sativa is highly sensitive to soil residual levels of many acetolactate synthesis (ALS) inhibitor herbicides. In areas where certain types of these herbicides are used, camelina cannot be grown at a commercially acceptable level until the herbicide residues are degraded in the soil. Factors that affect herbicide degradation include climate factors such as moisture and temperature, as well as soil pH. Thus, in areas of North America the period of time in which the soil contains herbicide residues may last several years.

Consequently, there is a real need to develop camelina varieties with beneficial properties, such as herbicide resistance, for commercial production.

### SUMMARY

The present disclosure relates to methods for producing novel camelina plants, cultivars, and varieties with increased tolerance or resistance to Group 2 herbicides. In particular, the present disclosure relates to variant Camelina sativa polypeptides and polynucleotides giving rise to herbicide resistance in camelina, and plants, seeds, tissues, and cells containing these variant polypeptides and/or polynucleotides. The camelina plants, plant parts and cells disclosed herein contain variant camelina acetohydroxyacid synthase (AHAS) genes and proteins that provide resistance to Group 2 herbicides that normally inhibit the AHAS enzyme.

In an embodiment, the present disclosure relates to a Camelina sativa acetohydroxyacid synthase (CsAHAS) polypeptide variant comprising a substitution of amino acid P194, wherein amino acid position is determined by alignment with a wildtype CsAHAS polypeptide of SEQ ID NO: 1 or 2. In an embodiment, the substitution is P194S.

In an embodiment, the CsAHAS polypeptide variant comprises or consists of the amino acid sequence of SEQ ID NO: 7.

In an embodiment, the CsAHAS polypeptide variant comprises or consists of the amino acid sequence of SEQ ID NO: 8.

In an embodiment, the present disclosure relates to a polynucleotide encoding the CsAHAS polypeptide variant as described herein. In an embodiment, the polynucleotide comprises a nucleotide substitution of cytosine to thymine at position 580, wherein the nucleotide position is determined by alignment with a wildtype CsAHAS nucleotide sequence of SEQ ID NO: 4 or 5.

In an embodiment, the CsAHAS polynucleotide of the present disclosure comprises or consists of the nucleotide sequence of SEQ ID NO: 9.

In an embodiment, the CsAHAS polynucleotide of the present disclosure comprises or consists of the nucleotide sequence of SEQ ID NO: 10.

In an embodiment, the present disclosure relates to a plant cell that expresses the CsAHAS polypeptide variant as described herein.

In an embodiment, the present disclosure relates to a plant cell comprising the CsAHAS polynucleotide as described herein.

In an embodiment, the present disclosure relates to a plant cell comprising one or more polynucleotides comprising the nucleotide sequence of SEQ ID NO: 9, the nucleotide sequence of SEQ ID NO: 10, or the nucleotide sequence of SEQ ID NOs: 9 and 10.

In an embodiment, the present disclosure relates to a plant cell from camelina sativa variety designated 12CS0365, 12CS0366, 12CS0389, 13CS0695, 13CS0781, 13CS0786, 14CS0851-01-14 or 17CS1115. Representative seed of varieties 12CS0365, 12CS0366, 12CS0389 and 14CS0851-01-14 has been deposited under ATCC Accession Numbers PTA-125493, PTA-125492, PTA-125494 and PTA-125495, respectively, on Dec. 3, 2018. Seed of varieties 13CS0695, 13CS0781, 13CS0786 and 17CS1115 is maintained by Linnaeus Plant Sciences, Inc., 2212-110 Gymnasium Place, Saskatoon, SK, S7N 0W9, Canada.

In an embodiment, the present disclosure relates to a plant cell from camelina sativa variety designated 14CS0851-01-14, wherein representative seed of said variety has been deposited under ATCC Accession Number PTA-125495.

In an embodiment, the present disclosure relates to a plant, or part thereof, comprising the plant cell as described herein.

In an embodiment, the present disclosure relates to a seed that expresses the CsAHAS polypeptide variant as described herein.

In an embodiment, the present disclosure relates to a seed comprising the CsAHAS polynucleotide as described herein.

In an embodiment, the present disclosure relates to a seed comprising one or more polynucleotides comprising the nucleotide sequence of SEQ ID NO: 9, the nucleotide sequence of SEQ ID NO: 10, or the nucleotide sequence of SEQ ID NOs: 9 and 10.

In an embodiment, the present disclosure relates to a seed of camelina sativa variety designated 12CS0365, 12CS0366, 12CS0389, 13CS0695, 13CS0781, 13CS0786, 14CS0851-01-14 or 17CS1115. Representative seed of varieties 12CS0365, 12CS0366, 12CS0389 and 14CS0851-01-14 has been deposited under ATCC Accession Numbers PTA-125493, PTA-125492, PTA-125494 and PTA-125495, respectively. Seed of varieties 13CS0695, 13CS0781, 13CS0786 and 17CS1115 is maintained by Linnaeus Plant Sciences, Inc., 2212-110 Gymnasium Place, Saskatoon, SK, S7N 0W9, Canada.

In an embodiment, the present disclosure relates to a seed of camelina sativa variety designated 14CS0851-01-14, wherein representative seed of said variety has been deposited under ATCC Accession Number PTA-125495.

In an embodiment, the present disclosure relates to a camelina sativa plant, or part thereof, produced by growing the seed as described herein.

In an embodiment, the present disclosure relates to the use of the plant or seed as described herein for producing progeny, for growing plants in a field, or for introgression of the herbicide resistance trait into another camelina variety.

In an embodiment, the present disclosure relates to the use of the plant or seed as described herein for producing a plant oil or seed oil.

In an embodiment, the present disclosure relates to the use of the plant as described herein for producing seed.

In an embodiment, the present disclosure relates to the use of the seed as described herein for producing a plant.

### BRIEF DESCRIPTION OF THE DRAWINGS

Further advantages, permutations and combinations of the present disclosure will now appear from the above and from the following detailed description of the various particular embodiments of the present disclosure taken together with the accompanying drawings, each of which are intended to be non-limiting, in which:

FIG. 1 shows tolerance to Refine® SG of five varieties disclosed herein, namely 12CS0363, 12CS0364, 12CS0365, 12CS0366, and 11CS0111 along side SRS-934 control.

FIG. 2 shows the effect of thifensulfuron-methyl rate on the height of four camelina lines 21 days after application. Height depicted on the y-axis is expressed as a % of the untreated check. Rate of thifensulfuron-methyl in g ai/ha is shown on the x-axis.

FIG. 3 shows the effect of thifensulfuron-methyl rate on dry biomass of four camelina lines 21 days after application. Biomass depicted on the y-axis is expressed as a % of the untreated check. Rate of thifensulfuron-methyl in g ai/ha is shown on the x-axis.

FIG. 4 depicts a partial DNA sequence clustal alignment of mutant lines 12CS0366 and 12CS0365 compared to C. sativa wild type orthologues.

FIG. 5 depicts a partial alignment of the translated amino acid sequences of wild-type CsAHAS gene orthologues 1, 2 and 3 from C. sativa and mutant lines 12CS0365 and 12CS0366.

FIG. 6 shows a clustal alignment of the full DNA sequence of the mutated AHAS genes of mutant lines 12CS0366 and 12CS0365 compared to C. sativa wildtype AHAS orthologues.

FIG. 7 shows a clustal alignment of the translated amino acid sequences of wild-type CsAHAS gene orthologues 1, 2 and 3 from C. sativa and camelina mutant lines 12CS0365 and 12CS0366.

FIG. 8 shows the complete DNA sequence of housekeeping gene glyceraldehyde-3-phophate dehydrogenase (GAPC-1), including primer (square boxes) and probe (underlined) sequences used in the RT-qPCR assays as described in Example 9.

FIG. 9 shows the in vitro inhibition of AHAS activity in 14CS0851-01-14 (squares), SRS 934 (stars) and MIDAS™ (circles) by addition of leucine at 1 mM, 10 mM, and 100 mM final concentration in assay. 100% activity conditions (control) contain 100 mM pyruvate with no added leucine. Absorbance readings were converted to AHAS activity as % of control. Lines represent the fitted line of data from three independent experiments with three replications in each.

FIG. 10 shows the in vitro inhibition of AHAS activity in 14CS0851-01-14 (squares), SRS 934 (circles) and MIDAS™ (stars) by addition of isoleucine at 1 mM, 10 mM, and 100 mM final concentration in assay. 100% activity conditions (control) contain 100 mM pyruvate with no added isoleucine. Absorbance readings were converted to AHAS activity as % of control. Lines represent the fitted line of data from three independent experiments with three replications in each.

FIG. 11 shows the in vitro inhibition of AHAS activity in 14CS0851-01-14 (squares), SRS 934 (circles) and MIDAS™ (stars) by addition of valine at 1 mM, 10 mM, and 100 mM final concentration in assay. 100% activity conditions (control) contain 100 mM pyruvate with no added valine. Absorbance readings were converted to AHAS activity as % of control. Lines represent the fitted line of data from three independent experiments with three replications in each.

### DETAILED DESCRIPTION

Unless otherwise defined, all technical and scientific terms used herein generally have the same meaning as commonly understood by one of ordinary skill in the art to which this disclosure pertains. Exemplary terms are defined below for ease in understanding the subject matter of the present disclosure.

### Definitions

The term “a” or “an” refers to one or more of that entity; for example, “a gene” refers to one or more genes or at least one gene. As such, the terms “a” (or “an”), “one or more” and “at least one” are used interchangeably herein. In addition, reference to an element or feature by the indefinite article “a” or “an” does not exclude the possibility that more than one of the elements or features are present, unless the context clearly requires that there is one and only one of the elements.

“About”, when referring to a measurable value such an amount of a compound or agent, does, time, temperature, and the like, is meant to encompass variations of ±20%, ±10%, ±5%, ±1%, ±0.5% or ±0.1% of the specified amount. When the value is a whole number, the term about is meant to encompass decimal values, as well the degree of variation just described.

“And/or” refers to and encompasses any and all possible combinations of one or more of the associated listed items (e.g. one or the other, or both), as well as the lack of combinations when interrupted in the alternative (or).

“Backcross” or “backcrossing” refer to a process in which progeny plants are crossed back to one of the parents one or more times, for example, a first generation hybrid F1 with one of the parental genotype of the F1 hybrid. In a backcrossing scheme, the “donor” parent refers to the parental plant with the desired gene or locus to be introduced. The “recipient” parent (used one or more times) or “recurrent” parent (used two or more times) refers to the parental plant into which the gene or locus is being introduced.

“Comprise” as is used in this description and in the claims, and its conjugations, are used in its non-limiting sense to mean that items following the word are included, but items not specifically mentioned are not excluded.

“Corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. It does not mean that the given amino acid or polynucleotide sequence is necessarily 100% identical in sequence to the reference sequence outside the aligned position being referenced.

“Cross”, “crossing”, “cross-pollination” or “cross-breeding” refer to the process by which pollen from one flower on one plant is applied or transferred (artificially or naturally) to the ovule (stigma) of a flower on another plant.

“Days to first flowering” refers to the number of days after planting when 10% of plants have one or more open flower. In an embodiment, it is assessed three times weekly, but it may be assessed more or less frequently.

“Days to 50% flowering” refers to the number of days after planting when 50% of flowers have opened. In an embodiment, it is assessed three times weekly, but it may be assessed more or less frequently.

“Days to end of flowering” or “days to final flowering” refers to the number of days after planting when no flowers remain open. In an embodiment, it is assessed three times weekly, but it may be assessed more or less frequently.

“Days to maturity” refers to the number of days after planting when 50% of the plants in a plot have changed color. In an embodiment, it is assessed three times weekly, but it may be assessed more or less frequently.

“Gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. The term “gene” may refer to the segment of DNA when it is within a cell, e.g. a plant cell, or in an isolated or purified form. Genes can be obtained from a variety of sources, including cloning from a source of interest or synthesizing from known or predicted sequence information, and may include sequences designed to have desired parameters.

“Genotype” refers to genetic makeup.

“Improved tolerance” or “increased tolerance”, used interchangeably, refer to the ability of plants to avoid (even slightly) the negative impact of herbicides. This may be observed in the phenotype of a plant by a reduction in the appearance of symptoms of herbicide damage, such as stunting or malformation, as compared to wildtype plants. Increased tolerance does not necessarily mean that the plant is 100% immune to the herbicide (asymptomatic).

“Introgression”, as used herein, refers to the transfer of genetic information from one plant species to another as a result of hybridization or crossing and repeated backcrossing.

“Isolated” refers to a nucleic acid, polynucleotide, polypeptide, protein, or other component that is partially or completely separated from components with which it is normally associated (such as other proteins, nucleic acids, cells, plant materials or plant parts, etc.).

“Maturity” refers to the stage when the plants have begun to change colour and/or the seeds of the plant are harvestable.

“Oil content” refers to the fraction of total oil contained in the mature seed, or a particular quantity of the mature seed. It is typically measured as a percentage of dry mass (DM).

“Percent identity”, “% identity”, “percent identical” and “% identical” are used interchangeably herein to refer to the percent amino acid sequence identity that is obtained by ClustalW analysis (version W 1.8 available from European Bioinformatics Institute, Cambridge, UK), counting the number of identical matches in the alignment and dividing such number of identical matches by the length of the reference sequence, and using the following default ClustalW parameters to achieve slow/accurate pairwise optimal alignments—Gap Open Penalty: 10; Gap Extension Penalty: 0.10; Protein weight matrix: Gonnet series; DNA weight matrix: IUB; Toggle Slow/Fast pairwise alignments=SLOW or FULL Alignment.

“Phenotype” refers to the detectable characteristics of a camelina plant. These characteristics often are manifestations of the genotype/environment interaction.

“Plant” refers to any living organism belonging to the kingdom Plantae (i.e., any genus/species in the Plant Kingdom). For example, the plant is a species in the tribe of Camelineae, such as C. alyssum, C. anomala, C. grandiflora, C. hispida, C. laxa, C. microcarpa, C. microphylla, C. persistens, C. rumelica, C. sativa, C. Stiefelhagenii, or any hybrid thereof. The term “plant” is intended to encompass plants at any stage of maturity or development, including a plant from which seed has been removed.

“Plant cell” includes plant cells whether isolated, in tissue culture or incorporated in a plant or plant part.

“Plant height” refers to the height of the plant at the time of measurement from the ground base where it is being grown to the top of the plant. The plant height is often measured in centimeters (cm). The top of the plant is typically the tip of the main shoot. In an embodiment, the plant height is measured at plant maturity, but it may be measured at any time.

“Plant part” refers to any part of a plant including but not limited to the anthers, shoots, roots, stems, seeds, racemes, stipules, leaves, petals, flowers, ovules, bracts, branches, petioles, internodes, tiller, pollen, stamen, embryos, tissues, cells and the like. The two main parts of plants grown in some sort of media, such as soil, are often referred to as the “above-ground” part, also often referred to as the “shoots”, and the “below-ground” part, often referred to as the “roots”.

“Pod number” refers to the total number of pods in the plant bearing seeds.

“Progeny” refers to the offspring derived from either an artificial cross between two plants or a natural cross between two plants.

“Resistance” or “resistant”, used interchangeably herein, refer to the ability of plants to avoid the negative impact of herbicides such that the growth characteristics of the plant appear substantially unaffected by the application of herbicide.

“Seed increase” refers to the process of sowing, growing and harvesting seed from a specific plant material for the purpose of creating a larger volume of seed.

“Seeds per pod” refers to the number of fully developed seeds contained inside a pod.

“Seeds per plant” refers to the total number of fully developed seeds that the plant has produced.

“Selfing” or “self-fertilization” refers to the manifestation of the process of self-pollination, which in turn refers to the transfer of pollen from the anther of a flower to the stigma of the same flower or different flowers on the same plant. It is the union of male and female gametes and/or nuclei from the same organism. Selfing often results in the loss of genetic variation within an individual (offspring) because many of the genetic loci that were heterozygous become homozygous.

“Single plant selection” refers to a form of selection in which plants with specific desirable attributes are identified and individually selected.

“Variant” refers to an acetohydroxyacid synthase (AHAS) polypeptide or polynucleotide encoding the AHAS polypeptide comprising one or more modifications such as substitutions, deletions and/or insertions of one or more specific amino acid residues or of one or more specific nucleotides or codons in the polypeptide or polynucleotide. The term “variant” as used herein is one that does not appear in a wildtype, naturally occurring polynucleotide or polypeptide.

“Variety” or “Cultivar” refer to a group of similar plants that by structural or genetic features and/or performance can be distinguished from other varieties within the same species.

“Wildtype” refers to a naturally occurring organism or lifeform, such as a plant, as found in nature. When used in reference to polynucleotides or polypeptides, “wildtype” refers to the native (unmodified) form of the polynucleotide or polypeptide as found within, or expressed by, the wildtype organism.

The present disclosure relates to methods for producing novel camelina plants, cultivars and varieties with increased tolerance or resistance to Group 2 herbicides. In particular, the present disclosure relates to variant Camelina sativa polypeptides and polynucleotides giving rise to herbicide resistance in camelina, and plants, seeds, tissues and cells containing these variant polypeptides and/or polynucleotides. The camelina plants, plant parts and cells disclosed herein contain variant acetohydroxyacid synthase (AHAS) genes and proteins that provide resistance to Group 2 herbicides that normally inhibit the AHAS enzyme.

Camelina sativa

Camelina sativa, usually known in English as camelina, gold-of-pleasure, or false flax, also occasionally wild flax, linseed dodder, German sesame, and Siberian oilseed, is a flowering plant in the family Brassicaceae which includes mustard, cabbage, rapeseed, broccoli, cauliflower, kale, and brussel sprouts. It is native to Northern Europe and to Central Asian areas, but has been introduced to North America.

The taxonomy of Camelina sativa is:
Kingdom: Plantae (plants)
Subkingdom: Tracheobionta (vascular plants)
Superdivision: Spermatophyta (seed plants)
Division: Magnoliophyta (flowering plants)
Class: Magnoliopsida (dicotyledons)
Subclass: Dilleniidae
Order: Capparales
Family: Brassicaceae (mustard family)
Tribe: Camelineae
Genus: Camelina Crantz (false flax)
Species: Camelina sativa (L.) Crantz (gold-of-pleasure)

Camelina is grown commercially for its high-value oil that contains exceptionally high levels (up to 45%) of omega-3 fatty acids, which is uncommon in vegetable sources. Camelina has a fatty acid composition with high levels of both polyunsaturated fatty acids such as 18:2 and 18:3, as well as long chain fatty acids such as 20:1 and 22:1. Over 50% of the fatty acids in cold-pressed camelina oil are polyunsaturated. In particular, camelina oil is high in α-linolenic acid and eicosenoic acid. The oil is also very rich in natural antioxidants, such as tocopherols, making this highly stable oil very resistant to oxidation and rancidity. It is also naturally low in erucic acid. These features, among others, render camelina oil well-suited for a variety of food, feed and non-food applications. For example, it is well suited for use as a cooking oil with its almond-like flavor and aroma. It may become more commonly known and become an important food oil for the future.

Cold-pressed camelina oil is mainly used as a sustainable replacement for fish oil in the aquaculture industry and is also used for cosmetics, as an industrial feedstock, and also for human consumption. The co-product of the crushing process, camelina pressed cake or meal, has been approved for poultry at 12% of the total feed ration for broilers and at 10% of the ration for laying hens. In the US and Canada, there are companies marketing camelina oil as a Low Risk Veterinary Health Product for horses, dogs, and cats. Approvals for camelina meal as a feed ingredient for other livestock, such as dairy, are expected in the coming years.

Camelina is also being grown for its potential as a biofuel, biolubricant, and biodiesel, including as a jet fuel.

Camelina is a short-season crop (85-100 days). It is best adapted to cool, semi-arid climatic zones, however it is able to grow in most soil types except heavy clay and peat soil. As a summer or winter annual plant, camelina grows to heights of about 30-120 cm, with branching stems which become woody at maturity. The leaves are alternate on the stem, with a length from 2-8 cm and a width of 2-10 mm. Leaves and stems may be partially hairy. It blooms typically between June and July, but this depends on geography and climate. Its four-petaled flowers are pale yellow in colour, and cross-shaped. The seeds are brown, or orange in colour and a length typically of 2-3 mm.

In Canada, over 50,000 acres have been cultivated with camelina and predictions suggest that this could reach 1 to 3 million acres in the future, depending on the availability of successful varieties with appropriate agronomic attributes.

### Acetohydroxyacid Synthase

Acetohydroxyacid synthase (AHAS), also known as acetolactate synthase (ALS), is an enzyme found in microorganisms and plants, but not in animals. It functions as a homodimer and catalyzes the condensation of two molecules of pyruvate to yield acetolactate, and the condensation of pyruvate and 2-ketobutyrate to yield 2-aceto2-hydroxybutyrate:

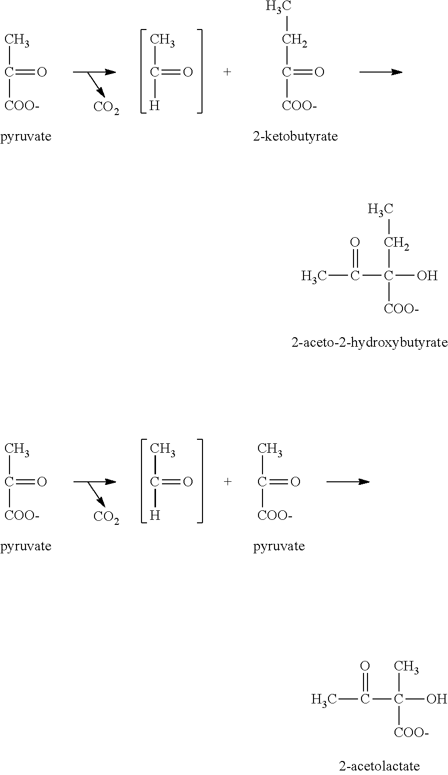

With this, AHAS catalyzes the first reaction of a common pathway that leads to the synthesis of the branched-chain amino acids valine, leucine, and isoleucine. AHAS is therefore a critical enzyme that is necessary for the synthesis of these amino acids in plants.

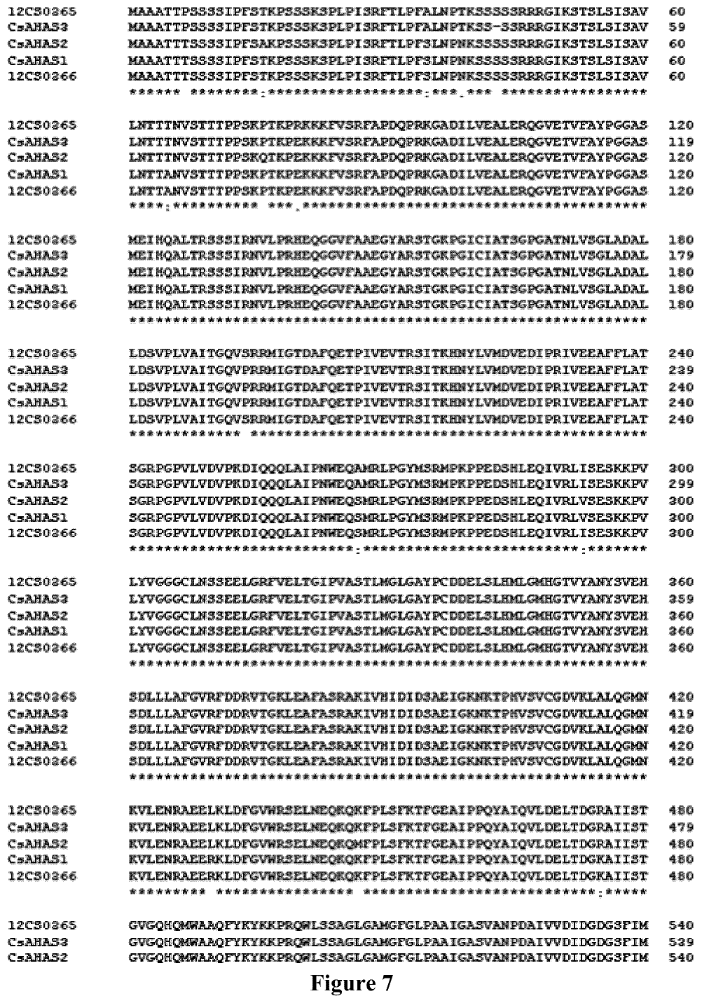

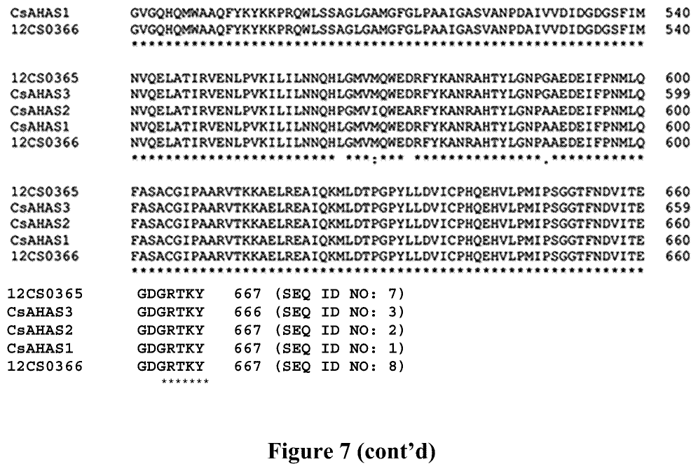

Camelina sativa is a hexaploid species and possesses in total three AHAS orthologues (CsAHAS1, CsAHAS2, and CsAHAS3), having the following wildtype amino acid sequences, which are also shown in FIG. 7:

CsAHAS1 (667 amino acids):
(SEQ ID NO: 1)
MAAATTTSSSSIPFSTKPSSSKSPLPISRFTLPESLNPNKSSSSSRRRGIKSTSLSISAV
LNTTANVSTTTPPSKPTKPEKKKFVSRFAPDQPRKGADILVEALERQGVETVFAYPGGAS
MEIHQALTRSSSIRNVLPRHEQGGVFAAEGYARSTGKPGICIATSGPGATNLVSGLADAL
LDSVPLVAITGQVPRRMIGTDAFQETPIVEVTRSITKHNYLVMDVEDIPRIVEEAFFLAT
SGRPGPVLVDVPKDIQQQLAIPNWEQSMRLPGYMSRMPKPPEDSHLEQIVRLVSESKKPV
LYVGGGCLNSSEELGRFVELTGIPVASTLMGLGAYPCDDELSLHMLGMHGTVYANYSVEH
SDLLLAFGVREDDRVTGKLEAFASRAKIVHIDIDSAEIGKNKTPHVSVCGDVKLALQGMN
KVLENRAEERKLDFGVWRSELNEQKQKFPLSFKTFGEAIPPQYAIQVLDELTDGKAIIST
GVGQHQMWAAQFYKYKKPRQWLSSAGLGAMGFGLPAAIGASVANPDAIVVDIDGDGSFIM
NVQELATIRVENLPVKILILNNQHLGMVMQWEDRFYKANRAHTYLGNPAAEDEIFPNMLQ
FASACGIPAARVTKKAELREAIQKMLDTPGPYLLDVICPHQEHVLPMIPSGGTENDVITE
GDGRTKY
CsAHAS2 (667 amino acids):
(SEQ ID NO: 2)
MAAATTTSSSSIPFSAKPSSSKSPLPISRFTLPFSLNPNKSSSSSRRRGIKSTSLSISAV
LNTTTNVSTTTPPSKQTKPEKKKFVSRFAPDQPRKGADILVEALERQGVETVFAYPGGAS
MEIHQALTRSSSIRNVLPRHEQGGVFAAEGYARSTGKPGICIATSGPGATNLVSGLADAL
LDSVPLVAITGQVPRRMIGTDAFQETPIVEVTRSITKHNYLVMDVEDIPRIVEEAFFLAT
SGRPGPVLVDVPKDIQQQLAIPNWEQSMRLPGYMSRMPKPPEDSHLEQIVRLVSESKKPV
LYVGGGCLNSSEELGREVELTGIPVASTLMGLGAYPCDDELSLHMLGMHGTVYANYSVEH
SDLLLAFGVREDDRVTGKLEAFASRAKIVHIDIDSAEIGKNKTPHVSVCGDVKLALQGMN
KVLENRAEELKLDFGVWRSELNEQKQMFPLSFKTFGEAIPPQYAIQVLDELTDGRAIIST
GVGQHQMWAAQFYKYKKPRQWLSSAGLGAMGFGLPAAIGASVANPDAIVVDIDGDGSFIM
NVQELATIRVENLPVKILILNNQHPGMVIQWEARFYKANRAHTYLGNPAAEDEIFPNMLQ
FASACGIPAARVTKKAELREAIQKMLDTPGPYLLDVICPHQEHVLPMIPSGGTENDVITE
GDGRTKY
CsAHAS3 (666 amino acids):
(SEQ ID NO: 3)
MAAATTPSSSSIPESTKPSSSKSPLPISRFTLPFALNPTKSSSSRRRGIKSTSLSISAVL
NTTTNVSTTTPPSKPTKPEKKKFVSRFAPDQPRKGADILVEALERQGVETVFAYPGGASM
EIHQALTRSSSIRNVLPRHEQGGVFAAEGYARSTGKPGICIATSGPGATNLVSGLADALL
DSVPLVAITGQVPRRMIGTDAFQETPIVEVTRSITKHNYLVMDVEDIPRIVEEAFFLATS
GRPGPVLVDVPKDIQQQLAIPNWEQAMRLPGYMSRMPKPPEDSHLEQIVRLISESKKPVL
YVGGGCLNSSEELGREVELTGIPVASTLMGLGAYPCDDELSLHMLGMHGTVYANYSVEHS
DLLLAFGVREDDRVTGKLEAFASRAKIVHIDIDSAEIGKNKTPHVSVCGDVKLALQGMNK
VLENRAEELKLDFGVWRSELNEQKQKFPLSFKTFGEAIPPQYAIQVLDELTDGRAIISTG
VGQHQMWAAQFYKYKKPRQWLSSAGLGAMGFGLPAAIGASVANPDAIVVDIDGDGSFIMN
VQELATIRVENLPVKILILNNQHLGMVMQWEDRFYKANRAHTYLGNPGAEDEIFPNMLQF
ASACGIPAARVTKKAELREAIQKMLDTPGPYLLDVICPHQEHVLPMIPSGGTENDVITEG
DGRTKY

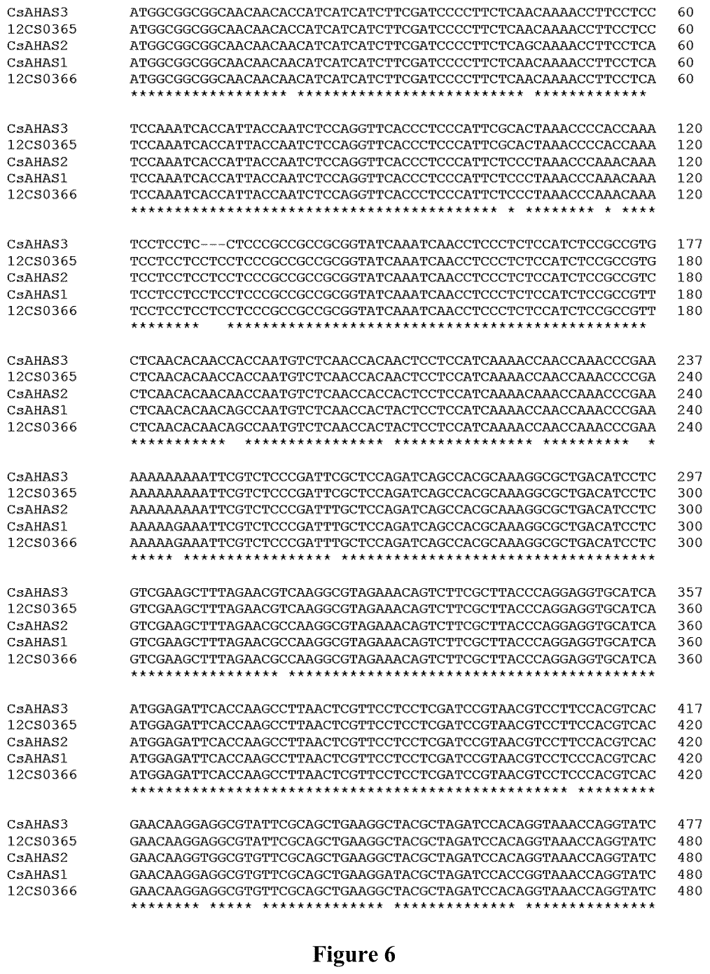

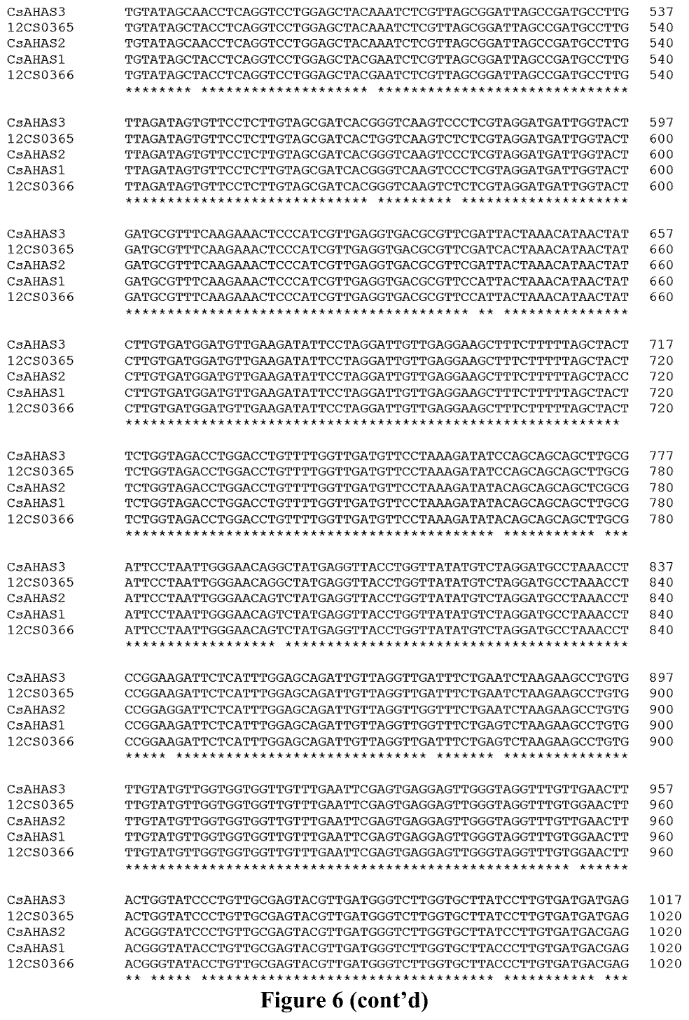

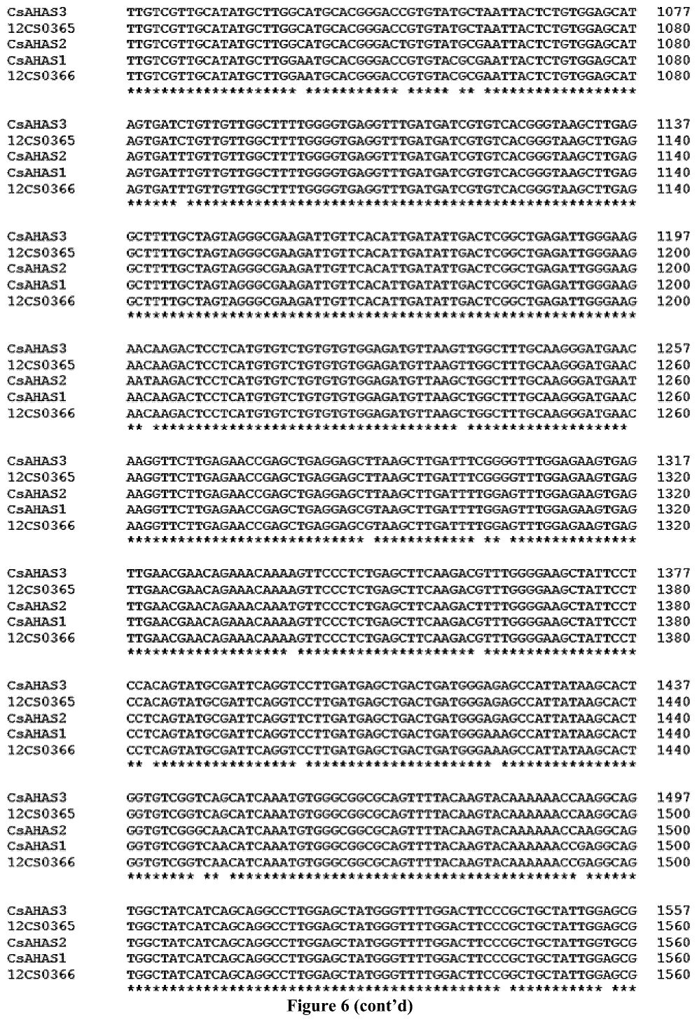

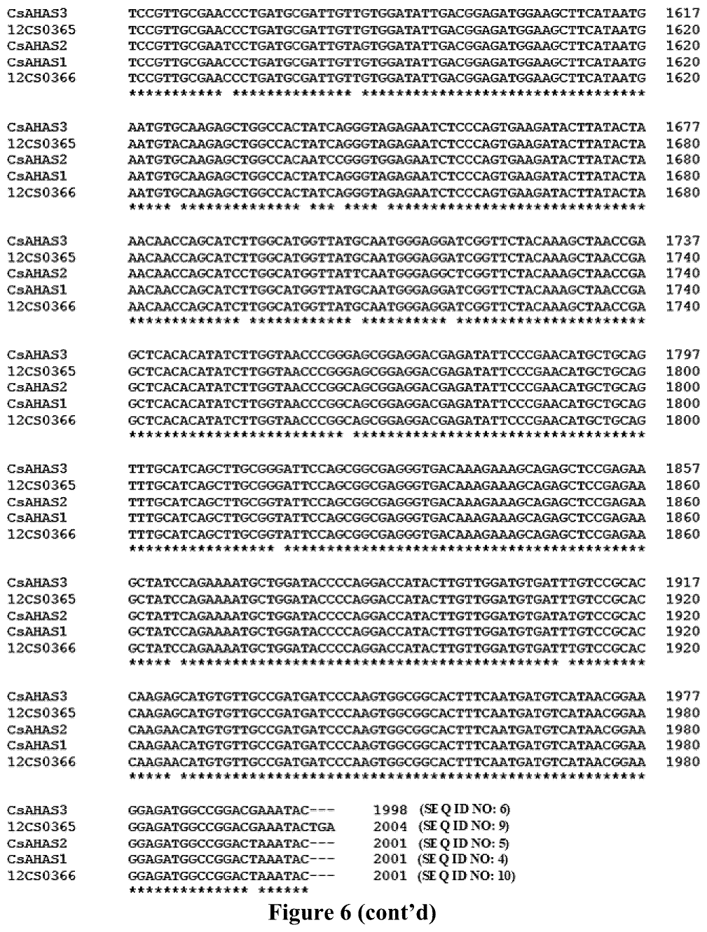

The polynucleotide sequence of each of the wildtype CsAHAS orthologues is shown in FIG. 6 as SEQ ID NOs: 4, 5 and 6, respectively.

### AHAS-Targeting Herbicides

AHAS is the target site for many commercial herbicides, generally spanning five structurally distinct classes of chemicals, namely: (i) sulfonylureas (SU); (ii) imidazolinones (IMI); (iii) sulfonylaminocarbonyltriazolinones (SCT); (iv) triazolopyrimidines (TP); and pyrimidinylthiobenzoates (PTB). These herbicides are commonly referred to as Group 2 herbicides. Without limitation, exemplary embodiments of such herbicides are provided in Table 1 below.

TABLE 1

| Group 2 | Compound identity | Commercial herbicide | | | |
|---|---|---|---|---|---|
| Imidazolinones | AC299, 263, 120 AS | Altitude FX ® | | | |
| | imazamethabenz | Assert ® 300 | Avert | | |
| | imazamox | Ares ™ | Daval ® | Salute ™ | Tensile ™ |
| | | Altitude FX ™ | Mzuna ™ | Solo ® ADV | Viper ® ADV |
| | imazamox + | Duet ® | Odessey ® | Odyssey ® | |
| | imazethapyr | | | ULTRA | |
| | | | | Odyssey ® | |
| | | | | ULTRA NXT | |
| | imazapyr | Ares ™ | Arsenal ® | SaluteTM | |
| | imazethapyr | Gladiator ® | MPower ® | Phantom ® | |
| | | Multistar | Kamikaze ® | Pursuit ® | |
| | | | Nu-Image ® | | |
| Sulfonylaminocarbonyltriazolinones | flucarbazone sodium | Everest ® | Inferno ™ Duo | | |
| Sulfonylureas | ethametsulfuron | Muster ® | | | |
| | methyl | | | | |
| | metsulfuronmethyl | Accurate ® | Escort ® | Nuance ® Pro | Travelias ® |
| | | Ally ® | Express ® Pro | Reclaim ™ | |
| | | Toss-N-Go ® | | | |
| | rimsulfuron | Prism ™ | Titus ™ Pro | | |
| | thifensulfuronmethyl | Barricade ® | Deploy ® | Refine ® SG | Triton ™ |
| | | Boost | Nimble ® | Retain ® | Pinnacle ® SG |
| | | Broadside ® | Predicate ® | Travellas ® | |
| | tribenuronmethyl | Barricade ® | Express ® SG | MPower ®R | Refine ® SG |
| | | Boost | FirstStep ™ | MPower ®K | Refine ® M |
| | | Broadside ® | InfernoTM Duo | Nimble ® | Retain ® |
| | | Deploy ® | InfernoTM WDG | Nuance ® | Signal FSU |
| | | Express ® FX | K_{D}Act ™ | Nuance ® Pro | TritonTM C |
| | | Express ® Pack | Luxxur ® | Predicate ® | TritonTM K |
| | | Express ® Pro | | | |
| | triflusulfuron methyl | UpBeet ® | | | |
| Pyrazole | halosulfuron | Permit ® | | | |
| Triazolpyramidjines | florasulm | Benchmark ® | Frontline ® | Paradigm ™ | Spitfire ® |
| | | Blitz ® | Frontline ® 2,4-D | PrePass ™ | Stellar ™ |
| | | Broadband ® | Hotshot ® | PrePass ™ Flex | Stellar ™ XL |
| | | Cirpreme ™ | Korrex ™ II | Priority ® | Topline ® |
| | | FirstPass ™ | Outshine ® | Spectrum | |
| | pyroxsulam | GoDRI ™ | Simplicity ™ | Tandem ® | |
| Triazolones | thiencarbazonemethyl | Luxxur ® | Predicate ® | Varro ™ | Velocity ® m3 |

Inhibition of AHAS decreases pools of essential branched-chain amino acids, thereby causing inhibition of protein formation. This typically leads to the slow death of the plant. For instance, sulfonylurea herbicides inhibit the AHAS enzyme by blocking substrate access to the active site and thus starve affected plants of branched-chain amino acids leading to symptoms ranging from stunting and malformation to death.

Plants resistant to Group 2 herbicides have been identified and developed. In the majority of cases, increased tolerance or resistance to Group 2 herbicides is due to altered forms of the AHAS enzyme, creating a protein that is less sensitive to inhibition by one or more AHAS-targeted herbicides.

The present disclosure relates to Camelina sativa plants resistant to AHAS-inhibiting herbicides, as well as the variant polynucleotide and polypeptide Camelina sativa AHAS genes and proteins that provide for such resistance.

### Methods for Producing Mutant Camelina Lines

In one aspect of the present disclosure, methods for developing novel plant types are disclosed whereby increased tolerance to AHAS-targeting herbicides has been introduced through conventional mutagenesis, followed by crossing of camelina mutant lines, and subsequent repeated selfing to develop stable lines.

In an embodiment, methods of producing mutant camelina lines of the present disclosure may follow the protocols described in Examples 1-3. For example, the methods may comprise:
(i) employing an ethylmethanesulfonate (EMS) seed mutagenesis approach on wildtype camelina seed, such as for example the seed of SRS 934, to produce mutagenized seed;
(ii) growing plants from the mutagenized seed to maturity to produce M2 seed, and harvesting the M2 seed;
(iii) seeding the M2 seed in a field and spraying the field with a Group 2 herbicide;
(iv) selecting plants that display increased tolerance to the Group 2 herbicide and harvesting seed of the more tolerant/resistant plants to obtain mutant lines;
(v) crossing mutant lines and subsequently stabilizing the herbicide-resistant trait through traditional breeding techniques, such as selfing.

In an embodiment, the EMS seed mutagenesis protocol involves incubating seeds at room temperature in a 0.4% EMS solution for about 8 hours. The seeds are then washed with water and planted into soil in a field or pot. Once the plants reach maturity, seed is bulk-harvested without any application of herbicide (M2 seed).

In an embodiment, plants grown from the M2 seed are sprayed with any one or more Group 2 herbicides. In an embodiment, the seeds are sprayed with a commercial Group 2 herbicide as described herein. In a particular embodiment, the seeds are sprayed with a sulfonylurea herbicide, such as for example Refine™ SG (DuPont) or Pinnacle™ SG (DuPont). Refine™ SG is a Group 2 herbicide comprising the active agents thifensulfuron-methyl and tribenuron. Pinnacle™ SG is a Group 2 herbicide comprising the active agent thifensulfuron-methyl.

It is within the ability of the skilled person to determine the quantity of herbicide to be sprayed on the plants. In an embodiment, the herbicide may be applied at a 1×, 2× or greater field rate. The herbicide may preferably be sprayed on plants at the 2-3 leaf stage or the 3-4 leaf stage; however, this again is within the ability of the skilled person and may be adjusted as appropriate depending e.g. on geographical region and/or growing conditions. In an embodiment, the spray rate, time of application and number of applications should be sufficient to provide a reduction of biomass in a herbicide susceptible camelina variety of about 50%, about 55%, about 60%, about 65%, about 70%, about 75%, about 80%, about 85% or about 90% after about 7 days after application (daa) 8 daa, 9 daa, 10 daa, 11 daa, 12 daa, 13 daa, 14 daa, 15 daa, 16 daa, 17 daa, 18 daa, 19 daa, 20 daa or 21 daa. In a preferred embodiment, the spray rate, time of application and number of applications should be sufficient to provide a reduction of biomass in a herbicide susceptible camelina variety of about 75% after 14 daa.

In an embodiment, the step of selecting plants that display tolerance to the Group 2 herbicide involves rating plants for symptoms of herbicide effect, such as stunting, chlorosis and malformation, and selecting plants which display these symptoms to the lowest degree or not at all. Seed is harvested from the selected plants and the process of selection (step (iii) above) may be repeated any number of desired times by further seeding, growing (under herbicide application) and harvesting of subsequent generation plants.

Having obtained plant lines that display increased tolerance to Group 2 herbicides, an embodiment of the methods disclosed herein involves crossing two or more of the obtained mutant lines to enhance and/or stabilize the herbicide resistance trait (e.g. pedigree breeding).

Pedigree breeding is used commonly for the improvement of self-pollinating crops or inbred lines of cross-pollinating crops. Two parents which possess favorable, complementary traits are crossed to produce an F₁. An F₂ population is produced by selfing one or several F₁'s or by intercrossing two F₁'s (sib mating). Selection of the best individuals is usually begun in the F₂ population; then, beginning in the F₃, the best individuals in the best families are usually selected. Replicated testing of families, or hybrid combinations involving individuals of these families, often follows in the F₄ generation to improve the effectiveness of selection for traits with low heritability. At an advanced stage of inbreeding (e.g., F₅ and onwards), the best lines or mixtures of phenotypically similar lines are tested for potential release as new cultivars.

In an embodiment, crossing of the mutant lines produces a double mutant line with an enhanced tolerance to the herbicide. The hybrid plants generated by the cross may be further crossed with other obtained mutant lines to further enhance and/or stabilize the herbicide resistance trait.

In an embodiment, plants obtained by crossing mutant lines may be selfed for any number of generations in order to stabilize the herbicide resistance trait. In an embodiment, the mutant lines may be selfed 1 time, 2 times, 3 times, 4 times, 5 times, 6 times, 7 times, 8 times, 9 times or 10 times. In a particular embodiment, the mutant line may be selfed 4 or 5 times.

In an embodiment, the methods disclosed herein may further comprise backcrossing. Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

In addition to phenotypic observations (e.g. increased tolerance to herbicide), the genotype of the plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

In an embodiment, analysis of the molecular profile of the generated mutant lines of camelina may be performed in order to determine the source of the increased tolerance to AHAS-targeting herbicides.

Descriptions of other breeding methods that are commonly used for different traits and crops can be found in one of several reference books (e.g., Principles of Plant Breeding, John Wiley and Son, pp. 115-161, 1960).

Camelina Mutant Lines with Increased Tolerance to Group 2 Herbicides

In an embodiment, the present disclosure relates to Camelina sativa mutant lines (cultivars) that have increased tolerance of or resistance to AHAS-targeting herbicides.

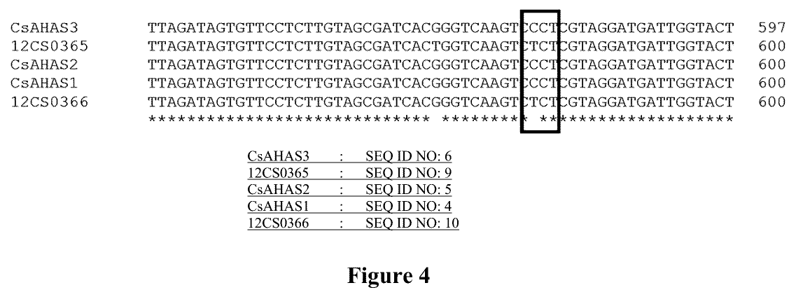

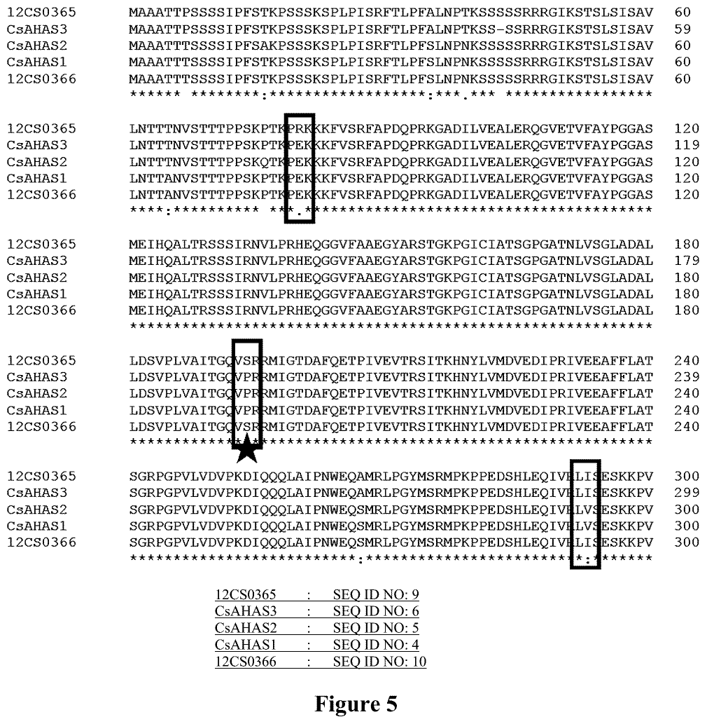

As disclosed herein, camelina mutant lines 12CS0365 and 12CS0366 were derived from mutagenizing camelina accession SRS 934 using the methods described herein. Both of 12CS0365 and 12CS0366 show increased tolerance to Group 2 herbicides. At the molecular level, a comparison of the CsAHAS sequences of 12CS0365 and 12CS0366 to wild-type CsAHAS sequences revealed a single mutation of orthologues CsAHAS1 for 12CS0366 and CsAHAS3 for 12CS0365. In these camelina lines, CsAHAS1 and CsAHAS3 each contain a single point mutation that comprises a single nucleotide change at position 580 in the gene (CCT to TCT) resulting in an amino acid substitution at position 194 from Proline to Serine (see FIGS. 4 and 5).

In an embodiment, the present disclosure relates to a plant of cultivar 12CS0365 or a plant part or seed therefrom, or a plant, plant part or seed from any subsequent generation of 12CS0365 (e.g. by selfing or crossing). In an embodiment, the present disclosure relates to a plant cell from cultivar 12CS0365 or from a plant part or seed therefrom, or a plant cell from a plant, plant part or seed from any subsequent generation of 12CS0365 (e.g. by selfing or crossing). A deposit of the seed of Camelina sativa (L.) variety 12CS0365 is maintained by Linnaeus Plant Sciences, Inc., 2212-110 Gymnasium Place, Saskatoon, SK, S7N 0W9, Canada and has also been deposited under ATCC Accession Number PTA-125493 on Dec. 3, 2018.

In an embodiment, the present disclosure relates to a plant of cultivar 12CS0366 or a plant part or seed therefrom, or a plant, plant part or seed from any subsequent generation of 12CS0366 (e.g. by selfing or crossing). In an embodiment, the present disclosure relates to a plant cell from cultivar 12CS0366 or from a plant part or seed therefrom, or a plant cell from a plant, plant part or seed from any subsequent generation of 12CS0366 (e.g. by selfing or crossing). A deposit of the seed of Camelina sativa (L.) variety 12CS0366 is maintained by Linnaeus Plant Sciences, Inc., 2212-110 Gymnasium Place, Saskatoon, SK, S7N 0W9, Canada and has also been deposited under ATCC Accession Number PTA-125492 on Dec. 3, 2018.

To produce a double mutant with two independent resistance genes, cultivars 12CS0365 (female) and 12CS0366 (male) were crossed to produce F₁ seed. The resistance trait was stabilized through repeated selfing (F₁→F₂→F₃→F₄→F₅). The F₁ seed received accession number 12CS0389, the F₂ seed received accession number 13CS0695, the F₃ seed received accession number 13CS0781, the F₄ seed received accession number 13CS0786, and the F₅ seed received the accession number 14CS0851. A bulk of 14CS0851 produced separately in the greenhouse received the accession number 14CS0851-01-14. The pedigree of camelina double mutant line 14CS0851-01-14 is shown in Schematic 1 in Example 3.

In an embodiment, the present disclosure relates to a plant of cultivar 12CS0389 or a plant part or seed therefrom, or a plant, plant part or seed from any subsequent generation of 12CS0389 (e.g. by selfing or crossing). In an embodiment, the present disclosure relates to a plant cell from cultivar 12CS0389 or from a plant part or seed therefrom, or a plant cell from a plant, plant part or seed from any subsequent generation of 12CS0389 (e.g. by selfing or crossing). A deposit of the seed of Camelina sativa (L.) variety 12CS0389 is maintained by Linnaeus Plant Sciences, Inc., 2212-110 Gymnasium Place, Saskatoon, SK, S7N 0W9, Canada and has also been deposited under ATCC Accession Number PTA-125494 on Dec. 3, 2018.

In an embodiment, the present disclosure relates to a plant of cultivar 13CS0695 or a plant part or seed therefrom, or a plant, plant part or seed from any subsequent generation of 13CS0695 (e.g. by selfing or crossing). In an embodiment, the present disclosure relates to a plant cell from cultivar 12CS0695 or from a plant part or seed therefrom, or a plant cell from a plant, plant part or seed from any subsequent generation of 13CS0695 (e.g. by selfing or crossing). A deposit of the seed of Camelina sativa (L.) variety 13CS0695 is maintained by Linnaeus Plant Sciences, Inc., 2212-110 Gymnasium Place, Saskatoon, SK, S7N 0W9, Canada.

In an embodiment, the present disclosure relates to a plant of cultivar 13CS0781 or a plant part or seed therefrom, or a plant, plant part or seed from any subsequent generation of 13CS0781 (e.g. by selfing or crossing). In an embodiment, the present disclosure relates to a plant cell from cultivar 13CS0781 or from a plant part or seed therefrom, or a plant cell from a plant, plant part or seed from any subsequent generation of 13CS0781 (e.g. by selfing or crossing). A deposit of the seed of Camelina sativa (L.) variety 13CS0781 is maintained by Linnaeus Plant Sciences, Inc., 2212-110 Gymnasium Place, Saskatoon, SK, S7N 0W9, Canada.

In an embodiment, the present disclosure relates to a plant of cultivar 13CS0786 or a plant part or seed therefrom, or a plant, plant part or seed from any subsequent generation of 13CS0786 (e.g. by selfing or crossing). In an embodiment, the present disclosure relates to a plant cell from cultivar 13CS0786 or from a plant part or seed therefrom, or a plant cell from a plant, plant part or seed from any subsequent generation of 13CS0786 (e.g. by selfing or crossing). A deposit of the seed of Camelina sativa (L.) variety 13CS0786 is maintained by Linnaeus Plant Sciences, Inc., 2212-110 Gymnasium Place, Saskatoon, SK, S7N 0W9, Canada.

In an embodiment, the present disclosure relates to a plant of cultivar 14CS0851 or a plant part or seed therefrom, or a plant, plant part or seed from any subsequent generation of 14CS0851 (e.g. by selfing or crossing). In an embodiment, the present disclosure relates to a plant cell from cultivar 14CS0851 or from a plant part or seed therefrom, or a plant cell from a plant, plant part or seed from any subsequent generation of 14CS0851 (e.g. by selfing or crossing). A deposit of the seed of Camelina sativa (L.) variety 14CS0851 is maintained by Linnaeus Plant Sciences, Inc., 2212-110 Gymnasium Place, Saskatoon, SK, S7N 0W9, Canada.

In an embodiment, the present disclosure relates to a plant of cultivar 14CS0851-01-14 or a plant part or seed therefrom, or a plant, plant part or seed from any subsequent generation of 14CS0851-01-14 (e.g. by selfing or crossing). In an embodiment, the present disclosure relates to a plant cell from cultivar 14CS0851-01-14 or from a plant part or seed therefrom, or a plant cell from a plant, plant part or seed from any subsequent generation of 14CS0851-01-14 (e.g. by selfing or crossing). A deposit of the seed of Camelina sativa (L.) variety 14CS0851-01-14 is maintained by Linnaeus Plant Sciences, Inc., 2212-110 Gymnasium Place, Saskatoon, SK, S7N 0W9, Canada. A representative sample of seeds of ‘14CS0851-01-14’ has been deposited under ATCC Accession No. PTA-125495 on Dec. 3, 2018.

Camelina sativa 14CS0851-01-14 was thus developed through crossing of the two camelina mutant lines 12CS0365 and 12CS0366, both derived from mutagenizing camelina accession SRS 934, and subsequent repeated selfing. Camelina line 14CS0851-01-14 possesses a single point mutation in both the CsAHAS1 and CsAHAS3 genes. As described above, this single nucleotide change at position 580 in the CsAHAS1 and CsAHAS3 genes results in an amino acid substitution at position 194 from Proline to Serine.

Camelina line 14CS0851-01-14 has increased tolerance to Group 2 herbicides compared to conventional camelina varieties. In particular, and without limitation, camelina line 14CS0851-01-14 was observed to have significantly increased tolerance to sulfonylurea herbicide Pinnacle™ SG (thifensulfuron-methyl) and sulfonylaminocarbonyl triazolinone herbicide Everest™ (flucarbazone-sodium), at commercially acceptable levels (see Example 4).

Further, dose-response data from greenhouse experiments using different rates of thifensulfuron-methyl show that the direct progenitor of 14CS0851-01-14 (13CS0786, same source of resistance) is about 1,000× more resistant than that of the camelina germplasm that was originally mutated (SRS 934) when considering plant biomass (see Example 6; Table 11).

By providing a non-GMO Group 2 herbicide-resistant camelina variety, growers will benefit from vastly improved weed control, which will result in increased yield and much wider crop adoption. Further, Group 2 herbicide resistance will alleviate current re-cropping restrictions and will allow camelina to be used as a rotation crop for the first time on the over 4 million acres of lentils grown in Western Canada that leave Group 2 residual in the soil, a game changer for the crop.

In an embodiment, in addition to increased tolerance or resistance to Group 2 herbicides, the plants disclosed herein have additional phenotypic characteristics that are desirable for growing Camelina sativa plants, such as for its high-value oil.

As disclosed herein, a nutritional evaluation of cultivar 14CS0851-01-14 was compared to wildtype SRS 934 and commercial line MIDAS™. As used herein, “MIDAS™”, “Midas”, “MIDAS” or “Midas™” refers to a camelina cultivar released by Smart Earth Seeds (parent: Linnaeus Plant Sciences) in the spring of 2013. MIDAS™ is the tradename for PBR variety AAC 10CS0048. This elite camelina variety was developed in Saskatoon, SK, Canada at the Agriculture and Agri-Food Canada Research Station. MIDAS™ is a spring-type Camelina cultivar with high seed yield and high oil content. In performance evaluations in central and southern Saskatchewan and Alberta, MIDAS™ yielded over 35 bu/acre on average, with an oil content of 41 to 42% at 14 separate locations. MIDAS™ grows to medium heights (26-34 inches), and it flowers, depending on the weather conditions, after about 45 days. The crop reaches maturity 85 to 100 days after seeding. Unique to MIDAS™ is its partial resistance to downy mildew, the most important pathogen in camelina production. With this, MIDAS™ has a competitive advantage over other Camelina cultivars.

Whole seeds of each of these lines (14CS0851-01-14, SRS 934 and MIDAS™) were analyzed for crude protein, crude fibre, crude fat, ash, moisture, acid detergent fibre (ADF), neutral detergent fibre (NDF), minerals (phosphorous and calcium), and amino acids. In addition, an evaluation of the antinutritionals glucosinolates, tannins, sinapine, trypsin inhibitors, and phytic acid was performed. Further, oil extracts of whole seeds were analyzed for fatty acids and tocopherols (vitamin E). Although statistically significant differences were observed, the differences were not pronounced. Thus, products derived from cultivar 14CS0851-01-14 and its derivatives are not anticipated to be any different than products derived from current camelina varieties when grown for commercial purposes.

In an embodiment, the herbicide-resistant plant cultivar of the present disclosure comprises a proximate composition (ash, acid detergent fibre, neutral detergent fibre and non-fibre carbohydrates) that is substantially similar to a commercial camelina variety, such as MIDAS™. By “substantially similar”, it is meant that the quantity of proximates does not differ by such an extent to render the plants unsuitable for any commercial use. In an embodiment, “substantially similar” means that the quantity does not differ by more than 10% from that of a commercial variety, such as MIDAS™.

In an embodiment, the herbicide-resistant plant cultivar of the present disclosure comprises a seed oil content that is substantially similar to a commercial camelina variety, such as MIDAS™. By “substantially similar”, it is meant that the quantity of seed oil does not differ by such an extent to render the plants unsuitable for any commercial use. In an embodiment, “substantially similar” means that the quantity does not differ by more than 10% from that of a commercial variety, such as MIDAS™.

In an embodiment, the herbicide-resistant plant cultivar of the present disclosure comprises a seed oil having a fatty acid content that is substantially similar to a commercial camelina variety, such as MIDAS™. By “substantially similar”, it is meant that the quantity of fatty acids in the seed oil does not differ by such an extent to render the plants unsuitable for any commercial use. In an embodiment, “substantially similar” means that the quantity does not differ by more than 10% from that of a commercial variety, such as MIDAS™.

In an embodiment, the herbicide-resistant plant cultivar of the present disclosure comprises a seed oil having a fatty acid content that is substantially similar to a commercial camelina variety, such as MIDAS™. By “substantially similar”, it is meant that the quantity of fatty acids in the seed oil does not differ by such an extent to render the plants unsuitable for any commercial use. In an embodiment, “substantially similar” means that the quantity does not differ by more than 10% from that of a commercial variety, such as MIDAS™.

In an embodiment, the herbicide-resistant plant cultivar of the present disclosure comprises a mineral (e.g. calcium and phosphorous) and/or antinutritionals (sinapine, phytate, trypsin inhibitors, tannins and glucosinolates) content that is substantially similar to a commercial camelina variety, such as MIDAS™. By “substantially similar”, it is meant that the quantity of minerals and/or antinutritionals does not differ by such an extent to render the plants unsuitable for any commercial use. In an embodiment, “substantially similar” means that the quantity does not differ by more than 10% from that of a commercial variety, such as MIDAS™.

In an embodiment, the herbicide-resistant plant cultivar of the present disclosure has a germination and seedling vigor that is substantially similar to a commercial camelina variety, such as MIDAS™.

Further disclosed herein are camelina variants in which the herbicide-resistant trait has been introduced into the elite camelina cultivar MIDAS™. In an embodiment, the herbicide-resistance trait is introduced into MIDAS™ by introgression. In an embodiment, the herbicide-resistant MIDAS™ plant cultivar is generated by crossing MIDAS™ with a single or double mutant plant cultivar of the present disclosure, such as for example and without limitation: 11CS0111, 12CS0363, 12CS0364, 12CS0365, 12CS0366, 12CS0389, 13CS0695, 13CS0781, 13CS0786, 13CS0787, 14CS0814, 14CS0851, 14CS0851-01-14, 13CS0777-02, 13CS0778-02, 13CS0779-02, 13CS0780-02, 13CS0783-02, 13CS0784-02, 13CS0785-02, 13CS0787-02 or 14CS0852-01-12. In an embodiment, MIDAS™ is crossed with 13CS0786, 14CS0814, 14CS0851, 14CS0851-01-14, 13CS0777-02, 13CS0778-02, 13CS0779-02 or 13CS0780-02.

In an embodiment, the herbicide-resistant MIDAS™ plant cultivar is an F₁ plant derived from crossing MIDAS™ with a single or double mutant plant cultivar of the present disclosure, or a progeny thereof. In an embodiment, the herbicide-resistant MIDAS™ plant cultivar is that of succession 14CS0903 (Example 18), or a progeny thereof.

In an embodiment, one or more successive backcrosses are performed to obtain the herbicide-resistant MIDAS™ plant cultivar.

In an embodiment, the herbicide-resistant MIDAS™ plant cultivar is a BC1F1 plant derived from back-crossing F₁ plants with MIDAS™, or a progeny thereof. In an embodiment, the herbicide-resistant MIDAS™ plant cultivar is that of succession 14CS0909 (Example 18), or a progeny thereof.

In an embodiment, the herbicide-resistant MIDAS™ plant cultivar is a BC2F1 plant derived from back-crossing BC1 plants with MIDAS™, or a progeny thereof. In an embodiment, the herbicide-resistant MIDAS™ plant cultivar is that of succession 15CS0985 (Example 18), or a progeny thereof.

In an embodiment, the herbicide-resistant MIDAS™ plant cultivar is a BC3F1 plant derived from back-crossing BC2 plants with MIDAS™, or a progeny thereof. In an embodiment, the herbicide-resistant MIDAS™ plant cultivar is that of succession 15CS1007 (Example 18), or a progeny thereof.

In an embodiment, the herbicide-resistant MIDAS™ plant cultivar is a BC4F1 plant derived from back-crossing BC3 plants with MIDAS™, or a progeny thereof. In an embodiment, the herbicide-resistant MIDAS™ plant cultivar is that of succession 15CS1018 (Example 18), or a progeny thereof. In an embodiment, the herbicide-resistant MIDAS™ plant cultivar is a BC4F2 generation plant or a progeny thereof. In an embodiment, the herbicide-resistant MIDAS™ plant cultivar is that of succession 16CS1054 (Example 18), or a progeny thereof. In an embodiment, the herbicide-resistant MIDAS™ plant cultivar is a BC4F3 generation plant or a progeny thereof. In an embodiment, the herbicide-resistant MIDAS™ plant cultivar is that of succession 16CS1068 (Example 18), or a progeny thereof.

In an embodiment, the herbicide-resistant MIDAS™ plant cultivar is a BC4F4 generation plant or a progeny thereof. In an embodiment, the herbicide-resistant MIDAS™ plant cultivar is that of succession 17CS1115 (Example 18), or a progeny thereof.

In an embodiment, the herbicide-resistant MIDAS™ plant cultivar is cultivar 17CS1115. A deposit of the seed of Camelina sativa (L.) variety 17CS1115 is maintained by Linnaeus Plant Sciences, Inc., 2212-110 Gymnasium Place, Saskatoon, SK, S7N 0W9, Canada.

Further disclosed herein are camelina variants in which the herbicide-resistant trait has been introduced into the elite camelina cultivar CYPRESS™. As used herein, “CYPRESS™”, “CYPRESS”, “Cypress™” or “Cypress” refers to Linnaeus Plant Sciences' variety SES0787LS, Plant Breeders Rights application #16-8839. The seed of CYPRESS™ camelina is 40% larger than all other commercial varieties. In addition, the leaves exhibit a more pronounced pubescence, the infructescence shows stronger branching, and the pods are larger. CYPRESS™ camelina possesses superior emergence, establishment, and higher yields than other commercial varieties.

In an embodiment, the herbicide-resistance trait is introduced into CYPRESS™ by introgression. In an embodiment, the herbicide-resistant CYPRESS™ plant cultivar is generated by crossing CYPRESS™ with a single or double mutant plant cultivar of the present disclosure, such as for example and without limitation: 11CS0111, 12CS0363, 12CS0364, 12CS0365, 12CS0366, 12CS0389, 13CS0695, 13CS0781, 13CS0786, 13CS0787, 14CS0814, 14CS0851, 14CS0851-01-14, 13CS0777-02, 13CS0778-02, 13CS0779-02, 13CS0780-02, 13CS0783-02, 13CS0784-02, 13CS0785-02, 13CS0787-02 or 14CS0852-01-12. In an embodiment, CYPRESS™ is crossed with 13CS0786, 14CS0851-01-14 or 14CS0851.

In an embodiment, the herbicide-resistant CYPRESS™ plant cultivar is an F1 plant derived from crossing CYPRESS™ with a single or double mutant plant cultivar of the present disclosure, or a progeny thereof. In an embodiment, the herbicide-resistant CYPRESS™ plant cultivar is that of succession 15CS0999 (Example 19), or a progeny thereof.

In an embodiment, one or more successive backcrosses are performed to obtain the herbicide-resistant CYPRESS™ plant cultivar.

In an embodiment, the herbicide-resistant CYPRESS™ plant cultivar is a BC1F1 plant derived from back-crossing F1 plants with CYPRESS™, or a progeny thereof. In an embodiment, the herbicide-resistant CYPRESS™ plant cultivar is that of succession 15CS1020 (Example 19), or a progeny thereof.

In an embodiment, the herbicide-resistant CYPRESS™ plant cultivar is a BC2F1 plant derived from back-crossing BC1 plants with CYPRESS™, or a progeny thereof. In an embodiment, the herbicide-resistant CYPRESS™ plant cultivar is that of succession 16CS1056 (Example 19), or a progeny thereof.

In an embodiment, the herbicide-resistant CYPRESS™ plant cultivar is a BC3F1 plant derived from back-crossing BC2 plants with CYPRESS™, or a progeny thereof. In an embodiment, the herbicide-resistant CYPRESS™ plant cultivar is that of succession 16CS1070 (Example 19), or a progeny thereof.

In an embodiment, the herbicide-resistant CYPRESS™ plant cultivar is a BC4F1 plant derived from back-crossing BC3 plants with CYPRESS™, or a progeny thereof. In an embodiment, the herbicide-resistant CYPRESS™ plant cultivar is that of succession 17CS1088 (Example 19), or a progeny thereof. In an embodiment, the herbicide-resistant CYPRESS™ plant cultivar is a BC4F2 generation plant or a progeny thereof. In an embodiment, the herbicide-resistant CYPRESS™ plant cultivar is that of succession 17CS1112 (Example 19), or a progeny thereof. In an embodiment, the herbicide-resistant CYPRESS™ plant cultivar is a BC4F3 generation plant or a progeny thereof. In an embodiment, the herbicide-resistant CYPRESS™ plant cultivar is that of succession 17CS1131 (Example 19), or a progeny thereof.

In an embodiment, the herbicide-resistant CYPRESS™ plant cultivar is a BC4F4 generation plant or a progeny thereof. In an embodiment, the herbicide-resistant CYPRESS™ plant cultivar is that of succession 18CS1152, 18CS1153, 18CS1154, 18CS1155 or 18CS1156 (Example 19), or a progeny thereof.

Further disclosed herein are camelina variants in which the herbicide-resistant trait has been introduced into the elite camelina cultivar PEARL™. As used herein, “PEARL™”, “PEARL”, “Pearl™” or “Pearl” refers to Linnaeus Plant Sciences' variety SES0877IOR, Plant Breeders Rights application #16-8840. PEARL™ fatty acid profile of the seed oil contains less linoleic acid, and more oleic acid than other commercial varieties. The omega-3: omega-6 ratio is considerably higher than other commercial varieties such as MIDAS™, ranging 2.0-2.5 for PEARL™ compared to 1.1-1.6 for MIDAS™ seed oil. In addition, plant height is shorter and pods are bigger than MIDAS. Arrangement of pods on branches is very dense, resembling pearls on a string.

In an embodiment, the herbicide-resistance trait is introduced into PEARL™ by introgression. In an embodiment, the herbicide-resistant PEARL™ plant cultivar is generated by crossing PEARL™ with a single or double mutant plant cultivar of the present disclosure, such as for example and without limitation: 11CS0111, 12CS0363, 12CS0364, 12CS0365, 12CS0366, 12CS0389, 13CS0695, 13CS0781, 13CS0786, 13CS0787, 14CS0814, 14CS0851, 14CS0851-01-14, 13CS0777-02, 13CS0778-02, 13CS0779-02, 13CS0780-02, 13CS0783-02, 13CS0784-02, 13CS0785-02, 13CS0787-02 or 14CS0852-01-12. In an embodiment, the herbicide-resistance PEARL™ cultivar is any F1, F2, F3, F4, F5, BC1, BC2, BC3 or BC4 generation plant, or any progeny thereof.

### Variant Acetohydroxyacid Synthase (AHAS) Polypeptides

The present disclosure relates to novel Camelina sativa AHAS polypeptides that provide camelina plants with improved tolerance and/or resistance to Group 2 herbicides, such as for example sulfonylureas. The Camelina sativa AHAS polypeptides are variants of one or more of the three AHAS orthologues (CsAHAS1, CsAHAS2, and CsAHAS3) that are found in camelina. In an embodiment, the variant is a CsAHAS1 polypeptide. In an embodiment, the variant is a CsAHAS2 polypeptide. In an embodiment, the variant is a CsAHAS3 polypeptide. In an embodiment, the plant or cells thereof comprises variant CsAHAS polypeptides of two or more different orthologues, such as for example CsAHAS1 and CsAHAS3, or any other combination.

The AHAS variants of the present disclosure comprise a substitution of the proline at a position corresponding to position 194 in SEQ ID NO: 1 and 2 (position 193 in SEQ ID NO: 3).

In an embodiment, the substitution of P194 in CsAHAS is a substitution of proline with any other amino acid. In an embodiment, the substitution of P194 is a conservative amino acid substitution, such as substitution of proline with serine (P194S), alanine (P194A), cysteine (P194C), asparagine (P194N), threonine (P194T), tryptophan (P194W) or tyrosine (P194Y).

In a particular embodiment, the substitution of P194 in CsAHAS is a substitution of proline with serine (P194S).

AHAS polypeptides of the present disclosure include variant AHAS polypeptides comprising an amino acid sequence that is at least 75% identical to CsAHAS1 (SEQ ID NO: 1), CsAHAS2 (SEQ ID NO: 2) or CsAHAS3 (SEQ ID NO: 3), and having at least a substitution of P194 as described herein. In an embodiment, the variant AHAS polypeptide of the present disclosure is at least 75%, at least 80%, at least 85%, at least 90% or at least 95% identical to the sequence of CsAHAS1, CsAHAS2 or CsAHAS3 (SEQ ID NO: 1, 2 or 3, respectively) and having a substitution at a position corresponding to P194 in SEQ ID NO: 1 or 2.

In an embodiment, in addition to the substitution at P194, the variant CsAHAS polypeptide may comprise one or more additional modifications as compared to the corresponding wildtype CsAHAS. In some embodiments, the one or more additional modifications include further amino acid substitutions, deletions and/or insertions. In an embodiment, the additional modifications are amino acid substitutions. For example, and without limitation, the additional modification may be a substitution of arginine at position 80 and/or valine at position 293, wherein the amino acid positions are determined by alignment with SEQ ID NO: 1 or 2. In an embodiment, the arginine at position 80 is substituted with glutamate (R80E). In an embodiment, the valine at position 293 is substituted with isoleucine (V293I). These exemplary additional amino acid substitutions are shown in FIG. 5 in respect of the camelina lines 12CS0365 and 12CS0366 of the present disclosure.

In an embodiment, the variant CsAHAS polypeptide of the
present disclosure comprises or consists of an amino acid
sequence of SEQ ID NO: 7:
(SEQ ID NO: 7)
MAAATTPSSSSIPFSTKPSSSKSPLPISRFTLPFALNPTKSSSSSRRRGIKSTSLSISAV
LNTTTNVSTTTPPSKPTKPRKKKFVSRFAPDQPRKGADILVEALERQGVETVFAYPGGAS
MEIHQALTRSSSIRNVLPRHEQGGVFAAEGYARSTGKPGICIATSGPGATNLVSGLADAL
LDSVPLVAITGQVSRRMIGTDAFQETPIVEVTRSITKHNYLVMDVEDIPRIVEEAFFLAT
SGRPGPVLVDVPKDIQQQLAIPNWEQAMRLPGYMSRMPKPPEDSHLEQIVRLISESKKPV
LYVGGGCLNSSEELGREVELTGIPVASTLMGLGAYPCDDELSLHMLGMHGTVYANYSVEH
SDLLLAFGVREDDRVTGKLEAFASRAKIVHIDIDSAEIGKNKTPHVSVCGDVKLALQGMN
KVLENRAEELKLDFGVWRSELNEQKQKFPLSFKTFGEAIPPQYAIQVLDELTDGRAIIST
GVGQHQMWAAQFYKYKKPRQWLSSAGLGAMGFGLPAAIGASVANPDAIVVDIDGDGSFIM
NVQELATIRVENLPVKILILNNQHLGMVMQWEDRFYKANRAHTYLGNPGAEDEIFPNMLQ
FASACGIPAARVTKKAELREAIQKMLDTPGPYLLDVICPHQEHVLPMIPSGGTENDVITE
GDGRTKY
In an embodiment, the variant CsAHAS polypeptide of the
present disclosure comprises or consists of an amino acid
sequence of SEQ ID NO: 8:
(SEQ ID NO: 8)
MAAATTTSSSSIPESTKPSSSKSPLPISRFTLPFSLNPNKSSSSSRRRGIKSTSLSISAV
LNTTANVSTTTPPSKPTKPEKKKFVSRFAPDQPRKGADILVEALERQGVETVFAYPGGAS
MEIHQALTRSSSIRNVLPRHEQGGVFAAEGYARSTGKPGICIATSGPGATNLVSGLADAL
LDSVPLVAITGQVSRRMIGTDAFQETPIVEVTRSITKHNYLVMDVEDIPRIVEEAFFLAT
SGRPGPVLVDVPKDIQQQLAIPNWEQSMRLPGYMSRMPKPPEDSHLEQIVRLISESKKPV
LYVGGGCLNSSEELGREVELTGIPVASTLMGLGAYPCDDELSLHMLGMHGTVYANYSVEH
SDLLLAFGVREDDRVTGKLEAFASRAKIVHIDIDSAEIGKNKTPHVSVCGDVKLALQGMN
KVLENRAEERKLDFGVWRSELNEQKQKFPLSFKTFGEAIPPQYAIQVLDELTDGKAIIST
GVGQHQMWAAQFYKYKKPRQWLSSAGLGAMGFGLPAAIGASVANPDAIVVDIDGDGSFIM
NVQELATIRVENLPVKILILNNQHLGMVMQWEDRFYKANRAHTYLGNPAAEDEIFPNMLQ
FASACGIPAARVTKKAELREAIQKMLDTPGPYLLDVICPHQEHVLPMIPSGGTENDVITE
GDGRTKY

The variant CsAHAS polypeptides of the present disclosure may be isolated or may be present within the plant or plant cell. The variant CsAHAS polypeptides of the present disclosure may, for example, be produced by recombinant means.

### Variant Acetohydroxyacid Synthase (AHAS) Polynucleotides

The present disclosure relates to polynucleotides that encode any of the above-described CsAHAS variant polypeptides of the present disclosure.

Those having ordinary skill in the art will readily appreciate that due to the degeneracy of the genetic code, a multitude of nucleotide sequences encoding CsAHAS polypeptides of the present disclosure may exist. The Codon Table below provides the synonymous codons for each amino acid. It is understood that U in an RNA sequence corresponds to T in a DNA sequence.

TABLE 2

| Amino acids | | | Codon | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

For example, the codons AGA, AGG, CGA, CGC, CGG, and CGU all encode the amino acid arginine. Thus, at every position in the nucleic acids of the present disclosure where an arginine is specified by a codon, the codon can be altered to any of the corresponding codons described above without altering the encoded polypeptide. This is likewise the situation for other codons as shown above.

Such “silent variations” are one species of “conservative” variation. One of ordinary skill in the art will recognize that each codon in a nucleic acid (except AUG, which is ordinarily the only codon for methionine) can be modified by standard techniques to encode a functionally identical polypeptide. Accordingly, each silent variation of a nucleic acid which encodes a polypeptide is implicit in any described sequence. The present disclosure contemplates and relates to each and every possible variation of nucleic acid sequence encoding a polypeptide of the present disclosure that could be made by selecting combinations based on possible codon choices. These combinations are made in accordance with the standard triplet genetic code (set forth above), as applied to the polynucleotide sequences of the present disclosure.

In an embodiment, the CsAHAS polynucleotides of the present disclosure include any polynucleotide that encodes any of the above-described CsAHAS variant polypeptides. In a particular embodiment, the CsAHAS polynucleotide is one comprising a nucleotide substitution of cytosine (C) to thymine (T) at position 580, wherein the nucleotide position is determined by alignment with a wildtype CsAHAS nucleotide sequence of SEQ ID NO: 4 or 5. In the wildtype CsAHAS genes, this modification results in a codon change from CCT to TCT, thereby resulting in an amino acid substitution from Proline to Serine (see FIGS. 4 and 5). As will be appreciated, the codons AGC, AGT, TCA, TCC and TCG also code for serine. Thus, the variant codon TCT at position 580-582 of the CsAHAS polynucleotides of the present disclosure could equally be replaced by AGC, AGT, TCA, TCC and TCG. This would be a silent variation since the encoded amino acid remains serine.

Exemplary CsAHAS polynucleotides of the present disclosure include those corresponding to SEQ ID NO: 9 and 10, as shown in FIG. 6. Thus, in an embodiment, the CsAHAS polynucleotide comprises or consists of the nucleotide sequence of SEQ ID NO: 9. In another embodiment, the CsAHAS polynucleotide comprises or consists of the nucleotide sequence of SEQ ID NO: 10. As described above, these sequences may for example be modified taking into account the degeneracy of the genetic code, including without limitation the replacement of codon TCT with AGC, AGT, TCA, TCC or TCG at position 580-582, wherein the nucleotide position is determined by alignment with a wildtype CsAHAS nucleotide sequence of SEQ ID NO: 4 or 5.

The variant CsAHAS polynucleotides of the present disclosure may be isolated or may be present within the plant or a plant cell. The variant CsAHAS polynucleotides of the present disclosure may, for example, be produced by recombinant means.

For example, polynucleotides of the present disclosure can be prepared using methods that are well known in the art. Typically, oligonucleotides of up to about 120 bases are individually synthesized, then joined (e.g., by enzymatic or chemical ligation methods, or polymerase-mediated methods) to form essentially any desired continuous sequence. For example, polynucleotides of the present disclosure can be prepared by chemical synthesis using, for example, the classical phosphoramidite method described by Beaucage, et al. (1981) Tetrahedron Letters, 22:1859-69, or the method described by Matthes, et al. (1984) EMBO J., 3:801-05. These methods are typically practiced in automated synthetic methods. According to the phosphoramidite method, oligonucleotides are synthesized, e.g., in an automatic DNA synthesizer, purified, annealed, ligated and cloned in appropriate vectors.

In addition, essentially any nucleic acid can be custom ordered from any of a variety of commercial sources, such as The Midland Certified Reagent Company (Midland, TX), The Great American Gene Company (Ramona, CA), ExpressGen Inc. (Chicago, IL), Operon Technologies Inc. (Alameda, CA), and many others.

Polynucleotides may also be synthesized by well-known techniques as described in the technical literature. See, e.g., Carruthers, et al., Cold Spring Harbor Symp. Quant. Biol, 47:411-418 (1982) and Adams, et al, J. Am. Chem. Soc, 105:661 (1983). Double stranded DNA fragments may then be obtained either by synthesizing the complementary strand and annealing the strands together under appropriate conditions, or by adding the complementary strand using DNA polymerase with an appropriate primer sequence.

### Plant Parts, Seeds and Plant Cells

The present disclosure further relates to plant parts of the camelina plants of the present disclosure. In some embodiments, the plant part is the shoot, root, stem, seeds, racemes, stipules, leaves, petals, flowers, ovules, bracts, branches, petioles, internodes, pollen, stamen, or the like.

In some embodiments, the plant part is a seed. The seed comprises a CsAHAS polynucleotide variant as described herein. The CsAHAS polynucleotide may for example, and without limitation, be a CsAHAS polynucleotide comprising the sequence of SEQ ID NO: 9 or 10. In an embodiment, the seed comprises both the CsAHAS polynucleotides of SEQ ID NO: 9 and 10. In an embodiment, the seed expresses (or is capable of expressing) the CsAHAS polypeptide variant as described herein, such as for example the CsAHAS polypeptide of one or both of SEQ ID NO: 7 and 8.

In some embodiments, the seed is of the camelina plant designated as 12CS0365, 12CS0366, 12CS0389, 13CS0695, 13CS0781, 13CS0786, 14CS0851-01-14 or 17CS1115. Representative seed of varieties 12CS0365, 12CS0366, 12CS0389 and 14CS0851-01-14 has been deposited under ATCC Accession Numbers PTA-125493, PTA-125492, PTA-125494, and PTA-125495, respectively. Seed of varieties 13CS0695, 13CS0781, 13CS0786 and 17CS1115 is maintained by Linnaeus Plant Sciences, Inc., 2212-110 Gymnasium Place, Saskatoon, SK, S7N 0W9, Canada. In a particular embodiment, the seed of the camelina plant designated as 14CS0851-01-14, wherein representative seed of said variety has been deposited under ATCC Accession Number PTA-125495.

In an embodiment, the present disclosure relates to a Camelina sativa plant, or part thereof, produced by growing the seed as described above.

The present disclosure also relates to plant cells of the camelina plants of the present disclosure. In some embodiments, the plant cell can be cultured and used to produce a camelina plant having one or more, or all the physiological and morphological characteristics of the camelina plants of the present disclosure, including herbicide resistance.

The plant cell seed comprises a CsAHAS polynucleotide variant as described herein. The CsAHAS polynucleotide may for example, and without limitation, be a CsAHAS polynucleotide comprising the sequence of SEQ ID NO: 9 or 10. In an embodiment, the plant cell comprises both the CsAHAS polynucleotides of SEQ ID NO: 9 and 10. In an embodiment, the plant cell expresses (or is capable of expressing) the CsAHAS polypeptide variant as described herein, such as for example the CsAHAS polypeptide of one or both of SEQ ID NO: 7 and 8.

In some embodiments, the plant cell from a camelina plant designated as 12CS0365, 12CS0366, 12CS0389, 13CS0695, 13CS0781, 13CS0786, 14CS0851-01-14 or 17CS1115. Representative seed of varieties 12CS0365, 12CS0366, 12CS0389 and 14CS0851-01-14 has been deposited under ATCC Accession Numbers PTA-125493, PTA-125492, PTA-125494, and PTA-125495, respectively. Seed of varieties 13CS0695, 13CS0781, 13CS0786 and 17CS1115 is maintained by Linnaeus Plant Sciences, Inc., 2212-110 Gymnasium Place, Saskatoon, SK, S7N 0W9, Canada. In a particular embodiment, the plant cell is from the camelina plant designated as 14CS0851-01-14, wherein representative seed of said variety has been deposited under ATCC Accession Number PTA-125495.

In an embodiment, the present disclosure relates to a plant, or part thereof, comprising the plant cell as described above. In an embodiment, the plant is resistant to acetolactate synthase inhibiting herbicides, and more particularly to sulfonylamino-carbonyltriazolinones and/or sulfonylureas. In an embodiment, the plant is resistant to thifensulfuron-methyl. In an embodiment, the plant is resistant to flucarbazone-sodium.

The present disclosure also relates to tissue culture of the camelina plants of the present disclosure. In some embodiments, the tissue culture are produced from a plant part selected from the group consisting of embryos, meristematic cells, leaves, pollen, root, root tips, stems, anther, pistils, pods, flowers, and seeds. In some embodiments, the tissue culture can be used to regenerate a Camelina sativa (L.) plant, said plant having the morphological and physiological characteristics of Camelina sativa plants of the present disclosure, including herbicide resistance.

### Uses

The present disclosure further relates to methods for producing a camelina seed. In some embodiments, said methods comprise crossing a first parent camelina plant with a second parent camelina plant and harvesting the resultant hybrid seed, wherein said first parent camelina plant or second parent camelina plant is a Camelina sativa plant of the present disclosure, such as for example cultivar 14CS0851-01-14.

The present disclosure also relates to methods for introducing one or more desired traits into camelina plants of the present disclosure, such as into cultivar 14CS0851-01-14. In some embodiments, the methods comprise introducing one or more transgenes into the camelina plants of the present disclosure. In some other embodiments, the introducing step comprises crossing or backcrossing the camelina plants of the present disclosure (e.g. 14CS0851-01-14) to one or more other camelina plants having desirable traits. In some embodiments, the desirable trait is increased tolerance or resistance to a disease (e.g. downy mildew, e.g. such as caused by Peronospora camelinae; or sclerotinia stem rot, e.g. such as caused by Sclerotinia sclerotiorum) or an environmental stressor (e.g. drought tolerance, heat tolerance, cold tolerance, improved nutritional quality or oil quality, etc.). Thus, in an embodiment, the present disclosure relates to uses of the plants of the disclosure for introgression of the herbicide-resistance trait into another camelina variety.

In some embodiments, the present disclosure relates to for the use of the plants of the present disclosure for producing progeny. Progeny may be produced by any method in the art, such as for example by crossing, selfing, backcrossing, etc. The process of producing progeny may be by natural or artificial means.

In some embodiments, the present disclosure relates to for the use of the plants of the present disclosure for growing plants in a field. In related embodiments, the present disclosure relates to for the use of the plants of the present disclosure for producing a plant oil or seed oil, such as for example plant or seed oils containing high levels of α-linolenic acid, eicosenoic acid, and tocopherols.

### DEPOSIT INFORMATION

A deposit of the seed of each of the Camelina sativa (L.) varieties disclosed herein is maintained by Linnaeus Plant Sciences, Inc., 2212-110 Gymnasium Place, Saskatoon, SK, S7N 0W9, Canada. In particular, and without limitation, a deposit of the seed of Camelina sativa (L.) varieties 12CS0365, 12CS0366, 12CS0389, 13CS0695, 13CS0781, 13CS0786, 14CS0851-01-14 and 17CS1115 is maintained by Linnaeus Plant Sciences, Inc., 2212-110 Gymnasium Place, Saskatoon, SK, S7N 0W9, Canada. In addition, a sample of the seed of Camelina sativa (L.) varieties 12CS0365, 12CS0366, 12CS0389 and 14CS0851-01-14 has been deposited by Linnaeus Plant Sciences, Inc. with American Type Culture Collection (ATCC), 10801 University Blvd. Manassas, VA, 20110-2209, USA, under ATCC Accession Nos. PTA-125493, PTA-125492, PTA-125494, and PTA-125495, respectively, on Dec. 3, 2018.

To satisfy the requirements of 35 U.S.C. § 112, and to certify that the deposit of the seeds of the present disclosure meets the criteria set forth in 37 C.F.R. § 1.801-1.809, Applicant hereby makes the following statements regarding the deposited seed of Camelina sativa (L.) varieties 12CS0365, 12CS0366, 12CS0389 and 14CS0851-01-14 (deposited as ATCC Accession Nos. PTA-125493, PTA-125492, PTA-125494, and PTA-125495, respectively, on Dec. 3, 2018):
1. During the pendency of this application, access to the seeds will be afforded to the Commissioner upon request;
2. Upon granting of the patent the strain will be available to the public under conditions specified in 37 CFR 1.808;
3. The deposit will be maintained in a public repository for a period of 30 years or 5 years after the last request or for the enforceable life of the patent, whichever is longer;
4. The viability of the biological material at the time of deposit will be tested; and
5. The deposit will be replaced if it should ever become unavailable.

Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the variety will be irrevocably removed by affording access to a deposit of at least 2,500 seeds of the same seed source with ATCC.

The following examples are provided to more fully describe the present disclosure and are presented for non-limiting illustrative purposes.

### EXAMPLES

### Example 1: Mutagenesis Procedure

An ethyl methanesulfonate (EMS) seed mutagenesis approach was pursued to develop a Group 2 herbicide-resistant camelina line.

EMS treatment: Camelina accession SRS 934, obtained from Plant Gene Resources of Canada, PGRC, was imbibed with the mutagen ethyl methanesulfonate (EMS) according to the teaching in Kim et al. (2006). Seeds (generation: M1) were imbibed in 100 mM phosphate buffer (pH 7.5) at 4° C. overnight. After decanting of the buffer, fresh buffer was added along with EMS to a final concentration of 0.4%. Seeds were then incubated at room temperature for 8 hours. Seeds were then washed thoroughly with water and immediately planted into soil in the field. Plants were covered with a tent for reproductive isolation and grown to maturity to produce M2 seed. M2 seed was bulk-harvested.

### Example 2: Selection for Herbicide Tolerance

Experiment #1: A bulk of camelina M2 seed from Example 1 was seeded on an area of 0.25 ha at the University of Alberta, Edmonton, and sprayed with Refine® SG (9.88 g active ingredient (ai)/ha thifensulfuron-methyl+4.94 g ai/ha tribenuron) at the 2-3 leaf stage at a ⅛th× field rate. Several hundred plants survived the treatment. 200 M2 plants were harvested individually and the M3 seed of each line was seeded in individual rows. M3 plants were treated with a 1× field rate Refine® SG and a clear difference between poorly performing and tolerant plants was observed. In total, 15 M3 plants survived and were harvested separately. M4 seed was sown in the greenhouse and sprayed with a 2× field rate of Refine® SG. 6 plants survived and were harvested separately (M5), then the seeds of all lines were mixed into one bulk (Linnaeus accession number: 11CS0111). All experiments were conducted at the U of A, Edmonton (Linda Hall).
Experiment #2: A bulk of camelina M2 seed from Example 1 was seeded on an area of 0.25 ha at the University of Alberta, Edmonton, and sprayed with a 2× field rate of Refine® SG (59.28 g product ha⁻¹, 19.76 g active ingredient (ai)/ha thifensulfuron-methyl+9.88 g ai/ha tribenuron) at the 2-3 leaf stage. Throughout the ensuing growing season, plants were monitored for the development of symptoms typical for Group 2 herbicide damage, such as stunting and chlorosis. 60 plants showed tolerance and were individually harvested and the M3 seeds sent to Saskatoon (Linnaeus Plant Sciences) to be re-tested under controlled conditions. For each line/population, 8 plants were seeded in trays and sprayed at the 3-4 leaf stage with a 1/128th rate of Refine® SG (0.0772 g ai/ha thifensulfuron-methyl+0.0386 g ai/ha tribenuron). In previous experiments, this rate was high enough to result in a reduction of biomass in an herbicide susceptible camelina variety of 75% after 14 daa. Plants were rated for symptoms, such as stunting and chlorosis at 7 daa. In total, 31 lines/populations showed a consistently tolerant phenotype in growth chamber screenings. 1 plant of each of the tolerant lines was transferred to the greenhouse for production of M4 seed. 4 M4 mutant lines with superior herbicide tolerance observed in the previous generation (M3) were selected for field evaluation (confined research field trials 13-LIN1-484-CAM01-1763-SK001-2 and 13-LIN1-484-CAM01-1763-SK003-01): 12CS0363, 12CS0364, 12CS0365, and 12CS0366, plus line 11CS0111 from Experiment #1.

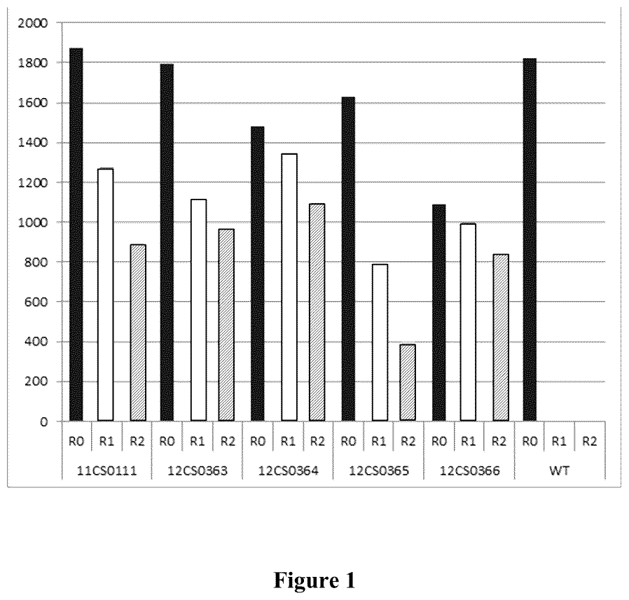

Seeds of all 5 lines were planted alongside a susceptible check (wild-type camelina SRS 934) in 20-foot plots in a randomized complete block design with 3 replicates. Plantlets were sprayed with a 0, 1× and 2× rate of Refine® SG, respectively, at the 3-4 leaf stage. Herbicide damage (stunting, chlorosis) was rated in 1-week intervals (7, 14, 21 daa, etc.). Increased tolerance to Refine® SG was confirmed for all 4 lines (see FIG. 1); however, the level of tolerance was not at a commercially acceptable level.

Crosses were conducted between all mutant lines and the F2 progeny was tested for segregation of 2 independent resistance genes (segregation of resistant: susceptible, 15:1). 6 F2 populations (including both reciprocal crosses) showed segregation of 2 resistance genes. 1 plant of each F2 population showing superior tolerance (combination of 2 resistance genes) was self-bagged to produce F3 seed. Subsequently, 20 F3 plants were grown and selfed in the greenhouse to produce F4 seed. A number of F3 families originating from each cross, were tested again for segregation and 6 F3 families were identified that showed no segregation, suggesting the combination of two resistance genes in a homozygous state. Two F3 families were advanced to the F4 generation in the greenhouse:

TABLE 3

| Cross | Accession number F₄ |
|---|---|
| 12CS0365 × 12CS0366 | 13CS0786 |
| 12CS0365 × 11CS0111 | 14CS0814 |

### Example 3: Camelina Line 14CS0851-01-14

Camelina line 14CS0851-01-14 was developed by crossing camelina mutant lines 12CS0365 (female) and 12CS0366 (male) from Example 2, and subsequent stabilizing of the trait through selfing. For hybridization, flower buds of 12CS0365 were opened, the anthers removed and pollen from 12CS0366 manually transferred onto the stigma of 12CS0365 plants. Pollinated buds were covered with crossing bags to avoid uncontrolled cross-pollination. F1 seeds were harvested and assigned accession number 12CS0389. F1 plants were bag-selfed and harvested. The F2 seed received accession number 13CS0695. F2 (13CS0695), F3 (13CS0781), and F4 (13CS0786) plants were bag-selfed and harvested accordingly. F5 seed received the accession number 14CS0851. A bulk of 14CS0851 that had been produced separately in the greenhouse received the accession number 14CS0851-01-14. The pedigree of camelina double mutant line 14CS0851-01-14 is shown below in Table 4.

TABLE 4
Pedigree of camelina double mutant line 14CS0851-01-14.

| 12CS0365 × 12CS0366 | P |
|---|---|
| 12CS0389 | F₁ |
| 13CS0695 | F₂ |
| 13CS0781 | F₃ |
| 13CS0786 | F₄ |
| 14CS0851-01-14 | F₅ |

### Example 4: Herbicide Resistance of Camelina Line 14CS0851-01-14

Camelina line 14CS0851-01-14, its parents 12CS0365 and 12CS0366 and susceptible check SRS 934 were planted in 20-foot plots in a randomized design with 4 replicates (confined research field trial 2014-ACS1-016-CAM01-1763-SK001-01). Plants were sprayed with a 1× and 2× rate of Refine® SG, respectively, at the 3-4 leaf stage and rated for herbicide injury symptoms in weekly intervals. The line carrying 2 mutant genes (14CS0851-01-14) exhibited a higher level of tolerance to the herbicide compared to the single mutated lines (12CS0365 and 12CS0366) and the wild-type (SRS 934); however, the level of tolerance of 14CS0851-01-14 was not commercially acceptable.

Based on these results, tolerance of 14CS0851-01-14 to a suite of 7 different Group 2 herbicides was tested (confined research field trial 15-ACS1-016-CAM01-1763-SK001-01). The 7 different Group 2 herbicides were: Refine® SG (tribenuron+thifensulfuron), Express (tribenuron), Pinnacle SG (thifensulfuron), Solo (imazamox), Frontline (fluorasulam), Everest (flucarbazone) and Pursuit (imazethapyr). The study included seeds of:
(i) 14CS0851-01-14
(ii) SRS 934 (wild-type)
(iii) 13CS0783-02 (F4, double mutant from cross: 12CS0363×12CS0365)
(iv) 13CS0784-02 (F4, double mutant from cross: 12CS0363×12CS0366)
(v) 13CS0785-02 (F4, double mutant from cross: 12CS0364×12CS0365)
(vi) 13CS0787-02 (F4, double mutant from cross: 12CS0364×12CS0363)
(vii) 14CS0852-01-12 (F5, double mutant from cross: 12CS0365×11CS0111)

Seeds of 14CS0851-01-14 alongside a susceptible check (SRS 934) and the five other double mutants were seeded in 2-row 20-foot plots, in 3 replicates for each chemical (1 range per chemical). At the 3-4 leaf stage, for each range (chemical), the front and the back of the plot (each approximately 6 feet) were sprayed with a 1× and 2× rate, respectively, while the middle 6 feet of each plot were left untreated. Herbicide damage was monitored in weekly intervals. Superior and commercially acceptable levels of tolerance to thifensulfuron-methyl (Pinnacle® SG) were observed for line 14CS0851-01-14. 14CS0851-01-14 also exhibited commercially acceptable levels of tolerance to flucarbazone (herbicide Everest® 2.0) in the same confined research field trial (Tables 5 and 6).

TABLE 5
Observed herbicide tolerance to Pinnacle SG and Everest

| | | | Original | | Observed Herbicide |
|---|---|---|---|---|---|
| Plot | Rep | Accession # | Cross | Chemical | Tolerance |
| 115 | 1 | wild-type | 1 | Pinnacle SG | Dead |
| 116 | 1 | 13CS0784-02 | 12CS0363 × 12CS0366 | Pinnacle SG | Very good |
| 117 | 1 | 13CS0783-02 | 12CS0363 × 12CS0365 | Pinnacle SG | Affected at both rates |
| 118 | 1 | 14CS0852-01-12 | 12CS0365 × 11CS0111 | Pinnacle SG | Very good |
| 119 | 1 | 13CS0785-02 | 12CS0364 × 12CS0365 | Pinnacle SG | Very good |
| 120 | 1 | 14CS0851-01-14 | 12CS0365 × 12CS0366 | Pinnacle SG | Very good |
| 121 | 1 | 13CS0787-02 | 12CS0364 × 12CS0363 | Pinnacle SG | Very good |
| 215 | 2 | 14CS0851-01-14 | 12CS0365 × 12CS0366 | Pinnacle SG | Very good |
| 216 | 2 | 13CS0783-02 | 12CS0363 × 12CS0365 | Pinnacle SG | Affected at both rates |
| 217 | 2 | 13CS0785-02 | 12CS0364 × 12CS0365 | Pinnacle SG | Slightly affected at 2x |
| 218 | 2 | 14CS0852-01-12 | 12CS0365 × 11CS0111 | Pinnacle SG | Very good |
| 219 | 2 | 13CS0787-02 | 12CS0364 × 12CS0363 | Pinnacle SG | Slightly affected at 2x |
| 220 | 2 | 13CS0784-02 | 12CS0363 × 12CS0366 | Pinnacle SG | Slightly affected at 2x |
| 221 | 2 | wild-type | / | Pinnacle SG | Dead |
| 315 | 3 | 13CS0783-02 | 12CS0363 × 12CS0365 | Pinnacle SG | Affected at both rates |
| 316 | 3 | wild-type | / | Pinnacle SG | Dead |
| 317 | 3 | 13CS0784-02 | 12CS0363 × 12CS0366 | Pinnacle SG | Slightly affected at 2x |
| 318 | 3 | 13CS0787-02 | 12CS0364 × 12CS0363 | Pinnacle SG | Slightly affected at 2x |
| 319 | 3 | 14CS0851-01-14 | 12CS0365 × 12CS0366 | Pinnacle SG | Very good |
| 320 | 3 | 14CS0852-01-12 | 12CS0365 × 11CS0111 | Pinnacle SG | Very good |
| 321 | 3 | 13CS0785-02 | 12CS0364 × 12CS0365 | Pinnacle SG | Slightly affected at 2x |
| 136 | 1 | 13CS0784-02 | 12CS0363 × 12CS0366 | Everest | Very good |
| 137 | 1 | 13CS0785-02 | 12CS0364 × 12CS0365 | Everest | Slightly affected at 2x |
| 138 | 1 | 14CS0851-01-14 | 12CS0365 × 12CS0366 | Everest | Very good |
| 139 | 1 | 13CS0787-02 | 12CS0364 × 12CS0363 | Everest | Slightly affected at 2x |
| 140 | 1 | 14CS0852-01-12 | 12CS0365 × 11CS0111 | Everest | Very good |
| 141 | 1 | 13CS0783-02 | 12CS0363 × 12CS0365 | Everest | Affected at both rates |
| 142 | 1 | wild-type | / | Everest | Dead |
| 236 | 2 | wild-type | / | Everest | Dead |
| 237 | 2 | 14CS0852-01-12 | 12CS0365 × 11CS0111 | Everest | Very good |
| 238 | 2 | 14CS0851-01-14 | 12CS0365 × 12CS0366 | Everest | Very good |
| 239 | 2 | 13CS0785-02 | 12CS0364 × 12CS0365 | Everest | Good to slightly |
| | | | | | affected at 2x |
| 240 | 2 | 13CS0787-02 | 12CS0364 × 12CS0363 | Everest | Slightly affected at 2x |
| 241 | 2 | 13CS0784-02 | 12CS0363 × 12CS0366 | Everest | Slightly affected at 2x |
| 242 | 2 | 13CS0783-02 | 12CS0363 × 12CS0365 | Everest | Affected at both rates |
| 336 | 3 | 14CS0852-01-12 | 12CS0365 × 11CS0111 | Everest | Very good |
| 337 | 3 | wild-type | / | Everest | Dead |
| 338 | 3 | 13CS0785-02 | 12CS0364 × 12CS0365 | Everest | Good to slightly |
| | | | | | affected at 2x |
| 339 | 3 | 13CS0787-02 | 12CS0364 × 12CS0363 | Everest | Good |
| 340 | 3 | 13CS0783-02 | 12CS0363 × 12CS0365 | Everest | Affected at both rates |
| 341 | 3 | 13CS0784-02 | 12CS0363 × 12CS0366 | Everest | Slightly affected at 2x |
| 342 | 3 | 14CS0851-01-14 | 12CS0365 × 12CS0366 | Everest | Very good |

TABLE 6
Weights of plants in response to application
of Pinnacle SG and Everest

| | | Accession | Weight | SUM |
|---|---|---|---|---|
| Plot | Herbicide | number | (g) | (g) |
| 115 | Pinnacle SG | wild-type | 332.4 | |
| 142 | Everest | wild-type | 295.0 | |
| 221 | Pinnacle SG | wild-type | 519.3 | |
| 236 | Everest | wild-type | 356.0 | |
| 316 | Pinnacle SG | wild-type | 665.0 | |
| 337 | Everest | wild-type | 417.9 | 2585.6 |
| 121 | Pinnacle SG | 13CS0787-02 | 1044.3 | |
| 139 | Everest | 13CS0787-02 | 1071.8 | |
| 219 | Pinnacle SG | 13CS0787-02 | 1123.3 | |
| 240 | Everest | 13CS0787-02 | 1121.9 | |
| 318 | Pinnacle SG | 13CS0787-02 | 1201.6 | |
| 339 | Everest | 13CS0787-02 | 968.7 | 6531.6 |
| 117 | Pinnacle SG | 13CS0783-02 | 1120.0 | |
| 141 | Everest | 13CS0783-02 | 805.5 | |
| 216 | Pinnacle SG | 13CS0783-02 | 1046.8 | |
| 242 | Everest | 13CS0783-02 | 1005.1 | |
| 315 | Pinnacle SG | 13CS0783-02 | 955.3 | |
| 340 | Everest | 13CS0783-02 | 1121.7 | 6054.4 |
| 116 | Pinnacle SG | 13CS0784-02 | 1024.0 | |
| 136 | Everest | 13CS0784-02 | 928.8 | |
| 220 | Pinnacle SG | 13CS0784-02 | 930.2 | |
| 241 | Everest | 13CS0784-02 | 896.7 | |
| 317 | Pinnacle SG | 13CS0784-02 | 1235.1 | |
| 341 | Everest | 13CS0784-02 | 942.2 | 5957.0 |
| 119 | Pinnacle SG | 13CS0785-02 | 1016.3 | |
| 137 | Everest | 13CS0785-02 | 946.0 | |
| 217 | Pinnacle SG | 13CS0785-02 | 1014.4 | |
| 239 | Everest | 13CS0785-02 | 783.1 | |
| 321 | Pinnacle SG | 13CS0785-02 | 978.7 | |
| 338 | Everest | 13CS0785-02 | 935.7 | 5674.2 |
| 120 | Pinnacle SG | 14CS0851-01-14 | 997.9 | |
| 138 | Everest | 14CS0851-01-14 | 1053.2 | |
| 215 | Pinnacle SG | 14CS0851-01-14 | 1036.6 | |
| 238 | Everest | 14CS0851-01-14 | 967.7 | |
| 319 | Pinnacle SG | 14CS0851-01-14 | 1186.5 | |
| 342 | Everest | 14CS0851-01-14 | 898.0 | 6139.9 |
| 118 | Pinnacle SG | 14CS0852-01-12 | 587.3 | |
| 140 | Everest | 14CS0852-01-12 | 568.7 | |
| 218 | Pinnacle SG | 14CS0852-01-12 | 582.0 | |
| 237 | Everest | 14CS0852-01-12 | 592.6 | |
| 320 | Pinnacle SG | 14CS0852-01-12 | 718.1 | |
| 336 | Everest | 14CS0852-01-12 | 410.3 | 3459.0 |
| Waste | | | 1261.8 | |

The results from the field trial were confirmed in greenhouse experiments using line 14CS0851-01-14, the parent lines (12CS0365 and 12CS0366) and the wild-type (SRS 934).

### Example 5: Inheritance and Stability of the Herbicide Trait Across Generations

To determine the genetic control of resistance to thifensulfuron, reciprocal crosses between herbicide-susceptible camelina cultivar 10CS0048 (MIDAS™) and mutant lines 12CS0365 and 12CS0366, respectively, were made. From each cross combination, randomly selected F1 plants were selfed for the development of F2 populations segregating for 1 resistance gene.

Further, mutant lines 12CS0365 and 12CS0366 were crossed with each other and a randomly selected F1 plant selfed for the development of an F2 population segregating for 2 independent resistance genes. F4 progeny of F2 plants homozygous for both resistance genes (13CS0786) was backcrossed to 10CS0048 to form a backcross (BC1F1) population. Further backcrossing to the recurrent parent 10CS0048 was conducted until stabilization of the trait in the BC4F3 generation (homozygous for 2 resistance genes).

At a thifensulfuron-methyl application rate of 2.5 g ai/ha, plants from parental, F2, BC1F1, and BC4F2 populations could easily be scored into one of two discrete phenotypic classes (R, resistant or S, susceptible) 7 d after herbicide application. Resistant lines (12CS0365 or 12CS0366 for test of segregation in F2 population segregating for 1 resistance gene; 13CS0786 for test of segregation in F2, BC1F1 and BC4F2 populations segregating for 2 resistance genes) were used as controls in all experiments and consistently produced a resistant phenotype when sprayed with 2.5 g ai/ha of thifensulfuron-methyl. In all experiments, susceptible controls (SRS 934, 10CS0048) were either killed or greatly damaged by application of thifensulfuron at 7 d after application (daa).

Assuming inheritance in a Mendelian manner, the expected genotypic segregation ratio in an F2 population segregating for a single resistance gene would be 1(RR): 2(Rr): 1(rr), or 3(RR, Rr): 1(rr).

If two unlinked genes for resistance are segregating in an F2 population, the expected genotypic segregation ratio would be 9 (R1-R2-): 2 (R1r1r2r2): 2 (r1r1R2r2): 1 (R1R1r2r2): 1 (r1r1R2R2): 1 (r1r1r2r2), or 15 (R1-R2-, R1r1r2r2, r1r1R2r2, R1R1r2r2, r1r1R2R2): 1 (r1r1r2r2). The expected genotypes in the BC1F1 population would be R1r1R2r2, R1r1r2r2, r1r1R2r2, and r1r1r2r2, each produced in equal frequency, resulting in a segregation ratio of 1 (R1r1R2r2): 2 (R1r1r2r2, r1r1R2r2): 1 (r1r1r2r2), or 3 (R1r1R2r2, R1r1r2r2, r1r1R2r2): 1 (r1r1r2r2). The expected segregation ratio in the BC4F2 generation would be the same as that observed in the F2 population.

The F2 population resulting from the reciprocal cross of 10CS0048 with 12CS0365 and 12CS0366, respectively, the F2 population resulting from the cross 12CS0365/12CS0366 and the BC1F1 and BC4F2 populations resulting from the cross of F4 plants homozygous for 2 resistance genes (13CS0786) with recurrent parent 10CS0048, gave good fit to the expected segregation ratios (Table 7).

TABLE 7
Evaluation of resistance to thifensulfuron-methyl in different families and Chi-
square test of single-locus and two-locus models for control of resistance.

| | | Resistant | Susceptible | Ratio tested | X² |
|---|---|---|---|---|---|
| Cross | Population | (R) | (S) | (R:S) | P value^{†} |
| 12CS0365 × | F₂ | 555 (568)^{‡} | 202 (189) | 3:1 | 0.28 |
| 10CS0048 | | | | | |
| (reciprocal) | | 549 (540) | | 3:1 | 0.44 |
| 12CS0366 × | F₂ | | 171 (180) | | |
| 10CS0048 | | | | | |
| (reciprocal) | | 307 (306.6) | | 15:1 | 0.92 |
| 12CS0365 × | F₂ | | 20 (20.4) | | |
| 12CS0366 | | | | | |
| 10CS0048 × | BC₁F₁ | 27 (27) | 9 (9) | 3:1 | 1.0 |
| 13CS0786 | | | | | |
| 10CS0048 × | BC₄F₂ | 147 (147.2) | 10 (9.8) | 15:1 | 0.95 |
| 13CS0786 | | | | | |

†Chi-square P value representing the probability that deviations from the expected ratio are due to chance alone. Chi-square P values greater than 0.05 indicate that observed values were not significantly different from expected values.
^{‡}Values in brackets present the number of plants expected in each phenotypic class based on the segregation ratio tested.

### Example 6: Characterization of the Herbicide Resistance Trait

In order to characterize the herbicide resistance trait, a dose-response experiment was conducted in the greenhouse, comparing the response of SRS 934 and camelina mutant lines to increasing levels of thifensulfuron-methyl with regards to reduction in plant height and biomass, respectively.

The camelina lines used were the wild-type SRS 934 and mutant lines 12CS0365, 12CS0366, and 13CS0786 (direct progenitor of line 14CS0851-01-14). The wild-type is the original germplasm that was mutated through EMS to produce lines 12CS0365 and 12CS0366, each of which has one resistant AHAS gene. As described above, these two lines were then crossed to produce 13CS0786 (2 resistance genes, selfing of 13CS0786 yielded seeds of 14CS0851-01-14).

Seeds of all lines were planted in 6-cm diameter pots at ½ cm deep in soilless media. Plants were grown in a greenhouse with a 16-hour photoperiod. At the 3-4 leaf stage, ten different treatments of thifensulfuron-methyl were applied to the four lines: 0, 0.08, 0.24, 0.72, 2.2, 6.5, 19.4, 58.3, 175, and 525 g ai/ha, respectively. All plants were sprayed in a spray chamber at a volume of 200 L/ha. AgSurf II adjuvant was added at a rate of 1 mL per 1 L of spray solution. Treatments were arranged in a randomized complete block with 4 replications. A total of 640 plants were used (4 camelina lines, with 4 plants per line and treatment, 4 replications). Plants were harvested 21 days after application of thifensulfuron-methyl. Individual plant heights were measured using a ruler and plants placed in a paper bag. Plants were dried for at least 24 hours at 60° C. to ensure that all water was removed from the plant tissue. Plants were then weighed individually to measure above-ground dry biomass.

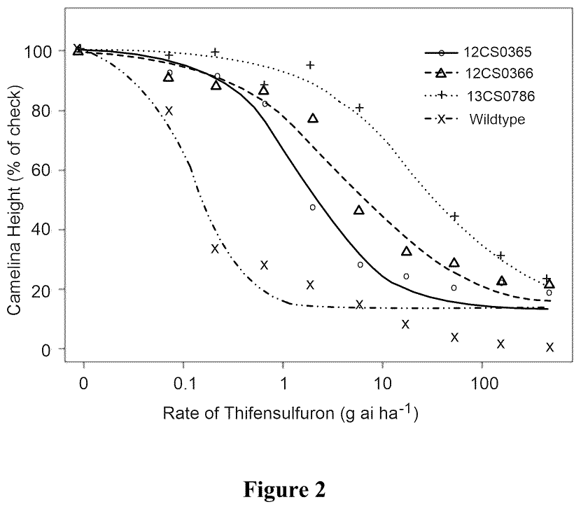

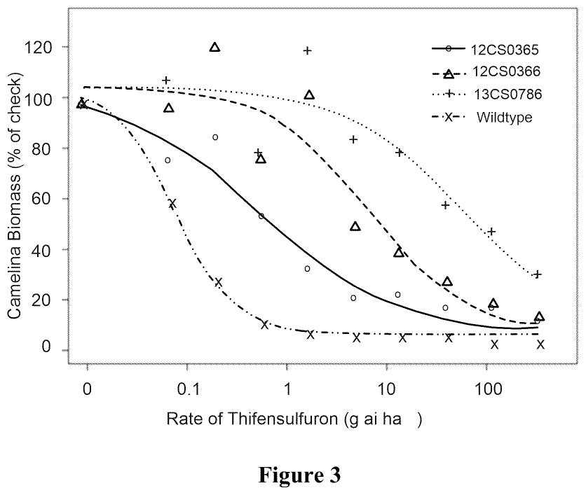

For both height and biomass, a dose-response curve was established and an ED₅₀ value determined (ED₅₀ height and ED₅₀ biomass). The ED₅₀ is the dose required to affect plant response 50% relative to the upper and lower limit. It is also known as the point of inflection which is the point where the dose-response curve switches from a concave to a convex orientation. ED₅₀ height values are shown in Table 8 and ED₅₀ biomass values are shown in Table 9. Dose-response curves for height and biomass are shown in FIGS. 2 and 3, respectively.

TABLE 8
ED₅₀ height values for camelina lines 21 days after
application of thifensulfuron-methyl at rates
ranging from 0 to 525 g ai/ha (n = 16).

| | ED₅₀ height | Standard |
|---|---|---|
| Entry | (g ai/ha) | error |
| SRS 934 | 0.14 | 0.02 |
| 12CS0365 | 1.81 | 0.37 |
| 12CS0366 | 5.25 | 1.35 |
| 13CS0786 | 26.36 | 6.63 |

TABLE 9
ED₅₀ biomass values for camelina lines 21 days
after application of thifensulfuron-methyl at
rates ranging from 0 to 525 g ai/ha (n = 16).

| | ED₅₀ biomass | Standard |
|---|---|---|
| Entry | (g ai/ha) | error |
| SRS 934 | 0.09 | 0.02 |
| 12CS0365 | 0.69 | 0.22 |
| 12CS0366 | 8.67 | 2.32 |
| 13CS0786 | 89.61 | 27.0 |

Ratios of ED₅₀ values for plant height between lines are shown in Table 10. The p-values indicate that the level of thifensulfuron-methyl resistance is significantly different between all four lines when considering reduction of height after herbicide application. 13CS0786 proved to be significantly more resistant than the other three lines with approximately 185× the resistance level of SRS 934.

TABLE 10
Ratio of ED₅₀ height values between each of the
tested lines (n = 16), F values and p values.

| Genotype comparison | estimate | F value | p value |
|---|---|---|---|
| 12CS0365/12CS0366 | 0.35 | −6.61 | <0.0001 |
| 12CS0365/13CS0786 | 0.02 | −47.66 | <0.0001 |
| 12CS0365/SRS 934 | 12.72 | 3.91 | 0.0005 |
| 12CS0366/13CS0786 | 0.20 | −13.02 | <0.0001 |
| 12CS0366/SRS 934 | 36.9 | 3.54 | 0.0013 |
| 13CS0786/SRS 934 | 185.36 | 3.67 | 0.0009 |

Table 11 compares the ratio of ED₅₀ biomass values between each of the tested lines. Again, the p-values indicate that there is a significant difference in resistance to thifensulfuron-methyl between all four lines when evaluated based on reduction of biomass. 13CS0786 proved to be significantly more resistant than the other lines and was approximately 1000× more resistant than the susceptible line SRS 934.

TABLE 11
Ratio of ED₅₀ biomass values between each of the
tested lines (n = 16), F values and p values.

| Genotype comparison | estimate | F value | p value |
|---|---|---|---|
| 12CS0365/12CS0366 | 0.08 | −32.05 | <0.0001 |
| 12CS0365/13CS0786 | 0.008 | −334.97 | <0.0001 |
| 12CS0365/SRS 934 | 7.93 | 2.38 | 0.0184 |
| 12CS0366/13CS0786 | 0.0968 | −26.60 | <0.0001 |
| 12CS0366/SRS 934 | 99.70 | 3.04 | 0.0028 |
| 13CS0786/SRS 934 | 1030.40 | 2.83 | 0.0053 |

### Example 7: Replicated Herbicide Tolerance Field Trials

Field trials of 14CS0851-01-14 along with parent line SRS 934 and commercial variety MIDAS™ were also completed in 5 locations in 2016 and 3 locations in 2017 using 3 rates of thifensulfuron-methyl: 0, 6, and 12 grams of active ingredient/ha, corresponding to 0, 1× and 2× label rate. Of the 5 locations in 2016, data for 4 are provided below. Data for Box Elder is not included due to presence of Roundup™ herbicide contamination.

### Protocol:

1. Trial design: RCBD or split plot.

TABLE 12
Treatment list:

| Treatment ID | Camelina Variety | Herbicide Treatment |
|---|---|---|
| 1 | Midas | Untreated Control |
| 2 | Midas | 1x Pinnacle SG* |
| | | (thifensulfuron-methyl) |
| 3 | Midas | 2x Pinnacle SG** |
| | | (thifensulfuron-methyl) |
| 4 | Parent (SRS934) | Untreated Control |
| 5 | Parent (SRS934) | 1x Pinnacle SG* |
| | | (thifensulfuron-methyl) |
| 6 | Parent (SRS934) | 2x Pinnacle SG** |
| | | (thifensulfuron-methyl) |
| 7 | 14CS0851-01-14 | Untreated Control |
| 8 | 14CS0851-01-14 | 1x Pinnacle SG* |
| | | (thifensulfuron-methyl) |
| 9 | 14CS0851-01-14 | 2x Pinnacle SG** |
| | | (thifensulfuron-methyl) |

*Pinnacle SG, 1x rate: 4.8 gr. product/acre or 6.0 gr. active ingredient/ha
**Pinnacle SG, 2x rate: 9.6 gr. product/acre or 12.0 gr. active ingredient/ha

a. # of Replicates: 4
b. Randomization provided by: Contractor
c. Guard seed provided by: Linnaeus
d. Plot size (m²): To be chosen by contractor
e. Seed packaging provided by: Linnaeus
2. Fertilizer: As per recent soil test, recommendations for 40 bu/ac canola.
3. Site Preparation:
   a. Prepare weed-free trial site with seedbed suitable for small seed (shallow placement). Select a site that has not had any Group 2 herbicides in the past 2 years of cropping history, or significant history of other potentially residual Group 2 active ingredients e.g. chemical families Imidazolinones, Sulfonylaminocarbonyltriazolinones, Amides, Sulfonylureas, Pyrazoles, Triazolpyramidines, and Triazolones.
   b. Select a site with cereal stubble that had good weed control in 2015. For disease management purposes, avoid a site that had Brassica crops in 2014 or 2015.
   c. Note that Kochia may be difficult to control; select a site where Kochia is not a problem.
   d. Apply Treflan or Edge (ethalfluralin or trifluralin) and incorporate prior to seeding with fertilizer.
4. Site Maintenance:
   a. Grassy weeds: Any Group 1 product registered on canola. Assure II is preferred, as it has Minor Use registration for use on camelina.
   b. Broadleaf weeds: Hand weed as needed (at least once at herbicide application timing, once later).
5. Ratings and Rating Scales on all plots:
   a. Plant stand (% plot fill, ˜3-4 leaf stage)
   b. Plant vigour (1-5, 1 is poorest, 5 is best, ˜3-4 leaf stage)
   c. Days to first flowering (days after planting when 10% of plants have one or more open flower, assessed 3× weekly)
   d. Days to 50% flowering (days after planting when 50% of flowers have opened, assessed 3× weekly)
   e. Days to end of flowering (days after planting when no flowers remain open, assessed 3× weekly)
   f. Days to maturity (days after planting when 50% of the plant has changed color on average across the plot, assessed 3× weekly)
   g. Plant height at maturity (cm, to top of plants)
   h. Herbicide injury rating (primary effect is stunting, assess as per scale shown below, at intervals listed, is shown in Table 13.

TABLE 13

| Percent Stunting (%) | Control | Interval Size |
|---|---|---|
| 0-9 | Slight stunting | 2% |
| 10 | Just acceptable | 0% |
| 11-30 | Not acceptable | 5% |
| >60 | Severe | 10% |

i. 7-14 days after Pinnacle SG application
ii. 21-35 days after Pinnacle SG application
iii. 42-56 days after Pinnacle SG application
i. Yield (g/plot)
j. Grain moisture (%)
k. Yield (kg/ha) adjusted for grain moisture
5. Ratings and Rating Scales on Untreated Control plots only:
   a. Biotic and abiotic stress (at seedling stage, rosette stage, bolt/flowering and pre-maturity stage). 0-10 scale (0 is no effect from stressor, 10 is dead/dying from stressor).
      i. Biotic stressors include insect pests and diseases (downy mildew, aster yellows, sclerotinia stem rot)
      ii. Abiotic stressors include excess moisture, drought, heat, cold, salinity, etc.
6. Harvesting: Plants should be straight combined. Maturity times are similar to B. rapa. Camelina pods are generally ready before the plants appear to be ready to harvest. As soon as the plants start to senesce, start checking the pods for dry yellow-brown seeds. Shattering may occur if harvest is delayed. Spraying a desiccant is recommended to dry down the stems if necessary to facilitate combining. Reglone (diquat) can be used at the recommended rate for desiccation at a high water volume.

### Results:

The results of these trials demonstrating the level of herbicide injury are shown below in Tables 14-20. Additional phenotypic characteristics as described above were measured and recorded (data not shown).

TABLE 14
Field Trial Data at Morris, MN in 2016.

| | | Herbicide injury (% stunting) | | |
|---|---|---|---|---|
| Line | Treatment | 13 daa | 29 daa | 46 daa |
| SRS 934 | 1x | 90 | 20 | 20 |
| SRS 934 | 2x | 90 | 35 | 35 |
| SRS 934 | 0 | Control | | |
| 14CS0851-01-14 | 1x | 5 | 15 | 2 |
| 14CS0851-01-14 | 0 | Control | | |
| 14CS0851-01-14 | 2x | 3 | 20 | 10 |
| Midas | 0 | Control | | |
| Midas | 1x | 80 | 45 | 20 |
| Midas | 2x | 90 | 60 | 40 |
| Midas | 2x | 90 | 70 | 40 |
| Midas | 0 | Control | | |
| Midas | 1x | 80 | 45 | 35 |
| SRS 934 | 0 | Control | | |
| SRS 934 | 1x | 80 | 20 | 15 |
| SRS 934 | 2x | 90 | 20 | 20 |
| 14CS0851-01-14 | 0 | Control | | |
| 14CS0851-01-14 | 1x | 3 | 3 | 2 |
| 14CS0851-01-14 | 2x | 15 | 10 | 15 |
| SRS 934 | 0 | Control | | |
| SRS 934 | 1x | 80 | 30 | 40 |
| SRS 934 | 2x | 90 | 30 | 40 |
| 14CS0851-01-14 | 1x | 0 | 10 | 10 |
| 14CS0851-01-14 | 2x | 11 | 9 | 35 |
| 14CS0851-01-14 | 0 | Control | | |
| Midas | 1x | 80 | 45 | 30 |
| Midas | 0 | Control | | |
| Midas | 2x | 85 | 55 | 40 |
| 14CS0851-01-14 | 1x | 11 | 10 | 10 |
| 14CS0851-01-14 | 2x | 30 | 12 | 15 |
| 14CS0851-01-14 | 0 | Control | | |
| Midas | 0 | Control | | |
| Midas | 2x | 90 | 60 | 40 |
| Midas | 1x | 70 | 45 | 40 |
| SRS 934 | 2x | 90 | 45 | 20 |
| SRS 934 | 0 | Control | | |
| SRS 934 | 1x | 80 | 40 | 20 |

TABLE 15
Field Trial Data at Huntley, MT in 2016.

| | | Herbicide | Herbicide | Herbicide | Herbicide |
|---|---|---|---|---|---|
| | | Injury (%) | Injury (%) | Injury (%) | Injury (%) |
| Line | Treatment | 9 daa | 22 daa | 37 daa | 61 daa |
| SRS 934 | 1x | 60 | 90 | 85 | 80 |
| SRS 934 | 2x | 70 | 85 | 80 | 80 |
| SRS 934 | 0 | 0 | 0 | 0 | 0 |
| 14CS0851-01-14 | 0 | 0 | 0 | 0 | 0 |
| 14CS0851-01-14 | 1x | 0 | 0 | 0 | 0 |
| 14CS0851-01-14 | 2x | 0 | 0 | 0 | 0 |
| Midas | 1x | 55 | 90 | 85 | 80 |
| Midas | 0 | 0 | 0 | 0 | 0 |
| Midas | 2x | 75 | 95 | 90 | 85 |
| 14CS0851-01-14 | 0 | 0 | 0 | 0 | 0 |
| 14CS0851-01-14 | 1x | 0 | 0 | 0 | 0 |
| 14CS0851-01-14 | 2x | 0 | 0 | 0 | 0 |
| Midas | 1x | 50 | 90 | 85 | 80 |
| Midas | 0 | 0 | 0 | 0 | 0 |
| Midas | 2x | 70 | 95 | 85 | 80 |
| SRS 934 | 0 | 0 | 0 | 0 | 0 |
| SRS 934 | 1x | 50 | 80 | 75 | 70 |
| SRS 934 | 2x | 75 | 90 | 80 | 80 |
| Midas | 0 | 0 | 0 | 0 | 0 |
| Midas | 2x | 75 | 95 | 90 | 85 |
| Midas | 1x | 45 | 90 | 85 | 80 |
| SRS 934 | 0 | 0 | 0 | 0 | 0 |
| SRS 934 | 2x | 65 | 90 | 85 | 80 |
| SRS 934 | 1x | 55 | 85 | 85 | 80 |
| 14CS0851-01-14 | 1x | 0 | 0 | 0 | 0 |
| 14CS0851-01-14 | 0 | 0 | 0 | 0 | 0 |
| 14CS0851-01-14 | 2x | 0 | 0 | 0 | 0 |
| SRS 934 | 1x | 65 | 90 | 90 | 85 |
| SRS 934 | 2x | 65 | 90 | 85 | 80 |
| SRS 934 | 0 | 0 | 0 | 0 | 0 |
| 14CS0851-01-14 | 0 | 0 | 0 | 0 | 0 |
| 14CS0851-01-14 | 1x | 0 | 0 | 0 | 0 |
| 14CS0851-01-14 | 2x | 0 | 0 | 0 | 0 |
| Midas | 1x | 50 | 85 | 85 | 80 |
| Midas | 0 | 0 | 0 | 0 | 0 |
| Midas | 2x | 80 | 90 | 90 | 85 |

TABLE 16
Field Trial Data at North Dakota State University (Fargo, ND) in 2016.

| Crop Name | | | | | | | Camelina | Camelina | Camelina |
|---|---|---|---|---|---|---|---|---|---|
| Rating Date | | | | | | | Jul. 2, 2016 | Jul. 22, 2016 | Aug. 1, 2016 |
| | | | | | | | (9 daa) | (29 daa) | (39 daa) |
| Rating Type | | | | | | | Stunt | Stunt | Stunt |
| Rating Unit | | | | | | | Percent | Percent | Percent |
| Number of Decimals | | | | | | | 0 | 0 | 0 |
| Trt | Treatment | | Rate | Growth | Appl | | | | |
| No. | Name | Rate | Unit | Stage | Code | Plot | | | |
| 1 | 14CS0851- | | | | | 108 | 0 | 0 | 0 |
| | 01-14 | | | | | | | | |
| | Untreated | | | | | 204 | 0 | 0 | 0 |
| | | | | | | 312 | 0 | 0 | 0 |
| | | | | | | 402 | 0 | 0 | 0 |
| Mean = | | | | | | | 0 | 0 | 0 |
| 2 | 14CS0851- | | | | | 105 | 20 | 4 | 6 |
| | 01-14 | | | | | | | | |
| | Harmony | 6 | g | 3-4 leaf | A | 203 | 25 | 4 | 4 |
| | | | ai/ha | | | | | | |
| | NIS | 0.1 | % v/v | 3-4 leaf | A | 311 | 15 | 4 | 4 |
| | | | | | | 401 | 25 | 4 | 4 |
| Mean = | | | | | | | 21 | 4 | 5 |
| 3 | 14CS0851- | | | | | 106 | 30 | 8 | 10 |
| | 01-14 | | | | | | | | |
| | Harmony | 12 | g | 3-4 leaf | A | 201 | 30 | 8 | 8 |
| | | | ai/ha | | | | | | |
| | NIS | 0.1 | % v/v | 3-4 leaf | A | 310 | 25 | 8 | 8 |
| | | | | | | 404 | 30 | 8 | 8 |
| Mean = | | | | | | | 29 | 8 | 9 |
| 4 | 14CS0851- | | | | | 107 | 40 | 10 | 15 |
| | 01-14 | | | | | | | | |
| | Harmony | 24 | g | 3-4 leaf | A | 202 | 50 | 10 | 10 |
| | | | ai/ha | | | | | | |
| | NIS | 0.1 | % v/v | 3-4 leaf | A | 309 | 40 | 10 | 10 |
| | | | | | | 403 | 50 | 10 | 10 |
| Mean = | | | | | | | 45 | 10 | 11 |
| 5 | SRS 934 | | | | | 102 | 0 | 0 | 0 |
| | Untreated | | | | | 205 | 0 | 0 | 0 |
| | | | | | | 301 | 0 | 0 | 0 |
| | | | | | | 407 | 0 | 0 | 0 |
| Mean = | | | | | | | 0 | 0 | 0 |
| 6 | SRS 934 | | | | | 103 | 50 | 50 | 50 |
| | Harmony | 6 | g | 3-4 leaf | A | 207 | 60 | 70 | 70 |
| | | | ai/ha | | | | | | |
| | NIS | 0.1 | % v/v | 3-4 leaf | A | 302 | 50 | 60 | 60 |
| | | | | | | 405 | 60 | 70 | 70 |
| Mean = | | | | | | | 55 | 63 | 63 |
| 7 | SRS 934 | | | | | 101 | 60 | 60 | 60 |
| | Harmony | 12 | g | 3-4 leaf | A | 208 | 70 | 80 | 80 |
| | | | ai/ha | | | | | | |
| | NIS | 0.1 | % v/v | 3-4 leaf | A | 303 | 60 | 70 | 70 |
| | | | | | | 406 | 70 | 80 | 80 |
| Mean = | | | | | | | 65 | 73 | 73 |
| 8 | SRS 934 | | | | | 104 | 70 | 80 | 80 |
| | Harmony | 24 | g | 3-4 leaf | A | 206 | 70 | 90 | 90 |
| | | | ai/ha | | | | | | |
| | NIS | 0.1 | % v/v | 3-4 leaf | A | 304 | 70 | 90 | 90 |
| | | | | | | 408 | 70 | 90 | 95 |
| Mean = | | | | | | | 70 | 88 | 89 |
| 9 | Midas | | | | | 110 | 0 | 0 | 0 |
| | Untreated | | | | | 212 | 0 | 0 | 0 |
| | | | | | | 308 | 0 | 0 | 0 |
| | | | | | | 410 | 0 | 0 | 0 |
| Mean = | | | | | | | 0 | 0 | 0 |
| 10 | Midas | | | | | 109 | 50 | 60 | 60 |
| | Harmony | 6 | g | 3-4 leaf | A | 211 | 50 | 60 | 60 |
| | | | ai/ha | | | | | | |
| | NIS | 0.1 | % v/v | 3-4 leaf | A | 306 | 50 | 60 | 60 |
| | | | | | | 409 | 60 | 70 | 65 |
| Mean = | | | | | | | 53 | 63 | 61 |
| 11 | Midas | | | | | 112 | 60 | 90 | 90 |
| | Harmony | 12 | g | 3-4 leaf | A | 209 | 60 | 70 | 70 |
| | | | ai/ha | | | | | | |
| | NIS | 0.1 | % v/v | 3-4 leaf | A | 305 | 60 | 80 | 80 |
| | | | | | | 411 | 70 | 80 | 80 |
| Mean = | | | | | | | 63 | 80 | 80 |
| 12 | Midas | | | | | 111 | 60 | 90 | 90 |
| | Harmony | 24 | g | 3-4 leaf | A | 210 | 70 | 90 | 90 |
| | | | ai/ha | | | | | | |
| | NIS | 0.1 | % v/v | 3-4 leaf | A | 307 | 70 | 90 | 90 |
| | | | | | | 412 | 80 | 90 | 95 |

| | | | |
|---|---|---|---|
| Mean = | 70 | 90 | 91 |

TABLE 17
Field Trial Data at Taber, AB in 2016.

| | | | | Herbicide injury | | | |
|---|---|---|---|---|---|---|---|
| | | | | (% stunting) | | | |
| | | | | 7-14 | 21-35 | 42-56 | |
| Plot | Rep | Line | Treatment | daa | daa | daa | Comments |
| 101 | 1 | Midas | 1× | 90 | 90 | 80 | 25% spray |
| | | | | | | | missed |
| 102 | 1 | Midas | 2× | 80 | 90 | 80 | 25% spray |
| | | | | | | | missed |
| 103 | 1 | Midas | 0 | 0 | 0 | 0 | |
| 104 | 1 | 14CS0851- | 0 | 0 | 0 | 0 | |
| | | 01-14 | | | | | |
| 105 | 1 | 14CS0851- | 1× | 0 | 10 | 0 | 15% spray |
| | | 01-14 | | | | | missed |
| 106 | 1 | 14CS0851- | 2× | 0 | 10 | 0 | 15% spray |
| | | 01-14 | | | | | missed |
| 107 | 1 | SRS 934 | 0 | 0 | 0 | 0 | |
| 108 | 1 | SRS 934 | 1× | 80 | 80 | 70 | 5% spray |
| | | | | | | | missed |
| 109 | 1 | SRS 934 | 2× | 90 | 90 | 85 | 5% spray |
| | | | | | | | missed |
| 201 | 2 | Midas | 0 | 0 | 0 | 0 | |
| 202 | 2 | Midas | 1× | 90 | 90 | 90 | |
| 203 | 2 | Midas | 2× | 80 | 90 | 90 | |
| 204 | 2 | SRS 934 | 2× | 90 | 90 | 70 | 10% spray |
| | | | | | | | missed |
| 205 | 2 | SRS 934 | 1× | 80 | 90 | 90 | 10% spray |
| | | | | | | | missed |
| 206 | 2 | SRS 934 | 0 | 0 | 0 | 80 | |
| 207 | 2 | 14CS0851- | 2× | 0 | 15 | 15 | |
| | | 01-14 | | | | | |
| 208 | 2 | 14CS0851- | 0 | 0 | 0 | 0 | |
| | | 01-14 | | | | | |
| 209 | 2 | 14CS0851- | 1× | 0 | 15 | 0 | |
| | | 01-14 | | | | | |
| 301 | 3 | 14CS0851- | 1× | 10 | 20 | 0 | |
| | | 01-14 | | | | | |
| 302 | 3 | 14CS0851- | 2× | 0 | 15 | 0 | |
| | | 01-14 | | | | | |
| 303 | 3 | 14CS0851- | 0 | 0 | 0 | 0 | |
| | | 01-14 | | | | | |
| 304 | 3 | Midas | 0 | 0 | 0 | 0 | |
| 305 | 3 | Midas | 1× | 90 | 90 | 75 | 10% spray |
| | | | | | | | missed |
| 306 | 3 | Midas | 2× | 70 | 70 | 70 | 10% spray |
| | | | | | | | missed |
| 307 | 3 | SRS 934 | 2× | 90 | 90 | 80 | 10% spray |
| | | | | | | | missed |
| 308 | 3 | SRS 934 | 1× | 90 | 90 | 80 | 10% spray |
| | | | | | | | missed |
| 309 | 3 | SRS 934 | 0 | 0 | 0 | 0 | |
| 401 | 4 | SRS 934 | 1× | 90 | 90 | 90 | 10% spray |
| | | | | | | | missed |
| 402 | 4 | SRS 934 | 2× | 90 | 90 | 90 | 10% spray |
| | | | | | | | missed |
| 403 | 4 | SRS 934 | 0 | 0 | 0 | 0 | |
| 404 | 4 | Midas | 1× | 90 | 90 | 85 | 10% spray |
| | | | | | | | missed |
| 405 | 4 | Midas | 0 | 0 | 0 | 0 | |
| 406 | 4 | Midas | 2× | 90 | 90 | 90 | 15% spray |
| | | | | | | | missed |
| 407 | 4 | 14CS0851- | 0 | 0 | 0 | 0 | |
| | | 01-14 | | | | | |
| 408 | 4 | 14CS0851- | 2× | 0 | 20 | 0 | |
| | | 01-14 | | | | | |
| 409 | 4 | 14CS0851- | 1× | 0 | 15 | 0 | |
| | | 01-14 | | | | | |

TABLE 18
Field Trial Data at Saskatoon, SK in 2017.

| | Treat- | | | Herbicide injury (% stunting) | | |
|---|---|---|---|---|---|---|
| Rep | ment | Entry | Rate | 7 daa | 21 daa | 42 daa |
| 1 | 2 | SRS 934 | 2× | 100 | 98 | 98 |
| 1 | 2 | SRS 934 | 0 | n/a. | n/a. | n/a. |
| 1 | 2 | SRS 934 | 1× | 98 | 95 | 95 |
| 1 | 1 | 14CS0851-01-14 | 1× | 2 | 2 | 0 |
| 1 | 1 | 14CS0851-01-14 | 0 | n/a. | n/a. | n/a. |
| 1 | 1 | 14CS0851-01-14 | 2× | 2 | 5 | 5 |
| 1 | 3 | Midas | 0 | n/a. | n/a. | n/a. |
| 1 | 3 | Midas | 2× | 90 | 95 | 95 |
| 1 | 3 | Midas | 1× | 85 | 90 | 90 |
| 2 | 1 | 14CS0851-01-14 | 0 | n/a. | n/a. | n/a. |
| 2 | 1 | 14CS0851-01-14 | 1× | 0 | 0 | 0 |
| 2 | 1 | 14CS0851-01-14 | 2× | 2 | 2 | 2 |
| 2 | 3 | Midas | 1× | 85 | 95 | 95 |
| 2 | 3 | Midas | 2× | 95 | 98 | 98 |
| 2 | 3 | Midas | 0 | n/a. | n/a. | n/a. |
| 2 | 2 | SRS 934 | 2× | 100 | 98 | 98 |
| 2 | 2 | SRS 934 | 0 | n/a. | n/a. | n/a. |
| 2 | 2 | SRS 934 | 1× | 100 | 95 | 95 |
| 3 | 3 | Midas | 2× | 90 | 98 | 98 |
| 3 | 3 | Midas | 0 | n/a. | n/a. | n/a. |
| 3 | 3 | Midas | 1× | 80 | 85 | 85 |
| 3 | 2 | SRS 934 | 1× | 100 | 95 | 95 |
| 3 | 2 | SRS 934 | 0 | n/a. | n/a. | n/a. |
| 3 | 2 | SRS 934 | 2× | 100 | 98 | 98 |
| 3 | 1 | 14CS0851-01-14 | 1× | 2 | 2 | 0 |
| 3 | 1 | 14CS0851-01-14 | 2× | 5 | 5 | 5 |
| 3 | 1 | 14CS0851-01-14 | 0 | n/a. | n/a. | n/a. |
| 4 | 3 | Midas | 1× | 80 | 85 | 85 |
| 4 | 3 | Midas | 0 | n/a. | n/a. | n/a. |
| 4 | 3 | Midas | 2× | 90 | 95 | 95 |
| 4 | 2 | SRS 934 | 1× | 95 | 95 | 95 |
| 4 | 2 | SRS 934 | 2× | 100 | 98 | 98 |
| 4 | 2 | SRS 934 | 0 | n/a. | n/a. | n/a. |
| 4 | 1 | 14CS0851-01-14 | 0 | n/a. | n/a. | n/a. |
| 4 | 1 | 14CS0851-01-14 | 1× | 0 | 0 | 0 |
| 4 | 1 | 14CS0851-01-14 | 2× | 2 | 0 | 0 |

TABLE 19
Field Trial Data at Huntley, MT in 2017.

| | | | Herbicide | Herbicide |
|---|---|---|---|---|
| | | | injury at | injury at |
| Variety | Herbicide | Rep | 15 daa | 45 daa |
| 14CS0851-01 | Nontreated | 1 | 0 | 0 |
| | | 2 | 0 | 0 |
| | | 3 | 0 | 0 |
| | | 4 | 0 | 0 |
| | 1X Harmony | 1 | 0 | 0 |
| | | 2 | 0 | 0 |
| | | 3 | 0 | 0 |
| | | 4 | 0 | 0 |
| | 2 X Harmony | 1 | 0 | 0 |
| | | 2 | 0 | 0 |
| | | 3 | 0 | 0 |
| | | 4 | 0 | 0 |
| SRS 934 | Nontreated | 1 | 0 | 0 |
| | | 2 | 0 | 0 |
| | | 3 | 0 | 0 |
| | | 4 | 0 | 0 |
| | 1X Harmony | 1 | 50 | 60 |
| | | 2 | 60 | 50 |
| | | 3 | 60 | 75 |
| | | 4 | 55 | 65 |
| | 2 X Harmony | 1 | 60 | 80 |
| | | 2 | 65 | 70 |
| | | 3 | 60 | 80 |
| | | 4 | 65 | 70 |
| Midas | Nontreated | 1 | 0 | 0 |
| | | 2 | 0 | 0 |
| | | 3 | 0 | 0 |
| | | 4 | 0 | 0 |
| | 1X Harmony | 1 | 50 | 85 |
| | | 2 | 60 | 75 |
| | | 3 | 50 | 60 |
| | | 4 | 55 | 70 |
| | 2 X Harmony | 1 | 70 | 80 |
| | | 2 | 70 | 80 |
| | | 3 | 60 | 65 |
| | | 4 | 65 | 50 |

TABLE 20
Field Trial Data at Morris, MD in 2017.

| | | | Herbicide injury (% stunting) | | |
|---|---|---|---|---|---|
| Block | Variety | Treatment | 13 daa | 32 daa | 53 daa |
| 1 | SRS 934 | 1x | 90 | 80 | 40 |
| 1 | SRS 934 | 2x | 90 | 70 | 50 |
| 1 | SRS 934 | 0 | Control | Control | Control |
| 1 | 14CS0851 | 0 | Control | Control | Control |
| 1 | 14CS0851 | 2x | 6 | 4 | 4 |
| 1 | 14CS0851 | 1x | 0 | 0 | 0 |
| 1 | Midas | 1x | 90 | 70 | 40 |
| 1 | Midas | 2x | 90 | 70 | 40 |
| 1 | Midas | 0 | Control | Control | Control |
| 2 | 14CS0851 | 0 | Control | Control | Control |
| 2 | 14CS0851 | 1x | 6 | 6 | 6 |
| 2 | 14CS0851 | 2x | 20 | 4 | 4 |
| 2 | SRS 934 | 2x | 90 | 70 | 50 |
| 2 | SRS 934 | 0 | Control | Control | Control |
| 2 | SRS 934 | 1x | 90 | 30 | 20 |
| 2 | Midas | 1x | 90 | 50 | 30 |
| 2 | Midas | 2x | 90 | 50 | 40 |
| 2 | Midas | 0 | Control | Control | Control |
| 3 | Midas | 0 | Control | Control | Control |
| 3 | Midas | 1x | 90 | 70 | 40 |
| 3 | Midas | 2x | 90 | 70 | 40 |
| 3 | 14CS0851 | 2x | 30 | 4 | 6 |
| 3 | 14CS0851 | 0 | Control | Control | Control |
| 3 | 14CS0851 | 1x | 2 | 2 | 0 |
| 3 | SRS 934 | 1x | 90 | 40 | 20 |
| 3 | SRS 934 | 2x | 90 | 70 | 40 |
| 3 | SRS 934 | 0 | Control | Control | Control |
| 4 | SRS 934 | 2x | 90 | 40 | 30 |
| 4 | SRS 934 | 1x | 90 | 60 | 30 |
| 4 | SRS 934 | 0 | Control | Control | Control |
| 4 | Midas | 2x | 90 | 60 | 40 |
| 4 | Midas | 0 | Control | Control | Control |
| 4 | Midas | 1x | 90 | 50 | 40 |
| 4 | 14CS0851 | 0 | Control | Control | Control |
| 4 | 14CS0851 | 1x | 0 | 4 | 2 |
| 4 | 14CS0851 | 2x | 30 | 6 | 4 |

The data demonstrates that the modified camelina plants of the present disclosure exhibit significantly increased tolerance or resistance to Group 2 herbicides.

### Example 8: Identification and Characterization of AHAS as the Modified Gene Product

The enzyme acetohydroxyacid synthase (AHAS) catalyzes the condensation of two molecules of pyruvate to yield acetolactate, and the condensation of pyruvate and 2-ketobutyrate to yield 2-aceto2-hydroxybutyrate:

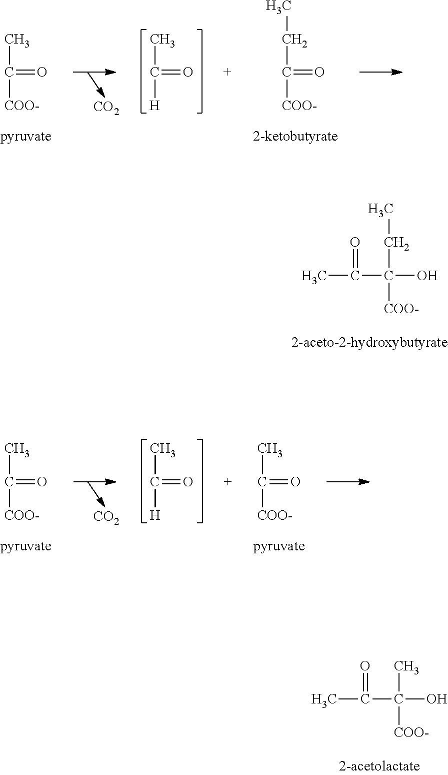

With this, AHAS catalyzes the first reaction of a common pathway that leads to the synthesis of the branched-chain amino acids valine, leucine, and isoleucine. Sulfonylurea herbicides inhibit the AHAS enzyme by blocking substrate access to the active site and thus starve affected plants of branched-chain amino acids leading to symptoms ranging from stunting and malformation to death.

As described in Examples 1-3, the sulfonylurea tolerance trait in both 12CS0365 and 12CS0366 was introduced through chemical mutagenesis of C. sativa accession SRS 934 (Plant Genetic Resources of Canada, PGRC) using ethyl methane sulfonate (EMS), and subsequently stabilized using traditional breeding methods.

Initial molecular characterization was achieved by DNA sequencing of the AHAS gene and aligning the DNA sequence of wild-type C. sativa AHAS genes with DNA sequences of 12CS0365 and 12CS0366.

DNA was isolated from leaf tissue samples of 3 plants of each SRS 934, 12CS0365 and 12CS0366 by a modification of the Dellaporta DNA extraction method for maize (Dellaporta, 1994). Briefly, approximately 150 mg of young leaf tissue we ground with mortar and pestle in liquid nitrogen. 1 mL extraction buffer (100 mM Tris-HCl pH8.0, 50 mM EDTA, 500 mM NaCl, 10 mM mercaptoethanol) was added to the frozen tissue and grinding continued. Slurry was poured into 2.2 mL microcentrifuge tubes, to which 75 μL 20% sodium dodecyl sulfate was added. After vortex mixing, tubes were incubated at 65° C. for 15 minutes, then 375 μL 5 M potassium acetate was added, mixed well, and incubated on ice for 20 minutes, then centrifuged at 10,000×g for 15 minutes at 4° C. The supernatant was transferred to a new tube and 0.6 volumes of isopropanol were added to precipitate the DNA. The samples were mixed gently and incubated at −20° C. for at least 30 minutes. The tubes were then centrifuged at 10,000×g for 15 minutes at 4° C., the DNA pellet washed with 75% ethanol, and allowed to dry. The DNA pellet was resuspended in 450 μL TE buffer (50 mM Tris-HCl, 10 mM EDTA pH 8.0) and 10 μg RNAse A added. After incubating at 37° C. for 1 hour, the DNA solution was further cleaned by extracting with equal amount of phenol: chloroform: isoamyl alcohol (25:24:1v/v), then with chloroform: isoamyl alcohol (24:1v/v). The aqueous phase was transferred to a new 1.5 mL microcentrifuge tube, and DNA precipitated with 2.5 volumes ethanol and 0.1 volume 3 M sodium acetate. The tubes were then centrifuged at 10,000×g for 5 minutes at 4° C., the supernatant discarded, and the DNA pellet was washed in 500 μL 70% ethanol and air-dried. The DNA pellet was resuspended in 100 μL of TE buffer (10 mM Tris-HCl and 1 mM EDTA, pH 8.0) and 100 ng of each sample used in PCR reaction.

PCR primers ALS fwd and ALS rev were designed to flank the camelina AHAS genes, producing an amplicon of 2,360 bp for all three orthologues (Table 21). The PCR reaction was carried out in 50 μL volumes using 100 ng of genomic DNA, 2.5 units of PfuUltra® II Fusion HS DNA Polymerase (Agilent Technologies, Santa Clara, CA, USA), 1×PFU II reaction buffer, 0.2 mM of each dNTP and 0.5 μM of each primer pair. After an initial denaturing step at 95° C. for 2 min, 35 cycles were performed of 30s at 94° C., 30s at 55° C. and 2 min at 72° C., followed by a final extension of 4 min at 72° C. PCR products were separated by electrophoresis in 0.8% (w/v) agarose gel stained with ethidium bromide. The desired DNA fragments were recovered by excision from the gel and purified by QIAquick® Gel Extraction Kit (Qiagen, Inc., Valencia, CA, USA) following the manufacturer's protocol. The purified fragments were ligated using pCR4 Blunt vector kit (Life Technologies) and 4 μL of the ligation mixture was transformed to 100 μL of DH5α electrocompetent E. coli cells. Six positive clones for each plant line with the desired fragment were grown overnight in 2 mL of liquid Luria-Bertani (LB) broth supplemented with 100 μg mL-1 ampicillin. Plasmid DNA was extracted using QIAprep® Spin Miniprep® Kit (Qiagen, Inc., Valencia, CA, USA). Recombinant clones were sent to the NRC DNA Sequencing Lab (Saskatoon, SK) using sequencing primers M13 FWD-20, M13 REV-20 for vector sequences flanking the insert, and ALS FWD2, ALS REV2, and ALS REV3, designed to amplify and overlap to obtain full contigs of all three AHAS orthologues (Table 21). Sequences were assembled and aligned to all three wildtype C. sativa AHAS sequences in order to identify the bp mutation(s) responsible for reduced sensitivity of the AHAS enzyme to the herbicide thifensulfuron-methyl. Wild-type sequences that were used for alignment were kindly provided by Dr. Isobel Parkin (AAFC-SRDC, Saskatoon). These sequences were obtained during sequencing of the C. sativa genome (Kagale et al., 2015).

Based on the sequencing and alignment results (18 full sequences per plant line), the mutated AHAS gene in camelina line 12CS0365 is most similar to orthologue 3 of the wildtype (CsAHAS3) and the mutated AHAS gene in 12CS0366 is most similar to orthologue 1 of the wildtype (CsAHAS1), as identified by Parkin et al. (unpublished data).

TABLE 21
Primers used for the amplification of all three orthologues of the complete CSAHAS
gene (ALS fwd and ALS rev) and primers used to completely sequence the AHAS genes of mutant
lines 12CS0365 and 12CS0366 (M13 FWD-20, M13 REV-20, ALS FWD2, ALS REV2, and ALS REV3).

| Primer | Sequence |
|---|---|
| ALS fwd | CTGGCTTCGTCTTTCTCCTG (SEQ ID NO: 12) |
| ALS rev | GTTGGAGAGACATGAACGGAG (SEQ ID NO: 13) |
| M13 FWD-20 | TGTAAAACGACGGCCAGT (SEQ ID NO: 14) |
| M13 REV-20 | CAGGAAACAGCTATGAC (SEQ ID NO: 15) |
| ALS FWD2 | ATGTTGGTGGTGGTTGTTTG (anneals at 905-924 bp of the ORF) (SEQ ID NO: 16) |
| ALS REV2 | AAGCACCAAGACCCATCAAC (anneals at 985-1003 bp of the ORF) (SEQ ID NO: 17) |
| ALS REV3 | GCCATCTCCTTCCGTTATGA (anneals at 1,970-1,989 bp of the ORF) (SEQ ID NO: 18) |

The AHAS nucleotide sequences (FIG. 4) and amino acid sequences (FIG. 5) of 12CS0365 and 12CS0366 were aligned with the wild-type sequences of all three AHAS orthologues obtained from the Parkin lab (AAFC-SRDC, Saskatoon) for comparison using Clustal Omega. In FIG. 4, the box shows the single base change from C to T in both 12CS0366 and 12CS0365 at position 580. In FIG. 5, the starred mutation in both 12CS0365 and 12CS0366 resulted in an amino acid substitution from Proline (P) to Serine(S) at position 194 which is homologues to the well characterized Pro 197 mutation.

As shown in FIG. 5, mutant lines 12CS0365 and 12CS0366 possess the Pro-197 mutation in two different CsAHAS orthologues, CsAHAS3 and CsAHAS1, respectively. The mutation in line 12CS0365 at position 80 (arginine [R] vs. glutamate [E]) is not significant because it is within the first 85 amino acids, which is a chloroplast transit peptide, not part of the mature protein, and does not affect the activity of the AHAS enzyme (Example 10). The mutation in line 12CS0366 at position 293 (valine [V] to isoleucine [I] is not significant, as the amino acids are the same as in wild-type CsAHAS orthologue 3. The complete DNA sequence alignment can be found in FIG. 6 and the complete amino acid sequence alignment can be found in the FIG. 7.

As described in Example 6, camelina line 14CS0851-01-14 was developed by crossing camelina mutant lines 12CS0365 and 12CS0366 and subsequent stabilizing of the trait through traditional breeding techniques.

### Example 9: Expression Level of CsAHAS Gene(s) in Leaves and Seeds

Expression of the endogenous AHAS genes is important for the synthesis of the branched-chain amino acids leucine, isoleucine, and valine. Base pair changes (mutations) in the DNA template will cause changes to the RNA during transcription and to the amino acid composition of the protein during translation. Consequently, any mutations in the DNA may affect the functionality of the AHAS protein. In order to detect changes at the level of transcription, a Reverse Transcription-Quantitative Polymerase Chain Reaction (RT-qPCR) assay was performed on 14CS0851-01-14 (PNT), SRS 934 and commercial variety MIDAS™. The assay was performed using RNA as the starting material, which is reverse-transcribed into cDNA. The cDNA was quantitatively amplified using the probe-based TaqMan® Multiplex Gene Expression Assay, which consists of a pair of unlabeled PCR primers and a TaqMan® FAM™ dye labelled probe complementary to the gene of interest and normalized with a TaqMan®HEX™ dye labelled probe complementary to the housekeeping gene glyceraldehyde-3-phophate dehydrogenase (GAPC-1) (Thellin, 1999). The target gene(s) as well as an internal control (housekeeping gene) were co-amplified in the same reaction, eliminating the well-to-well variability that would occur if separate amplification reactions were carried out. The Multiplex RT-qPCR analysis was performed using a Qiagen® Rotor-Gene-Q instrument and all data was analyzed using Rotor-Q software version Jan2009 following the Qiagen® Rotogene qPCR handbook

Detailed Method: Total RNA was extracted from combined pools of seed and leaf tissue from lines 14CS0851-01-14 (PNT), SRS 934 (parent) and commercial variety MIDAS™. Seed pools from 4 plots of each line were collected from a replicated field trial grown in Saskatoon, while pooled leaf tissue was obtained from 6 plants of each line grown in a greenhouse at 22° C. under natural light conditions supplemented with high pressure sodium lights with a 16-h photoperiod.

The extraction method was modified from Carpenter and Simon (1998). Young leaves and mature seeds were harvested, frozen in liquid nitrogen, and RNA extracted. Briefly, 400 mg young plant material was ground to a fine powder in liquid nitrogen, and 1 mL of RNA extraction buffer consisting of 0.4 M LiCl, 0.2M Tris (pH 8.0), 25 mM EDTA, and 1% sodium dodecyl sulfate was added and mixed in a mortar with a pestle. 500 μL of slurry was transferred to micro-centrifuge tubes and extracted twice with equal amounts of phenol, then once with an equal amount of chloroform. Nucleic acids were precipitated by adding 55 μL 3 M sodium acetate and 900 μL 95% ethanol, at −80° C. for 30 minutes, and centrifuged at 12,000 rpm for 5 minutes. Pellet was washed twice with 300 μL of 2 M LiCl and supernatant discarded after each wash. Pellet was resuspended in 300 μL RNAse-free ddH20 and re-precipitated with 30 μL 3M sodium acetate and 700 μL 95% ethanol. After chilling for 30 min at −80° C., solution was centrifuged 5 minutes at 12,000 rpm, washed twice with 75% ethanol, and resuspended in 50 μL RNAse-free H₂0.

Residual genomic DNA was removed by treating 10 μg of each RNA sample with DNAse1 (protocol: New England Biolabs M0303, New England Biolabs, Ipswich, MA, USA) and first strand cDNA was synthesized using Superscript II™ (Invitrogen, Carlsbad, CA, USA) and treated with RNAse H, using product protocols.

TABLE 22
Primers and probe sets for all 3 orthologues of the C. sativa AHAS gene and housekeeping
gene glyceraldehyde-3-phophate dehydrogenase (GAPC-1) were designed using IDT PrimerQuest
software.

| Primer/Probe | Sequence | Position |
|---|---|---|
| Ortho123 FWD | ATC TCG TTA GCG GAT TAG CC | 512-531 (SEQ ID NO: 19) |
| Ortho123 REV | CCT CAA CGA TGG GAG TTT CTT | 631-611 (SEQ ID NO: 20) |
| FAM Probe | FAM/TGTTCCTCT/ZEN/TGTAGCGATCACGGG/3IABkFQ | 549-572 (SEQ ID NO: 21) |
| GAPC-11 FWD | AGA GCC AGT CAA GTC CCT CA | 1624-1644 (SEQ ID NO: 21) |
| GAPC-11 REV | GAC AAG CTT GGG CTT CAC TC | 1723-1703 (SEQ ID NO: 22) |
| HEX Probe GAPC-1 | HEX/TCCGCCAAC/ZEN/GACACTGGTACATTC/3IABkFQ | 1678-1701 (SEQ ID NO: 23) |

RT-qPCR was performed on the gDNA-free cDNA using primers as listed in Table 22 with qPCR Roto-Gene (Qiagen, Hilden, Germany) instrument under the following conditions: 1 μL (5 ng) cDNA template was used in qPCR reactions along with 12.5 μL 2× Qiagen® Rotor-Gene Mutiplex PCR Master Mix, 1.25 μL AHAS Primer-FAM Probe mix, 1.25 μL CsGAPC-1 primer-HEX probe mix (10 μM Forward, 10 μM Reverse, and 5 μM probe), and 9 μL RNAse-free H₂O. All reactions were performed in triplicate using the following program: 95° C. 5 min, then 40 cycles of 95° C. 25 sec, 60° C. 25 sec.

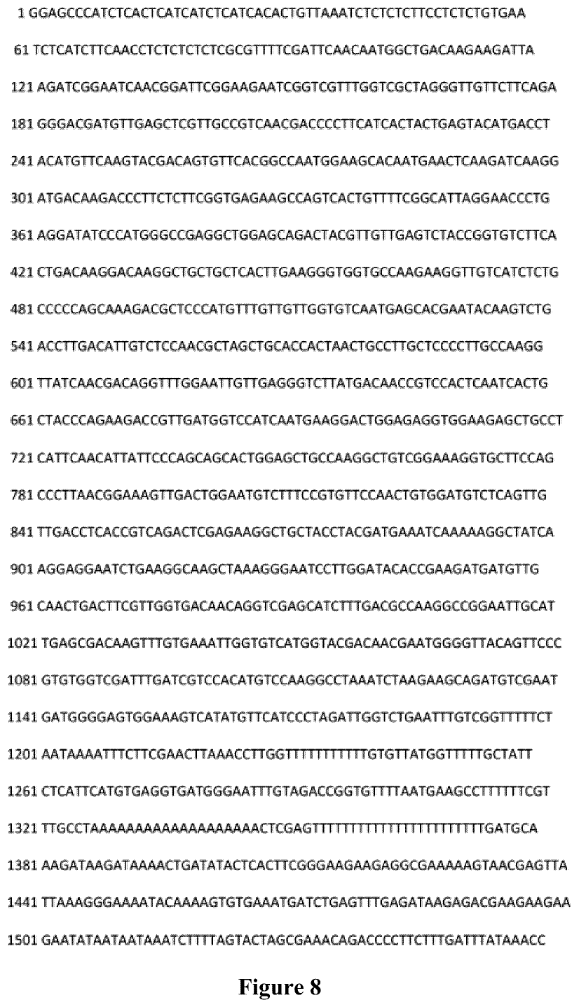

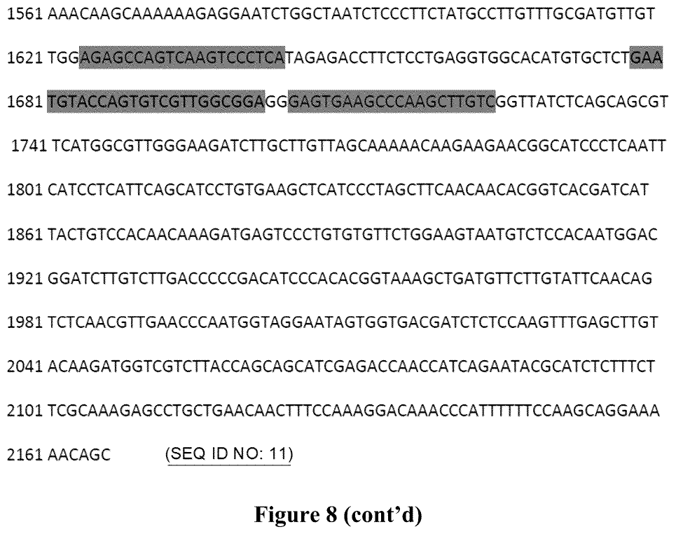

In order to rate the efficiency of the qPCR reaction, a standard curve was prepared using serial dilutions of MIDAS™ leaf cDNA at 1:1, 1:10, 1:100, 1:1000, 1:10000 for AHAS (gene of interest), and also for GAPC-1, the housekeeping gene used to normalize the data (Vandesompele et al, 2002) (data not shown). The complete sequence of CsGAPC-1 can be found in FIG. 8. Percent efficiency was 1.0 for AHAS and 1.09 for GAPC-1. Efficiency between 0.9 and 1.1 is considered acceptable for validating the results of the test material.

Results: Table 23 below shows the average relative expression of the camelina AHAS cDNA for seed and leaf material, after normalization with housekeeping gene GAPC-1. The PCR reaction was performed in triplicate and analyzed using the Qiagen Roto-gene analysis software. No-template-controls and RNA controls were included and results of these controls were negative, as expected. Raw data can be found in Tables 24 and 25.

TABLE 23
Relative AHAS gene expression of 14CS0851-01-14 (PNT), SRS
934 and commercial variety MIDAS ™ seed and leaf
tissue, determined by RT-qPCR. Each sample was analyzed in triplicate.

| Sample | Relative expression |
|---|---|
| 14CS0851-01-14 seed | 5.99 A |
| SRS 934 seed | 6.18 A |
| MIDAS ™ seed | 5.65 A |
| 14CS0851-01-14 leaf | 0.81 B |
| SRS 934 leaf | 0.63 B |
| MIDAS ™ leaf | 1.07 B |

TABLE 24
Raw data of cycle threshold values (Ct) for standard curve serial dilutions, no-template-controls (NTC), and seed
and leaf material of 14CS0851-01-14 (PNT), SRS 934 and commercial variety Midas ™. All tests were performed
in triplicate on Qiagen Rotor-Gene-Q and all data was analyzed using Rotor-Q software version Jan2009.

| | | | | Given | Calc | | | GOI | HKG |
|---|---|---|---|---|---|---|---|---|---|
| Relative AHAS Gene Expression by RT-qPCR | | | | Conc | Conc | GOI | HKG | Rep. Ct | Rep. Ct |
| Sample Name | GOI Ct | HKG Ct | HKG-GOI | (ng/ul) | (ng/ul) | Rep. Ct | Rep. Ct | Std. Dev. | Std. Dev. |
| 1:1 SRS934 Leaf | 22.41 | 24.04 | 1.63 | 10,000 | 8,698 | 22.2 | 23.94 | 0.3 | 0.14 |
| 1:1 SRS934 Leaf | 21.86 | 23.97 | 2.11 | 10,000 | 12,738 | 21.86 | 23.97 | | |
| 1:1 SRS934 Leaf | 21.99 | 23.85 | 1.86 | 10,000 | 11,695 | | | | |
| 1:10 SRS934 Leaf | 24.98 | 27.24 | 2.26 | 1,000 | 1,483 | 25.72 | 27.24 | 0.66 | 0.26 |
| 1:10 SRS934 Leaf | 25.92 | 26.98 | 1.06 | 1,000 | 773 | | | | |
| 1:10 SRS934 Leaf | 26.26 | 27.5 | 1.74 | 1,000 | 612 | | | | |
| 1:100 SRS934 Leaf | 28.88 | 30.59 | 1.71 | 100 | 100 | 29.01 | 30.82 | 0.25 | 0.31 |
| 1:100 SRS934 Leaf | 29.29 | 31.16 | 1.87 | 100 | 75 | | | | |
| 1:100 SRS934 Leaf | 28.85 | 30.69 | 1.84 | 100 | 102 | | | | |
| 1:1000 SRS934 Leaf | 32.28 | 34.46 | 2.18 | 10 | 10 | 31.94 | 34.3 | 0.42 | 0.18 |
| 1:1000 SRS934 Leaf | 31.48 | 34.32 | 2.84 | 10 | 17 | | | | |
| 1:1000 SRS934 Leaf | 32.08 | 34.11 | 2.03 | 10 | 11 | | | | |
| 1:10000 SRS934 Leaf | 34.63 | 35.87 | 1.24 | 1 | 2 | 35.65 | 35.67 | 1.22 | 0.28 |
| 1:10000 SRS934 Leaf | 35.3 | 35.47 | 0.17 | 1 | 1 | | | | |
| 1:10000 SRS934 Leaf | 36.99 | | 0 | 1 | 0 | | | | |
| NTC | | | 0 | | | | | | |
| NTC | 22.79 | | 0 | 6,730 | | | | | |
| NTC | 22.71 | | 0 | 7,0 [symbol] 4 | | | | | |
| 1/100 cDNA 14CS0851-01-14 seed | 29.5 | 33.86 | 4.3 [symbol] | | 65 | 29.8 | 33.75 | 0.34 | 0.3 |
| 1/100 cDNA 14CS0851-01-14 seed | 30.16 | 33.99 | 3.83 | | 41 | | | | |
| 1/100 cDNA 14CS0851-01-14 seed | 29.75 | 33.41 | 3.66 | | 55 | | | | |
| 1/100 cDNA SRS934 seed | 31.51 | 35.03 | 3.52 | | 16 | 31.23 | 35.13 | 0.4 | 0.37 |
| 1/100 cDNA SRS934 seed | 30.76 | 34.81 | 4.05 | | 27 | | | | |
| 1/100 cDNA SRS934 seed | 31.41 | 35.54 | 4.13 | | 17 | | | | |
| 1/100 cDNA Midas seed | 31.2 [symbol] | 34.75 | 3.49 | | 19 | 31.04 | 34.83 | 0.64 | 0.37 |
| 1/100 cDNA Midas seed | 30.32 | 34.5 | 4.18 | | 37 | | | | |
| 1/100 cDNA Midas seed | 31.54 | 35.23 | 3.69 | | 16 | | | | |
| 1/100 cDNA 14CS0851-01-14 | 32.24 | 33.41 | 1.17 | | 10 | 32.34 | 33.41 | 0.1 | 0.11 |
| 1/100 cDNA 14CS0851-01-14 | 32.34 | 33.53 | 1.19 | | 9 | | | | |
| 1/100 cDNA 14CS0851-01-14 | 32.43 | 33.3 | 0.87 | | 9 | | | | |
| 1/100 cDNA SRS934 leaf | 34.2 | 33.79 | 0 | | 3 | 32.38 | 33.13 | 1.68 | 0.61 |
| 1/100 cDNA SRS934 leaf | 30.9 | 32.59 | 1.69 | | 25 | | | | |
| 1/100 cDNA SRS934 leaf | 32.05 | 33.01 | 0.96 | | 11 | | | | |
| 1/100 cDNA Midas leaf | 32.26 | 33.58 | 1.32 | | 10 | 32.55 | 33.99 | 0.5 | 0.54 |
| 1/100 cDNA Midas leaf | 32.26 | 33.78 | 1.52 | | 10 | | | | |
| 1/100 cDNA Midas leaf | 33.13 | 34.6 | 1.47 | | 5 | | | | |
| 1/100 RNA 14CS0851-01-14 seed | 34.78 | | 0 | | 2 | 34.78 | | | |
| 1/100 RNA SRS934 seed | 34.73 | | 0 | | 2 | 34.73 | | | |
| 1/100 RNA Midas seed | 35.05 | | 0 | | 1 | 33.05 | | | |
| 1/100 RNA 14CS0851-01-14 seed | 35.92 | | 0 | | 1 | 35.92 | | | |
| 1/100 RNA SRS934 leaf | 35.51 | | 0 | | 1 | 33.51 | | | |
| 1/100 RNA Midas leaf | 33.22 | | 0 | | 5 | 33.22 | | | |
| BLK 1 | 0 | | 0 | | | | | | |
| BLK 2 | 0 | | 0 | | | | | | |
| BLK 3 | 0 | | 0 | | | | | | |

[symbol]

indicates data missing or illegible when filed

TABLE 25
Summary of average raw data of cycle threshold values (Ct) for standard
curve serial dilutions, no-template-controls (NTC), and seed and leaf
material of 14CS0851-01-14 (PNT), SRS 934 and commercial variety Midas ™. All
tests were performed in triplicate on Qiagen Rotor-Gene-Q and all data
was analyzed using Rotor-Q software version January 2009.

| | GOI | GOI | Norm. | Norm. | Relative |
|---|---|---|---|---|---|
| Replicate Name | Conc. | Count | Conc. | Count | Conc. |
| 1:1 SRS 934 Leaf | 10,086 | 2 | 12,341 | 2 | 0.82 |
| 1:1 SRS 934Leaf | 12,738 | 1 | 12,083 | 1 | 1.05 |
| 1:10 SRS 934 Leaf | 889 | 3 | 1,081 | 3 | 0.82 |
| 1:100 SRS 934 Leaf | 92 | 3 | 77 | 3 | 1.19 |
| 1:1000 SRS 934 Leaf | 12 | 3 | 6 | 3 | 2.04 |
| 1:10000 SRS 934 Leaf | 1 | 3 | 2 | 2 | 0.44 |
| NTC | 6,910 | 2 | | 0 | |
| 1/100 cDNA 14CS0851-01-14 seed | 53 | 3 | 9 | 3 | 5.99 |
| 1/100 cDNA SRS 934 seed | 20 | 3 | 3 | 3 | 6.18 |
| 1/100 cDNA MIDASTM seed | 23 | 3 | 4 | 3 | 5.65 |
| 1/100 cDNA 14CS0851-01-14 leaf | 9 | 3 | 11 | 3 | 0.81 |
| 1/100 cDNA SRS 934 leaf | 9 | 3 | 14 | 3 | 0.63 |
| 1/100 cDNA MIDASTM leaf | 8 | 3 | 7 | 3 | 1.07 |
| 1/100 RNA 14CS0851-01-14 seed | 2 | 1 | | 0 | |
| 1/100 RNA SRS 934 seed | 2 | 1 | | 0 | |
| 1/100 RNA MIDASTM seed | 1 | 1 | | 0 | |
| 1/100 RNA 14CS0851-01-14 leaf | 1 | 1 | | 0 | |
| 1/100 RNA SRS 934 leaf | 1 | 1 | | 0 | |
| 1/100 RNA MIDASTM leaf | 5 | 1 | | 0 | |
| BLK 1 | | 0 | | 0 | |
| BLK 2 | | 0 | | 0 | |
| BLK 3 | | 0 | | 0 | |

Based on the RT-qPCR assays of the AHAS cDNA, there are no significant differences between the expression levels of the CsAHAS genes in mutant line 14CS0851-01-14 (PNT), SRS 934 and commercial variety MIDAS™. The relative expression of CsAHAS in the seed is approximately 6-fold greater than in the leaf tissue for all 3 comparators.

### Example 10: In Vitro CsAHAS Activity of 14CS0851-01-14 vs. Comparators and Product Feedback Inhibition of CsAHAS

AHAS, also known as acetolactate synthase (ALS, EC 4.1.3.18) is the first enzyme unique to the biosynthesis of the branched-chain amino acids valine, leucine, and isoleucine. This enzyme is under feedback regulation by these amino acids in plants: as the amount of product (branched-chain amino acids) increases, the AHAS enzyme will be inhibited. A number of studies in different plant species comparing the branched-chain amino acid physiology between plants resistant or sensitive to ALS inhibitors have found that AHAS from resistant biotypes was less sensitive to feedback inhibition by branched-chain amino acids than that from the sensitive biotype, resulting in greater accumulation of branched-chain amino acids (Eberlein et al., 1999; Dyer et al., 1993; Thompson et al., 1994a). These findings suggest a possible fitness advantage of resistant plants in the absence of herbicide selection as greater accumulation of branched-chain amino acids may result in more rapid germination of seeds of resistant biotypes, particularly under cool temperatures. The purpose of this study was therefore to determine whether there are any significant difference between AHAS activity and feedback inhibition between the PNT line 14CS0851-01-14 and its parent, SRS 934 or the commercial variety MIDAS™

Experiments were conducted with the three comparators: PNT line 14CS0851-01-14 in parallel with its parent accession SRS 934 and commercial variety MIDAS™.

Briefly, for each of the three comparators, leaf and stem (petiole) material was bulk-harvested at the 3-4 leaf stage from at least 30 plantlets, snap-frozen in liquid nitrogen, and stored at −80° C. The AHAS in vitro assay was conducted according to the method of Singh et al (1988), with modifications by Yu (2010) and Rustgi (2014). For each comparator, 4 grams of frozen material were ground to a fine powder with a mortar and pestle in liquid nitrogen and homogenized in 1 volume of cold extraction buffer containing 100 mM potassium phosphate buffer (pH7.5), 10 mM sodium pyruvate, 5 mM MgCl2, 100 μM flavin adenine dinucleotide (FAD), and 10% glycerol. Four 2-mL microcentrifuge tubes were used for each comparator. The homogenate was centrifuged at 13,000 rpm for 10 min at 4° C., supernatant collected and re-centrifuged for an additional 10 minutes under the same conditions. For each tube, 1 mL of supernatant was pipetted into a new 2-mL microcentrifuge tube, and protein was precipitated by the addition of 1 mL of saturated ammonium sulfate. The solution was mixed well by vortex and allowed to stand on ice for 20 minutes, then centrifuged at 13,000 rpm at 4° C. The protein pellet was resuspended in 300 μL of reaction buffer containing 50 mM potassium phosphate buffer (pH7.5), 100 mM sodium pyruvate, 10 mM MgCl2, 1 mM EDTA, 10 μM FAD, 100 mM NaCl, and 1 mM thiamine pyrophosphate. Tubes of each comparator were combined, mixed well, kept on ice and immediately used in the AHAS activity and feedback inhibition assays.

Total protein concentration of the crude extract was determined by the Bradford method (Bradford, 1976). All assays were conducted using 300 μg of total protein each.

The enzyme activity assay was performed in triplicate using pyruvate as the substrate, which is provided in the resuspension buffer. 100 μL of extract was incubated for one hour at 37° C. for production of acetolactate from pyruvate. The acetolactate end product was converted to acetoin by adding 20 μL 6N H₂SO₄, incubating 15 min at 60° C. Final color was developed by the addition of 95 μL of 0.55% creatine and 95 μL freshly made 5.5% a-naphthol, incubated a further 15 min at 60° C. and then read on Nanodrop® UV/VIS mode at 530 nm, against a blank where 20 μL 6 N H₂SO₄ was added prior to incubation. The amount of acetoin formed was determined through the use of a standard curve using commercial acetoin and values converted to AHAS activity (μmoles acetoin mg protein⁻¹ h⁻¹) (data not shown). Raw data can be found in Table 26 below.

TABLE 26
Extractable AHAS Activity (μmol acetoin/mg protein/hour).
Three independent experiments were performed, as described, on
mutant camelina 14CS0851-01-14, SRS 934 and commercial variety
MIDAS ™. Following incubation of the enzyme with the substrate
(pyruvate), the end product acetolactate is converted to acetoin
by decarboxylation with sulfuric acid and high temperature. Acetoin
is detected by formation of a creatineand a-napthol complex which
can be measured at 530 nm. The amount of product produced in each
sample was interpolated from an acetoin standard curve.
Extractable AHAS Activity (umol Acetoin/
mg protein/hr) (no amino acids added)

| | A530 | | | mM | umol/mg |
|---|---|---|---|---|---|
| | nm | m | b | Acetoin | protein/hr |
| Experiment 1 | | total vol = 310 uL, prot/assay = 300 ug | | | |
| 14CS0851-01-14-1 | 0.435 | 0.1734 | 0.0079 | 2.50 | 2.59 |
| 14CS0851-01-14-2 | 0.401 | 0.1734 | 0.0079 | 2.31 | 2.39 |
| AVG | 0.418 | 0.1734 | 0.0079 | 2.41 | 2.49 |
| SAS934-1 | 0.397 | 0.1734 | 0.0079 | 2.29 | 2.36 |
| SRS934-2 | 0.395 | 0.1734 | 0.0079 | 2.27 | 2.35 |
| AVG | 0.396 | 0.1734 | 0.0079 | 2.28 | 2.36 |
| Midas-1 | 0.445 | 0.1734 | 0.0079 | 2.56 | 2.65 |
| Midas-2 | 0.446 | 0.1734 | 0.0079 | 2.57 | 2.65 |
| AVG | 0.446 | 0.1734 | 0.008 | 2.56 | 2.65 |
| Experiment 2 | | total vol = 310 uL, prot/assay = 300 ug | | | |
| 14CS0851-01-14-1 | 0.252 | 0.1734 | 0.0079 | 1.45 | 1.49 |
| 14CS0851-01-14-2 | 0.216 | 0.1734 | 0.0079 | 1.24 | 1.28 |
| AVG | 0.234 | 0.1734 | 0.0079 | 1.45 | 1.49 |
| SRS934-1 | 0.292 | 0.1734 | 0.0079 | 1.68 | 1.73 |
| SRS934-2 | 0.162 | 0.1734 | 0.0079 | 0.98 | 0.99 |
| AVG | 0.230 | 0.1734 | 0.0079 | 1.45 | 1.49 |
| Midas-1 | 0.331 | 0.1734 | 0.0079 | 1.90 | 1.96 |
| Midas-2 | 0.237 | 0.1734 | 0.0079 | 1.36 | 1.40 |
| AVG | 0.284 | 0.1734 | 0.0079 | 1.63 | 1.68 |
| Experiment 3 | | total vol = 310 uuL, prot/assay = 350 ug | | | |
| 14CS0851-01-14-1 | 0.159 | 0.1779 | 0.0045 | 0.89 | 0.79 |
| 14CS0851-01-14-2 | 0.165 | 0.1779 | 0.0045 | 0.92 | 0.82 |
| 14CS0851-01-14-3 | 0.166 | 0.1779 | 0.0045 | 0.93 | 0.82 |
| AVG | 0.163 | 0.1779 | 0.0045 | 0.91 | 0.81 |
| SRS934-1 | 0.190 | 0.1779 | 0.0045 | 1.06 | 0.94 |
| SRS934-2 | 0.208 | 0.1779 | 0.0045 | 1.16 | 1.03 |
| SRS934-3 | 0.193 | 0.1779 | 0.0045 | 1.08 | 0.96 |
| AVG | 0.197 | 0.1779 | 0.0045 | 1.10 | 0.98 |
| Midas-1 | 0.230 | 0.1779 | 0.0045 | 1.29 | 1.14 |
| Midas-2 | 0.237 | 0.1779 | 0.0045 | 1.33 | 1.18 |
| Midas-3 | 0.240 | 0.1779 | 0.0045 | 1.34 | 1.19 |
| AVG | 0.236 | 0.1779 | 0.0045 | 1.32 | 1.17 |

The feedback inhibition assay was performed by combining equal amounts of extract and amino acids leucine, isoleucine, or valine, respectively, in concentrations of 0.1 mM, 1 mM, 10 mM and 100 mM, according to Yu et al. (2010). The reaction mixture contained 50 μL enzyme extract and 50 μL 100 mM sodium pyruvate and inhibitor amino acid. The reaction was incubated at 37° C. for 60 minutes, then stopped and acetolactate converted to acetoin with the addition of 20 μL of 6 N H₂SO₄ and incubated at 60° C. for 15 minutes. A separate background “blank” was included for each sample group by adding 20 μL of 6 N H₂SO₄ prior to the addition of the enzyme extracts. Then, 95 μL of 0.55% w/v creatine solution and 95 μL of α-naphthol solution (5.5% w/v in 5 N NaOH) were added and the mixture incubated at 60° C. for a further 15 minutes. After cooling to room temperature for 15 minutes, enzyme activity was determined colorimetrically by reading the absorbance at 530 nm. Three independent experiments were conducted. Experiment 1 and 2 each contained 2 technical replicates, while experiment 3 included 3 technical replicates. Raw data is shown in Table 27 below.

TABLE 27
AHAS product feedback inhibition assay. Inhibition of AHAS activity by addition of leucine, isoleucine, and valine at 1 mM,
10 mM, and 100 mM final concentration in assay. 100% activity conditions (control) contain 100 mM substrate pyruvate with
no added leucine, isoleucine, or valine. Absorbance readings were converted to AHAS Activity % of control.

| | | Pyruvate | Leucine | | | Isoleucine | | | Valine | | |
|---|---|---|---|---|---|---|---|---|---|---|---|
| | | 0 | 1 | 10 | 100 | 1 | 30 | 100 | 1 | 10 | 100 |
| Experiment 1 | | | | | | | | | | | |
| Rep1 | 14CS0851-01-14-1 | 0.435 | 0.214 | 0.183 | 0.14 [symbol] | 0.30 [symbol] | 0.217 | 0.188 | 0.216 | 0.20 [symbol] | 1.55 |
| Rep2 | 14CS0851-01-14-2 | 0.401 | 0.194 | 0.165 | 0.14 | 6.287 | 0.201 | 0.176 | 0.21 | 0.1 [symbol] | 0.142 |
| | AVG | 0.418 | 0.204 | 0.174 | 0.143 | 0.296 | 0.209 | 0.182 | 0.213 | 0.1 [symbol] 6 | 0.1485 |
| Rep1 | SRS934-1 | 0.3 [symbol] 7 | 0.2 | 0.17 | 0.154 | 0.251 | 0.233 | 0.196 | 0.236 | 0.186 | 0.168 |
| Rep2 | SRS934-2 | 0.395 | 0.208 | 0.187 | 0.141 | 0.256 | 0.23 [symbol] | 0.176 | 0.226 | 0.182 | 0.157 |
| | AVG | 0.39 [symbol] | 0.204 | 0.1785 | 0.1475 | 0.253 [symbol] | 0.234 | 0.186 | 0.231 | 0.184 | 0.1625 |
| Rep1 | Midas-1 | 0.445 | 0.215 | 0.178 | 0.142 | 0.306 | 0.275 | 0.194 | 0.252 | 0.205 | 0.174 |
| Rep2 | Midas-2 | 0.446 | 0.234 | 0.177 | 0.154 | 0.348 | 0.26 | 0.196 | 0.243 | 0.223 | 0.189 |
| | AVG | 0.446 | 0.2245 | 0.1775 | 0.148 | 0.327 | 0.2675 | 0.195 | 0.2475 | 0.214 | 0.1815 |
| Experiment 2 | | | | | | | | | | | |
| Rep1 | 14CS0851-01-14-1 | 0.252 | 0.162 | 0.151 | 0.138 | 0.238 | 0.200 | 0.170 | 0.227 | 0.190 | 0.176 |
| Rep2 | 14CS0851-01-14-2 | 0.21 [symbol] | 0.136 | 0.120 | 0.105 | 0.220 | 0.171 | 0.120 | 0.187 | 0.1 [symbol] | 0.333 |
| | AVG | 0.234 | 0.149 | 0.136 | 0.122 | 0.229 | 0.186 | 0.145 | 0.207 | 0.178 | 0.155 |
| Rep1 | SRS934-1 | 0.292 | 0.225 | 0.183 | 0.167 | 0.245 | 0.219 | 0.177 | 0.198 | 0.206 | 0.184 |
| Rep2 | SRS934-2 | 0.187 | 0.158 | 0.140 | 0.104 | 0.203 | 0.154 | 0.134 | 0.163 | 0.151 | 0.100 |
| | AVG | 0.230 | 0.192 | 0.162 | 0.136 | 0.224 | 0.187 | 0.156 | 0.181 | 0.179 | 0.142 |
| Rep1 | Midas-1 | 0.331 | 0.1 [symbol] 8 | 0.181 | 0.153 | 0.271 | 0.208 | 0.176 | 0.239 | 0.217 | 0.171 |
| Rep2 | Midas-2 | 0.237 | 0.162 | 0.150 | 0.150 | 0.204 | 0.176 | 0.149 | 0.166 | 0.165 | 0.161 |
| | AVG | 0.284 | 0.180 | 0.16 [symbol] | 0.152 | 0.238 | 0.1 [symbol] 2 | 0.163 | 0.203 | 0.191 | 0.166 |
| Experiment 3 | | | | | | | | | | | |
| Rep1 | 14CS0851-01-14-1 | 0.159 | 0.079 | 0.066 | 0.051 | 0.106 | 0.087 | 0.061 | 0.079 | 0.057 | 0.054 |
| Rep2 | 14CS0851-01-14-2 | 0.1 [symbol] | 0.099 | 0.061 | 0.060 | fail | 0.082 | 0.072 | 0.080 | 0.084 | 0.061 |
| Rep3 | 14CS0851-01-14-3 | 0.1 [symbol] | 0.106 | 0.067 | 0.057 | 0.104 | 0.094 | 0.052 | 0.082 | 0.080 [symbol] | 0.0 [symbol] |
| | AVG | 0.163 | 0.094 | 0.064 | 0.056 | 0.105 | 0.088 | 0.061 | 0.080 | 0.073 | 0.057 |
| Rep1 | SRS934-1 | 0.190 | 0.097 | 0.092 | 0.084 | 0.120 | 0.0 [symbol] 1 | 0.065 | 0.086 | 0.091 | 0.071 |
| Rep2 | SRS934-2 | 0.208 | 0.085 | 0.091 | 0.068 | 0.111 | 0.0 [symbol] | 0.0 [symbol] | 0.0 [symbol] 8 | 0.0 [symbol] | 0.072 |
| Rep3 | SRS934-3 | 0.193 | 0.086 | 0.071 | 0.089 | 0.114 | 0.100 | 0.073 | 0.100 | 0.091 | 0.067 |
| | AVG | 0.197 | 0.089 | 0.085 | 0.080 | 0.115 | 0.033 | 0.069 | 0.094 | 0.093 | 0.070 |
| Rep 1 | Midas-1 | 0.230 | 0.102 | 0.096 | 0.078 | 0.136 | 0.100 | 0.080 | 0.115 | 0.133 | 0.084 |
| Reg 2 | Midas-2 | 0.237 | 0.105 | 0.88 | 0.096 | 0.138 | 0.107 | 0.075 | 0.116 | 0.110 | 0.091 |
| Rep 3 | Midas-3 | 0.240 | 0.112 | 0.096 | 0.096 | 0.149 | 0.102 | 0.078 | 0.122 | 0.112 | 0.0 [symbol] 2 |
| | AVG | 0.235 | 0.106 | 0.093 | 0.090 | 0.141 | 0.103 | 0.077 | 0.117 | 0.112 | 0.0 [symbol] |

[symbol]

indicates data missing or illegible when filed

Results: AHAS activity assay—There was no significant difference in extractable AHAS activity of the mutant camelina line 14CS0851-01-14 compared to line SRS 934 or commercial variety MIDAS™ (Table 28).

TABLE 28
Results of the spectrophotometric assay for assessing AHAS activity
of camelina mutant line 14CS0851-01-14, line SRS 934 and commercial
check Midas ™ through indirect detection of the enzyme
product, acetolactate. The amount of product produced in each
sample was interpolated from an acetoin standard curve. Values
followed by the same letters are not statistically different.
N = 2 for experiments 1 and 2; n = 3 for experiment 3.

| Entry | Experiment 1 | Experiment 2 | Experiment 3 |
|---|---|---|---|
| 14CS0851-01-14 | 2.49 A | 1.49 A | 0.81 A |
| SRS 934 | 2.36 A | 1.49 A | 0.98 A |
| MIDAS ™ | 2.65 A | 1.68 A | 1.17 A |

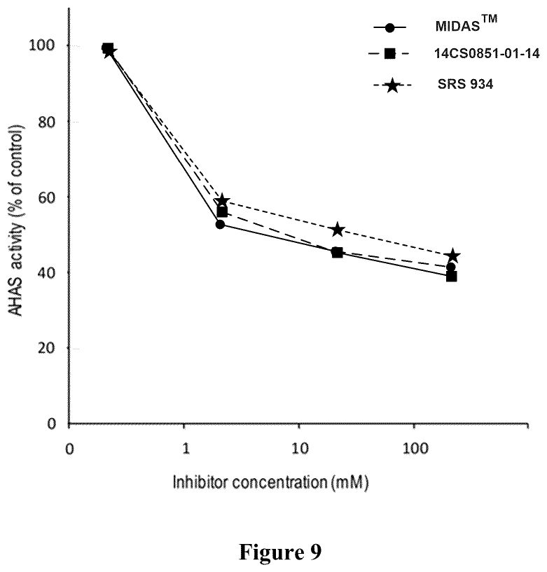

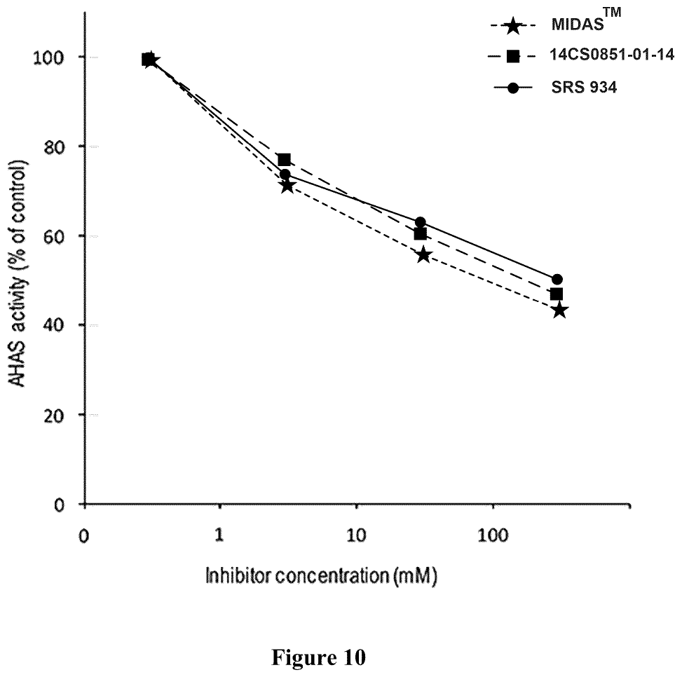

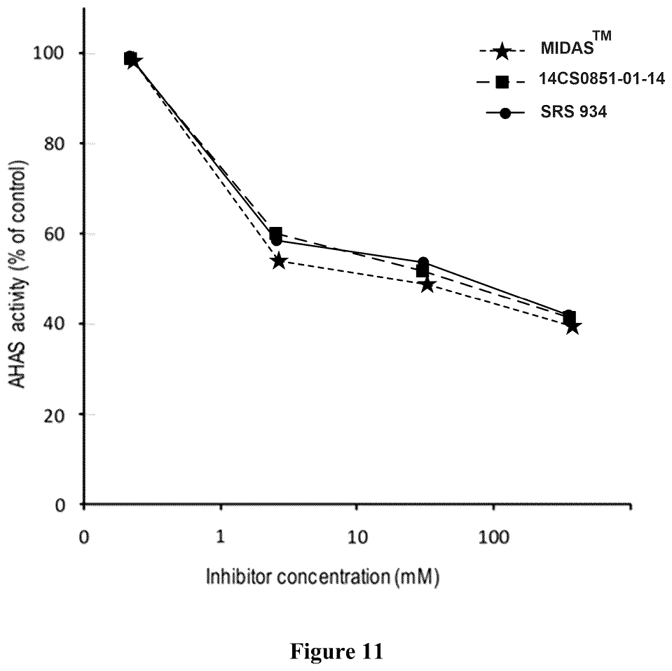

Feedback inhibition assay—The enzyme inhibition assay results are shown as % activity, with sodium pyruvate substrate alone designated as 100% activity. The results show that there is no significant difference in the sensitivity to feedback inhibition by the branched-chain amino acids leucine, isoleucine, and valine when comparing the enzyme extracts of 14CS0841-01-14, SRS 934, and MIDAS™ (Table 29 and FIGS. 9-11).

TABLE 29
Inhibition of AHAS activity by addition of leucine, isoleucine
and valine, respectively, at 1 mM, 10 mM, and 100 mM final
concentration in assay in 14CS0851-01-14, SRS 934 and Midas ™. 100%
activity conditions (control) contain 100 mM pyruvate with
no added branched-chain amino acids. Absorbance readings were
converted to AHAS activity as % of control. Values represent
the average of 3 replicates for each experiment.

| | | % activity | | |
|---|---|---|---|---|
| Exp. | Sample | 1 mM Leu | 10 mM Leu | 100 mM Leu |
| 1 | 14CS0851-01-14 | 48.8 | 41.6 | 34.2 |
| 1 | SRS 934 | 51.5 | 45.1 | 37.2 |
| 1 | Midas ™ | 50.4 | 39.8 | 33.2 |
| 2 | 14CS0851-01-14 | 63.7 | 58.1 | 52.1 |
| 2 | SRS 934 | 83.5 | 70.4 | 59.1 |
| 2 | Midas ™ | 63.2 | 58.2 | 53.3 |
| 3 | 14CS0851-01-14 | 57.9 | 39.5 | 34.2 |
| 3 | SRS 934 | 45.2 | 43.0 | 40.6 |
| 3 | Midas ™ | 45.2 | 39.6 | 38.1 |
| means | 14CS0851-01-14 | 56.8 | 46.4 | 40.2 |
| | SRS934 | 60.1 | 52.8 | 45.7 |
| | Midas | 52.9 | 45.9 | 41.6 |
| | Anova P value (rep)† | 0.95 | 0.90 | 0.79 |
| | Anova P value (line)‡ | 0.85 | 0.79 | 0.85 |

| Exp. | Sample | 1 mM Ile | 10 mM Ile | 100 mM Ile |
|---|---|---|---|---|
| 1 | 14CS0851-01-14 | 70.8 | 50.0 | 43.5 |
| 1 | SRS 934 | 64.0 | 59.1 | 47.0 |
| 1 | Midas ™ | 73.4 | 60.0 | 43.8 |
| 2 | 14CS0851-01-14 | 97.9 | 79.5 | 62.0 |
| 2 | SRS 934 | 97.4 | 81.3 | 67.8 |
| 2 | Midas ™ | 83.5 | 67.4 | 57.2 |
| 3 | 14CS0851-01-14 | 64.4 | 53.8 | 37.6 |
| 3 | SRS 934 | 58.3 | 47.4 | 34.9 |
| 3 | Midas ™ | 59.8 | 43.7 | 32.9 |
| means | 14CS0851-01-14 | 77.7 | 61.1 | 47.7 |
| | SRS 934 | 73.2 | 62.6 | 49.9 |
| | Midas ™ | 72.2 | 57.0 | 44.6 |
| | Anova P value (rep) | 0.59 | 0.57 | 0.45 |
| | Anova P value (line) | 0.93 | 0.91 | 0.91 |

| Exp. | Sample | 1 mM Val | 10 mM Val | 100 mM Val |
|---|---|---|---|---|
| 1 | 14CS0851-01-14 | 51.0 | 46.9 | 35.5 |
| 1 | SRS 934 | 58.3 | 46.5 | 41.0 |
| 1 | Midas ™ | 55.6 | 48.0 | 40.7 |
| 2 | 14CS0851-01-14 | 49.1 | 45.0 | 34.8 |
| 2 | SRS 934 | 47.9 | 47.0 | 35.4 |
| 2 | Midas ™ | 49.8 | 47.5 | 37.8 |
| 3 | 14CS0851-01-14 | 88.5 | 76.1 | 66.2 |
| 3 | SRS 934 | 78.7 | 77.8 | 61.7 |
| 3 | Midas ™ | 71.2 | 67.0 | 58.2 |
| means | 14CS0851-01-14 | 62.8 | 56.0 | 45.5 |
| | SRS 934 | 61.7 | 57.1 | 46.1 |
| | Midas ™ | 58.9 | 54.2 | 45.6 |
| | Anova P value (rep) | 0.07 | 0.02 | 0.03 |
| | Anova P value (line) | 0.93 | 0.93 | 1.00 |

†Anova P value (rep) for analysis of variance between experiments.
‡Apova P value (line) for analysis of variance between lines.

### Example 11: Methods of Identification & Detection

Herbicide screening—The mutant camelina line 14CS0851-01-14 can be easily distinguished from wild-type camelina types by spraying with the herbicide Pinnacle SG® (thifensulfuron-methyl) or Everest® (flucarbazone-sodium) at the 3-4 leaf stage, as described in Example 6. At present, since there are no other thifensulfuron-methyl tolerant or flucarbazone sodium tolerant camelina lines, screening by this method should be sufficient to identify 14CS0851-01-14 contamination of wild-type camelina grain.

DNA-based screening—On a molecular level, the mutation can be detected through DNA sequencing, as described in Example 8.

### Example 12: Toxicity of Modified AHAS Gene Products

A BLAST similarity search was conducted in the Toxin and Toxin Target Database (Wishart, 2015) using the amino acid sequences of 12CS0365 and 12CS0366 as well as the non-mutated camelina AHAS amino acid sequences (CsAHAS1, CsAHAS2, CsAHAS3). The sequences for each can be found in FIG. 7. The search revealed no hits using the following BLAST parameters:
Cost to open a gap: −1
Cost to extend a gap: −1
Penalty for mismatch: −3
Reward for mismatch: 1
Expectation value: 0.00001

AHAS protein is present in all plants and is not considered to be a toxin. The mutated CsAHAS protein of the present disclosure is not expected to behave differently than the generic protein in respect of toxicity. Furthermore, unintended effects related to the mutant line 14CS0851-01-14 have been investigated by analyzing the nutritional and anti-nutritional composition of the whole seeds and oil, presented here in Example 14.

### Example 13

A similarity search was conducted in the Allergenicity database (Pearson, 1988) using the mutated 12CS0365 and 12CS0366 and non-mutated camelina AHAS amino acid sequences (CsAHAS1, CsAHAS2, CsAHAS3). The sequences for each can be found in FIG. 7. The searches revealed no hits for either the full sequence, 80-mer sliding window, or 8-mer exact match searches. An additional search with the database AllerBase_BLAST (Altschul et al, 1997) also revealed no hits.

### Results—Allergenicity Database:

### i) Full Sequence

1>>>12CS0365-667 aa
Library: fasta/version17.fasta 481602 residues in 2035 sequences
481602 residues in 2035 sequences
Statistics: Expectation_n fit: rho(ln(x))=4.2363+/−0.00372; mu=19.0156+/−0.191
mean_var=67.5800+/−17.258, 0's: 1 Z-trim: 1 B-trim: 166 in 1/43
Lambda=0.156015
Algorithm: FASTA (3.5 Sep. 2006) [optimized]
Parameters: BL50 matrix (15: −5) ktup: 2
join: 38, opt: 26, open/ext:−10/−2, width: 16
Scan time: 1.000
!! No sequences with E( )<1.000000
667 residues in 1 query sequences
481602 residues in 2035 library sequences
Scomplib [35.04]
start: Wed December 13 15:51:15 2017 done: Wed December 13 15:51:16 2017
Total Scan time: 1.000 Total Display time: 0.000
1>>>12CS0366-667 aa
Library: fasta/version17.fasta 481602 residues in 2035 sequences
481602 residues in 2035 sequences
Statistics: Expectation_n fit: rho(ln(x))=4.2363+/−0.00372; mu=19.0156+/−0.191
mean_var=67.5800+/−17.258, 0's: 1 Z-trim: 1 B-trim: 166 in 1/43
Lambda=0.156015
Algorithm: FASTA (3.5 Sep. 2006) [optimized]
Parameters: BL50 matrix (15:−5) ktup: 2
join: 38, opt: 26, open/ext:−10/−2, width: 16
Scan time: 1.000
!! No sequences with E( )<1.000000

TABLE 30
AllergenOnline Database:

| | |
|---|---|
| Database | AllergenOnline Database v17 (Jan. 18, 2017) |
| Input Query | >12CS0365 |
| | MAAATTPSSSSIPFSTKPSSSKSPLPISRFTLPFALNPTKSSSSSRRRGIKSTS |
| | LSISAVLNTTTNVSTTTPPSKPTKPRKKKFVSRFAPDQPRKGADILVEALERQG |
| | VETVFAYPGGASMEIHQALTRSSSIRNVLPRHEQGGVFAAEGYARSTGKPGICI |
| | ATSGPGATNLVSGLADALDSVPLVAITGQVSRRMIGTDAFQETPIVEVTRSITK |
| | HNYLVMDVEDIPRIVEEAFFLATSGRPGPVLVDVPKDIQQQLAIPNWEQAMRLP |
| | GYMSRMPKPPEDSHLEQIVRLISESKKPVLYVGGGCLNSSEELGRFVELTGIPV |
| | ASTLMGLGAYPCDDELSLHMLGMHGTVYANYSVEHSDLLLAFGVRFDDRVTGKL |
| | EAFASRAKIVHIDIDSAEIGKNKTPHVSVCGDVKLALQGMNKVLENRAEELKLD |
| | FGVWRSELNEQKQKFPLSFKTFGEAIPPQYAIQVLDELTDGRAIISTGVGQHQM |
| | WAAQFYKYKKPRQWLSSAGLGAMGFGLPAAIGASVANPDAIVVDIDGDGSFIM |
| | NVQELATIRVENLPVKILILNNQHLGMVMQWEDRFYKANRAHTYLGNPGAEDEI |
| | FPNMLQFASACGIPAARVTKKAELREAIQKMLDTPGPYLLDVICPHQEHVLPMI |
| | PSGGTFNDVITEGDGRTKY (SEQ ID NO: 7) |
| Length | 667 |
| Number of | 588 |
| 80 mers | |
| Number | 0 |
| of | |
| Sequences | |
| with hits | |

80 mer Sliding Window Search Results

No Matches of Greater than 35% Identity Found

TABLE 31
AllergenOnline Database:

| | |
|---|---|
| Database | AllergenOnline Database v17 (Jan. 18, 2017) |
| Input Query | >12CS0366 |
| | MAAATTTSSSSIPFSTKPSSSKSPLPISRFTLPFSLNPNKSSSSSRRRGIKSTSL |
| | SISAVLNTTANVSTTTPPSKPTKPEKKKFVSRFAPDQPRKGADILVEALERQGVE |
| | TVFAYPGGASMEIHQALTRSSSIRNVLPRHEQGGVFAAEGYARSTGKPGICIATS |
| | GPGATNLVSGLADALLDSVPLVAITGQVSRRMIGTDAFQETPIVEVTRSITKHNY |
| | LVMDVEDIPRIVEEAFFLATSGRPGPVLVDVPKDIQQQLAIPNWEQSMRLPGYMS |
| | RMPKPPEDSHLEQIVRLISESKKPVLYVGGGCLNSSEELGRFVELTGIPVASTLM |
| | GLGAYPCDDELSLHMLGMHGTVYANYSVEHSDLLLAFGVREDDRVTGKLEAFASR |
| | AKIVHIDIDSAEIGKNKTPHVSVCGDVKLALQGMNKVLENRAEERKLDFGVWRSE |
| | LNEQKQKFPLSFKTFGEAIPPQYAIQVLDELTDGKAIISTGVGQHQMWAAQFYKY |
| | KKPRQWLSSAGLGAMGFGLPAAIGASVANPDAIVVDIDGDGSFIMNVQELATIRV |
| | ENLPVKILILNNQHLGMVMQWEDRFYKANRAHTYLGNPAAEDEIFPNMLQFASAC |
| | GIPAARVTKKAELREAIQKMLDTPGPYLLDVICPHQEHVLPMIPSGGTENDVITE |
| | GDGRTKY (SEQ ID NO: 8) |
| Length | 667 |
| Number of | 588 |
| 80 mers | |
| Number | 0 |
| of | |
| Sequences | |
| with hits | |

No Matches of Greater than 35% Identity Found

iii) 8mer Exact Match Search
>12CS0365
Number of 8mers=660
No sequences found with an exact 8mer match
>12CS0366
Number of 8mers=660
No sequences found with an exact 8mer match

### Results—AllerBase BLAST:

Query=12CS0365
Length=667
***** No hits found ******
Query=12CS0366
Length=667
****** No hits found ******

AHAS protein is present in all plants and is not considered to be an allergen. The mutated CsAHAS protein of the present disclosure is not expected to behave different than the endogenous protein. Results from the database searches can be found in the Appendix.

Further, mutant line 14CS0851-01-14 is not significantly different than non-mutated camelina types with regards to the amount of glucosinolates, as detailed in Example 14 herein. Camelina accumulates three different glucosinolates in its seeds (Daxenbichler et al., 1991; Lange et al., 1995; Schuster and Friedt, 1998):
glucoarabin (9-(methylsulfinyl) nonylglucosinolate—GS9),
glucocamelinine (10-(methylsulfinyl) decylglucosinolate—GS10), and
11-(methylsulfinyl) undecylglucosinolate (GS11)
   Whereas the first two glucosinolates have also been identified in other cruciferous plants, such as Alpine rock-cress and shepherd's purse, GS11 has only been detected in camelina. The levels of glucosinolates accumulated in seeds are affected by genotype and environmental conditions (Farnham et al., 2004); however, overall, when compared with other oilseeds of the Brassicaceae family, the content of glucosinolates in camelina seeds is moderate to low.

It is not known if camelina glucosinolates have an antinutritive effect when used as a feed ingredient, is unknown.

Because of the long side-chains of the compounds, enzymatic hydrolysis of camelina glucosinolates produces exclusively non-volatile and thus near-odorless isothiocyanates. Further, in contrast to canola seeds, seeds of camelina contain no progoitrin, which forms the toxic goitrin. The formation of goitrin homologues is unlikely, because the aglucones of glucosinolates from camelina contain no-OH groups.

Thus, from a nutritional point of view, it can with some certainty be concluded that the glucosinolates of camelina have a lower antinutritive effect than those of canola (Schumann and Stölken, 1996).

Camelina glucosinolates may further potentially be anti-cancer nutraceuticals in both animal and human diets (Berhow et al., 2013). The structure of the camelina glucosinolates is similar to that of glucoraphanin (4-(methylsulfinyl) butylglucosinolate), the difference being only the length of the aliphatic side chain. In theory, the degradation products of GS9, GS10, and GS11 should behave in a similar fashion to that of sulforaphane, the degradation product of glucoraphanin, which is an anticancer compound produced in broccoli and other crucifer vegetables (Shapiro et al., 2001; Talalay and Fahey, 2001; Fahey et al., 2003).

### Example 14: Nutritional Evaluation of Camelina Line 14CS0851-01-14

Selection of counterparts: Mutant camelina line 14CS0851-01-14 was developed by EMS mutagenesis of camelina accession SRS 934, as described in Example 1. Therefore, SRS 934 was chosen as the main comparator. However, SRS 934 is not commercially grown in Canada; therefore, a second comparator, commercial camelina variety MIDAS™, was also included in the nutritional evaluation.

Selection of field trial locations: Compositional analysis was undertaken on 3 different plot samples of 14CS0851-01-14, SRS 934, and MIDAS™ from 3 locations in a single year. The field trial locations were in proximity to Saskatoon, S K., Taber, A B., and Morris, MN. These 3 locations were chosen because they are in the regions where camelina is currently being grown (Saskatoon, Taber) or in agro-ecological zones that are similar to those where camelina is currently being grown in Canada. Thus, Morris, MN is located in Zone 5 according to Health Canada directive DIR2010-05: Revisions to the Residue Chemistry Crop Field Trials Requirements. Zone 5 stretches into Manitoba where camelina has been and is currently grown by farmers and in field trials.

Saskatoon (Aq-Quest farm) is in the dark brown soil zone; Taber, AB is in the brown soil zone; and the trial in Morris, MN was conducted at Swan Lake Research Farm on a soil classified as a Barnes loam soil. In Morris, the summers are long and warm; the winters are freezing, snowy, and windy; and it is partly cloudy year round. Over the course of the year, the temperature typically varies from −15° C. to 28° C. and is rarely below −27° C. or above 30° C. This is very similar to the climate in Southern Manitoba, a camelina growing area. For the purpose of demonstrating the equivalency of the climate in Morris, MN with the climate in a Canadian camelina growing area, a comparison was made to Carman, MB, which is also located in Zone 5. In Carman, MB, over the course of the year, the temperature typically varies from −20° C. to 26° C. and is rarely below-32° C. or above 31° C.

For Morris, MN and Carman, MB, the warm season lasts for about 4 months, from the middle of May until the third week in September, with an average daily high temperature above 21° C. in Morris, MN and above 19° C. in Carman, MB. For both locations, the hottest day of the year is in mid-end July (July 18 for Morris, MN, with an average high of 28° C. and low of 16° C.; July 25 for Carman, MB, with an average high of 26° C. and low of 14° C.

The rainy period of the year for both sites last from March to November (Morris, MN: March 11 to November 16; Carman, MB: March 24 to November 7). The most rain falls during the 31 days centered around June 18 and June 20, respectively, with an average total accumulation of rainfall during that period of 88.9 mm for Morris, MN and 79 mm for Carman, MB.

The growing season in Morris typically lasts for 4.8 months (149 days), from around May 3 to around September 29, rarely starting before April 12 or after May 22, and rarely ending before September 11 or after October 17. The growing season in Carman, MB is a bit shorter: it typically lasts for 4.1 months (127 days), from around May 19 to around September 23, rarely starting before April 30 or after June 6, and rarely ending before September 7 or after October 11.

In both locations, the major diseases that threaten camelina production are downy mildew (causal agent: Peronospora camelinae) and sclerotinia stem rot, caused by Sclerotinia sclerotiorum. In both regions, conservation tillage is commonly practiced and camelina is used mainly in rotation with cereal crops.

In the past 4 years of growing camelina commercially and in field trials in Canada from Ft. St. John, BC to Fredericton, NB and in the US in Washington State, Montana, Minnesota, North Dakota, South Dakota, and Texas, it was noted that camelina grows well in most soil types, provided they are well-drained.

Selection of analytes: Since there are no consensus documents published for camelina, reference was made to the Revised Consensus Document on Compositional Considerations for New Varieties of Low Erucic Acid Rapeseed (Canola): Key Food and Feed Nutrients, Anti-Nutrients and Toxicants, ENV/JM/MONO (2011) 55, Organization for Economic Co-operation and Development (OECD).

Whole seeds were analyzed for crude protein, crude fat, ash, moisture, acid detergent fibre (ADF), neutral detergent fibre (NDF), non-fibre carbohydrates (NFC), minerals (phosphorous and calcium), amino acids, glucosinolates, tannins, sinapine, trypsin inhibitors, and phytic acid.

Oil extracts of the whole seeds were analyzed for fatty acids and tocopherols (vitamin E), since those analytes are concentrated in the oil. Although vitamin K was included in the OECD consensus document for canola, a literature search for the presence and/or content of vitamin K in camelina did not produce any information on this analyte. Vitamin K is a fat-soluble vitamin found mostly in leafy green vegetables and is required for proper blood clotting function (Ferland, 2012.), and camelina is not known to contain significant amounts of Vitamin K.

Statistical Analysis: All statistical analyses were conducted using PROC Mixed (SAS Institute, 2009). The model is:

$${Y}_{ijk} = mu … + {r}_{i} , \_ + t \cdot {je}_{ijk}$$

Where Yᵢⱼₖ is the variable of interest, mu is the overall mean, rᵢ is the ᵢth, t is the jth entry and the eᵢⱼₖ is error.

Values represent the average of three samples for each location. Values followed by the same letters are not significantly different. Different letters denote statistically different least-squares means (P<0.05).

Nutritional Content of Camelina: At present, camelina oil is cold-pressed, non-solvent extracted. The extraction process uses only the heat generated by the press. No antioxidants are added. Camelina has a unique seed oil composition (Vollmann and Eynck, 2015), with a high content of α-linolenic acid (20 to >35%), eicosenoic acid (11-19%) and tocopherols (Vitamin E) (Zubr and Matthäus, 2002) as well as a naturally low level of the undesirable fatty acid erucic acid (<4%), rendering camelina oil well-suited for a variety of food, feed and non-food applications.

Nutritional Content of 14CS0851-01-14: The nutritional data herein on camelina oil and meal demonstrate that AHAS-mutant camelina variety 14CS0851-01-14 does not show any significant difference in composition in comparison to its parent SRS 934 or to commercial variety MIDAS™.

### Compositional Analysis of Camelina Seed

i) Proximate Composition: Proximate composition of camelina seed samples was analyzed by Cumberland Valley Analytical Services Inc., 4999 Zane A. Miller Drive, Waynesboro, PA 17268. Reference methods are as follows:
Ash: Ash in Animal Feed (942.05). Official Methods of Analysis, 17th Edition. 2000. Association of Official Analytical Chemists. Modification: 1.5 g sample weight, 4 hour ash time, hot weigh.
Moisture: Moisture in Animal Feed (930.15) Official Methods of Analysis, 18th Edition. 2006. Association of Official Analytical Chemists. Drying at 135° C.
Crude Fat: Crude Fat in Feeds, Cereal Grains, and Forages (2003.05) Official Methods of Analysis, 18th Edition. 2006. Association of Official Analytical Chemists. Tecator Soxtec System HT 1043 Extraction unit. Tecator, Foss NA 76822 Executive Drive, Eden Prairie, MN 55344.
Acid Detergent Fibre (ADF): Fibre (Acid Detergent) and Lignin in Animal Feed (973.18). Official Methods of Analysis, 17th Edition. 2000. Association of Official Analytical Chemists.
Neutral Detergent Fibre (NDF): Van Soest et al, 1991. Modification: Whatman 934-AH glass micro-fibre filters with 1.5 μm particle retention.
Non-Fibre Carbohydrates (NFCs): NFCs are made up of starch, simple sugars, and soluble fibre. NFC is calculated by subtracting % NDF, % CP, % Fat and % Ash from 100% [100%−(% NDF+% CP+% Fat+% Ash)]. NFCs are sometimes called non-structural carbohydrates (NSC) and usually make up 35-40% of the dry matter in a dairy ration designed for high milk production.

### Results:

Significant differences were observed only for acid detergent fibre (ADF) at Saskatoon (Table 32). ADF expressed as % of dry matter was similar for 14CS0851-01-14 and MIDAS™ and both were significantly higher than ADF of SRS 934. Neutral detergent fibre (NDF) and ash did not differ between entries or locations. The raw data can be found below in Table 33.

TABLE 32
Ash, Fibre (acid detergent fibre, ADF, and neutral detergent
fibre, NDF), and Non-Fibre Carbohydrate (NFC) content in seed
of 14CS0851-01-14, SRS 934 and Midas ™ at Morris, MN,
Saskatoon, SK and Taber, AB. Expressed as % of dry matter. Values
represent the average of three samples for each location. Values
followed by the same letters are not significantly different.

| | Ash | ADF | NDF | NFC |
|---|---|---|---|---|
| Location/Entry | (% DM) | (% DM) | (% DM) | (% DM) |
| Morris, Minnesota, 2016 | | | | |
| 14CS0851-01-14 | 4.1 A | 24.8 A | 29.7 AB | 34.8 A |
| MIDAS ™ | 4.4 A | 25.9 A | 35.0 A | 31.3 A |
| SRS 934 | 4.2 A | 21.2 A | 25.9 B | 37.8 A |
| Std. error | 0.2 | 3.6 | 3.0 | 3.1 |
| Anova P-value | 0.57 | 0.44 | 0.06 | 0.20 |
| Saskatoon, | | | | |
| Saskatchewan, 2016 | | | | |
| 14CS0851-01-14 | 3.4 A | 25.1 A | 33.2 A | 34.0 A |
| MIDAS ™ | 3.5 A | 25.0 A | 36.5 A | 32.6 A |
| SRS 934 | 3.6 A | 21.8 B | 32.6 A | 34.3 A |
| Std. error | 0.1 | 1.1 | 3.2 | 3.2 |
| Anova P-value | 0.39 | 0.05 | 0.48 | 0.85 |
| Taber, Alberta, 2016 | | | | |
| 14CS0851-01-14 | 4.4 AB | 22.3 A | 30.6 A | 32.2 A |
| MIDAS ™ | 4.4 A | 21.7 A | 28.8 A | 36.5 A |
| SRS 934 | 4.0 B | 21.1 A | 28.0 A | 35.4 A |
| Std. error | 0.2 | 2.0 | 1.9 | 1.9 |
| Anova P-value | 0.08 | 0.83 | 0.45 | 0.18 |

TABLE 33
Raw data - ash, fibre (acid detergent fibre, ADF, and neutral detergent fibre, NDF), and
non-fibre carbohydrate (NFC) content in seed of 14CS0851-01-14, SRS 934 and Midas ™
at Morris, MN, Saskatoon, SK and Taber, AB. Expressed as % of dry matter. Results from
Cumberland Valley Analytical Services.

| | | | | | | | | NonFiber |
|---|---|---|---|---|---|---|---|---|
| | | | Dry | ADF | ADF | aNDF | Ash | Carbohydrates |
| Plot | Entry | Moisture | Matter | (% NDF) | (% DM) | (% DM) | (% DM) | (% DM) |
| Morris, MN | | | | | | | | |
| 107 | Midas | 4.0 | 96.0 | 74.2 | 26.6 | 35.9 | 4.89 | 29.8 |
| 202 | Midas | 4.1 | 95.9 | 78.6 | 29.9 | 38.1 | 4.18 | 29 |
| 308 | Midas | 4.1 | 95.9 | 68.2 | 21.1 | 31.0 | 4.1 | 35.2 |
| 105 | 14Cs0851-01-14 | 3.7 | 96.3 | 88.6 | 25.2 | 28.4 | 3.95 | 36.1 |
| 207 | 14Cs0851-01-14 | 3.8 | 96.2 | 74.1 | 20.0 | 27.0 | 4.09 | 37.8 |
| 306 | 14Cs0851-01-14 | 4.1 | 95.9 | 86.7 | 29.1 | 33.6 | 4.32 | 30.4 |
| 103 | SRS934 | 3.8 | 96.2 | 77.8 | 16.5 | 21.2 | 4.29 | 42.5 |
| 204 | SRS934 | 3.8 | 96.2 | 83.3 | 23.8 | 28.6 | 4.38 | 35.0 |
| 301 | SRS934 | 3.9 | 96.3 | 83.6 | 23.3 | 27.8 | 3.96 | 35.8 |
| Saskatoon, SK | | | | | | | | |
| 301 | Midas | 4.5 | 95.5 | 65.7 | 24.9 | 37.9 | 3.56 | 31.0 |
| 202 | Midas | 4.6 | 95.4 | 68.6 | 25.S | 37.1 | 3.50 | 31.8 |
| 101 | Midas | 4.5 | 95.5 | 71.3 | 24.6 | 34.5 | 3.37 | 34.9 |
| 303 | 14Cs0851-01-14 | 4.8 | 95.2 | 67.2 | 26.5 | 39.5 | 3.35 | 27.9 |
| 201 | 14Cs0851-01-14 | 4.7 | 95.3 | 75.4 | 25.0 | 33.2 | 3.32 | 34.0 |
| 103 | 14Cs0851-01-14 | 4.8 | 95.3 | 87.5 | 23.7 | 27.0 | 3.61 | 40.1 |
| 302 | SRS934 | 4.7 | 95.3 | 73.1 | 23.1 | 31.6 | 3.46 | 35.7 |
| 203 | SRS934 | 4.8 | 95.2 | 62.7 | 19.6 | 31.3 | 3.55 | 35.1 |
| 102 | SRS934 | 4.7 | 95.3 | 65.2 | 22.8 | 35.0 | 3.79 | 32.1 |
| Taber, AB | | | | | | | | |
| 304 | Midas | 4.7 | 95.3 | 73.1 | 19.3 | 26.4 | 4.21 | 39.0 |
| 201 | Midas | 7.9 | 92.1 | 74.9 | 20.6 | 27.5 | 4.44 | 37.6 |
| 103 | Midas | 4.6 | 95.4 | 77.2 | 25.1 | 32.5 | 4.50 | 33.0 |
| 208 | 14Cs0851-01-14 | 4.9 | 95.1 | 77.6 | 23.3 | 30.0 | 4.34 | 32.7 |
| 303 | 14Cs0851-01-14 | 4.9 | 95.1 | 75.5 | 22.8 | 30.1 | 4.10 | 33.1 |
| 104 | 14Cs0851-01-14 | 4.9 | 95.1 | 66.0 | 20.9 | 31.7 | 4.65 | 30.8 |
| 309 | SRS934 | 4.7 | 95.3 | 66.0 | 19.3 | 29.3 | 4.05 | 34.3 |
| 206 | SRS934 | 4.6 | 95.4 | 84.2 | 19.5 | 23.2 | 4.08 | 40.4 |
| 107 | SRS934 | 4.6 | 95.4 | 77.4 | 24.5 | 31.6 | 3.80 | 31.5 |

ii) Crude oil and protein: Seed oil and total protein were analyzed at Agriculture and Agri-Food Canada, Saskatoon Research and Development Center by near infrared (NIR). Seed oil content is determined by near-infrared reflectance according to AOCS standard procedure Am 1-92: Determination of oil, moisture and volatile matter, and protein by near-infrared reflectance. A Foss NIRSystems® Model 6500 analyzer calibrated with appropriate oilseed samples extracted with hexane was used, according to Raney et al (1987), with modifications. Results are reported as a percentage on a whole seed dry matter (zero moisture) basis.

Seed protein content was also determined by near-infrared reflectance according to AOCS standard procedure Am 1-92: Determination of oil, moisture and volatile matter, and protein by near-infrared reflectance. Results are reported as a percentage, N×6.25, calculated on a whole seed dry matter (zero moisture) basis. As for the determination of seed oil content, a Foss NIRSystems® Model 6500 analyzer calibrated with appropriate oilseed samples was used. Calibration of the NIRSystems® Model 6500 is performed with oilseed samples whose protein contents were determined by the AOCS official method Ba 4e-93, revised 2003: Generic combustion method for determination of crude protein using a LECO® FP-528 Protein Analyzer.

### Results:

Significant differences were observed for seed oil and protein contents across all three locations (Morris, MN, Saskatoon, S K and Taber, AB) (Table 34).

TABLE 34
Crude oil (% DM) and protein (% DM) contents in seeds
of 14CS0851-01-14, SRS 934 and Midas ™ at Morris,
MN, Saskatoon, SK, and Taber, AB. Values represent averages
of three samples for each site. Values followed with
the same letter are not significantly different.

| Location/Entry | Oil (% DM) | Protein (% DM) |
|---|---|---|
| Morris, Minnesota, 2016 | | |
| 14CS0851-01-14 | 35.0 A | 32.2 A |
| MIDASTMTM | 37.7 B | 29.9 B |
| SRS 934 | 36.7 B | 31.6 A |
| Std. error | 0.5 | 0.3 |
| Anova P-value | 0.005 | 0.002 |
| Saskatoon, Saskatchewan, 2016 | | |
| 14CS0851-01-14 | 37.9 A | 29.4 A |
| MIDASTMTM | 39.9 B | 27.7 B |
| SRS 934 | 38.0 A | 29.3 A |
| Std. error | 0.1 | 0.2 |
| Anova P-value | 0.000 | 0.000 |
| Taber, Alberta, 2016 | | |
| 14CS0851-01-14 | 36.7 A | 30.8 C |
| MIDASTMTM | 38.6 B | 28.9 A |
| SRS 934 | 36.3 A | 31.7 B |
| Std. error | 0.3 | 0.3 |
| Anova P-value | 0.001 | 0.000 |

Seed oil contents of 14CS0851-01-14 were lower than those of both checks at Morris and lower than that of MIDAS™ at Saskatoon and Taber. The protein content of 14CS0851-01-14 was higher than that of MIDAS™ at all three locations. The raw data can be found in Table 35.

TABLE 35
Crude oil (% DM) and protein (% DM) contents in seeds
of 14CS0851-01-14, SRS 934 and MIDAS ™ at
Morris, MN, Saskatoon, SK, and Taber, AB. Results from
Agriculture and Agri-Food Canada, Saskatoon Research Centre.

| | Plot | Entry | Oil (%) | Protein (%) |
|---|---|---|---|---|
| Morris, MN | | | | |
| | 105 | 14CS0851-01-14 | 37.12 | 31.06 |
| | 207 | 14CS0851-01-14 | 36.26 | 30.74 |
| | 306 | 14CS0851-01-14 | 36.76 | 30.54 |
| | 103 | SRS 934 | 36.09 | 32.03 |
| | 204 | SRS 934 | 36.92 | 30.96 |
| | 301 | SRS 934 | 35.76 | 32.02 |
| | 107 | Midas | 38.28 | 29.05 |
| | 202 | Midas | 39.4 | 28.35 |
| | 308 | Midas | 38.19 | 29.23 |
| Saskatoon, SK | | | | |
| | 103 | 14CS0851-01-14 | 37.65 | 29.52 |
| | 201 | 14CS0851-01-14 | 37.68 | 29.47 |
| | 303 | 14CS0851-01-14 | 38.37 | 29.26 |
| | 102 | SRS 934 | 37.97 | 29.12 |
| | 203 | SRS 934 | 37.75 | 29.55 |
| | 302 | SRS 934 | 38.38 | 29.2 |
| | 101 | Midas | 39.84 | 28.02 |
| | 202 | Midas | 39.62 | 27.42 |
| | 301 | Midas | 40.17 | 27.74 |
| Taber, AB | | | | |
| | 104 | 14CS0851-01-14 | 35.05 | 32.3 |
| | 208 | 14CS0851-01-14 | 35.23 | 31.72 |
| | 303 | 14CS0851-01-14 | 34.82 | 32.69 |
| | 107 | SRS 934 | 36.22 | 31.74 |
| | 206 | SRS 934 | 36.59 | 31.72 |
| | 309 | SRS 934 | 37.25 | 31.3 |
| | 103 | Midas | 37.92 | 29.83 |
| | 201 | Midas | 37.19 | 30.12 |
| | 304 | Midas | 38.06 | 29.95 |

iii) Amino acid profile: Amino acid profiles of 3 seed samples for each comparator from 3 locations were analyzed by the University of Missouri using AOAC Official Method 982.30 E (a,b,c), chp. 45.3.05, 2006.

As mentioned in Example 10, branched-chain amino acids (BCAAs) have been associated with germination. Tranel and Wright (2002) suggested that a reduction in feedback inhibition (decreased enzyme sensitivity) of the AHAS enzyme could result in higher concentrations of BCAAs in seeds, thereby positively affecting germination. This association has been observed by Dyer et al. (1993) in kochia when comparing plants with a mutated AHAS gene to wild-type plants. As such, the three amino acids (AA) implicated by these studies were analyzed: valine, isoleucine and leucine.

### Results:

Significant differences between the entries were observed for all three branched-chain AAs at all locations (Table 36). Both 14CS0851-01-14 and SRS 934 had higher BCAA levels than MIDAS™ at all locations. At all locations, the amount of BCAAs in 14CS0851-01-14 was not significantly different than those in SRS 934, except for the valine and isoleucine contents at Morris, MN: here, the content in 14CS0851-01-14 was significantly lower than in SRS 934. The complete amino acid profiles can be found in Table 37.

TABLE 36
Amino acid profiles (g/100 g seed) of 14CS0851-01-14, SRS 934
and MIDAS ™ at Morris, MN, Saskatoon, SK and Taber,
AB. Values represent averages of three samples for each locations.
Values followed by the same letters are not significantly different.

| Location/Entry | Valine | Isoleucine | Leucine |
|---|---|---|---|
| Morris, Minnesota, 2016 | | | |
| 14CS0851-01-14 | 1.81 B | 1.27 B | 2.10 A |
| MIDASTM | 1.70 C | 1.19 C | 1.94 B |
| SRS 934 | 1.87 A | 1.30 A | 2.15 A |
| Std. error | 0.01 | 0.01 | 0.02 |
| Anova P-value | 0.00 | 0.00 | 0.00 |
| Saskatoon, Saskatchewan, 2016 | | | |
| 14CS0851-01-14 | 1.63 A | 1.15 A | 1.89 A |
| MIDASTM | 1.52 B | 1.07 B | 1.75 B |
| SRS 934 | 1.61 A | 1.15 A | 1.88 A |
| Std. error | 0.02 | 0.01 | 0.02 |
| Anova P-value | 0.00 | 0.00 | 0.00 |
| Taber, Alberta, 2016 | | | |
| 14CS0851-01-14 | 1.79 A | 1.26 A | 2.11 A |
| MIDASTM | 1.66 B | 1.18 B | 1.94 B |
| SRS 934 | 1.78 A | 1.26 A | 2.09 A |
| Std. error | 0.02 | 0.01 | 0.02 |
| Anova P-value | 0.00 | 0.00 | 0.00 |

TABLE 37
Amino acid profiles (g/100 g seed) of 14CS0851-01-14, SRS 934 and MIDAS ™ at Saskatoon, SK (16C503AQSA),
Taber, AB (16C503TA), and Morris, MN, (16C503MN). Results from Cumberland Valley Analytical Services.

| | |
|---|---|
| [symbol] | CUMBERLAND VALLEY ANALYTICAL SERVICES, INC. mail@ [symbol] .com www. [symbol] .com |

Linnaeus Plant Sciences, Inc. Amino Acid Profile Spreadsheet

| | |
|---|---|
| 2017 Mar. 22 | Results are on a Dry Matter Basis (w/w %) |

Batch 21545 w/w % = grams per 100 grams of sample

| | Sample | | | | | | | |
|---|---|---|---|---|---|---|---|---|
| | 3 | 4 | 5 | | | | | |
| | 16C503AQSA | 16C503AQSA | 16C503AQSA | 6 | 7 | 8 | 9 | 10 |
| | 14CS0851- | 14CS0851- | 14CS0851- | 16C503AQSA | 16C503AQSA | 16C503AQSA | 16C503AQSA | 16C503AQSA |
| | 01-14 | 01-14 | 01-14 | SRS934 | SRS934 | SRS934 | Midas | Midas |
| | PLOT-103 | PLOT-201 | PLOT-303 | PLOT-102 | PLOT-203 | PLOT-302 | PLOT-101 | PLOT-202 |
| Description | TRT-1 | TRT-1 | TRT-1 | TRT-2 | TRT-2 | TRT-2 | TRT-3 | TRT-3 |
| Taurine | 0.01 | 0.01 | 0.01 | 0.01 | 0.01 | 0.01 | 0.01 | 0.01 |
| Hydroxy- | 0.31 | 0.32 | 0.33 | 0.27 | 0.28 | 0.26 | 0.28 | 0.33 |
| proline | | | | | | | | |
| Aspartic | 2.36 | 2.40 | 2.38 | 2.44 | 2.48 | 2.46 | 2.27 | 2.22 |
| acid | | | | | | | | |
| Threonine | 1.17 | 1.18 | 1.17 | 1.16 | 1.19 | 1.17 | 1.14 | 1.11 |
| Serine | 1.16 | 1.17 | 1.15 | 1.15 | 1.17 | 1.18 | 1.12 | 1.09 |
| Glutamic | 4.55 | 4.70 | 4.59 | 4.47 | 4.53 | 4.61 | 4.27 | 4.07 |
| Acid | | | | | | | | |
| Proline | 1.66 | 1.70 | 1.63 | 1.58 | 1.61 | 1.59 | 1.60 | 1.47 |
| Lanthionine | 0.00 | 0.00 | 0.00 | 0.00 | 0.00 | 0.00 | 0.00 | 0.00 |
| Glycine | 1.52 | 1.55 | 1.54 | 1.55 | 1.56 | 1.55 | 1.51 | 1.38 |
| Alanine | 1.27 | 1.29 | 1.28 | 1.29 | 1.31 | 1.31 | 1.22 | 1.17 |
| Cysteine | 0.73 | 0.75 | 0.72 | 0.70 | 0.70 | 0.71 | 0.66 | 0.62 |
| Valine | 1.60 | 1.64 | 1.64 | 1.61 | 1.62 | 1.59 | 1.54 | 1.49 |
| Methionine | 0.52 | 0.54 | 0.53 | 0.52 | 0.52 | 0.52 | 0.50 | 0.48 |
| Isoleucine | 1.14 | 1.16 | 1.16 | 1.14 | 1.16 | 1.14 | 1.09 | 1.05 |
| Leucine | 1.86 | 1.92 | 1.89 | 1.86 | 1.88 | 1.90 | 1.77 | 1.71 |
| Tyrosine | 0.80 | 0.80 | 0.78 | 0.78 | 0.78 | 0.81 | 0.78 | 0.75 |
| Phenyl- | 1.29 | 1.32 | 1.30 | 1.28 | 1.29 | 1.30 | 1.23 | 1.20 |
| alanine | | | | | | | | |
| Hydroxy- | 0.06 | 0.06 | 0.06 | 0.06 | 0.06 | 0.06 | 0.06 | 0.05 |
| lysine | | | | | | | | |
| Ornithine | 0.01 | 0.01 | 0.01 | 0.01 | 0.01 | 0.01 | 0.00 | 0.01 |
| Lysine | 1.46 | 1.48 | 1.48 | 1.48 | 1.49 | 1.46 | 1.42 | 1.40 |
| Histidine | 0.68 | 0.70 | 0.69 | 0.68 | 0.69 | 0.69 | 0.63 | 0.62 |
| Arginine | 2.39 | 2.48 | 2.41 | 2.39 | 2.42 | 2.46 | 2.27 | 2.19 |
| Tryptophan | 0.27 | 0.27 | 0.25 | 0.28 | 0.28 | 0.27 | 0.24 | 0.24 |
| Total | 26.82 | 27.47 | 27.01 | 26.73 | 27.04 | 27.06 | 25.64 | 24.69 |

Batch 21545 w/w % = grams per 100 grams of sample

| | Sample | | | | | | | |
|---|---|---|---|---|---|---|---|---|
| | | 12 | 13 | 14 | | | | |
| | 11 | 16C503TA | 16C503TA | 16C503TA | 15 | 16 | 17 | 18 |
| | 16C503AQSA | 14CS0851- | 14CS0851- | 14CS0851- | 16C503TA | 16C503TA | 16C503TA | 16C503TA |
| | Midas | 01-14 | 01-14 | 01-14 | SRS934 | SRS934 | SRS934 | Midas |
| | PLOT-301 | PLOT-104 | PLOT-208 | PLOT-303 | PLOT-107- | PLOT-206 | PLOT-309 | PLOT-103 |
| Description | TRT-3 | TRT-1 | TRT-1 | TRT-1 | TRT-2 | TRT2 | TRT-2 | TRT-3 |
| Taurine | 0.01 | 0.01 | 0.01 | 0.01 | 0.01 | 0.01 | 0.01 | 0.01 |
| Hydroxy- | 0.30 | 0.30 | 0.31 | 0.27 | 0.26 | 0.24 | 0.28 | 0.31 |
| proline | | | | | | | | |
| Aspartic | 2.25 | 2.60 | 2.57 | 2.60 | 2.65 | 2.60 | 2.67 | 2.45 |
| acid | | | | | | | | |
| Threonine | 1.13 | 1.26 | 1.25 | 1.26 | 1.24 | 1.23 | 1.25 | 1.20 |
| Serine | 1.12 | 1.26 | 1.25 | 1.28 | 1.24 | 1.23 | 1.25 | 1.19 |
| Glutamic | 4.25 | 5.28 | 5.17 | 5.32 | 5.14 | 5.00 | 5.12 | 4.82 |
| Acid | | | | | | | | |
| Proline | 1.58 | 1.89 | 1.78 | 1.87 | 1.82 | 1.78 | 1.81 | 1.71 |
| Lanthionine | 0.00 | 0.00 | 0.00 | 0.00 | 0.00 | 0.00 | 0.00 | 0.00 |
| Glycine | 1.52 | 1.69 | 1.66 | 1.70 | 1.70 | 1.68 | 1.71 | 1.64 |
| Alanine | 1.23 | 1.42 | 1.41 | 1.43 | 1.41 | 1.39 | 1.40 | 1.32 |
| Cysteine | 0.66 | 0.77 | 0.78 | 0.77 | 0.74 | 0.74 | 0.76 | 0.73 |
| Valine | 1.54 | 1.80 | 1.77 | 1.81 | 1.81 | 1.76 | 1.78 | 1.66 |
| Methionine | 0.50 | 0.57 | 0.57 | 0.56 | 0.55 | 0.55 | 0.57 | 0.53 |
| Isoleucine | 1.08 | 1.27 | 1.25 | 1.27 | 1.28 | 1.25 | 1.26 | 1.18 |
| Leucine | 1.76 | 2.11 | 2.08 | 2.13 | 2.12 | 2.06 | 2.08 | 1.94 |
| Tyrosine | 0.78 | 0.90 | 0.88 | 0.88 | 0.86 | 0.85 | 0.86 | 0.80 |
| Phenyl- | 1.22 | 1.43 | 1.41 | 1.44 | 1.45 | 1.42 | 1.43 | 1.32 |
| alanine | | | | | | | | |
| Hydroxy- | 0.06 | 0.07 | 0.07 | 0.07 | 0.06 | 0.06 | 0.07 | 0.06 |
| lysine | | | | | | | | |
| Ornithine | 0.00 | 0.01 | 0.00 | 0.01 | 0.01 | 0.01 | 0.01 | 0.01 |
| Lysine | 1.43 | 1.59 | 1.54 | 1.58 | 1.55 | 1.53 | 1.54 | 1.46 |
| Histidine | 0.63 | 0.76 | 0.75 | 0.76 | 0.76 | 0.75 | 0.75 | 0.70 |
| Arginine | 2.27 | 2.76 | 2.73 | 2.78 | 2.74 | 2.68 | 2.70 | 2.50 |
| Tryptophan | 0.24 | 0.27 | 0.25 | 0.27 | 0.25 | 0.32 | 0.30 | 0.27 |
| Total | 25.58 | 30.03 | 29.50 | 30.09 | 29.64 | 29.14 | 29.61 | 27.81 |

Batch 21545 w/w % = grams per 100 grams of sample

| | Sample | | | | | | | |
|---|---|---|---|---|---|---|---|---|
| | | | 22 | 23 | 24 | | | |
| | 19 | 21 | 16C506MN | 16C503MN | 16C503MN | 25 | 26 | 27 |
| | 16C503TA | 16C503TA | 14CS0851- | 14CS0851- | 14CS0851- | 16C503MN | 16C503MN | 16C503MN |
| | Midas | Midas | 01-14 | 01-14 | 01-14 | SRS934 | SRS934 | SRS934 |
| | PLOT-201 | PLOT-304 | PLOT-105 | PLOT-207 | PLOT-306 | PLOT-103 | PLOT-204 | PLOT-301 |
| Description | TRT-3 | TRT-3 | TRT-1 | TRT-1 | TRT-1 | TRT-2 | TRT-2 | TRT-2 |
| Taurine | 0.01 | 0.01 | 0.02 | 0.03 | 0.02 | 0.02 | 0.02 | 0.01 |
| Hydroxy- | 0.26 | 0.31 | 0.29 | 0.31 | 0.33 | 0.25 | 0.27 | 0.28 |
| proline | | | | | | | | |
| Aspartic | 2.52 | 2.36 | 2.66 | 2.66 | 2.64 | 2.87 | 2.79 | 2.77 |
| acid | | | | | | | | |
| Threonine | 1.22 | 1.17 | 1.28 | 1.29 | 1.28 | 1.33 | 1.28 | 1.29 |
| Serine | 1.21 | 1.17 | 1.26 | 1.28 | 1.29 | 1.36 | 1.28 | 1.30 |
| Glutamic | 4.91 | 4.69 | 4.69 | 4.66 | 4.67 | 4.86 | 4.69 | 4.79 |
| Acid | | | | | | | | |
| Proline | 1.76 | 1.70 | 1.68 | 1.67 | 1.72 | 1.71 | 1.67 | 1.72 |
| Lanthionine | 0.00 | 0.00 | 0.00 | 0.00 | 0.00 | 0.00 | 0.00 | 0.00 |
| Glycine | 1.67 | 1.58 | 1.66 | 1.67 | 1.67 | 1.76 | 1.72 | 1.73 |
| Alanine | 1.34 | 1.28 | 1.40 | 1.43 | 1.43 | 1.51 | 1.46 | 1.49 |
| Cysteine | 0.75 | 0.70 | 0.59 | 0.58 | 0.58 | 0.61 | 0.58 | 0.60 |
| Valine | 1.69 | 1.64 | 1.82 | 1.81 | 1.81 | 1.88 | 1.85 | 1.86 |
| Methionine | 0.55 | 0.51 | 0.50 | 0.50 | 0.49 | 0.52 | 0.50 | 0.52 |
| Isoleucine | 1.19 | 1.17 | 1.28 | 1.27 | 1.26 | 1.31 | 1.29 | 1.30 |
| Leucine | 1.97 | 1.92 | 2.12 | 2.09 | 2.09 | 2.19 | 2.12 | 2.13 |
| Tyrosine | 0.84 | 0.82 | 0.88 | 0.88 | 0.89 | 0.91 | 0.88 | 0.89 |
| Phenyl- | 1.34 | 1.32 | 1.48 | 1.48 | 1.48 | 1.55 | 1.50 | 1.51 |
| alanine | | | | | | | | |
| Hydroxy- | 0.06 | 0.06 | 0.07 | 0.07 | 0.07 | 0.07 | 0.07 | 0.07 |
| lysine | | | | | | | | |
| Ornithine | 0.01 | 0.01 | 0.00 | 0.01 | 0.00 | 0.00 | 0.01 | 0.01 |
| Lysine | 1.49 | 1.46 | 1.54 | 1.56 | 1.57 | 1.61 | 1.59 | 1.59 |
| Histidine | 0.71 | 0.69 | 0.73 | 0.74 | 0.73 | 0.77 | 0.74 | 0.75 |
| Arginine | 2.58 | 2.48 | 2.50 | 2.47 | 2.48 | 2.63 | 2.52 | 2.58 |
| Tryptophan | 0.28 | 0.30 | 0.28 | 0.32 | 0.30 | 0.39 | 0.32 | 0.32 |
| Total | 28.36 | 27.34 | 28.72 | 28.77 | 28.79 | 30.12 | 29.14 | 29.49 |

Batch 21545 w/w % = grams per 100 grams of sample

| | Sample | | | | | | | |
|---|---|---|---|---|---|---|---|---|
| | 28 | 29 | 30 | | | | | |
| | Midas | Midas | Midas | | | | | |
| | 16C506MN | 16C506MN | 16C506MN | | | | | |
| | PLOT-107 | PLOT-202 | PLOT-308 | | | | | |
| Description | TRT-3 | TRT-3 | TRT-1 | | | | | |
| Taurine | 0.02 | 0.02 | 0.02 | | | | | |
| Hydroxy- | 0.31 | 0.29 | 0.32 | | | | | |
| proline | | | | | | | | |
| Aspartic | 2.51 | 2.44 | 2.55 | | | | | |
| acid | | | | | | | | |
| Threonine | 1.23 | 1.20 | 1.25 | | | | | |
| Serine | 1.23 | 1.20 | 1.24 | | | | | |
| Glutamic | 4.35 | 4.19 | 4.27 | | | | | |
| Acid | | | | | | | | |
| Proline | 1.57 | 1.46 | 1.52 | | | | | |
| Lanthionine | 0.00 | 0.00 | 0.00 | | | | | |
| Glycine | 1.64 | 1.60 | 1.62 | | | | | |
| Alanine | 1.43 | 1.38 | 1.41 | | | | | |
| Cysteine | 0.55 | 0.52 | 0.53 | | | | | |
| Valine | 1.72 | 1.67 | 1.71 | | | | | |
| Methionine | 0.46 | 0.46 | 0.46 | | | | | |
| Isoleucine | 1.21 | 1.16 | 1.19 | | | | | |
| Leucine | 1.96 | 1.89 | 1.96 | | | | | |
| Tyrosine | 0.85 | 0.82 | 0.84 | | | | | |
| Phenyl- | 1.39 | 1.35 | 1.40 | | | | | |
| alanine | | | | | | | | |
| Hydroxy- | 0.07 | 0.06 | 0.07 | | | | | |
| lysine | | | | | | | | |
| Ornithine | 0.01 | 0.01 | 0.01 | | | | | |
| Lysine | 1.50 | 1.45 | 1.49 | | | | | |
| Histidine | 0.69 | 0.67 | 0.69 | | | | | |
| Arginine | 2.32 | 2.23 | 2.30 | | | | | |
| Tryptophan | 0.30 | 0.26 | 0.31 | | | | | |
| Total | 27.33 | 26.32 | 27.16 | 0.00 | 0.00 | 0.00 | 0.00 | 0.00 |

[symbol]

indicates data missing or illegible when filed

iv) Fatty acid profile: For determination of total seed fatty acid composition, acid-catalysed transesterification, using methanolic hydrogen chloride was performed (Puttick et al, 2009). In the presence of a large excess of methanol, the equilibrium point of the reaction is shifted so that esterification of the fatty acids proceeds virtually to completion and the derivatized fatty acid methyl ester (FAME) is detected by gas chromatography. Approximately 30 seeds from each sample plot were placed in Pyrex® screw cap tubes with 3 mL 1M HCl in methanol and 500 mL of hexane. The tubes were tightly capped and heated at 80° C. overnight. After cooling, 3 mL of 0.9% NaCl and 1.5 mL hexane was added and fatty acid methyl esters (FAMEs) were recovered by collecting the hexane phase. Gas chromatography of FAMES was conducted using an Agilent 6890N GC fitted with a DB-23 capillary column (0.25 mm×30 m, 0.25 UM thickness; J & W, Folsom, CA, USA) and flame ionization detector, as described by Kunst et al (1992). Results are expressed as % of total area. A fatty acid standard mix C4:0-C22:6 (GLC-607, Nu-Chek Prep, Elysian MN, USA) was initially run to verify peak identities.

### Results:

Significant differences were observed at all three sites for all FAMES measured, save for poly-unsaturated fatty acids (PUFAs) at Morris, where there were no differences between the entries (Table 38). 14CS0851-01-14 was similar to MIDAS™ in PUFAs but approximately one percent higher than SRS 934 at Saskatoon and Taber. Mono-unsaturated fatty acids (MUFAs) were similar for the checks save for Taber and higher than for 14CS0851-01-14 at all three sites. C20:1 was lower in 14CS0851-01-14 than in the checks. The erucic acid (C22:1) levels for 14CS0851-01-14 were generally lower than for MIDAS™ and SRS 934 or similar to that of SRS 934. MIDAS™ exhibited the highest erucic acid levels. Total saturated fatty acid levels differed by location. At Morris, the total saturated fatty acid content of 14CS0851-01-14 was higher than that of both checks, while at Saskatoon and Taber, the content of 14CS0851-01-14 was similar to or marginally higher than that of SRS 934 and higher than that of MIDAS™. Complete fatty acid profiles can be found in Table 39.

TABLE 38
Alpha-Linolenic acid (C18:3), gondoic acid (C20:1), erucic acid (C22:1) content as
well as total saturated fatty acid (SATS), mono-unsaturated fatty acid (MUFA) and
polyunsaturated fatty acid (PUFA) content (% of total fatty acids of seed oil) of
14CS0851-01-14, SRS 934 and MIDAS ™ at Morris, MN, Saskatoon, SK, and
Taber, AB. Values represent averages of three samples for each location. Values
followed by the same letters are not significantly different.

| Location/Entry | C18:3 | C20:1 | C22:1 | SATS | MUFA | PUFA |
|---|---|---|---|---|---|---|
| Morris, Minnesota, | | | | | | |
| 2016 | | | | | | |
| 14CS0851-01-14 | 31.7 A | 12.0 B | 2.5 B | 12.7 A | 30.8 B | 56.1 A |
| MIDASTM | 30.2 B | 12.9 A | 2.8 A | 11.2 B | 32.7 A | 55.7 AB |
| SRS 934 | 32.1 A | 12.6 C | 2.4 B | 11.5 B | 32.7 A | 55.4 B |
| Std. error | 0.30 | 0.13 | 0.08 | 0.22 | 0.20 | 0.24 |
| Anova P-value | 0. [symbol] | 0.001 | 0.003 | 0.000 | 0.000 | 0.070 |
| Saskatoon, | | | | | | |
| Saskatchewan, | | | | | | |
| 2016 | | | | | | |
| 14CS0851-01-14 | 34.6 A | 12.8 B | 2.7 B | 12.5 A | 30.1 B | 56.8 A |
| MIDASTM | 32.9 B | 13.6 A | 3.1 A | 10.6 C | 31.8 A | 57.0 A |
| SRS 934 | 33.3 B | 13.6 A | 2.5 B | 11.7 B | 32.0 A | 55.7 B |
| Std. error | 0.49 | 0.16 | 0.11 | 0.31 | 0.19 | 0.14 |
| Anova P-value | 0.019 | 0.003 | 0.002 | 0.003 | <.0001 | 0.000 |
| Taber, Alberta, | | | | | | |
| 2016 | | | | | | |
| 14CS0851-01-14 | 34.2 AB | 12.2 AB | 2.6 A | 12.8 A | 29.9 C | 56.7 A |
| MIDASTM | 33.4 B | 12.8 A | 2.7 A | 10.8 B | 31.7 B | 56.7 A |
| SRS 934 | 34.2 A | 12.9 A | 2.3 B | 11.5 B | 32.5 A | 55.5 B |
| Std. error | 0.32 | 0.17 | 0.07 | 0.28 | 0.23 | 0.26 |
| Anova P-value | 0.077 | 0.018 | 0.000 | 0.000 | <.0001 | 0.005 |

[symbol]

indicates data missing or illegible when filed

Complete fatty acid profiles (% of total fatty acid methyl esters) of camelina see oil of 14CS0851-01-14, SRS 934 and MIDAS ™
at Morris, MN, Saskatoon, SK, and Taber, AB. Results from Linnaeus Plant Sciences Inc. in Saskatoon.

| Plot | Entry | 16:8 | 18:0 | 18:1-8 | 18:1-11 | 18:2 | 18:3 | 20:0 | 20:1-11 | 20:1-13 |
|---|---|---|---|---|---|---|---|---|---|---|
| Morris, MN | | | | | | | | | | |
| 107 | Midas | 6.214 | 2.712 | 13.961 | 0.979 | 21.402 | 29.925 | 1.835 | 13.227 | 0.683 |
| 202 | Midas | 6.096 | 2.668 | 14.707 | 1.008 | 21.984 | 30.199 | 1.643 | 13.051 | 0.632 |
| 308 | Midas | 6.202 | 2.695 | 14.555 | 1.041 | 22.0 [symbol] 1 | 30.327 | 1.701 | 12.638 | 0.696 |
| 404 | Midas | 6.058 | 2.689 | 14.935 | 1.005 | 21.853 | 38.24 | 1.577 | 12.843 | 0.636 |
| 105 | 14CS0851-01-14 | 6.827 | 3.212 | 13.308 | 1.049 | 20.454 | 32.233 | 1.941 | 12.0 [symbol] 2 | 0.713 |
| 207 | 14CS0851-01-14 | 6.741 | 3.646 | 14.352 | 1.021 | 21.21 | 31.044 | 2.111 | 11.823 | 0.677 |
| 306 | 14CS0851-01-14 | 6.717 | 3.262 | 13.866 | 1.04 | 20. [symbol] 95 | 32.195 | 1.91 | 12.103 | 0.681 |
| 403 | 14CS0851-01-14 | 6.611 | 3.537 | 14.42 | 1.042 | 21.099 | 31.146 | 2.007 | 12.148 | 0.649 |
| 103 | SRS934 | 6.259 | 3.109 | 15.741 | 1.013 | 20.26 [symbol] | 31. [symbol] 47 | 1.594 | 12.715 | 0.518 |
| 204 | SRS934 | 6.202 | 2.956 | 16.141 | 1.019 | 20.17 | 32.315 | 1.494 | 12.457 | 0.5 |
| 301 | SRS934 | 5.261 | 3.176 | 14.982 | 1.022 | 19.998 | 32.545 | 1.558 | 12.494 | 0.656 |
| 408 | SRS934 | 6.412 | 3.27 | 35.12 | 1.007 | 20.319 | 31.802 | 1.747 | 12.793 | 0.527 |
| Saskatoon, SK | | | | | | | | | | |
| 401 | Midas | 5.79 | 2.973 | 13.152 | 0.851 | 19.608 | 33.489 | 1.456 | 13.217 | 0.617 |
| 301 | Midas | 5.967 | 2.885 | 12.348 | 0.821 | 20.511 | 32.123 | 1.635 | 13.855 | 0.614 |
| 202 | Midas | 5.341 | 2.679 | 13.134 | 0.861 | 19.705 | 32.88 | 1.455 | 13.702 | 0.626 |
| 101 | Midas | 5.655 | 2.681 | 13.0 [symbol] 5 | 0.849 | 19.686 | 33.137 | 1.46 | 13.785 | 0.622 |
| 402 | 14CS0851-01-14 | 6.511 | 3.901 | 12.229 | 0.862 | 18.566 | 34.158 | 2.028 | 12.473 | 0.594 |
| 303 | 14CS0851-01-14 | 6.371 | 3.462 | 12.508 | 0.83 | 18.283 | 34.769 | 1.701 | 13.103 | 0.576 |
| 201 | 14CS0851-01-14 | 5.374 | 3.768 | 12.552 | 0.841 | 18.291 | 34.35 | 1.925 | 12.761 | 0.59 |
| 103 | 14CS0851-01-14 | 6.517 | 3.5 [symbol] 3 | 12.461 | 0.874 | 17.627 | 35.028 | 1.824 | 12.758 | 0.611 |
| 403 | SRS934 | 6.748 | 3.686 | 12.803 | 0.88 | 20.238 | 31.9 | 1.756 | 13.547 | 0.486 |
| 302 | SRS934 | 5.884 | 3.203 | 14.177 | 0.874 | 18.209 | 34.105 | 1.478 | 13.649 | 0.511 |
| 203 | SRS934 | 6.078 | 3.599 | 14.038 | 0.857 | 18.229 | 33. [symbol] | 1.592 | 13.849 | 0.463 |
| 102 | SRS934 | 6.065 | 3.339 | 14.722 | 0.922 | 19.04 | 33.59 | 1.458 | 13.189 | 0.788 |
| Taber, AB | | | | | | | | | | |
| 304 | Midas | [symbol] .657 | 2.999 | 14.157 | 0.95 | 19.55 | 33.57 | 1. [symbol] 4 | 22.695 | 0.6 |
| 405 | Midas | 5.745 | 2.912 | 14.153 | 0.98 | 19.042 | 14.043 | 1.496 | 12.662 | 0.618 |
| 201 | Midas | 5.8 [symbol] 6 | 2.933 | 13.627 | 0.938 | 20.199 | 33.093 | 1.661 | 12.682 | 0.613 |
| 101 | Midas | 5.777 | 2.925 | 13.517 | 0.905 | 19.751 | 32.934 | 1.684 | 13.154 | 0.6 |
| 208 | 14CS0851-01-14 | 6.613 | 3.737 | 12.771 | 0.92 [symbol] | 19.927 | 33.189 | 2.019 | 12.21 | 0.59 |
| 303 | 14CS0851-01-14 | 6.322 | 3.702 | 12.484 | 0.928 | 18.201 | 35.116 | 2.031 | 12.279 | 0.615 |
| 407 | 14CS0851-01-14 | 6.254 | 3.795 | 12.911 | 0.911 | 18.102 | 34.931 | 1.99 | 12.273 | 0.609 |
| 104 | 14CS0851-01-14 | 6.39 | 3.96 | 13.25 | 0.921 | 19.314 | 33.395 | 2.096 | 12.19 | 0.587 |
| 309 | SRS934 | 5.734 | 3.211 | 15.18 | 0.981 | 19.046 | 34.524 | 1.438 | 12.376 | 0.484 |
| 206 | SRS934 | 5.795 | 3.33 | 15.753 | 0.959 | 17.96 | 34.452 | 1.502 | 12.996 | 0.479 |
| 403 | SRS934 | 6.6 | 3.609 | 14.601 | 0.917 | 12.633 | 34.533 | 1.597 | 13.211 | 0.471 |
| 107 | SRS934 | 6.097 | 3.355 | 15.167 | 0.977 | 18.932 | 33.464 | 1.538 | 12.87 | 0.501 |

| Plot | Entry | 20:2 | 20:3 | 22:0 | 22:1 | 22:3 | 24:0 | 24:1 | Total |
|---|---|---|---|---|---|---|---|---|---|
| Morris, MN | | | | | | | | | |
| 107 | Midas | 2.037 | 1.151 | 0.387 | 3.135 | 0.456 | 0.216 | 0.696 | 99.015 |
| 202 | Midas | 1.989 | 1.132 | 0.349 | 2.776 | 0.371 | 0.2 | 0.683 | 99.488 |
| 308 | Midas | 1.971 | 1.22 | 0.638 | 2.717 | 0.394 | 0.226 | 0.71 | 99.684 |
| 404 | Midas | 1.94 | 1.091 | 0.34 | 2.731 | 0.362 | 0.201 | 0.708 | 99.209 |
| 105 | 14CS0851-01-14 | 1.869 | 1.241 | 0.384 | 2.657 | 0.514 | 0.1 [symbol] 9 | 0.661 | 99.324 |
| 207 | 14CS0851-01-14 | 1.779 | 1.11 | 0.407 | 2.435 | 0.44 | 0.182 | 0.668 | 99.446 |
| 306 | 14CS0851-01-14 | 1.846 | 1.218 | 0.382 | 2.469 | 0.474 | 0.175 | 0.629 | 99.372 |
| 403 | 14CS0851-01-14 | 1.729 | 1.083 | 0.39 | 2.41 | 0.401 | 0.173 | 0.624 | 99.469 |
| 103 | SRS934 | 1.587 | 1.065 | 0.342 | 2.365 | 0.286 | 0.175 | 0.659 | 99.541 |
| 204 | SRS934 | 1.579 | 1.049 | 0.324 | 2.203 | 0.277 | 0.174 | 0.65 | 99.557 |
| 301 | SRS934 | 1.642 | 1.126 | 0.349 | 2.435 | 0.327 | 0.211 | 0.707 | 99.398 |
| 408 | SRS934 | 1.582 | 1.033 | 0.368 | 2.419 | 0.296 | 0.165 | 0.681 | 99. [symbol] 39 |
| Saskatoon, SK | | | | | | | | | |
| 401 | Midas | 2.0 [symbol] 3 | 1.388 | 0.321 | 2.868 | 0.444 | 0.213 | 0.728 | 99.198 |
| 301 | Midas | 2.197 | 1.342 | 0.357 | 3.247 | 0.482 | 0.21 | 0.743 | 99.337 |
| 202 | Midas | 2.179 | 1.401 | 0.323 | 3.038 | 0.466 | 0.20 [symbol] | 0.733 | 99.126 |
| 101 | Midas | 2.127 | 1.377 | 0.32 | 3.048 | 0.463 | 0.194 | 0.728 | 99.207 |
| 402 | 14CS0851-01-14 | 1.892 | 1.384 | 0.399 | 2.745 | 0.532 | 0.18 | 0.723 | 99.177 |
| 303 | 14CS0851-01-14 | 1.424 | 1.434 | 0.348 | 2.672 | 0.483 | 0.16 [symbol] | 0.695 | 99.354 |
| 201 | 14CS0851-01-14 | 1.906 | 1.419 | 0.376 | 2.747 | 0.544 | 0.177 | 0.719 | 99.34 |
| 103 | 14CS0851-01-14 | 1.898 | 1.476 | 0.366 | 2.659 | 0.528 | 0.173 | 0. [symbol] | 99.093 |
| 403 | SRS934 | 1.8 | 1.148 | 0.391 | 2.735 | 0.351 | 0.185 | 0.722 | 99.356 |
| 302 | SRS934 | 1.781 | 3.313 | 0.325 | 2.546 | 0.352 | 0.176 | 0.684 | 99.269 |
| 203 | SRS934 | 1.689 | 1.244 | 0.329 | 2.424 | 0.313 | 0.154 | 0.623 | 99.153 |
| 102 | SRS934 | 1.67 | 1.196 | 0.33 | 2.473 | 0.284 | 0.208 | 0.694 | 99.438 |
| Taber, AB | | | | | | | | | |
| 304 | Midas | 1.844 | 1.266 | 0.328 | 2.609 | 0.395 | 0.206 | 0.725 | 99.106 |
| 405 | Midas | 1.779 | 1.258 | 0.324 | 2.625 | 0.409 | 0.204 | 0.726 | 99.026 |
| 201 | Midas | 1.871 | 1.229 | 0.352 | 2.767 | 0.427 | 0.211 | 0.743 | 99.162 |
| 101 | Midas | 1.898 | 1.267 | 0.355 | 2.921 | 0.449 | 0.202 | 0.741 | 99.08 |
| 208 | 14CS0851-01-14 | 1.717 | 1.199 | 0.39 | 2.637 | 0.49 | 0.192 | 0.734 | 99.321 |
| 303 | 14CS0851-01-14 | 1.693 | 1.38 | 0.375 | 2.669 | 0.531 | 0.173 | 0.702 | 99.202 |
| 407 | 14CS0851-01-14 | 1.652 | 1329 | 0.375 | 2.621 | 0.524 | 0.179 | 0.683 | 99.139 |
| 104 | 14CS0851-01-14 | 1.653 | 1.196 | 0.399 | 2.521 | 0.452 | 0.206 | 0.677 | 99.207 |
| 309 | SRS934 | 1.502 | 1.121 | 0.321 | 2.181 | 0.286 | 0.209 | 0.754 | 99.348 |
| 206 | SRS934 | 1.417 | 1.103 | 0.316 | 2.233 | 0.27 | 0.166 | 0.671 | 99.402 |
| 403 | SRS934 | 1.40 [symbol] | 1.142 | 0.337 | 2.381 | 0.288 | 0.166 | 0.709 | 99.6 |
| 107 | SRS934 | 1.508 | 1.11 | 0.333 | 2.285 | 0.292 | 0.179 | 0.7 | 99.306 |

[symbol]

indicates data missing or illegible when filed

v) Vitamin E (Tocopherols): Vitamin E (tocopherol) profiles were analyzed by Intertek in Saskatoon, SK. (Method AOCS Ce 8-89 (MVITE-01) Detection Level 0.8 μg/g oil).

### Results:

Alpha, beta, gamma, delta and total tocopherol levels were not significantly different between entries at all locations, save for delta tocopherols at Saskatoon and alpha tocopherols at Taber (Table 40). The delta tocopherol level of 14CS0851-01-14 in Saskatoon was higher than that of both checks while the alpha tocopherol level in Taber was higher in SRS 934 than in 14CS0851-01-14 or in MIDAS™. The raw data can be found in Table 41.

TABLE 40
Vitamin E (tocopherol) content and profile in seeds of
14CS0851-01-14, SRS 934, and MIDAS ™ at Morris,
MN, Saskatoon, SK, and Taber, AB. Values represent averages
of three samples for each location. Values followed
by the same letters are not significantly different.

| Location/Entry | alpha | beta | gamma | delta | Total |
|---|---|---|---|---|---|
| Morris, Minnesota, 2016 | | | | | |
| 14CS0BS1-01-14 | 112.0 A | 1.0 A | 666.0 A | 4.1 A | 782.7 A |
| MIDASTM | 147.0 A | 0.6 A | 605.7 A | 7.4 A | 760.7 A |
| SRS 934 | 168.0 A | 1.2 A | 618.3 A | 5.6 A | 793.0 A |
| Std. error | 23.1 | 0.5 | 63.8 | 1.0 | 70.1 |
| Anova P-value | [symbol] 5 | 0.498 | 0.632 | 0.080 | 0.897 |
| Saskatoon, | | | | | |
| Saskatchewan, 2016 | | | | | |
| 14CS0851-01-14 | 99.9 A | 1.5 A | 639.0 A | 20.0 B | 760.7 A |
| MIDASTM | 119.0 A | 0.9 A | 545.7 A | 3.5 A | 668.7 A |
| SRS 934 | 104.1 A | 1.1 A | 660.3 A | 6.8 A | 772.7 A |
| Std. error | 22.5 | 0.4 | 74.0 | 3.7 | 82.8 |
| Anova P-value | 0.688 | 0.272 | 0.35 [symbol] | 0.010 | 0.461 |
| Taber, Alberta, 2016 | | | | | |
| 14CS0851-01-14 | 112.5 A | 1.1 A | 599.5 A | 5.9 A | 717.5 A |
| MIDASTM | 117.4 A | 0.7 A | 544.5 A | 3.4 A | 666.0 A |
| SRS 934 | 217.7 B | 1.1 A | 529.0 A | 8.5 A | 756.7 A |
| Std. error | 2.6 | 0.5 | 82.2 | 3.6 | 94.7 |
| Anova P-value | 0.001 | 0.696 | 0.686 | 0.512 | 0.850 |

[symbol]

indicates data missing or illegible when filed

TABLE 41
Vitamin E (tocopherol) content and profile in seeds of 14CS0851-01-14, SRS 934, and
MIDAS ™ at Morris, MN, Saskatoon, SK, and Taber, AB. Results from Intertek, Saskatoon.
INTERTEK ANALYSIS REPORT

| [symbol] | [symbol] | | | | | | [symbol] | [symbol] | |
|---|---|---|---|---|---|---|---|---|---|
| [symbol] | [symbol] | | | | | | [symbol] | [symbol] | |
| | [symbol] | | | | | | [symbol] | [symbol] | |
| | [symbol] | | | | | | [symbol] | [symbol] | |
| | [symbol] | | | | | | [symbol] | [symbol] | |
| | | | | | | | [symbol] | [symbol] | |
| [symbol] | [symbol] | | | | | | [symbol] | [symbol] | |
| [symbol] | [symbol] | | | | | | | | |
| | | | | [symbol] | | | | | |
| [symbol] | | | | | [symbol] | | | | |
| | | | | | [symbol] | | | | |
| [symbol] | | | | | [symbol] | | | | |
| | | | | | Total | Alpha | Beta | Gamma | Delta |
| | | PRODUCT INFORMATION | | | Vitamin E | Tocopherol | Tocopherol | Tocopherol | Tocopherol |
| [symbol] | | Trial ID | Plot | Entry | (μg/g of oil) | (μg/g of oil) | (μg/g of oil) | (μg/g of oil) | (μg/g of oil) |
| 170312-001 | | 16C503AQSA | 103 | 14CS0851-01-14 | 624 | 77.8 | 1.54 | 527 | 17 |
| 170312-002 | | 16C503AQSA | 201 | 14CS0851-01-14 | 861 | 103 | 2 | 731 | 25 |
| 170312-003 | | 16C503AQSA | 303 | 14CS0851-01-14 | 797 | 119 | 1 | 659 | 18.1 |
| 170312-004 | | 16C503AQSA | 102 | SRS-934 | 606 | 96.5 | 1.13 | 494 | 14.4 |
| 170312-005 | | 16C503AQSA | 203 | SRS-934 | 902 | 149 | 0.59 | 750 | 2.4 |
| 170312-006 | | 16C503AQSA | 302 | SRS-934 | 810 | 66.8 | 1.65 | 737 | 3.74 |
| 170312-007 | | 16C503AQSA | 101 | Midas | 708 | 131 | 0.78 | 574 | 2.48 |
| 170312-008 | | 16C503AQSA | 202 | Midas | 635 | 113 | 1.13 | 517 | 3.73 |
| 170312-009 | | 16C503AQSA | 301 | | 663 | 113 | 0.69 | 546 | 4.16 |
| 170312-010 | | 16C503TA | 104 | 14CS0851-01-14 | 613 | 127 | 1.05 | 476 | 9.58 |
| 170312-011 | | 16C503TA | 208 | 14CS0851-01-14 | 714 | 293 | 18.2 | 389 | 13.4 |
| 170312-012 | | 16C503TA | 303 | 14CS0851-01-14 | 822 | 97.9 | 1.15 | 723 | 2.16 |
| 170312-013 | | 16C503TA | 107 | SRS-934 | 717 | 225 | 0.52 | 480 | 10.4 |
| 170312-014 | | 16C503TA | 206 | SRS-934 | 758 | 239 | 0.88 | 513 | 4.37 |
| 170312-015 | | 16C503TA | 309 | SRS-934 | 795 | 189 | 1.94 | 594 | 10.6 |
| 170312-016 | | 16C503TA | 103 | Midas | 719 | 299 | 22.9 | 378 | 19.1 |
| 170312-017 | | 16C503TA | 201 | Midas | 219 | 140 | 0.95 | 576 | 2.57 |
| 170312-018 | | 16C503TA | 304 | Midas | 613 | 98.8 | 0.44 | 513 | 4.2 |
| 170312-019 | | 16C503MN | 105 | 14CS0851-01-14 | 775 | 83 | 1.81 | 690 | 0.86 |
| 170312-020 | | 16C503MN | 207 | 14CS0851-01-14 | 708 | 135 | 0.07 | 561 | 8.54 |
| 170312-021 | | 16C503MN | 306 | 14CS0851-01-14 | 868 | 118 | 1.09 | 747 | 2.78 |
| 170312-022 | | 16C503MN | 103 | SRS-934 | 831 | 205 | 1.85 | 622 | 2.51 |
| 170312-023 | | 16C503MN | 204 | SRS-934 | 806 | 162 | 1.28 | 632 | 11.5 |
| 170312-024 | | 16C503MN | 301 | SRS-934 | 742 | 137 | 0.54 | 601 | 2.67 |
| 170312-025 | | 16C503MN | 107 | MIdas | 858 | 161 | 0.61 | 692 | 4.86 |
| 170312-026 | | 16C503MN | 202 | Midas | 791 | 159 | 0.64 | 621 | 10.6 |
| 170312-027 | | 16C503MN | 308 | Midas | 633 | 121 | 0.6 | 504 | 6.72 |

[symbol]

indicates data missing or illegible when filed

vi) Minerals (Calcium, Phosphorous): Minerals were analyzed by Cumberland Valley Analytical Services. Reference: Metals and Other Elements in Plants (985.01). Official Methods of Analysis, 17^{th} Edition. 2000. Association of Official Analytical Chemists. Perkin Elmer 5300 DV ICP. Perkin Elmer, 710 Bridgeport Ave. Shelton, CT 0694.

Significant differences were observed for calcium at Morris and Taber but not at Saskatoon. At Morris, the calcium level of 14CS0851-01-14 was similar to that of SRS 934 but lower than that of MIDAS™. At Taber, the calcium contents of 14CS0851-01-14 and of MIDAS™ were similar and both were higher than that of SRS 934. The phosphorous contents of 14CS0851-01-14 did not differ significantly from those of SRS 934 or MIDAS™ at the Saskatoon and Morris sites. The phosphorus contents were different for all three entries at Taber. 14CS0851-01-14 had the highest phosphorus content (Table 42). The raw data can be found in Table 43.

TABLE 42
Calcium and phosphorous contents (% DM) for 14CS0851-01-14, SRS
934 and MIDAS ™ at Morris, MN, Saskatoon, SK and Taber,
AB. Values represent averages of three samples for each location.
Values followed by the same letter are not significantly different.

| | Calcium | Phosphorous |
|---|---|---|
| Location/Entry | (% DM) | (% DM) |
| Morris, Minnesota, 2016 | | |
| 14CS0851-01-14 | 0.30 B | 0.75 AB |
| MIDASTM | 0.34 A | 0.79 A |
| SRS 934 | 0.29 B | 0.73 B |
| Std. error | 0.01 | 0.02 |
| Anova P-value | 0.004 | 0.111 |
| Saskatoon, Saskatchewan, 2016 | | |
| 14CS0851-01-14 | 0.24 A | 0.62 A |
| MIDASTM | 0.25 A | 0.60 A |
| SRS 934 | 0.23 A | 0.61 A |
| Std. error | 0.01 | 0.01 |
| Anova P-value | 0.444 | 0.215 |
| Taber, Alberta, 2016 | | |
| 14CS0851-01-14 | 0.29 A | 0.82 A |
| MIDASTM | 0.28 A | 0.79 B |
| SRS 934 | 0.25 B | 0.75 C |
| Std. error | 0.00 | 0.01 |
| Anova P-value | 0.000 | 0.005 |

TABLE 43
Mineral content (% DM) for 14CS0851-01-14, SRS 934 and MIDAS ™ at Morris, MN,
Saskatoon, SK and Taber, AB. Results from Cumberland Valley Analytical Services.

| Plot | Entry | Ca (% DM) | P (% OM) | Mg (% DM) | K (% DM) | Na (% DM) | Fe (ppm) | Mn (ppm) | Zn (ppm) | Cu (ppm) |
|---|---|---|---|---|---|---|---|---|---|---|
| Morris, MN | | | | | | | | | | |
| 107 | Midas | 0.34 | 0.82 | 0.36 | 0.94 | 0.01 | 136 | 36 | 64 | 7 |
| 202 | Midas | 0.33 | 0.74 | 0.36 | 0.89 | 0.01 | 116 | 26 | 57 | |
| 308 | Midas | 0.36 | 0.81 | 0.36 | 0.94 | 0.01 | 111 | 25 | 53 | 8 |
| 105 | 14Cs0851-01-14 | 0.29 | 0.74 | 0.34 | 0.89 | 0.01 | 130 | 35 | 58 | 7 |
| 207 | 14Cs0851-01-14 | 0.29 | 0.77 | 0.36 | 0.92 | 0.01 | 129 | 37 | 55 | 7 |
| 306 | 14Cs0851-01-14 | 0.31 | 0.75 | 0.35 | 0.91 | 0.01 | 121 | 35 | 47 | 8 |
| 103 | SRS934 | 0.30 | 0.75 | 0.3 | 0.91 | 0.02 | 116 | 30 | 64 | 7 |
| 204 | SRS934 | 0.29 | 0.73 | 0.35 | 0.88 | 0.02 | 108 | 26 | 59 | 7 |
| 301 | SRS934 | 0.28 | 0.71 | 0.35 | 0.91 | 0.02 | 99 | 23 | 44 | 8 |
| Saskatoon, SK | | | | | | | | | | |
| 301 | Midas | 0.24 | 0.57 | 0.31 | 0.78 | 0.03 | 111 | 33 | 46 | 7 |
| 202 | Midas | 0.27 | 0.62 | 0.34 | 0.83 | 0.02 | 107 | 26 | 47 | 6 |
| 101 | Midas | 0.23 | 0.60 | 0.33 | 0.79 | 0.02 | 108 | 24 | 52 | |
| 303 | 14Cs0851-01-14 | 0.24 | 0.59 | 0.31 | 0.82 | 0.02 | 152 | 28 | 41 | 6 |
| 201 | 14Cs0851-01-14 | 0.24 | 0.62 | 0.34 | 0.83 | 0.02 | 120 | 28 | 45 | 7 |
| 103 | 14Cs0851-01-14 | 0.23 | 0.65 | 0.36 | 0.88 | 0.03 | 116 | 26 | 51 | 16 |
| 302 | SRS934 | 0.24 | 0.58 | 0.32 | 0.85 | 0.02 | 97 | 23 | 46 | 7 |
| 203 | SRS934 | 0.23 | 0.63 | 0.35 | 0.89 | 0.02 | 107 | 27 | 47 | 7 |
| 102 | SRS934 | 0.23 | 0.62 | 0.36 | 0.88 | 0.02 | 124 | 33 | 50 | 7 |
| Taber, AB | | | | | | | | | | |
| 304 | Midas | 0.27 | 0.79 | 0.38 | 0.91 | 0.02 | 123 | 27 | 52 | 8 |
| 201 | Midas | 0.29 | 0.79 | 0.37 | 0.93 | 0.02 | 133 | 32 | 53 | 9 |
| 103 | Midas | 0.29 | 0.79 | 0.35 | 0.92 | 0.01 | 137 | 31 | 54 | 8 |
| 208 | 14Cs0851-01-14 | 0.30 | 0.81 | 0.38 | 0.98 | 0.02 | 127 | 38 | 53 | 9 |
| 303 | 14Cs0851-01-14 | 0.28 | 0.83 | 0.38 | 0.95 | 0.03 | 133 | 43 | S1 | 9 |
| 104 | 14Cs0851-01-14 | 0.29 | 0.83 | 0.38 | 0.98 | 0.02 | 131 | 35 | 52 | 8 |
| 309 | SRS934 | 0.23 | 0.73 | 0.38 | 0.85 | 0.02 | 118 | 30 | 51 | 8 |
| 206 | SRS934 | 0.26 | 0.74 | 0.36 | 0.88 | 0.02 | 114 | 27 | 54 | 7 |
| 107 | SRS934 | 0.25 | 0.77 | 0.38 | 0.89 | 0.02 | 120 | 25 | 52 | 7 |

### vii) Antinutritionals (Sinapine, Phytate, Trypsin Inhibitors, Tannins, Glucosinolates)

### Sinapine

Sinapine is an alkaloidal amine found in some seeds, particularly oil seeds of plants in the family Brassicaceae (Niciforovic et al., 2014). Sinapine has several undesirable properties as a constituent in animal feeds. It is a bitter-tasting compound, making it less palatable to animals, while its presence in the diet of certain brown egg laying hens at levels exceeding 1 g/kg leads to a fishy odour or taste in the eggs (Butler et al., 1982).

Sinapine analysis was performed by the Lipids Quality and Utilization Lab, University of Saskatchewan by proton nuclear magnetic resonance (1H NMR). Camelina seeds were ground with mortar and pestle and extracted three times with 25 mL methanol. The methanol extract was concentrated on a rotary evaporator and then diluted with 50 mL water. Dimethyl formamide (DMF) (50 μL, 47 mg) was added into this aqueous solution as internal standard and the 1H NMR scan of this solution was recorded on a 500 MHz Bruker® NMR system (Billeria, MA, USA) using a water suppression protocol (Berhow et al., 2010). The singlet peaks recorded at 3.25, 3.17 and 3.11 ppm were identified as phenylpropanoid ester, betaine and choline.

### Results:

The sinapine content expressed as mg/g was similar between entries at all three sites (Table 44).

### Phytate

Phytic acid is considered an anti-nutritional factor because it lowers the bio-availability of certain minerals, such as calcium, iron, zinc, and magnesium (Schlemmer et al, 2009). Phytic acid bound to a mineral is known as phytate.

Analysis of phytate was performed by Eurofins Scientific, Inc. Nutrition Analysis Center, 2200 Rittenhouse Street, Des Moines, IA 50321. Reference method: Analytical Biochemistry Vol 77:536-539 (1977). Limit of quantification is 0.14%. Briefly, an aliquot of the seed sample is extracted with a sodium sulfate solution overnight and phytate is precipitated with ferric chloride. The precipitant is ashed and the phosphorous content in the precipitate is determined by ICP-OES method. The resultant phosphorous content is calculated as phytic acid.

### Results:

The percent phytic acid was similar between all three entries at Saskatoon and Morris. At Taber, the phytic acid level of 14CS0851-01-14 was similar to that of MIDAS™ and significantly higher than that of SRS 934 (Table 44).

### Trypsin Inhibitors

Trypsin inhibitors are a family of chemicals that reduce the activity of a digestive enzyme called trypsin, which is a protease enzyme necessary for the absorption and digestion of proteins (Budin, 1995). Since the test to determine the amount of trypsin inhibitors in a sample measures the sample's ability to inhibit activity, it is reported in Trypsin Inhibitor Units/gram (TIU/g).

Trypsin inhibitor analysis was performed by Eurofins Scientific, Inc. Nutrition Analysis Center, 2200 Rittenhouse Street, Des Moines, IA 50321. Reference method: AOCS Ba 12-75. Limit of Quantification is 1000 TIU/g. The sample is defatted and then extracted in a diluted NaOH solution. The solution is centrifuged, and an aliquot of the supernatant is reacted with acetic acid, trypsin solution, and N-α-benzoyl-DL-arginine-p-nitroanilide (BAPA). The sample is then read versus a blank and the TIU/g calculated.

### Results:

Significant differences for trypsin inhibitor activity (TIU/g) were observed at Morris and Taber but not at Saskatoon. Thus, TIU/g in 14CS0851-01-14 was similar to that in SRS 934 at both locations. It is interesting to note that TIU/g in 14CS0851-01-14 was higher than in MIDAS™ at Morris but lower at Taber (Table 44).

### Tannins

Condensed tannins, also known as proanthocyanidins, act as antinutrient compounds because they precipitate proteins, inhibit digestive enzymes and decrease the utilization of vitamins and minerals. Interestingly, tannins have also been shown to have anticarcinogenic and antimutagenic potential and antimicrobial properties (Amarowicz et al, 2010). Tannins were analyzed by Eurofins according to the following method: samples are defatted before extraction in methanol. The extract reacts with 0.5% vanillin to develop color, which is then measured spectrophotometrically. (Price et al, 1978). Limit of Quantification is 0.05%.

### Results:

No differences were noted for tannins (%) between the three different entries at all three locations (Table 44).

TABLE 44
Level of anti-nutritives sinapine, phytic acid, trypsin inhibitors
and tannins in seeds of 14CS0851-01-14, SRS 934 and MIDAS ™ at
Morris, MN, Saskatoon, SK and Taber, AB. Values represent
averages of three samples for each site. Values followed by
the same letter are not significantly different.

| | | Phytic | Trypsin | |
|---|---|---|---|---|
| | S | acid | inhibitors | Tannins |
| Location/Entry | [symbol] g) | (%) | TIU/g | (%) |
| Morris, Minnesota, 2016 | | | | |
| 14CS0851-01-14 | 1.44 A | 1.92 A | 11000 A | 0.05 A |
| MIDASTM | 1.31 A | 2.03 A | 9233 B | 0.05 A |
| SRS 934 | 1.43 A | 1.84 A | 10400 A | 0.05 A |
| Std. error | 0.06 | 0.06 | 257 | 0.00 |
| Anova P-value | 0.1 [symbol] | 0.06 | 0.01 | 0.44 |
| Saskatoon, Saskatchewan, 2016 | | | | |
| 14CS0851-01-14 | 1.58 A | 1.51 A | 14467 A | 0.05 B |
| MIDASTM | 1.36 A | 1.46 A | 15933 A | 0.06 A |
| SRS 934 | [symbol] .49 A | 1.50 A | 14200 A | 0.05 B |
| Std. error | 0.12 | 0.04 | 1330 | 0.00 |
| Anova P-value | 0.48 | 0.50 | 0.45 | 0.00 |
| Taber, Alberta, 2016 | | | | |
| 14CS0851-01-14 | 1.36 A | 2.10 A | 13467 B | 0.05 A |
| MIDASTM | 1.74 A | 2.09 A | 15900 A | 0.06 A |
| SRS 934 | 1.39 A | 1.89 B | 13167 B | 0.05 A |
| Std. error | 0.15 | 0.05 | 528 | 0.00 |
| Anova P-value | 0.08 | 0.03 | 0.01 | 0.19 |

[symbol]

indicates data missing or illegible when filed

The raw data for sinapine levels can be found in Table 45, and the raw data for phytic acid, trypsin inhibitors, and tannins can be found in Table 46.

TABLE 45
Sinapine levels in whole seeds of 14CS0851-01-14, SRS 934 and
MIDAS ™ at Saskatoon, SK (16C503AQSA), Taber, AB
(16C503TA), and Morris, MN (16C503MN). Each sample was assayed
in duplicate. Results from University of Saskatchewan, Saskatoon.

| Sam- | | | | Sina- | Sina- |
|---|---|---|---|---|---|
| ple | | | | pine | pine |
| ID | Trial ID | Plot | Entry | % | mg/g(g/Kg) |
| 1#-1 | 16C503AQSA | 103 | 14CS0851-01-14 | 0.18% | 1.84 |
| 1#-2 | 16C503AQSA | 103 | 14CS0851-01-14 | 0.19% | 1.91 |
| 2#-1 | 16C503AQSA | 201 | 14CS0851-01-14 | 0.12% | 1.22 |
| 2#-2 | 16C503AQSA | 201 | 14CS0851-01-14 | 0.13% | 1.25 |
| 3#-1 | 16C503AQSA | 303 | 14CS0851-01-14 | 0.16% | 1.6 |
| 3#-2 | 16C503AQSA | 303 | 14CS0851-01-14 | 0.17% | 1.68 |
| 4#-1 | 16C503AQSA | 102 | SRS 934 | 0.14% | 1.4 |
| 4#-2 | 16C503AQSA | 102 | SRS 934 | 0.14% | 1.44 |
| 5#-1 | 16C503AQSA | 203 | SRS 934 | 0.17% | 1.74 |
| 5#-2 | 16C503AQSA | 203 | SRS 934 | 0.16% | 1.63 |
| 6#-1 | 16C503AQSA | 302 | SRS 934 | 0.14% | 1.35 |
| 6#-2 | 16C503AQSA | 302 | SRS 934 | 0.14% | 1.39 |
| 7#-1 | 16C503AQSA | 101 | Midas | 0.13% | 1.3 |
| 7#-2 | 16C503AQSA | 101 | Midas | 0.14% | 1.44 |
| 8#-1 | 16C503AQSA | 202 | Midas | 0.13% | 1.34 |
| 8#-2 | 16C503AQSA | 202 | Midas | 0.12% | 1.2 |
| 9#-1 | 16C503AQSA | 301 | Midas | 0.14% | 1.44 |
| 9#-2 | 16C503AQSA | 301 | Midas | 0.14% | 1.43 |
| 10#-1 | 16C503TA | 104 | 14CS0851-01-14 | 0.15% | 1.46 |
| 10#-2 | 16C503TA | 104 | 14CS0851-01-14 | 0.15% | 1.51 |
| 11#-1 | 16C503TA | 208 | 14CS0851-01-14 | 0.12% | 1.18 |
| 11#-2 | 16C503TA | 208 | 14CS0851-01-14 | 0.11% | 1.08 |
| 12#-1 | 16C503TA | 303 | 14CS0851-01-14 | 0.16% | 1.55 |
| 12#-2 | 16C503TA | 303 | 14CS0851-01-14 | 0.14% | 1.38 |
| 13#-1 | 16C503TA | 107 | SRS 934 | 0.15% | 1.51 |
| 13#-2 | 16C503TA | 107 | SRS 934 | 0.14% | 1.37 |
| 14#-1 | 16C503TA | 206 | SRS 934 | 0.14% | 1.39 |
| 14#-2 | 16C503TA | 206 | SRS 934 | 0.14% | 1.35 |
| 15#-1 | 16C503TA | 309 | SRS 934 | 0.14% | 1.38 |
| 15#-2 | 16C503TA | 309 | SRS 934 | 0.14% | 1.35 |
| 16#-1 | 16C503TA | 103 | Midas | 0.14% | 1.43 |
| 16#-2 | 16C503TA | 103 | Midas | 0.15% | 1.48 |
| 17#-1 | 16C503TA | 201 | Midas | 0.18% | 1.78 |
| 17#-2 | 16C503TA | 201 | Midas | 0.19% | 1.94 |
| 18#-1 | 16C503TA | 304 | Midas | 0.19% | 1.89 |
| 18#-2 | 16C503TA | 304 | Midas | 0.20% | 1.97 |
| 19#-1 | 16C503MN | 105 | 14CS0851-01-14 | 0.14% | 1.42 |
| 19#-2 | 16C503MN | 105 | 14CS0851-01-14 | 0.15% | 1.45 |
| 20#-1 | 16C503MN | 207 | 14CS0851-01-14 | 0.15% | 1.52 |
| 20#-2 | 16C503MN | 207 | 14CS0851-01-14 | 0.14% | 1.38 |
| 21#-1 | 16C503MN | 306 | 14CS0851-01-14 | 0.14% | 1.41 |
| 21#-2 | 16C503MN | 306 | 14CS0851-01-14 | 0.15% | 1.48 |
| 22#-1 | 16C503MN | 103 | SRS 934 | 0.14% | 1.38 |
| 22#-2 | 16C503MN | 103 | SRS 934 | 0.14% | 1.39 |
| 23#-1 | 16C503MN | 204 | SRS 934 | 0.13% | 1.3 |
| 23#-2 | 16C503MN | 204 | SRS 934 | 0.14% | 1.37 |
| 24#-1 | 16C503MN | 301 | SRS 934 | 0.16% | 1.58 |
| 24#-2 | 16C503MN | 301 | SRS 934 | 0.16% | 1.55 |
| 25#-1 | 16C503MN | 107 | Midas | 0.13% | 1.33 |
| 25#-2 | 16C503MN | 107 | Midas | 0.13% | 1.33 |
| 26#-1 | 16C503MN | 202 | Midas | 0.14% | 1.36 |
| 26#-2 | 16C503MN | 202 | Midas | 0.14% | 1.36 |
| 27#-1 | 16C503MN | 308 | Midas | 0.12% | 1.2 |
| 27#-2 | 16C503MN | 308 | Midas | 0.13% | 1.25 |

TABLE 46
Levels of phytic acid, trypsin inhibitors and condensed
tannins in whole seeds of 14CS0851-01-14, SRS 934 and
MIDAS ™ at Morris, MN, Saskatoon, SK and Taber, AB.

| | | Phytic | Trypsin | Condensed |
|---|---|---|---|---|
| Plot | Entry | Acid % | Inhibitor TIU/g | Tannin % |
| Morris, MN | | | | |
| 105 | 14CS0851-01-14 | 1.85 | 11,700 | <0.050 |
| 207 | 14CS0851-01-14 | 1.99 | 10,100 | <0.050 |
| 306 | 14CS0851-01-14 | 1.92 | 11,200 | <0.050 |
| 107 | Midas | 2.08 | 10,100 | 0.052 |
| 202 | Midas | 1.95 | 8,700 | <0.050 |
| 308 | Midas | 2.06 | 8,900 | <0.050 |
| 103 | SRS 934 | 1.92 | 11,600 | <0.050 |
| 204 | SRS 934 | 1.85 | 9,500 | <0.050 |
| 301 | SRS 934 | 1.75 | 10,100 | <0.050 |
| Saskatoon, SK | | | | |
| 103 | 14CS0851-01-14 | 1.60 | 15,500 | <0.050 |
| 201 | 14CS0851-01-14 | 1.51 | 12,600 | <0.050 |
| 303 | 14CS0851-01-14 | 1.41 | 15,300 | <0.050 |
| 101 | Midas | 1.48 | 17,600 | 0.065 |
| 202 | Midas | 1.54 | 16,700 | 0.063 |
| 301 | Midas | 1.37 | 13,500 | 0.060 |
| 102 | SRS 934 | 1.60 | 15,300 | <0.050 |
| 203 | SRS 934 | 1.55 | 12,700 | <0.050 |
| 302 | SRS 934 | 1.36 | 14,600 | <0.050 |
| Taber, AB | | | | |
| 104 | 14CS0851-01-14 | 2.10 | 14,200 | <0.050 |
| 208 | 14CS0851-01-14 | 1.98 | 13,900 | <0.050 |
| 303 | 14CS0851-01-14 | 2.22 | 12,300 | <0.050 |
| 103 | Midas | 2.09 | 17,100 | 0.066 |
| 201 | Midas | 2.07 | 15,400 | 0.055 |
| 304 | Midas | 2.11 | 15,200 | 0.051 |
| 107 | SRS 934 | 1.95 | 13,900 | <0.050 |
| 206 | SRS 934 | 1.84 | 12,400 | 0.052 |
| 309 | SRS 934 | 1.87 | 13,200 | 0.052 |

### Glucosinolates

Glucosinolates are a class of secondary metabolites found mainly in the order Brassicales wherein they function in defense against pathogens and herbivores (De Vos et al., 2007). Livestock species fed rations with high glucosinolates may exhibit adverse effects, including reduced feed intake and growth, gastrointestinal irritation, goiter, anemia, and hepatic and renal lesions (Bischoff, 2016). As mentioned in Example 13, camelina accumulates three different glucosinolates in its seeds: glucoarabin (9-(methylsulfinyl) nonylglucosinolate—GS9), glucocamelinine (10-(methylsulfinyl) decylglucosinolate—GS10), and 11-(methylsulfinyl) undecylglucosinolate (GS11).

The glucosinolate content in seed was determined by capillary gas chromatography of the trimethylsilyl derivatives of the extracted and purified desulphoglucosinolates (Sosulski and Dabrowski, 1984). The sample preparation method is a compilation of several published methods adjusted for optimum indole glucosinolate detection. Intact glucosinolates are extracted from the seeds using 67% methanol and purified via the ion-exchange chromatography and “on-column” enzymatic desulfation method of Thies (1980). Preparation of trimethylsilyl derivatives utilizes the acetone and 1-methylimidazole-based method of Landerouin et al (1987). Benzyl glucosinolate or allyl glucosinolate or both is used as the internal standard. Results for each analysis are calculated to report individual glucosinolates and total glucosinolates as μmol g⁻¹ whole seed on a 4-5% moisture basis. (Thies, 1980; Sosulski, 1984; Landerouin, 1987).

### Results:

Significant differences were observed for all three major glucosinolates at all three locations (Morris, MN, Saskatoon, S K and Taber, AB) (Table 47). The three major glucosinolates measured were 9-(methylsufinyl) nonyl (GS 9), 10-(methylsulfinyl) decyl (GS 10) and 11-(methysulfinyl) undecyl (GS 11). The 9-(methylsufinyl) nonyl and 10-methylsulfinyl) decyl contents were lower in 14CS0851-01-14 than in SRS 934 at Morris and lower than that in both checks at Saskatoon and Taber. In contrast, the 11-methysulfinyl) undecyl content was higher in 14CS0851-01-14 than in MIDAS™ at all three locations. The raw data can be found in Table 48.

TABLE 47
Content of the different glucosinolate fractions and total glucosinolate
content (μmol/g seed) of 14CS0851-01-14, SRS 934 and MIDAS ™ at
Morris, MN, Saskatoon, SK and Taber, AB. Values represent the
averages of three samples for each location. Values followed
by the same letters are not significantly different.

| Location/Entry | Total | GS9 | GS10 | GS11 |
|---|---|---|---|---|
| Morris, Minnesota, 2016 | | | | |
| 14CS0851-01-14 | 16.8 | 2.9 A | 10.7 A | 3.2 A |
| MIDASTM | 15.4 | 3.1 A | 9.9 A | 2.4 B |
| SRS 934 | 20.3 | 4.3 B | 13.0 B | 2.9 A |
| Std. error | 1.5 | 0.3 | 1.0 | 0.2 |
| Anova P-value | 0. [symbol] | 0.01 | 0.04 | 0.04 |
| Saskatoon, Saskatchewan, 2016 | | | | |
| 14CS0851-01-14 | 26.2 | 5.1 A | 16.9 A | 4.2 A |
| MIDASTM | 29.4 | 6.5 A | 19.1 B | 3.7 B |
| SRS 934 | 31.5 | 7.0 A | 20.5 B | 4.0 AB |
| Std. error | 0.9 | 0.2 | 0.6 | 0.1 |
| Anova P-value | 0.003 | 0.000 | 0.003 | 0.029 |
| Taber, Alberta, 2016 | | | | |
| 14CS0851-01-14 | 26.1 | 5.5 A | 16.6 A | 4.1 A |
| MIDASTM | 29.5 | 7.0 B | 19.1 B | 3.4 B |
| SRS 934 | 31.2 | 7.2 B | 20.1 C | 3.9 A |
| Std. error | 0.4 | 0.1 | 0.2 | 0.1 |
| Anova P-value | 0.001 | 0.000 | 0.000 | 0.002 |

GS9 = glucoarabin (9-(methylsulfiny)nonylglucosinolate)
GS10 = glucocamelinine (10-(methylsulfinyl)decylglucosinolate)
GS11 = 11-(methylsulfinyl)undecylglucosinolate. [symbol]
indicates data missing or illegible when filed

TABLE 48
Content of the different glucosinolate fractions and total
glucosinolate content (μmol/g seed) of 14CS0851-01-14,
SRS 934 and MIDAS ™ at Morris, MN, Saskatoon, SK and Taber, AB.

| | | | | | Total |
|---|---|---|---|---|---|
| | | 9- | 10- | 11- | Gluco- |
| | | (Methyl- | (Methyl- | (Methyl- | sinolates |
| | | sulfinyl)- | sulfinyl)- | sulfinyl)- | (umol/g |
| Entry | Plot | nonyl | decyl | undecyl | seeds) |
| Morris, MN | | | | | |
| 14CS0851-01-14 | 105 | 3.00 | 10.96 | 3.23 | 17.19 |
| 14CS0851-01-14 | 207 | 2.76 | 10.09 | 2.95 | 15.80 |
| 14CS0851-01-14 | 306 | 2.98 | 11.05 | 3.30 | 17.33 |
| Midas | 107 | 3.62 | 11.29 | 2.66 | 17.56 |
| Midas | 202 | 3.13 | 10.08 | 2.46 | 15.67 |
| Midas | 308 | 2.59 | 8.43 | 2.07 | 13.10 |
| SRS934 | 103 | 4.34 | 13.15 | 2.94 | 20.44 |
| SRS934 | 204 | 3.81 | 11.63 | 2.58 | 18.03 |
| SRS934 | 301 | 4.74 | 14.36 | 3.21 | 22.32 |
| Saskatoon, SK | | | | | |
| 14CS0851-01-14 | 103 | 5.09 | 16.57 | 4.02 | 25.68 |
| 14CS0851-01-14 | 201 | 5.16 | 17.58 | 4.44 | 27.17 |
| 14CS0851-01-14 | 303 | 4.93 | 16.59 | 4.08 | 25.60 |
| Midas | 101 | 6.90 | 19.89 | 3.81 | 30.60 |
| Midas | 202 | 6.03 | 17.89 | 3.53 | 27.45 |
| Midas | 301 | 6.68 | 19.66 | 3.70 | 30.03 |
| SRS934 | 102 | 6.94 | 20.17 | 3.89 | 31.00 |
| SRS934 | 203 | 6.89 | 20.49 | 4.09 | 31.47 |
| SRS934 | 302 | 7.03 | 20.75 | 4.12 | 31.90 |
| Taber, AB | | | | | |
| 14CS0851-01-14 | 104 | 5.79 | 17.11 | 4.17 | 27.06 |
| 14CS0851-01-14 | 208 | 5.25 | 16.21 | 4.14 | 25.60 |
| 14CS0851-01-14 | 303 | 5.46 | 16.44 | 3.86 | 25.76 |
| Midas | 103 | 7.08 | 19.14 | 3.41 | 29.63 |
| Midas | 201 | 6.79 | 18.65 | 3.26 | 28.70 |
| Midas | 304 | 7.05 | 19.43 | 3.54 | 30.02 |
| SRS934 | 107 | 7.25 | 20.06 | 3.85 | 31.16 |
| SRS934 | 206 | 7.06 | 19.81 | 3.89 | 30.76 |
| SRS934 | 309 | 7.34 | 20.39 | 3.98 | 31.72 |

### SUMMARY

Camelina oil has a history of safe use for human consumption in Canada: it was approved by Health Canada as Novel Food in 2010. Camelina oil was further approved for salmonid juveniles in 2016. Camelina meal is approved for use as feed ingredient for both broilers and laying hens.

For each of the comparators—14CS0851-01-14, SRS 934 and MIDAS™—seed samples from 3 plots for each of 3 different locations—Morris, MN, Saskatoon, SK and Taber, AB—were used for the determination of the nutritional profile through accredited laboratories. The analytes evaluated were proximate composition (ash, acid detergent fibre, neutral detergent fibre and non-fibre carbohydrates), seed oil and protein content, amino and fatty acid profiles, vitamin E (tocopherols), minerals (calcium, phosphorous) and antinutritionals (sinapine, phytate, trypsin inhibitors, tannins, glucosinolates).

For proximates, the content of each of the analytes in 14CS0851-01-14 was equal to that in at least one of the checks (SRS 934, MIDAS™) at all test locations.

Seed oil contents of 14CS0851-01-14 were either lower than those of both checks or lower than that of MIDAS™ and equal to that of SRS 934. Correspondingly, the protein content of 14CS0851-01-14 was higher than that of MIDAS™ at all three locations.

Branched-chain amino acids (BCAAs) have been associated with the ability of seed to germinate. At all locations, the amount of BCAAs in 14CS0851-01-14 were significantly lower than in MIDAS™ and either equal to those of SRS 934 or also significantly lower.

Significant differences were observed between all three entries for all fatty acid fractions considered for this submission; however, no trend was observed that was consistent for all.

Alpha, beta, gamma, delta and total tocopherol levels were not significantly different between entries at all locations except for delta tocopherols at Saskatoon, SK and alpha tocopherols at Taber, AB.

Significant differences were observed for calcium at Morris, MN and Taber. At Morris, the calcium level of 14CS0851-01-14 was similar to that of SRS 934 but lower than that of MIDAS™. At Taber, the calcium contents of 14CS0851-01-14 and of MIDAS™ were similar and both were higher than that of SRS 934.

The phosphorus contents were different for all three entries only at Taber. 14CS0851-01-14 had the highest phosphorus content.

Sinapine and tannin contents were similar for all entries at all sites. The percent phytic acid was similar between all three entries at Saskatoon and Morris. At Taber, the phytic acid level of 14CS0851-01-14 was similar to that of MIDAS™ and significantly higher than that of SRS 934. Significant differences for trypsin inhibitor activity (TIU/g) were observed at Morris and Taber. TIU/g in 14CS0851-01-14 was similar to that in SRS 934 and higher than in MIDAS™ at Morris but lower at Taber. For gluscosinolates, the glucoarabin and glucocamelinine contents were lower in 14CS0851-01-14 than in SRS 934 at Morris and lower than in both checks at Saskatoon and Taber. In contrast, the 11-(methysulfinyl) undecyl content was higher in 14CS0851-01-14 than in MIDAS™ at all three locations.

Despite the fact that statistically significant differences between mutant line 14CS0851-01-14 and the checks SRS 934 and MIDAS™ were observed for a number of analytes, these differences were not pronounced. It is therefore anticipated that products derived from camelina line 14CS0851-01-14 and its derivatives would not be any different than products derived from currently available camelina varieties.

### Example 15: Comparative Analysis of Seedling Germination Following Pre-Chill

One factor that determines the competitiveness of a plant is its ability to germinate, particularly under cool conditions. The objective of this study was therefore to compare the ability of seeds from 14CS0851-01-14, SRS 934 and MIDAS™ (commercial variety) to germinate, following a pre-chill at 2° C. for 7 days.

Because there are no published seed germination assays for C. sativa, the CFIA seed testing guidelines developed for the closely related species Brassica napus (Argentine canola) were followed. Reference method: CFIA, Methods and Procedures for Testing Seed/4.6.2. Table 5. In contrast to the B. napus testing protocol, a lower pre-chill temperature was chosen for this study (2° C.) as camelina seeds germinate readily at 5° C.

For each entry—14CS0851-01-14, SRS 934 and MIDAS™—20 g of seed from each of 3 replicates from the field trial in Taber, AB, was weighed out and pooled together in a container and mixed thoroughly using the Hand Mixing Spoon Method (60 g. of seed per entry). Two layers of Whatman™ germination filter paper sheets were placed in a standard 100-seed germination box. Autoclaved water was poured over the filter paper until evenly saturated and excess water was drained off prior to seed plating. 100 seeds were counted with a vacuum seed counter and transferred to the germination box. This was repeated 4 times for each entry (14CS0851-01-14, SRS 934 and MIDAS™) for a total of 400 seeds per entry. Germination boxes were closed with lids, sealed with Parafilm® and placed in the fridge at 2° C. for 7 days (dark).

After 7 days, germination boxes were transferred to a growth chamber cycling between 10 hrs at 25° C. (light) and 14 hrs at 15° C. (dark) for 7 days. Lighting was provided from halogen and high-pressure sodium lights (750-1250 lux). Germination boxes were arranged in a completely randomized design (CRD).

Seedlings were evaluated as normal or abnormal using parameters outlined in the CFIA guidelines, Methods and Procedures for Testing Seed, under section 4.14. The first evaluation was conducted after 4 days and the final evaluation was performed at 7 days. The CFIA guidelines state that the final evaluation is to be conducted after 10 days; however, the camelina seedlings were already well-developed after 4 days and were beginning to show fungal contamination after 5 days.

Normal and decayed seedlings were removed at the first count to help reduce the risk of spreading contamination within the germination boxes, but abnormal seedlings, i.e. those with short or twisted hypocotyls, were left on the substrate until the final count.

### Results:

Camelina seeds from lines 14CS0851-01-14, SRS 934 and MIDAS™ germinated as detailed in Table 49. No significant differences between the lines were observed. The raw data is shown in Table 50.

TABLE 49
Germination of seeds of 14CS0851-01-14, SRS 934 and MIDAS ™ in
% after 4 days and 7 days, respectively, in an alternating
temperature regimen (10 hrs. 25° C. [light] and
14 hrs. 15° C. [dark]), following a pre-chill at 2° C. for 7 days.

| Sample | Day 0 (pre-chill) | Day 4 | Day 7 |
|---|---|---|---|
| 14CS0851-01-14-box 1 | 100 | 99 | 99 |
| 14CS0851-01-14-box 2 | 100 | 100 | 100 |
| 14CS0851-01-14-box 3 | 100 | 100 | 100 |
| 14CS0851-01-14-box 4 | 99 | 100 | 99 |
| Average % | 99.75 | 99.75 | 99.5 |
| SRS 934-box 1 | 100 | 99 | 97 |
| SRS 934-box 2 | 100 | 99 | 97 |
| SRS 934-box 3 | 100 | 100 | 100 |
| SRS 934-box 4 | 100 | 99 | 99 |
| Average % | 100 | 99.25 | 98.25 |
| MIDASTM -box 1 | 100 | 98 | 98 |
| MIDASTM -box 2 | 100 | 100 | 100 |
| MIDASTM -box 3 | 100 | 100 | 100 |
| MIDASTM -box 4 | 100 | 99 | 99 |
| Average % | 100 | 99.25 | 99.25 |

TABLE 50
Alternate Temperature Carmelina Germination Assay
Based on CHIA Methods and Procedures for Testing Seed Section A and ISTA guidelines for B. napus
Pre-chill: 2 Degrees for 7 days in the dark
Germination: In Growth Camber 1; 25 C 8 hours, 15C 16 hours for 7 days;
Halogen and high pressure sodium lights 50/50

| | Day 0 (prechill) | | | | | Day 4 | | | | | Day 7 | | | | |
|---|---|---|---|---|---|---|---|---|---|---|---|---|---|---|---|
| | Nor- | Abnor- | dead | fresh | | Nor- | Abnor- | dead | fresh | | Nor- | Abnor- | dead | fresh | |
| | mal | mal | hard | ungern. | total | mal | mal | hard | ungern. | total | mal | mal | hard | ungern. | total |
| 14CS0851- | 100 | 0 | 0 | 0 | 100 | 99 | 0 | 0 | 1 | 100 | 99 | 0 | 0 | 1 | 100 |
| 01-14-1 | | | | | | | | | | | | | | | |
| 14CS0851- | 100 | 0 | 0 | 0 | 100 | 100 | 0 | 0 | 0 | 100 | 100 | 0 | 0 | 0 | 100 |
| 01-14-2 | | | | | | | | | | | | | | | |
| 14CS0851- | 100 | 0 | 0 | 0 | 100 | 100 | 0 | 0 | 0 | 100 | 100 | 0 | 0 | 0 | 100 |
| 01-14-3 | | | | | | | | | | | | | | | |
| 14CS0851- | 99 | 0 | 0 | 1 | 100 | 100 | 0 | 0 | 0 | 100 | 99 | 1 | 0 | 0 | 100 |
| 01-14-4 | | | | | | | | | | | | | | | |
| Total % | 99.75 | 0 | 0 | 0.25 | 100 | 99.75 | 0 | 0 | 0.25 | 100 | 99.5 | 0.25 | 0 | 0.25 | 100 |
| SRS 934 | 100 | 0 | 0 | 0 | 100 | 99 | 1 | 0 | 0 | 100 | 97 | 2 | 0 | 1 | 100 |
| SRS 934 | 100 | 0 | 0 | 0 | 100 | 99 | 0 | 1 | 0 | 100 | 97 | 2 | 0 | 1 | 100 |
| SRS 934 | 100 | 0 | 0 | 0 | 100 | 100 | 0 | 0 | 0 | 100 | 100 | 0 | 0 | 0 | 100 |
| SRS 934 | 100 | 0 | 0 | 0 | 100 | 99 | 0 | 0 | 1 | 100 | 99 | 0 | 0 | 1 | 100 |
| Total % | 100 | 0 | 0 | 0 | 100 | 99.25 | 0.25 | 0.25 | 0.25 | 100 | 98.25 | 1 | 0 | 0.75 | 100 |
| Midas | 100 | 0 | 0 | 0 | 100 | 98 | 0 | 0 | 2 | 100 | 98 | 0 | 0 | 2 | 100 |
| Midas | 100 | 0 | 0 | 0 | 100 | 100 | 0 | 0 | 0 | 100 | 100 | 0 | 0 | 0 | 100 |
| Midas | 100 | 0 | 0 | 0 | 100 | 100 | 0 | 0 | 0 | 100 | 100 | 0 | 0 | 0 | 100 |
| Midas | 100 | 0 | 0 | 0 | 100 | 99 | 0 | 0 | 1 | 100 | 99 | 0 | 0 | 1 | 100 |
| Total % | 100 | 0 | 0 | 0 | 100 | 99.25 | 0 | 0 | 0.75 | 100 | 99.25 | 0 | 0 | 0.75 | 100 |

Notes:
Prechill at 2 degrees C. instead of 5 because camelina is able to germinate well at 5 degrees C.
After 7 days at 2 degrees, almost all seeds had small radicle emerging
After 7 days at 25 C./15 C.: Plants were getting very stressed and moldy therefore experiment was terminated.
Very little difference between Day 4 and Day 7 with regards to plant development
Abnormal = short, twisted hypocotyl formation

### Example 16: Effect of Temperature on Germination

It was investigated whether genotype response to temperature varies between 14CS0851-01-14, SRS 934 and MIDAS™ by recording percent germinated seeds daily until maximum germination, at 4 different temperatures, ranging from 4 to 30° C.

Seeds were plated on moist Whatman™ filter paper and then transferred to 4 different temperatures—4° C., 10° C., 20° C. and 30° C.—in the dark to germinate. Percent germination was recorded daily until 100% germination had occurred, or up to 12 days. Seeds were considered germinated when the radicle was at least twice the length of the seed (R2).

Similarly to the previously described germination assay involving pre-chill in Example 15, for each entry, 14CS0851-01-14, SRS 934 and MIDAS™, 20 g of seed from each of 3 replicates from the field trial in Taber, AB, was weighed out and pooled together in a container and mixed thoroughly using the Hand Mixing Spoon Method (60 g of seed per entry). Two layers of Whatman™ filter paper were placed in round Petri dishes (100 mm diameter) and moistened with autoclaved water. 20 seeds of each variety were manually placed in each Petri dish. Petri dishes were sealed with Parafilm®. Five Petri dishes per camelina line were prepared, for a total of 100 seeds per variety. Petri dishes were incubated in the dark at the temperatures described above in random order. The experiment was performed twice. Percentage of normal germination after no further germination occurred, was recorded. When all seeds in a single Petri dish reached the R2 stage (radicle twice length of seed), the Petri dish was removed from the incubator. Plates were removed from the incubator each day at the same time, and the seeds/seedlings were scored as NRS (no radicle, swollen), RSM (radicle small), RSS (radicle same size as seed), R2 (radicle twice length of seed; germinated).

Proc Glimmix in SAS for binomial data was used to perform the statistical analysis. To avoid problems with logit transformation (log(R2/(Total−R2)) at R2=0 and R2=20, 0.05 and 0.1 was added to the R2 and total, respectively. Each run was analyzed separately as days do not always line up in the separate runs. Only R2 was analyzed as it provides information on the rate of germination.

### Results:

Table 51 shows the germination over time for each line. As indicated above, prior to analysis, data were transformed to logits which is log(x/(1−x)) where x is the proportion. Mean separation is on the logit scale. For presentation, the means (on the logit scale) were back transformed to the original scale using exp(x)/[1+exp(x)]. Overall, final germination was not significantly different between the 3 lines. Most importantly, at 4° C., germination was not significantly different between lines at each time point except between SRS 934 and MIDAS™ at six days. The raw data is shown in Table 52.

TABLE 51
Effect of temperature on germination of 14CS0851-01-14, SRS 934 and MIDAS ™. A total
of 100 seeds of each line were germinated at 4° C., 10° C., 20° C., and 30° C. Prior to analysis, data
were transformed to logits. Mean separation is on the logit scale. For presentation, the means (on
the logit scale) were back transformed to the original scale using exp(x)/[1 + exp(x)]. Experiments
were performed twice. Values followed by the same letters are not significantly different.

| | 1 | | | 2 | | |
|---|---|---|---|---|---|---|
| Exp. Entry | day | logit | mean | day | logit | mean |

Germination at 4° C.

| | | | | | | | | |
|---|---|---|---|---|---|---|---|---|
| 14CS0851-01-14 | 5 | −3.090 | 0.04 | EF | 5 | −3.144 | 0.041 | EF |
| 14CS0851-01-14 | 6 | −1.202 | 0.23 | CD | 6 | −1.210 | 0.230 | CD |
| 14CS0851-01-14 | 7 | 1.379 | 0.799 | B | 7 | 1.388 | 0.800 | B |
| 14CS0851-01-14 | 8 | 2.710 | 0.938 | AB | 8 | 2.748 | 0.940 | AB |
| 14CS0851-01-14 | 9 | 2.710 | 0.938 | AB | 9 | 2.748 | 0.940 | AB |
| MIDAS ™ | 5 | −3.738 | 0.023 | F | 5 | −3.840 | 0.021 | F |
| MIDAS ™ | 6 | −1.433 | 0.193 | DE | 6 | −1.442 | 0.191 | DE |
| MIDAS ™ | 7 | 2.073 | 0.888 | AB | 7 | 2.093 | 0.890 | AB |
| MIDAS ™ | 8 | 3.738 | 0.977 | A | 8 | 3.841 | 0.979 | A |
| MIDAS ™ | 9 | 3.738 | 0.977 | A | 9 | 3.841 | 0.979 | A |
| SRS 934 | S | −5.321 | 0.005 | F | 5 | −5.748 | 0.003 | F |
| SRS 934 | 6 | −0.160 | 0.460 | C | 6 | −0.161 | 0.460 | C |
| SRS 934 | 7 | 2.068 | 0.888 | AB | 7 | 2.087 | 0.890 | AB |
| SRS 934 | 8 | 5.321 | 0.995 | A | 8 | 5.748 | 0.997 | A |
| SRS 934 | 9 | 5.321 | 0.995 | A | 9 | 5.748 | 0.997 | A |
| Anova P value | entry | 0.045 | | | entry | 0.041 | | |
| | day | <.0001 | | | day | <.0001 | | |
| | entry*day | 0.0109 | | | entry*day | 0.006 | | |

Germination at 10° C.

| | | | | | | | | |
|---|---|---|---|---|---|---|---|---|
| 14CS0851-01-14 | 2 | 3.732 | 0.023 | A | 4 | 0.726 | 0.674 | BD |
| 14CS0851-01-14 | 3 | 3.100 | 0.957 | B | 5 | 2.052 | 0.886 | AC |
| MIDAS ™ | 2 | 5.319 | 0.005 | A | 4 | 4.015 | 0.982 | ABC |
| MIDAS ™ | 3 | 2.871 | 0.946 | B | 5 | 4.015 | 0.982 | ABC |
| SRS 934 | 2 | 5.319 | 0.005 | A | 4 | 2.147 | 0.895 | CD |
| SRS 934 | 3 | 4.272 | 0.986 | B | 5 | 4.080 | 0.983 | AB |
| Anova P value | entry | 0.577 | | | entry | 0.078 | | |
| | day | 0.046 | | | day | 0.007 | | |
| | entry*day | 0.445 | | | entry*day | 0.170 | | |

Germination at 20° C.

| | | | | | | | | |
|---|---|---|---|---|---|---|---|---|
| 14CS0851-01-14 | 1 | 5.3117 | 0.00491 | A | 1 | 5.3188 | 0.00488 | B |
| 14CS0851-01-14 | 2 | 3.3641 | 0.9666 | A | 2 | 3.767 | 0.9774 | A |
| | | | | | 3 | 3.767 | 0.9774 | A |
| MIDAS ™ | 1 | 5.3162 | 0.00489 | A | 1 | 3.1155 | 0.04247 | B |
| MIDAS ™ | 2 | 4.2775 | 0.9863 | A | 2 | 4.3111 | 0.9868 | A |
| | | | | | 3 | 4.3111 | 0.9868 | A |
| SRS 934 | 1 | 3.0868 | 0.04366 | A | 1 | 4.3016 | 0.01337 | B |
| SRS 934 | 2 | 5.3104 | 0.9951 | A | 2 | 4.3138 | 0.9868 | A |
| | | | | | 3 | 4.3138 | 0.9868 | A |
| Anova P value | entry | 0.3312 | | | | 0.2577 | | |
| | day | 0.0466 | | | | <0.001 | | |
| | entry*day | 0.7957 | | | | 0.6873 | | |

Germination at 30° C.

| | | | | | | | | |
|---|---|---|---|---|---|---|---|---|
| 14CS0851-01-14 | 1 | 0.322 | 0.580 | B | 1 | 1.874 | 0.867 | A |
| 14CS0851-01-14 | 2 | 2.555 | 0.928 | A | 2 | 3.316 | 0.965 | A |
| 14CS0851-01-14 | 3 | 2.555 | 0.928 | A | 3 | 3.316 | 0.965 | A |
| MIDAS ™ | 1 | 1.790 | 0.857 | AB | 1 | 2.630 | 0.933 | A |
| MIDAS ™ | 2 | 2.796 | 0.943 | A | 2 | 3.841 | 0.979 | A |
| MIDAS ™ | 3 | 2.980 | 0.952 | A | 3 | 3.841 | 0.979 | A |
| SRS 934 | 1 | 2.395 | 0.917 | A | 1 | 2.826 | 0.944 | A |
| SRS 934 | 2 | 3.516 | 0.971 | A | 2 | 3.882 | 0.980 | A |
| SRS 934 | 3 | 3.516 | 0.971 | A | 3 | 3.882 | 0.980 | A |
| Anova P value | entry | 0.044 | | | entry | 0.523 | | |
| | | <0.00 | | | | | | |
| | day | 1 | | | day | 0.001 | | |
| | entry*day | 0.185 | | | entry*day | 0.989 | | |

TABLE 52
Germination at 4° C., 10° C., 20° C., and 30° C. (raw data).
Germination Study comparing 14CS0851-01-14, SRS934, and Midas at 4 different temperatures

| | Day 7 | | | | | Day 8 | | | | | Day 9 | | | | |
|---|---|---|---|---|---|---|---|---|---|---|---|---|---|---|---|
| | NRS | RSm | RSS | R2 | total | NRS | RSm | RSS | R2 | total | NRS | RSm | RSS | R2 | total |
| 4 degrees C.- 1st Rep | | | | | | | | | | | | | | | |
| 14CS0851-01-14-1 | 2 | 0 | 2 | 16 | 20 | 2 | 0 | 0 | 18 | 20 | 1 | 1 | 0 | 18 | 20 |
| 14CS0851-01-14-2 | 0 | 0 | 4 | 16 | 20 | 0 | 0 | 0 | 20 | 20 | 0 | 0 | 0 | 20 | 20 |
| 14CS0851-01-14-3 | 2 | 1 | 3 | 14 | 20 | 2 | 0 | 0 | 18 | 20 | 1 | 1 | 0 | 18 | 20 |
| 14CS0851-01-14-4 | 1 | 0 | 1 | 18 | 20 | 1 | 0 | 0 | 19 | 20 | 1 | 0 | 0 | 19 | 20 |
| 14CS0851-01-14-5 | 0 | 0 | 4 | 16 | 20 | 0 | 0 | 1 | 19 | 20 | 0 | 0 | 1 | 19 | 26 |
| Total % | 5 | 1 | 14 | 80 | 100 | 5 | 0 | 1 | 94 | 100 | 3 | 2 | 1 | 94 | 100 |
| SRS934-1 | 0 | 1 | 0 | 19 | 20 | 0 | 0 | 0 | 20 | 20 | 0 | 0 | 0 | 20 | 20 |
| SRS934-2 | 0 | 1 | 0 | 19 | 20 | 0 | 0 | 0 | 20 | 20 | 0 | 0 | 0 | 20 | 20 |
| SRS934-3 | 0 | 0 | 4 | 16 | 20 | 0 | 0 | 0 | 20 | 20 | 0 | 0 | 0 | 20 | 20 |
| SRS934-4 | 0 | 0 | 4 | 16 | 20 | 0 | 0 | 0 | 20 | 20 | 0 | 0 | 0 | 20 | 20 |
| SRS934-5 | 0 | 0 | 1 | 19 | 20 | 0 | 0 | 0 | 20 | 20 | 0 | 0 | 0 | 20 | 20 |
| Total % | 0 | 2 | 9 | 89 | 100 | 0 | 0 | 0 | 100 | 100 | 0 | 0 | 0 | 100 | 100 |
| Midas-1 | 0 | 1 | 2 | 17 | 20 | 0 | 1 | 0 | 19 | 20 | 0 | 1 | 0 | 19 | 20 |
| Midas-2 | 0 | 0 | 2 | 18 | 20 | 0 | 0 | 0 | 20 | 20 | 0 | 0 | 0 | 20 | 20 |
| Midas-3 | 0 | 0 | 3 | 17 | 20 | 0 | 0 | 0 | 20 | 20 | 0 | 0 | 0 | 20 | 20 |
| Midas-4 | 0 | 0 | 1 | 19 | 20 | 0 | 0 | 0 | 20 | 20 | 0 | 0 | 0 | 20 | 20 |
| Midas-5 | 1 | 0 | 1 | 18 | 20 | 0 | 1 | 0 | 19 | 20 | 0 | 1 | 0 | 19 | 20 |
| Total % | 1 | 1 | 9 | 89 | 100 | 0 | 2 | 0 | 98 | 100 | 0 | 2 | 0 | 98 | 100 |
| 4 degrees C.-2nd Rep | | | | | | | | | | | | | | | |
| 14CS0851-01-14-1 | 0 | 0 | 4 | 16 | 20 | 0 | 0 | 0 | 20 | 20 | 0 | 0 | 0 | 20 | 20 |
| 14CS0851-01-14-2 | 0 | 0 | 4 | 16 | 20 | 0 | 0 | 0 | 20 | 20 | 0 | 0 | 0 | 20 | 20 |
| 14CS0851-01-14-3 | 1 | 1 | 5 | 13 | 20 | 1 | 0 | 0 | 18 | 20 | 1 | 0 | 0 | 19 | 20 |
| 14CS0851-01-14-4 | 0 | 1 | 5 | 14 | 20 | 0 | 0 | 0 | 20 | 20 | 0 | 0 | 0 | 20 | 20 |
| 14CS0851-01-14-S | 0 | 0 | 2 | 18 | 20 | 0 | 0 | 0 | 20 | 20 | 0 | 0 | 0 | 20 | 20 |
| Total % | 1 | 2 | 20 | 77 | 100 | 1 | 0 | 0 | 99 | 100 | 1 | 0 | 0 | 99 | 100 |
| SRS934-1 | 2 | 9 | 9 | 0 | 20 | 2 | 3 | 11 | 4 | 20 | 1 | 1 | 4 | 14 | 20 |
| SRS934-2 | 0 | 4 | 6 | 10 | 20 | 0 | 0 | 3 | 17 | 20 | 0 | 0 | 1 | 19 | 20 |
| SRS934-3 | 0 | 1 | 7 | 12 | 20 | 0 | 0 | 0 | 20 | 20 | 0 | 0 | 0 | 20 | 20 |
| SRS934-4 | 0 | 0 | 1 | 19 | 20 | 0 | 0 | 0 | 20 | 20 | 0 | 0 | 0 | 20 | 20 |
| SRS934-5 | 0 | 6 | 8 | 6 | 20 | 0 | 1 | 2 | 17 | 20 | 0 | 1 | 0 | 19 | 20 |
| Total % | 2 | 20 | 31 | 47 | 100 | 2 | 4 | 16 | 78 | 100 | 1 | 2 | 5 | 92 | 100 |
| Midas-1 | 2 | 2 | 6 | 10 | 20 | 2 | 1 | 2 | 15 | 20 | 2 | 1 | 0 | 17 | 20 |
| Midas-2 | 0 | 0 | 1 | 19 | 20 | 0 | 0 | 0 | 20 | 20 | 0 | 0 | 0 | 20 | 20 |
| Midas-3 | 0 | 0 | 5 | 15 | 20 | 0 | 0 | 0 | 20 | 20 | 0 | 0 | 0 | 20 | 20 |
| Midas-4 | 0 | 0 | 2 | 18 | 20 | 0 | 0 | 0 | 20 | 20 | 0 | 0 | 0 | 20 | 20 |
| Midas-S | 0 | 0 | 0 | 20 | 20 | 0 | 0 | 0 | 20 | 20 | 0 | 0 | 0 | 20 | 20 |
| Total % | 2 | 2 | 14 | 82 | 100 | 2 | 1 | 2 | 95 | 100 | 2 | 1 | 0 | 97 | 100 |

| | Day 3 | | | | | Day 4 | | | | |
|---|---|---|---|---|---|---|---|---|---|---|
| 10 Degrees C.-1st Rep | NRS | RSm | ASS | R2 | total | NRS | RSm | RSS | R2 | total |
| 14CS0851-01-14-1 | 0 | 0 | 0 | 20 | 20 | 0 | 0 | 0 | 20 | 20 |
| 14CS0851-01-14-2 | 0 | 0 | 1 | 19 | 20 | 0 | 0 | 0 | 20 | 20 |
| 14CS0851-01-14-3 | 0 | 0 | 1 | 19 | 20 | 0 | 0 | 0 | 20 | 20 |
| 14CS0851-01-14-4 | 0 | 0 | 0 | 20 | 20 | 0 | 0 | 0 | 20 | 20 |
| 14CS0851-01-14-5 | 0 | 2 | 0 | 18 | 20 | 0 | 1 | 1 | 18 | 20 |
| Total % | 0 | 2 | 2 | 96 | 100 | 0 | 1 | 1 | 98 | 100 |
| SRS934-1 | 0 | 0 | 0 | 20 | 20 | 0 | 0 | 0 | 20 | 20 |
| SRS934-2 | 0 | 0 | 0 | 20 | 20 | 0 | 0 | 0 | 20 | 20 |
| SRS934-3 | 1 | 0 | 0 | 19 | 20 | 0 | 1 | 0 | 19 | 20 |
| SRS934-4 | 0 | 0 | 0 | 20 | 20 | 0 | 0 | 0 | 20 | 20 |
| SRS934-5 | 0 | 0 | 0 | 20 | 20 | 0 | 0 | 0 | 20 | 20 |
| Total % | 1 | 0 | 0 | 99 | 100 | 0 | 1 | 0 | 99 | 100 |
| Midas-1 | 0 | 1 | 0 | 19 | 20 | 0 | 1 | 0 | 19 | 20 |
| Midas-2 | 0 | 0 | 0 | 20 | 20 | 0 | 0 | 0 | 20 | 20 |
| Midas-3 | 2 | 1 | 0 | 17 | 20 | 2 | 1 | 0 | 17 | 20 |
| Midas-4 | 0 | 0 | 0 | 20 | 20 | 0 | 0 | 0 | 20 | 20 |
| Midas-S | 1 | 0 | 0 | 19 | 20 | 1 | 0 | 0 | 19 | 20 |
| Total % | 3 | 2 | 0 | 95 | 100 | 3 | 2 | 0 | 95 | 100 |
| | Day 4 | | | | | Day 5 | | | | |
| 10 Degrees C. -2nd Rep | NRS | RSm | RSS | R2 | total | NRS | RSm | RS | R2 | total |
| 14CS0851-01-14-1 | 0 | 11 | 5 | 4 | 20 | 0 | 2 | 1 | 17 | 20 |
| 14CS0851-01-14-2 | 0 | 4 | 3 | 13 | 20 | 0 | 1 | 2 | 17 | 20 |
| 14CS0851-01-14-3 | 1 | 1 | 0 | 18 | 20 | 1 | 0 | 0 | 19 | 20 |
| 14CS0851-01-14-4 | 2 | 0 | 0 | 18 | 20 | 2 | 0 | 0 | 18 | 20 |
| 14CS0851-01-14-5 | 1 | 4 | 3 | 12 | 20 | 0 | 2 | 2 | 15 | 20 |
| Total % | 4 | 20 | 11 | 65 | 100 | 4 | 5 | 5 | 86 | 100 |
| SRS934-1 | 0 | 0 | 0 | 20 | 20 | 0 | 0 | 0 | 20 | 20 |
| SRS934-2 | 0 | 2 | 2 | 16 | 20 | 0 | 0 | 1 | 19 | 20 |
| SRS934-3 | 2 | 4 | 10 | 4 | 20 | 2 | 0 | 0 | 15 | 20 |
| SRS934-4 | 0 | 0 | 0 | 20 | 20 | 0 | 0 | 0 | 20 | 20 |
| SRS934-5 | 0 | 0 | 0 | 20 | 20 | 0 | 0 | 0 | 20 | 20 |
| Total % | 2 | 6 | 12 | 80 | 100 | 2 | 0 | 0 | 94 | 100 |
| Midas-1 | 0 | 0 | 0 | 20 | 20 | 0 | 0 | 0 | 20 | 20 |
| Midas-2 | 1 | 0 | 0 | 19 | 20 | 1 | 0 | 0 | 19 | 20 |
| Midas-3 | 0 | 0 | 0 | 20 | 20 | 0 | 0 | 0 | 20 | 20 |
| Midas-4 | 1 | 0 | 0 | 19 | 20 | 1 | 0 | 0 | 19 | 20 |
| Midas-5 | 0 | 0 | 0 | 20 | 20 | 0 | 0 | 0 | 20 | 20 |
| Total % | 2 | 0 | 0 | 98 | 100 | 2 | 0 | 0 | 98 | 100 |
| | Day 1 | | | | | Day 2 | | | | |
| | NRS | RSm | RSS | R2 | total | NRS | RSm | RSS | R2 | total |
| 20 Degees C. - 1st Rep | | | | | | | | | | |
| 14CS08S1-01-14-1 | 0 | 13 | 7 | 0 | 20 | 0 | 0 | 0 | 20 | 20 |
| 14CS0851-01-14-2 | 1 | 17 | 2 | 0 | 20 | 1 | 0 | 0 | 19 | 20 |
| 14CS0851-01-14-3 | 0 | 16 | 4 | 0 | 20 | 0 | 0 | 2 | 18 | 20 |
| 14CS0851-01-14-4 | 0 | 17 | 3 | 0 | 20 | 0 | 0 | 0 | 20 | 20 |
| 14CS0851-01-14-5 | 0 | 14 | 6 | 0 | 20 | 0 | 0 | 0 | 20 | 20 |
| Total % | 1 | 77 | 22 | 0 | 100 | 1 | 0 | 2 | 97 | 100 |
| SRS934-1 | 0 | 12 | 8 | 0 | 20 | 0 | 0 | 0 | 20 | 20 |
| SRS934-2 | 0 | 5 | 15 | 0 | 20 | 0 | 0 | 0 | 20 | 20 |
| SRS934-3 | 0 | 8 | 12 | 0 | 20 | 0 | 0 | 0 | 20 | 20 |
| SRS934-4 | 0 | 8 | 10 | 2 | 20 | 0 | 0 | 0 | 20 | 20 |
| SRS934-5 | 0 | 0 | 18 | 2 | 20 | 0 | 0 | 0 | 20 | 20 |
| Total % | 0 | 33 | 63 | 4 | 100 | 0 | 0 | 0 | 100 | 100 |
| Midas-1 | 0 | 14 | 6 | 0 | 20 | 0 | 0 | 0 | 20 | 20 |
| Midas-2 | 0 | 17 | 3 | 0 | 20 | 0 | 0 | 0 | 20 | 20 |
| Midas-3 | 0 | 11 | 9 | 0 | 20 | 0 | 0 | 0 | 20 | 20 |
| Midas-4 | 0 | 8 | 12 | 0 | 20 | 0 | 0 | 0 | 20 | 20 |
| Midas-5 | 1 | 14 | 5 | 0 | 20 | 1 | 0 | 0 | 19 | 20 |
| Total | 1 | 64 | 35 | 0 | 100 | 1 | 0 | 0 | 99 | 100 |
| 20 Degees C. - 2nd Rep | | | | | | | | | | |
| 14CS0851-01-14-1 | 1 | 0 | 19 | 0 | 20 | 0 | 0 | 0 | 19 | 20 |
| 14CS0851-01-14-2 | 0 | 0 | 20 | 0 | 20 | 0 | 0 | 0 | 20 | 20 |
| 14CS0851-01-14-3 | 0 | 0 | 20 | 0 | 20 | 0 | 0 | 0 | 20 | 20 |
| 14CS0851-01-14-4 | 1 | 0 | 19 | 0 | 20 | 1 | 0 | 0 | 19 | 20 |
| 14CS0851-01-14-5 | 0 | 0 | 20 | 0 | 20 | 0 | 0 | 0 | 20 | 20 |
| Total % | 2 | 0 | 98 | 0 | 100 | 2 | 0 | 0 | 98 | 100 |
| SRS934-1 | 0 | 1 | 19 | 0 | 20 | 0 | 0 | 0 | 20 | 20 |
| SRS934-2 | 0 | 1 | 19 | 0 | 20 | 0 | 1 | 0 | 19 | 20 |
| SRS934-3 | 0 | 0 | 20 | 0 | 20 | 0 | 0 | 0 | 20 | 20 |
| SRS934-4 | 0 | 0 | 20 | 0 | 20 | 0 | 0 | 0 | 20 | 20 |
| SRS934-5 | 0 | 0 | 19 | 1 | 20 | 0 | 0 | 0 | 20 | 20 |
| Total % | 0 | 2 | 97 | 1 | 100 | 0 | 1 | 0 | 99 | 100 |
| Midas-1 | 0 | 0 | 20 | 0 | 20 | 0 | 0 | 0 | 20 | 20 |
| Midas-2 | 0 | 0 | 20 | 0 | 20 | 0 | 0 | 0 | 20 | 20 |
| Midas-3 | 1 | 0 | 18 | 1 | 20 | 1 | 0 | 0 | 19 | 20 |
| Midas-4 | 0 | 1 | 16 | 3 | 20 | 0 | 0 | 0 | 20 | 20 |
| Midas-S | 0 | 0 | 20 | 0 | 20 | 0 | 0 | 0 | 20 | 20 |
| Total % | 1 | 1 | 94 | 4 | 100 | 1 | 0 | 0 | 99 | 100 |
| 30 Degees C. - 1st Rep | | | | | | | | | | |
| 14CS0851-01-14-1 | 1 | 4 | 15 | 0 | 20 | 1 | 0 | 0 | 19 | 20 |
| 14CS0851-01-14-2 | 3 | 16 | 1 | 0 | 20 | 0 | 0 | 3 | 17 | 20 |
| 14CS0851-01-14-3 | 1 | 16 | 3 | 0 | 20 | 1 | 0 | 0 | 19 | 20 |
| 14CS0851-01-14-4 | 0 | 19 | 1 | 0 | 20 | 0 | 0 | 1 | 19 | 20 |
| 14CS0851-01-14-5 | 1 | 11 | 8 | 0 | 20 | 0 | 0 | 0 | 20 | 20 |
| Total % | 6 | 66 | 28 | 0 | 100 | 2 | 0 | 4 | 94 | 100 |
| SRS934-1 | 0 | 5 | 14 | 1 | 20 | 0 | 0 | 0 | 20 | 20 |
| SRS934-2 | 0 | 8 | 11 | 1 | 20 | 0 | 0 | 0 | 20 | 20 |
| SRS934-3 | 0 | 13 | 6 | 1 | 20 | 0 | 0 | 0 | 20 | 20 |
| SRS934-4 | 0 | 1 | 11 | 6 | 20 | 1 | 0 | 1 | 18 | 20 |
| SRS934-5 | 2 | 7 | 11 | 0 | 20 | 1 | 0 | 1 | 18 | 20 |
| Total % | 2 | 34 | 53 | 9 | 100 | 2 | 0 | 2 | 96 | 100 |
| Midas-1 | 1 | 15 | 4 | 0 | 20 | 1 | 0 | 0 | 19 | 20 |
| Midas-2 | 1 | 8 | 10 | 1 | 20 | 1 | 0 | 0 | 19 | 20 |
| Midas-3 | 1 | 10 | 7 | 2 | 20 | 1 | 0 | 0 | 19 | 20 |
| Midas-4 | 1 | 9 | 10 | 0 | 20 | 0 | 1 | 3 | 16 | 20 |
| Midas-5 | 0 | 7 | 11 | 2 | 20 | 0 | 0 | 0 | 20 | 20 |
| Total % | 4 | 49 | 42 | 5 | 100 | 3 | 1 | 3 | 93 | 100 |
| 30 Degees C. - 2nd Rep | | | | | | | | | | |
| 14CS0851-01-14-1 | 0 | 0 | 0 | 20 | 20 | 0 | 0 | 0 | 20 | 20 |
| 14CS0851-01-14-2 | 0 | 4 | 2 | 14 | 20 | 0 | 2 | 0 | 18 | 20 |
| 14CS0851-01-14-3 | 1 | 3 | 0 | 16 | 20 | 1 | 1 | 0 | 18 | 20 |
| 14CS0851-01-14-4 | 0 | 0 | 2 | 18 | 20 | 0 | 0 | 0 | 20 | 20 |
| 14CS0851-01-14-5 | 0 | 1 | 2 | 17 | 20 | 0 | 0 | 0 | 20 | 20 |
| Total % | 1 | 8 | 0 | 85 | 100 | 1 | 3 | 0 | 96 | 100 |
| SRS934-1 | 0 | 1. | 0 | 19 | 20 | 0 | 0 | 1 | 19 | 20 |
| SRS934-2 | 0 | 0 | 0 | 20 | 20 | 0 | 0 | 0 | 20 | 20 |
| SRS934-3 | 0 | 1 | 1 | 18 | 20 | 0 | 0 | 0 | 20 | 20 |
| SRS934-4 | 0 | 0 | 0 | 20 | 20 | 0 | 0 | 0 | 20 | 20 |
| SRS934-5 | 1 | 1 | 1 | 17 | 20 | 1 | 0 | 0 | 19 | 20 |
| Total % | 1 | 3 | 2 | 94 | 100 | 1 | 0 | 1 | 98 | 100 |
| Midas-1 | 1 | 0 | 1 | 18 | 20 | 1 | 0 | 0 | 19 | 20 |
| Midas-2 | 0 | 0 | 1 | 19 | 20 | 0 | 0 | 0 | 20 | 20 |
| Midas-3 | 0 | 1 | 0 | 18 | 20 | 0 | 0 | 0 | 20 | 20 |
| Midas-4 | 1 | 0 | 1 | 18 | 20 | 1 | 0 | 0 | 19 | 20 |
| Midas-5 | 0 | 0 | 0 | 20 | 20 | 0 | 0 | 0 | 20 | 20 |
| Total % | 2 | 1 | 4 | 93 | 100 | 2 | 0 | 0 | 98 | 100 |

### Example 17: Life History Traits-Comparative Field Trials

Selection of counterparts: Mutant camelina line 14CS0851-01-14 was developed by EMS mutagenesis of camelina accession SRS 934, as described in Example 1. Therefore, SRS 934 was chosen as the main comparator. However, SRS 934 is not a commercially grown variety in Canada; therefore, a second comparator, commercial camelina variety MIDAS™, was also included in the field trials.

Selection of field plot locations: In 2016, field trials were conducted at 8 sites: 4 sites were located in the Canadian Prairies (Taber, AB; Saskatoon, SK; Elm Creek, MB; and Minto, MB) and 4 sites were located in the Northern United States (Huntley, MT; Fargo, ND; Box Elder, SD; Morris, MN). In 2017, field trials were planted at Saskatoon, SK; Huntley, MT; and Morris, MN.

Statistical Analysis: All statistical analyses were conducted using PROC Mixed (SAS Institute, 2009). The model is:

$${Y}_{ijk} = mu … + {r}_{i} , \_ + t \cdot {je}_{ijk}$$

Where Yᵢⱼₖ is the variable of interest, mu is the overall mean, rᵢ is the ith, t is the jth entry and the eᵢⱼₖ is error.

Values represent the average of four replicated plot samples for each location. Values followed by the same letters are not significantly different. Different letters denote statistically different least-squares means (P<0.05).

Life History Traits that are being Compared:
(a) plant stand
(b) plant vigor
(c) days to first flowering
(d) days to 50% flowering
(e) days to final flowering
(f) days to maturity
(g) plant height at maturity
(h) seed yield
(i) abiotic stress (excessive moisture, heat, drought) and biotic stress (pathogens: downy mildew, aster yellows, sclerotinia stem rot) resistance).

The above traits were chosen based on CFIA Directive 94-08 Assessment Criteria for Determining Environmental Safety of Plants with Novel Traits and also on Directive 95-03 Guidelines for the Assessment of Novel Feeds: Plant Sources. As detailed below, by analysis of these life history traits it was shown that the mutagenized camelina line 14CS0851-01-14 confers the same characteristics as other camelina varieties.

### Materials and Methods:

Environmental assessment field trials were contracted in 10 locations in 2016 and 3 locations in 2017, and complete data sets were obtained for 8 sites in 2016 and 3 sites in 2017, for a total of 11 sites. Trial sites in Canada in 2016 were Elm Creek, MB (Ag-Quest), Minto, MB (Ag-Quest), Saskatoon, SK (Ag-Quest), and Taber, AB (Ag-Quest). Trial sites in the US were located at Morris, MN (United States Department of Agriculture, USDA), Huntley, MT (Montana State University), Fargo, ND (North Dakota State University), and Box Elder, SD (South Dakota State University). In 2017, trial sites included Saskatoon, SK (AAFC Research Farm), Morris, MN and Huntley MT. Field trials located in Canada were subject to CFIA Plant Biosafety Office authorization for confined research field testing terms and conditions (16-LIN1-478-CAM, 17-ACS1-536-CAM). A standard protocol for the trials was followed in all locations as described below:
1. Trial samples: Mutant camelina line 14CS0851-01-14 (PNT), SRS 934 and MIDAS™.
2. Trial design: randomized complete block design (RCBD) or split-plot design
   a. # of Replicated plots for each sample: 4
   b. Randomization provided by: Contractor
   c. Guard seed provided by: Linnaeus
   d. Plot size (m²): chosen by contractor, Linnaeus informed
   e. Seed packaging provided by: Linnaeus
3. Fertilizer: as per recent soil test, recommendations for 40 bu/ac canola
4. Site Preparation:
   a. Prepare weed-free trial site with seed bed suitable for small seed (shallow placement). Select a site that has not had any Group 2 herbicides in the past 2 years of cropping history, or significant history of other potentially residual Group 2 active ingredients e.g. chemical families imidazolinones, sulfonylaminocarbonyltriazolinones, amides, sulfonylureas, pyrazoles, triazolpyramidines, and triazolones.
   b. Select a site with cereal stubble that had good weed control. For disease management purposes, avoid a site that had Brassica crops in previous 2 years.
   c. Note that kochia may be difficult to control; select a site where kochia is not a problem.
   d. Apply Treflan® or Edge® (ethalfluralin or trifluralin) and incorporate prior to seeding with fertilizer.
5. Site Maintenance:
   a. Grassy weeds: Assure® II is preferred, as it has Minor Use registration for use on camelina.
   b. Broadleaf weeds: Hand-weed as needed (at least once at herbicide application timing, once later).
6. Ratings and rating scales on all plots:
   a. plant stand (% plot fill, ˜3-4 leaf stage)
   b. plant vigour (1-5, 1 is poorest, 5 is best, ˜3-4 leaf stage)
   c. days to first flowering (days after planting when 10% of plants have one or more open flower, assessed 3× weekly)
   d. days to 50% flowering (days after planting when 50% of flowers have opened, assessed 3× weekly)
   e. days to end of flowering (days after planting when no flowers remain open, assessed 3× weekly)
   f. days to maturity (days after planting when 50% of the plants in a plot have changed color, assessed 3× weekly)
   g. plant height at maturity (cm, to top of plants)
   h. yield (g/plot)
   i. grain moisture (%)
   j. yield (kg/ha), adjusted for grain moisture
   k. biotic and abiotic stress (at seedling stage, rosette stage, bolting/flowering and pre-maturity stage). 0-10 scale (0 is no effect from stressor, 10 is dead/dying from stressor).
      i. Biotic stressors include insect pests and diseases (downy mildew, aster yellows, sclerotinia stem rot)
      ii. Abiotic stressors include excess moisture, drought, heat, cold, salinity, etc.
7. Harvesting: Plants should be straight combined. Maturity times are similar to B. rapa. Camelina pods are generally ready before the plants appear to be ready to harvest. As soon as the plants start to senesce, start checking the pods for dry yellow-brown seeds. Shattering may occur if harvest is delayed. Spraying a desiccant is recommended to dry down the stems if necessary to facilitate combining. Reglone (diquat) can be used at the recommended rate for desiccation at a high water volume.
8. Subsampling and Shipping: A representative seed sample (˜1 kg) from each plot will be returned to Linnaeus. Do not pool replicates. All extra grain and residual material are to be destroyed.
9. Reports: A final report will be provided with the raw data in an Excel file, with an ANOVA analysis of the data. Details on the conduct of the trial, including agronomic details and rating scales, will accompany the data as part of the final report.

### Results/Summary for all Locations:

The results are shown in Table 53 below. With respect to the phenotypic life history traits, significantly lower seed yields were noted in 14CS0851-01-14 at Elm Creek, Minto, and Box Elder in 2016 and also in Huntley, Morris, and Saskatoon (AAFC Research Farm) in 2017. In Taber, the yield of 14CS0851-01-14 was similar to that of MIDAS™, while line SRS 934 yielded significantly lower. With regards to plant height, in some locations 14CS0851-01-14 was taller than both the comparators (Morris 2016, Fargo 2016), while in other locations 14CS0851-01-14 was shorter (Minto 2016, and Huntley 2016, 2017). In Saskatoon 2016 (Ag-Quest), 14CS0851-01-14 was similar in height to parent SRS 934 but significantly taller than MIDAS™. Days to maturity (DTM) were not significantly different for all three lines in Elm Creek 2016, Minto 2016, Huntley 2016, Morris 2017, and Saskatoon 2017. In 2 locations, DTM of 14CS0851-01-14 were equivalent to that of SRS 934 (Morris 2016, Box Elder 2016), and in 2 locations (Taber 2016 and Huntley 2017) the DTM were less than for both SRS 934 and MIDAS™.

In summary, differences in seed yield, plant height and days to maturity between the three comparators are not consistent and therefore, the phenotypic expression of these traits in 14CS0851-01-14 can be considered within normal ranges. The differences noted between locations were likely due to different environmental conditions during the life cycle of the plants.

Stand, Vigor, and Days to Flower (DTF 10, DTF 50, DTF 100) ratings were not significantly different between the comparators at any location.

Abiotic stress was only reported in Minto and Taber (hail); however, no significant differences in plant injury were noted between the lines.

The only notable biotic stressor was downy mildew (causal agent: Peronospora camelinae). Downy mildew was noted in Minto 2016, Morris 2016, and Saskatoon 2017 (AAFC Research Farm) and was most prevalent in 14CS0851-01-14 (rating of 3-4), followed by SRS 934 (rating of 2) and MIDAS™ appeared to be most resistant (rating of 1-2), where a rating of 0 means no effect, and 10 means dead/dying. The susceptibility of SRS 934 and 14CS0851-01-14 to downy mildew was also noted in the 2015 PNT field trial at the AAFC Research Farm (data not shown). MIDAS™ has been documented as having partial resistance to downy mildew, while other camelina varieties are quite susceptible to downy mildew. It is therefore not surprising that 14CS0851-01-14 and SRS 934 are more susceptible.

TABLE 53
Life history traits of 14CS0851-01-14, SRS 934 and MIDAS ™ at 11 locations in
Canada and the United States in 2016 and 2017. DTF10, DTF 50, DTF 100, and DTM are
presented in days from seeding. Values are averages of 4 replicates. Values followed by the
same letters are not significantly different.

| Stand | Vigor | DTF | DTF | DTF | | Height | Yield |
|---|---|---|---|---|---|---|---|
| (%) | (1-5) | 10 | 50 | 100 | DTM | (cm) | (kg/ha) |

Elm Creek, Manitoba, 2016

| | | | | | | | | |
|---|---|---|---|---|---|---|---|---|
| 14CS0851-01-14 | 82.8 A | 4.3 A | 38.8 A | nd | 56.0 A | 79.5 A | 75.0 A | 694 B |
| MIDAS ™ | 85.0 A | 4.8 A | 36.3 A | nd | 53.3 8 | 79.5 A | 75.0 A | 1185 A |
| SRS 934 | 92.5 A | 4.5 A | 38.8 A | nd | 55.3 A | 78.8 A | 72.3 A | 1053 A |
| Std. error | 7.2 | 0.3 | 0.4 | | 0.4 | 0.5 | 1.8 | 84 |
| Anova P-value | 0.3 | 0.2 | <.001 | | <.001 | 0.2 | 0.5 | <.001 |

Minto, Manitoba, 2016

| | | | | | | | | |
|---|---|---|---|---|---|---|---|---|
| 14CS0851-01-14 | 17.5 A | 2.5 A | 41.0 A | 49.0 8 | 61.0 A | 79.0 A | 62.8 C | 326 B |
| MIDAS ™ | 16.3 A | 2.5 A | 38.8 A | 47.0 A | 62.0 A | 81.8 A | 71.3 A | 561 A |
| SRS 934 | 25.0 A | 3.8A | 38.5 A | 47.5 A | 62.0 A | 80.0 A | 68.0 B | 550 A |
| Std. error | 4.1 | 0.4 | 0.5 | 0.2 | 0.6 | 0.5 | 1.7 | 53 |
| Anova P-value | 0.1 | 0.0 | <.001 | 3.001 | 0.2 | <.001 | <.001 | 0 |

Morris, Minnesota, 2016

| | | | | | | | | |
|---|---|---|---|---|---|---|---|---|
| 14CS0851-01-14 | 32.5 A | 4.8 A | 41.8 A | 44.5 A | 48.5 A | 79.3 B | 65.3 A | 1310 B |
| MIDAS ™ | 32.8 A | 5.0 A | 41.5 A | 43.8 A | 48.0 A | 80.8 A | 62.8 B | 1432 A |
| SRS 934 | 34.5 A | 5.0 A | 42.3 A | 44.5 A | 48.5 A | 79.8 B | 59.8 C | 1327 B |
| Std. error | 0.9 | 0.1 | 0.8 | 0.9 | 0.4 | 0.4 | 1.9 | 76 |
| Anova P-value | 0.1 | 0.4 | 0.7 | 0.8 | 0.6 | <0.001 | <0.001 | 0 |

Huntley, Montana, 2016

| | | | | | | | | |
|---|---|---|---|---|---|---|---|---|
| 14CSO851-01-14 | 98.0 A | 5.0 A | 47.0 A | 51.0A | 62.0 A | 70.0 A | 106.3 A | 486 A |
| MIDAS ™ | 97.8 A | 5.0 A | 47.0 A | 51.0 A | 62.0 A | 70.0 A | 108.5 A | 471 A |
| SRS 934 | 99.5 A | 5.0 4 | 47.0 A | 51.0A | 62.0 A | 70.0 A | 111.0 A | 478 A |
| Std. error | 0.7 | | | | | | 3.0 | 35 |
| Anova P-value | 0.1 | 1.0 | 1.0 | 1.0 | 1.0 | 1.0 | 0.5 | 1 |

Fargo, North Dakota, 2016

| | | | | | | | | |
|---|---|---|---|---|---|---|---|---|
| 14CS0851-01-14 | 97.8 A | 2.0 A | 45.3 A | 49.5 A | 61.0 B | 78.5 B | 64.5 A | 243 B |
| MIDAS ™ | 98.8 A | 2.0 A | 47.0 A | 52.3 A | 64.5 A | 82.0 A | 55.8 B | 332 A |
| SRS 934 | 98.8 A | 2.0 A | 47.3 A | 52.0 A | 64.5 A | 80.3 AB | 55.0 B | 246 B |
| Std. error | 0.6 | | 1.0 | 1.3 | 1.1 | 1.3 | 2.8 | 72 |
| Anova P-value | 0.4 | 1.0 | 0.2 | 0.2 | 0.0 | 0.0 | <.0001 | 0.0 |

Saskatoon, Saskatchewan, (Ag-Quest) 2016

| | | | | | | | | |
|---|---|---|---|---|---|---|---|---|
| 14CS0851-01-14 | 55.0 A | 2.4 A | 35.0A | 42.0 A | 68.8 A | 87.0 A | 93.5 A | 1147 B |
| MIDAS ™ | 48.8 A | 2.6 A | 35.0 A | 42.0 A | 65.3 A | 86.3 A | 87.5 B | 1281 A |
| SRS 934 | 63.8 A | 1.5 A | 35.0 A | 42.0 4 | 65.0 A | 84.3 B | 95.0 A | 1360 A |
| Std. error | 8.5 | 0.5 | | | 0.6 | 0.2 | 2.4 | 123 |
| Anova P-value | 0.3 | 0.0 | 1.0 | 0.6 | 0.2 | <.0001 | 0.0 | 0.0 |

Box Elder, South Dakota, 2016

| | | | | | | | | |
|---|---|---|---|---|---|---|---|---|
| 14CS0851-01-14 | 4.3 A | 3.7 A | 56.3 A | 59.0 A | 78.0 A | 85.0 A | 19.0 A | 217 B |
| MIDAS ™ | 3.6 A | 3.0 A | 58.3 A | 63.0 A | 76.3 B | 83.3 B | 19.0 A | 309 A |
| SRS 934 | 4.0 A | 3.8 A | 56.0 A | 59.5 A | 78.5 A | 85.5 A | 19.5 A | 282 A |
| Std. error | 0.4 | 0.5 | 1.4 | 1.6 | 0.7 | 0.7 | 0.5 | 28 |
| Anova P-value | 0.1 | 0.4 | 0.5 | 0.2 | 0.0 | 0.0 | 0.7 | <.0001 |

Taber, Alberta, 2016

| | | | | | | | | |
|---|---|---|---|---|---|---|---|---|
| 14CS0851-01-14 | 95.0 A | 5.0 A | 41.0 A | 45.3 B | 55.0 A | 78.0 B | nd | 3120 A |
| MIDAS ™ | 95.0 A | 5.0 A | 42.0 A | 46.3 A | 56.0 A | 79.3 A | nd | 3202 A |
| SRS 934 | 95.0 A | 5.0 A | 42.0 A | 46.3 A | 56.0 A | 79.3 A | nd | 2309 B |
| Std. error | | | | 0.3 | | 0.4 | | 166 |
| Anova P-value | 1.0 | 1.0 | 1.0 | 0.0 | 1.0 | 0.0 | | <.0010 |

Huntley, Montana, 2017

| | | | | | | | | |
|---|---|---|---|---|---|---|---|---|
| 14CS0851-01-14 | 80.0 B | 4.0 A | 53.0 A | 62.0 A | 79.8 A | 87.8 B | 57.1 B | 274 C |
| MIDAS ™ | 91.3 A | 4.3 A | 53.0 A | 62.0 A | 80.0 A | 90.8 A | 65.5 A | 387 A |
| SRS 934 | 77.5 B | 3.5 A | 53.0 A | 62.8 A | 80.5 A | 90.0 A | 59.6 B | 318 B |
| Std. error | 2.6 | 0.3 | 0.3 | 1.3 | 1.2 | 0.6 | 2.7 | 1.2 |
| Anova P-value | <0.0001 | 0.2 | 0.4 | 0.8 | 0.9 | <0.001 | 0.0 | <.0001 |

Morris, Minnesota, 2017

| | | | | | | | | |
|---|---|---|---|---|---|---|---|---|
| 0851-01-14 | 22.3 A | 4.5 A | 42.0 A | 43.8 A | 47.8 A | 76.8 A | 78.8 A | 1439 B |
| MIDAS> | 30.5 A | 5.0 A | 41.3 A | 43.3 A | 47.3 A | 76.5 A | 78.3 A | 1810 A |
| SRS 934 | 16.3 A | 4.5 A | 42.3 A | 44.5 A | 48.3 A | 76.0 A | 75.3 B | 1018 C |
| Std. error | 2.0 | 0.2 | 0.6 | 0.8 | 0.4 | 0.9 | 1.2 | 129 |
| Anova P-value | <0.0001 | 0.2 | 0.2 | 0.5 | 0.1 | 0.8 | <0.0001 | <0.0001 |

Saskatoon, Saskatchewan (AAFC Research Farm), 2017

| | | | | | | | | |
|---|---|---|---|---|---|---|---|---|
| 14CS0851-01-14 | 87.5 A | 4.0 A | 37.0 A | 44.8 A | 56.0 A | 78.8 A | 74.1 A | 1981 B |
| MIDAS ™ | 87.5 A | 4.0 A | 37.0 A | 45.0 A | 55.8 A | 79.5 A | 75.8 A | 2982 A |
| SRS 934 | 77.5 B | 4.0 A | 3700 A | 45.0 A | 55.8 A | 79.5 A | 72.5 A | 2717 A |
| Std. error | NO | — | — | 0.1 | 0.5 | 0.6 | 4.8 | 160 |
| Anova P-value | NO | — | — | 0.46 | 0.66 | 0.72 | 0.83 | <0.0001 |

DTF 10 = days to first flowering (days after planting when 10% of plants have one or more open flower)
DTF 50 = days to 50% flowering (days after planting when 50% of flowers have opened)
DFT 100 = days to end of flowering (days after planting when no flowers remain open)
DTM = days to maturity (days after planting when 50% of the plant in a plot have changed color)

The results of the Life History Trait study are described in greater detail below with respect to the specific field trial locations, including the conditions at each respective location and the raw data obtained from each location.

### Box Elder, South Dakota (2016)

Location: GPS 44° 6′25″N; 103°5′30″W
Soil: Type A (85% Kyle, 5% Lohmiller, 5% Hisle, 5% Swanboy). The Kyle series consists of very deep and well-drained soils formed in sediments weathered from clay shale on uplands. Permeability is very slow.
Climate: In Box Elder, the summers are warm and mostly clear and the winters are freezing, dry, windy, and partly cloudy. Over the course of the year, the temperature typically varies from −9°° C. to 31° C. and is rarely below −19° C. or above 37° C. The hot season lasts for 3.0 months, from June 13 to September 14, with an average daily high temperature above 25° C. The hottest day of the year is July 27, with an average high of 31° C. and low of 17° C. The cold season lasts for 3.5 months, from November 20 to March 5, with an average daily high temperature below 8° C. The coldest day of the year is January 1, with an average low of −9° F. and high of 2° C. The rainy period of the year lasts for 7.2 months, from March 26 to November 2. Box Elder, SD receives a yearly average of 432 mm of rain and 104 cm of snow. The growing season in Box Elder typically lasts for 5.0 months (154 days), from around May 5 to around October 5, rarely starting before April 14 or after May 23, and rarely ending before September 14 or after October 24. According to Health Canada directive DIR2010-05, Box Elder, SD is located in agro-ecological zone 7.
Weather during the growing season of 2016: Lower than average rainfall; cumulative rainfall 173 mm between Apr. 1 to Jul. 31, 2016 (average 304 mm); temperature range 8° C.-23° C. during growing season.
Seeding date: Apr. 11, 2016; harvest date: Jul. 20, 2016
Comments: Disease, insect and weed pressure: not an issue
Results: No differences were observed for seven of the eight parameters measured at the Box Elder site in 2016. The seed yield of 14CS0851-01-14 was significantly lower than that of the checks SRS 934 and MIDAS™ (Tables 53 and 54).

TABLE 54
Life history traits in Box Elder, South Dakota, 2016.

| Box Elder, South Dakota, 2016 | | | | | | | | plant | | | | |
|---|---|---|---|---|---|---|---|---|---|---|---|---|
| | | Early | Vigor | | | | | height | | | | |
| | | Season | (1-5) | days to | days | days to | | at | Yield | Yield | | |
| | | Stand | Early | first | to 50% | end of | days to | maturity | (grams/ | (lbs/ | Test Wt. | |
| Variety | Plot | (1-5) | Season | flowering | flowering | flowering | maturity | (inches) | plot) | Acre) | (lb/bu) | comment |
| 14CS0851-01-14 | 206 | 4.5 | 3 | 56 | 58 | 79 | 86 | 19 | 136.078 | 174.24 | 41.9 | |
| 14CS0851-01-14 | 303 | 4 | 4 | 58 | 59 | 78 | 85 | 20 | 181.437 | 232.32 | 43.2 | |
| 14CS0851-01-14 | 407 | 4.5 | 4 | 57 | 60 | 77 | 84 | 18 | 136.078 | 174.24 | 39.9 | |
| 14CS0851-01-14 | 106 | x | x | x | x | x | x | x | x | x | x | error |
| Midas | 108 | 4.5 | 4 | 56 | 60 | 76 | 83 | 20 | 181.437 | 232.32 | 44.1 | |
| Midas | 203 | 4 | 2 | 56 | 61 | 75 | 82 | 19 | 181.437 | 232.32 | 42.7 | |
| Midas | 307 | 2 | 2 | 65 | 70 | 77 | 84 | 18 | 272.155 | 348.48 | — | Roundup |
| | | | | | | | | | | | | Inj [symbol] |
| Midas | 401 | 4 | 4 | 56 | 61 | 77 | 84 | 19 | 226.796 | 290.4 | 42.5 | |
| SRS 934 | 101 | 4 | 3 | 56 | 62 | 80 | 87 | 19 | 181.437 | 232.32 | 34.9 | |
| SRS 934 | 209 | 4.5 | 4 | 56 | 58 | 80 | 87 | 21 | 181.437 | 232.3 | 40.3 | |
| SRS 934 | 305 | 3 | 3 | 56 | 59 | 77 | 84 | 19 | 0.000 | 0 | 0 | Roundup |
| | | | | | | | | | | | | Inj [symbol] |
| SRS 934 | 404 | 4.5 | 5 | 56 | 59 | 77 | 84 | 19 | 226.796 | 290.4 | 47.2 | |

[symbol]

indicates data missing or illegible when filed

### Elm Creek, Manitoba (2016)

Location: Legal land location SE 23-8-5 W1
Soil: 76% Sand, 13% Silt, 11% Clay, 2.7% organic matter (OM)
Climate: For Elm Creek, MB (as for Minto, MB) the published climate data from close by Carman, MB (distance: 12 km) are used as a representative site for Southern Manitoba. The summers are long and comfortable; the winters are frigid, snowy, and windy; and it is partly cloudy year round. Over the course of the year, the temperature typically varies from −20° C. to 26° C. and is rarely below −32° C. or above 31° C. The warm season lasts for 4.2 months, from May 15 to September 20, with an average daily high temperature above 19° C. The hottest day of the year is July 25, with an average high of 26° C. and low of 14° C. The cold season lasts for 3.3 months, from November 27 to March 6, with an average daily high temperature below −3° C. The coldest day of the year is January 15, with an average low of −20° C. and high of −11° C. The rainy period of the year lasts for 7.4 months, from March 24 to November 7. Elm Creek, MB receives a yearly average of 398 mm of rain and 146 cm of snow. The growing season in Southern Manitoba typically lasts for 4.1 months (127 days), from around May 19 to around September 23, rarely starting before April 30 or after June 6, and rarely ending before September 7 or after October 11. According to Health Canada directive DIR2010-05, Elm Creek, MB is located in agro-ecological zone 5.
Weather during growing season in 2016: Higher than average cumulative rainfall between May 31 and Aug. 31, 2016:435 mm; temperature range 7° C.-26° C. during growing season.
Seeding date: Jun. 7, 2016; harvest date: Sep. 14, 2016
Results: No differences were observed for stand, vigour, days to maturity (DTM), and height (HGT) at Elm Creek in 2016. 14CS0851-01-14 flowered approximately two days later (Days to Flower 10 (DTF 10) and DTF 100) than SRS 934. DTF 50 was not determined. The seed yield of 14CS0851-01-14 was lower than that of the checks (Tables 53 and 55).

TABLE 55
Life history traits in Elk Creek, Manitoba, 2016.

| Elm Creek, Manitoba, 2016 | | | | | | | | | | | | |
|---|---|---|---|---|---|---|---|---|---|---|---|---|
| | | Jun. 28, | Jun. 28, | Jul. 7, | Jul. 21, | | | | | | Sep. 14, | |
| | | 2016 | 2016 | 2016 | 2016 | | | | Avg Max | Sep. 14, | 2016 | Sep. 14, |
| | | plant | plant | water | water | Start of | End of | | Plant | 2016 | Grain | 2016 |
| | | stand | vigor | damage | damage | Flowering | Flowering | Maturity | height | WEIGHT | moisture % | YIELD |
| Entry | Plot | % | 1-5 | 0-10 | 0-10 | DAP | DAP | DAP | cm | g | % | KG/ha |
| 14CS0851-01-14 | 102 | 90 | 4 | 1 | 1 | 38 | 56 | 78 | 80 | 470 | 7.5 | 670.9 |
| 18CS0851-01-14 | 203 | 100 | 4 | 3 | 2 | 39 | 56 | 79 | 75 | 410 | 8.3 | 590 |
| 14CS0851-01-14 | 301 | 80 | 4 | 6 | 5 | 39 | 56 | 80 | 71 | 435 | 8.3 | 648 |
| 14CS0851-01-14 | 402 | 60 | | 1 | 2 | 39 | 56 | 81 | 74 | 610 | 7.9 | 867 |
| SRS 934 | 101 | 100 | 4 | 4 | 3 | 39 | 55 | 78 | 68 | 590 | 7.5 | 842.2 |
| SRS 934 | 202 | 90 | 4 | 1 | 2 | 39 | 56 | 79 | 77 | 705 | 8.2 | 1051.3 |
| SRS 934 | 303 | 90 | 5 | 3 | 2 | 39 | 55 | 79 | 75 | 750 | 7.7 | 1086.4 |
| SRS 934 | 403 | 90 | 5 | 4 | 2 | 38 | 55 | 79 | 69 | 850 | 7.3 | 1231.2 |
| Midas | 103 | 100 | 5 | 3 | 1 | 36 | 53 | 79 | 75 | 720 | 9.1 | 993.4 |
| Midas | 201 | 90 | 4 | 1 | 1 | 35 | 52 | 29 | 75 | 710 | 8.2 | 1040.5 |
| Midas | 302 | 90 | 5 | 1 | 1 | 36 | 53 | 79 | 72 | 860 | 8 | 1263.1 |
| Midas | 801 | 60 | 5 | 1 | 3 | 38 | 55 | 81 | 78 | 1055 | 8.4 | 1443.2 |

### Huntley, Montana (2016)

Location: Montana State University Research Farm, Southern Ag Research Center, Huntley, Montana.
Soil: Clay loam, pH 7.8. Site under no-tillage, wheat-summer fallow-camelina-summer fallow.
Climate: For Huntley, MT, the climate data published for close by Billings, MT (distance: 21 km) are used. The summers are short, hot, and mostly clear; the winters are freezing, windy, and partly cloudy; and it is dry year round. Over the course of the year, the temperature typically varies from −7° C. to 32° C. and is rarely below −19° C. or above 37° C. The hot season lasts for 2.9 months, from June 14 to September 10, with an average daily high temperature above 26° C. The hottest day of the year is July 27, with an average high of 32° C. and low of 17° C. The cold season lasts for 3.3 months, from November 18 to February 27, with an average daily high temperature below 8° C. The coldest day of the year is January 1, with an average low of −7° C. and high of 2° C. The rainy period of the year lasts for 7.2 months, from March 24 to October 29. Billings, MT receives a yearly average of 356 mm of rain and 125 cm of snow. The growing season in Billings typically lasts for 5.5 months (168 days), from around April 25 to around October 10, rarely starting before April 5 or after May 17, and rarely ending before September 19 or after October 30. According to Health Canada directive DIR2010-05, Huntley, MT is located in agro-ecological zone 5.
Weather during the 2016 growing season: Over the growing season, rainfall 168 mm cumulative, low 0.2° C., high 38° C., average low 11° C., average high 28° C.
Seeding date: Apr. 21, 2016; harvest date: Jul. 15, 2016
Results: No differences were observed for all eight parameters measured at Huntley, Montana in 2016 between camelina lines 14CS0851-01-14, SRS 934 and Midas™ (Tables 53 and 56).

TABLE 56
Life history traits in Huntley, Montana, 2016.

| 2016 Huntley, Montana | | | plant | | | | | | | | | |
|---|---|---|---|---|---|---|---|---|---|---|---|---|
| | | Plant stand | vigor | | | | | | Avg Max | Grain | | Yield |
| | | (no./meter | (1 to 5 | Plot | 10% | 50% | End of | | Plant height | moisture | Yields | (kg/ha) |
| Entry | Plot | row) | scale) | fill (%) | flowering | flowering | flowering | Maturity | (cm) | % | Kg/ha | adjusted |
| 14CS0851-01-14 | 104 | 45 | 5 | 98 | 47 DAP | 51 DAP | 62 DAP | 70 DAP | 110 | 6.7 | 513.6 | 550.812 |
| 14CS0851-01-14 | 201 | 52 | 5 | 98 | 47 DAP | 51 DAP | 62 DAP | 70 DAP | 109 | 6.4 | 440.4 | 473.789 |
| 14CS0851-01-14 | 308 | 58 | 5 | 98 | 47 DAP | 51 DAP | 62 DAP | 30 DAP | 97 | 6.4 | 494.6 | 532.117 |
| 14CS0851-01-14 | 404 | 57 | 5 | 98 | 47 DAP | 51 DAP | 62 DAP | 70 DAP | 109 | 6.7 | 495.5 | 531.432 |
| SRS 934 | 103 | 37 | 5 | 98 | 47 DAP | 51 DAP | 62 DAP | 70 DAP | 105 | 6.4 | 560.2 | 602.725 |
| SRS 934 | 207 | 34 | 5 | 100 | 47 DAP | 51 DAP | 62 DAP | 70 DAP | 111 | 6.6 | 511.7 | 549.360 |
| SRS 934 | 304 | 50 | 5 | 100 | 47 DAP | 51 DAP | 62 DAP | 70 DAP | 114 | 6.8 | 333.9 | 357.644 |
| SRS 934 | 403 | 56 | 5 | 100 | 47 DAP | 51 DAP | 62 DAP | 70 DAP | 114 | 6.5 | 507.9 | $45.860 |
| Midas | 108 | 37 | 5 | 100 | 47 DAP | 51 DAP | 62 DAP | 70 DAP | 98 | 6.4 | 451.8 | 486.069 |
| Midas | 205 | 27 | 5 | 95 | 47 DAP | 51 DAP | 62 DAP | 70 DAP | 115 | 6.7 | 396.6 | 425.349 |
| Midas | 301 | 36 | 5 | 98 | 47 DAP | 51 DAP | 62 DAP | 70 DAP | 109 | 6.4 | 511.7 | 550.537 |
| Midas | 408 | 61 | 5 | 98 | 47 DAP | 51 DAP | 62 DAP | 70 DAP | 112 | 6.7 | 525.0 | 563.052 |

### Minto, Manitoba (2016)

Location: Legal land location NW27-5-19W1
Soil: Black Soil Zone, Clay Loam soil pH of 7.8, 5% organic matter (OM)
Climate: For Minto, MB (as for Elm Creek, MB) the published climate data from close by Carman, MB are used as a representative site for Southern Manitoba. The summers are long and comfortable; the winters are frigid, snowy, and windy; and it is partly cloudy year round. Over the course of the year, the temperature typically varies from −20° C. to 26° C. and is rarely below −32° C. or above 31° C. The warm season lasts for 4.2 months, from May 15 to September 20, with an average daily high temperature above 19° C. The hottest day of the year is July 25, with an average high of 26° C. and low of 14° C. The cold season lasts for 3.3 months, from November 27 to March 6, with an average daily high temperature below −3° C. The coldest day of the year is January 15, with an average low of −20° C. and high of −11° C. The rainy period of the year lasts for 7.4 months, from March 24 to November 7. Minto, MB receives a yearly average of 385 mm of rain and 120 cm of snow. The growing season in Southern Manitoba typically lasts for 4.1 months (127 days), from around May 19 to around September 23, rarely starting before April 30 or after June 6, and rarely ending before September 7 or after October 11. According to Health Canada directive DIR2010-05, Minto, MB is located in agro-ecological zone 5.
Weather during the 2016 growing season: On Jul. 16, 2016 hail badly damaged the trial. Higher than average rainfall.
Seeding date: Jun. 5, 2016, harvest date: Sep. 14, 2016.
Comments: Hail damage appeared to affect all varieties equally, however downy mildew (biotic stress) was noted to be most prevalent in 14CS0851-01-14 (rating of 3), followed by SRS 934 (rating of 2) and MIDAS™ appeared to be most resistant (rating of 1), where 0 rating is no effect, and 10 is dead/dying.
Results: No differences were observed for Stand, Vigour, DTF 100, and Seed Yield at Minto in 2016. 14CS0851-01-14 flowered approximately two days later than the checks (DTF 10 and DTF 50) but was similar at DTF 100. 14CS0851-01-14 matured three days earlier than MIDAS™ (Tables 53 and 57).

TABLE 57
Life history traits in Minto, Manitoba, 2016.

| | | plant | | | | | | | | | | |
|---|---|---|---|---|---|---|---|---|---|---|---|---|
| | | stand | | | | | | | | | | |
| | | (% | plant | DTF 10 | DTF 10 | | | | | | | |
| | | plot | vigor | (before | (after hall) | | | DTM | | | | |
| | | fill, | (1-5, | hail) | (10% of | DTF 50 | | (50% | Plant | | | Yield |
| | | 3-4 | 3-4 | (10% of | plot | (50% of | DTF 100 | of plot | height | Seed | Grain | adjusted |
| | | leaf | leaf | plot | flowering) | plot | (end of | changed | (cm, at | yield | moisture | for |
| Entry | Plot | stage) | stage) | flowering) | | flowering) | flowering) | color) | maturity) | (g/plot) | (%) | moisture) |
| 14CS0851-01-14 | 101 | 10 | 3 | 32 | 40 | 49 | 61 | 79 | 59 | 134.6 | qns | qns |
| 14CS0851-01-14 | 203 | 25 | 2 | 33 | 42 | 49 | 62 | 79 | 65 | 198.5 | 8.6 | 326 |
| 14CS0851-01-14 | 301 | 15 | 2 | 33 | 42 | 49 | 61 | 79 | 53 | 74.7 | qns | qns |
| 14CS0851-01-14 | 403 | 20 | 3 | 33 | 30 | 49 | 60 | 79 | 64 | 111.2 | qns | qns |
| Midas | 103 | 10 | 2 | 32 | 38 | 47 | 63 | 81 | 74 | 416.9 | 9.3 | 537 |
| Midas | 202 | 15 | 3 | 32 | 40 | 47 | 61 | 83 | 75 | 435.2 | 9.7 | 728 |
| Midas | 302 | 25 | 3 | 32 | 39 | 47 | 62 | 83 | 68 | 260.2 | 11.8 | 417 |
| Midas | 403 | 15 | 2 | 32 | 38 | 47 | 62 | 80 | 68 | 292.3 | 11.4 | 462 |
| SRS 934 | 102 | 15 | 3 | 34 | 38 | 47 | 63 | 80 | 63 | 335 | 9.7 | 960 |
| SRS 934 | 201 | 40 | 5 | 34 | 39 | 47 | 61 | 79 | 71 | 289.2 | 8.8 | 509 |
| SRS 934 | 308 | 25 | 4 | 34 | 39 | 48 | 63 | 82 | 69 | 352.9 | 11.8 | 535 |
| SRS 934 | 402 | 20 | 3 | 34 | 38 | 48 | 61 | 79 | 69 | 452.4 | 11.6 | 597 |

### Morris, Minnesota (2016)

Location: Swan Lake Research Farm, Swan Lake Township, Stevens County (Approximately 6 miles north and 4 miles east of Morris, MN); 45° 41′ N Latitude and 95° 48′ W Longitude. Elevation 1211 feet.
Soil: Barnes loam soil (fine-loamy, mixed, superactive, frigid calcic hapludoll).
Climate: In Morris, the summers are long and warm; the winters are freezing, snowy, and windy; and it is partly cloudy year round. Over the course of the year, the temperature typically varies from −16° F. to 28° C. and is rarely below −27° C. or above 32° C. The warm season lasts for 4.1 months, from May 16 to September 20, with an average daily high temperature above 21° C. The hottest day of the year is July 18, with an average high of 28° C. and low of 16° C. The cold season lasts for 3.3 months, from November 27 to March 5, with an average daily high temperature below 1° C. The coldest day of the year is January 15, with an average low of −16° C. and high of −6° C. The rainy period of the year lasts for 8.2 months, from March 11 to November 16. Morris, MN receives a yearly average of 673 mm of rain and 119 cm of snow. The growing season in Morris typically lasts for 4.8 months (149 days), from around May 3 to around September 29, rarely starting before April 12 or after May 22, and rarely ending before September 11 or after October 17. According to Health Canada directive DIR2010-05, Morris, MN is located in agro-ecological zone 7.
Weather during 2016 growing season: Humid and above average rainfall, 375 mm cumulative, low 1° C., high 34° C., average high 25° C., average low 13° C.
Seeding date: May 4, 2016; harvest date: Jul. 29, 2016
Results: No differences were observed for Stand, Vigour, DTF 10, 50 or 100, and Seed Yield at Morris in 2016. 14CS0851-01-14 matured approximately two days earlier than MIDAS™ and was five cm taller than SRS 934 (Tables 53 and 58).

TABLE 58
Life history traits in Morris, Montana, 2016.

| Morris, Minnesota, 2016 | | | | | | | | | | | | |
|---|---|---|---|---|---|---|---|---|---|---|---|---|
| | | plant | | | | | | | | | | |
| | | stand | | | | | | | | | | |
| | | (% | plant | | | | | | | | | |
| | | plot | vigor | | | | DTM | | | | Yield | comments |
| | | fill, | (1-5, | DTF 10 | DTF 50 | | (50% | Plant | | | kg/ha, | regarding |
| | | 3-4 | 3-4 | (10% of | (50% of | DTF 100 | of plot | height | Seed | Grain | adj for | biotic or |
| | | leaf | leaf | plot | plot | (end of | changed | (cm, at | yield | moisture | 8% | abiotic |
| entry name | Plot | stage) | stage) | flowering) | flowering) | flowering) | color) | maturity) | (g/plot) | (%) | moisture | stress |
| 14CS0851-01-14 | 105 | 33 | 5 | 43 | $3 | 48 | 79 | 62 | 1433.1 | 11.06 | 1237.7 | *Jul. 3, 2016 |
| | | | | | | | | | | | | downy mildew 5% |
| 14CS0851-01-14 | 207 | 35 | 5 | 41 | 43 | 48 | 79 | 64 | 1580.3 | 12.52 | 1353.8 | *Jul. 3, 2016 |
| | | | | | | | | | | | | downy mildew 1% |
| 14CS0851-01-14 | 306 | 31 | 5 | 43 | 44 | 48 | 39 | 63 | 1665.4 | 12.34 | 1425.2 | *Jul. 3, 2016 |
| | | | | | | | | | | | | downy mildew 1-2%; |
| | | | | | | | | | | | | some lodging |
| 14CS0851-01-14 | 403 | 31 | 4 | 44 | 48 | 50 | 80 | 68 | 1376.6 | 18.48 | 1223.3 | *Jul. 3, 2016 |
| | | | | | | | | | | | | downy mildew <1% |
| Midas | 187 | 32 | 5 | 43 | 43 | 48 | 82 | 57 | 1723.5 | 18.44 | 1463.5 | *Jul. 3, 2016 |
| | | | | | | | | | | | | downy mildew <1% |
| Midas | 202 | 35 | 5 | 43 | 43 | 48 | 80 | 65 | 1861.9 | 16.66 | 1629.3 | *Jul. 3, 2016 |
| | | | | | | | | | | | | downy mildew 0% |
| Midas | 308 | 34 | 5 | 41 | 43 | 48 | 80 | 61 | 1338.2 | 19.01 | 1228.7 | *Jul. 3, 2016 |
| | | | | | | | | | | | | downy mildew 0% |
| Midas | 404 | 38 | 5 | 43 | 48 | 48 | 81 | 68 | 1572.6 | 41.19 | 1407.1 | *Jul. 3, 2016 |
| | | | | | | | | | | | | downy mildew 0% |
| SRS 934 | 203 | 34 | 5 | 43 | 45 | 50 | 81 | 55 | 1338.3 | 15.22 | 1459.4 | *Jul. 3, 2016 |
| | | | | | | | | | | | | downy mildew <1% |
| | | | | | | | | | | | | affected |
| SRS 934 | 204 | 35 | 5 | 42 | 45 | 48 | 79 | 60 | 1488.6 | 12.03 | 1413.7 | *Jul. 3, 2016 |
| | | | | | | | | | | | | downy mildew <1% |
| SRS 934 | 301 | 35 | 5 | 43 | 45 | 48 | 79 | 62 | 1551.8 | 38.72 | 1372.8 | *Jul. 3, 2016 |
| | | | | | | | | | | | | downy mildew 0%; |
| | | | | | | | | | | | | some lodging |
| SRS 934 | 408 | 33 | 5 | 41 | 43 | 48 | 80 | 62 | 1198.4 | 14.67 | 1061.2 | *Jul. 3, 2016 |
| | | | | | | | | | | | | downy mildew 0% |

### NDSU Fargo, North Dakota (2016)

Location: N 48° 10′ 34.9″ W 101° 17′ 40.5″
Soil: Loam, pH 5.4, organic matter 3.4%
Climate: In Fargo, the summers are long and warm; the winters are frigid, snowy, and windy; and it is partly cloudy year round. Over the course of the year, the temperature typically varies from −17° C. to 28° C. and is rarely below −28° C. or above 32° C. The warm season lasts for 4.1 months, from May 16 to September 19, with an average daily high temperature above 21° C. The hottest day of the year is July 24, with an average high of 28° C. and low of 16° C. The cold season lasts for 3.3 months, from November 27 to March 5, with an average daily high temperature below −1° C. The coldest day of the year is January 15, with an average low of −17° C. and high of −7° C. The rainy period of the year lasts for 7.9 months, from March 17 to November 14. Fargo, ND receives a yearly average of 674 mm of rain and 127 cm of snow. The growing season in Fargo typically lasts for 5.0 months (152 days), from around May 5 to around October 4, rarely starting before April 16 or after May 23, and rarely ending before September 15 or after October 24. According to Health Canada directive DIR2010-05, Fargo, ND is located in agro-ecological zone 5.
Weather during 2016 growing season: Total rainfall during growing season was 233 mm (normal 269 mm). Average high temperature was 25° C., range 21-28° C. (normal 19-27° C.), average low temperature was 18° C., range 14-21° C. (normal 1221° C.).
Seeding date: May 16, 2016; harvest date: Aug. 15, 2016
Results: No differences were observed for Stand, Vigour, DTF 10 or 50, DTM and Seed Yield at Fargo in 2016. 14CS0851-01-14 flowered approximately four days earlier than the checks at full flower (DTF 100). 14CS0851-01-14 was approximately 10 cm taller than SRS 934 and MIDAS™ (Tables 53 and 59).

TABLE 59
Life history traits in Fargo, North Dakota, 2016.

| North Dakota State University, 2016 | | | | | | | | | |
|---|---|---|---|---|---|---|---|---|---|
| | | Plotfill | Seedling vg | 10% bloom | 50% bloom | 90% bloom | Maturity | Height | Yield |
| Entry | Plot | Percent | 1-3 | DAP | DAP | DAP | DAP | cm | lb/A |
| 14CS0851-01-14 | 108 | 99 | 2 | 47 | 49 | 61 | 78 | 70 | 295 |
| 14CS0851-01-14 | 204 | 99 | 2 | 44 | 49 | 61 | 79 | 64 | 298 |
| 14CS0851-01-14 | 312 | 98 | 2 | 47 | 51 | 61 | 79 | 57 | 58 |
| 14CS0851-01-14 | 402 | 95 | 2 | 43 | 49 | 61 | 78 | 67 | 215 |
| SRS 934 | 102 | 99 | 2 | 46 | 49 | 63 | 78 | 61 | 401 |
| SRS 934 | 205 | 98 | 2 | 48 | 51 | 65 | 81 | 52 | 186 |
| SRS 934 | 301 | 99 | 2 | 48 | 55 | 67 | 83 | 50 | 208 |
| SRS 934 | 407 | 99 | 2 | 47 | 53 | 63 | 79 | 57 | 83 |
| Midas | 110 | 99 | 2 | 46 | 50 | 63 | 79 | 61 | 457 |
| Midas | 212 | 99 | 2 | 49 | 57 | 65 | 83 | 49 | 151 |
| Midas | 308 | 98 | 2 | 49 | 53 | 69 | 87 | 52 | 356 |
| Midas | 410 | 99 | 2 | 44 | 49 | 61 | 79 | 61 | 219 |

### Saskatoon, Saskatchewan (2016) (Ag-Quest)

Location: Legal land location SW 31-36-6 W3
Soil: Moist Dark Brown Loam, 40% sand, 40% silt, and 20% clay; good soil drainage.
Climate: In Saskatoon, the summers are long, comfortable, and partly cloudy and the winters are frigid, snowy, windy, and mostly cloudy. Over the course of the year, the temperature typically varies from −19° C. to 26° C. and is rarely below −33° C. or above 31° C. The warm season lasts for 4.1 months, from May 15 to September 18, with an average daily high temperature above 18° C. The hottest day of the year is July 27, with an average high of 26° C. and low of 13° C. The cold season lasts for 3.5 months, from November 23 to March 6, with an average daily high temperature below −3° C. The coldest day of the year is January 11, with an average low of −19° C. and high of −11° F. The rainy period of the year lasts for 6.2 months, from April 9 to October 16. Saskatoon, SK receives a yearly average of 280 mm of rain and 76 cm of snow. The growing season in Saskatoon typically lasts for 4.1 months (126 days), from around May 17 to around September 20, rarely starting before April 30 or after June 4, and rarely ending before September 5 or after October 6. According to Health Canada directive DIR2010-05, Saskatoon, SK is located in agro-ecological zone 5.
Weather during 2016 growing season: Between May 5 and August 27, there was 48 days of rain, for a total of 236 mm. Minimum temperature of 1° C., maximum 29° C., average low 12° C., average high 23° C.
Seeding date: May 27, 2016; harvest date: Aug. 27, 2016
Results: No differences were observed for Stand, Vigour, DTF 10, 50 or 100, and Seed Yield at Saskatoon in 2016. 14CS0851-01-14 matured approximately three days later than SRS 934 and was approximately seven cm taller than MIDAS™ (Tables 53 and 60).

TABLE 60
Life history traits in Saskatoon, Saskatchewan, 2016.

| Saskatoon, SK, 2016 AgQuest | | | | | | | | | | | | |
|---|---|---|---|---|---|---|---|---|---|---|---|---|
| | | Jun. 24, | Jun. 24, | | | | | | Sep. 27, | Sep. 27, | | |
| | | 2016 | 2016 | | | | | Aug. 26, | 2016 | 2016 | Sep. 27, | Sep. 27, |
| | | plant | plant | | | | | 2016 | Seed | Grain | 2016 | 2016 |
| | | stand | vigor | | | | | HEIGHT | yield | moist | Yield | Yield |
| Entry | Plot | % | 1-5 | DTF 10 | DTF 50 | DTF 100 | DTM | cm | g/plot | % | kg/ha | kg/ha |
| 14CS0851-01-14 | 103 | 40 | 2.5 | 35 | 42 | 66 | 87 | 86.6 | 968.5 | 9.2 | 1073.6 | 1073.6 |
| 14CS0851-01-14 | 201 | 90 | 1 | 35 | 42 | 65 | 87 | 101.8 | 1093 | 7.8 | 1230.3 | 1230.3 |
| 14CS0851-01-14 | 303 | 60 | 3 | 35 | 42 | 66 | 87 | 94.8 | 1278.8 | 8.8 | 1421.6 | 1421.6 |
| 14CS0851-01-14 | 402 | 30 | 3 | 35 | 42 | 69 | 87 | 90.8 | 776 | 9.1 | 861.2 | 1363.6* |
| SRS 934 | 102 | 50 | 2 | 35 | 42 | 66 | 84 | 92.2 | 1110.1 | 8.5 | 1240.1 | 1240.1 |
| SRS 934 | 203 | 75 | 1 | 35 | 42 | 66 | 84 | 96.6 | 1011.5 | 9.1 | 1122.5 | 1122.5 |
| SRS 934 | 302 | 70 | 1 | 35 | 42 | 66 | 84 | 95.4 | 1498.7 | 8.4 | 1676 | 1676 |
| SRS 934 | 403 | 60 | 2 | 35 | 42 | 66 | 85 | 95.6 | 1251.2 | 8.3 | 1400.8 | 1400.8 |
| Midas | 101 | 50 | 3 | 35 | 42 | 63 | 86 | 83.4 | 1413 | 8.6 | 1576.7 | 1576.7 |
| Midas | 202 | 55 | 1 | 35 | 42 | 66 | 86 | 93.4 | 919.4 | 8.3 | 1029.3 | 1029.3 |
| Midas | 301 | 40 | 3.5 | 35 | 42 | 66 | 87 | 89.6 | 970.2 | 7.5 | 1095.7 | 1095.7 |
| Midas | 401 | 50 | 3 | 35 | 42 | 66 | 87 | 83.6 | 1275 | 8.6 | 1422.8 | 1422.8 |

### Taber, Alberta (2016)

Location: SE 31 9 15 W4
Soil: Sandy clay loam, about 55% sand, 22% silt, 23% clay, zone 7a, pH 8.1
Climate: For Taber, AB, the published climate data for close by Lethbridge, AB are used. The summers are warm; the winters are freezing, dry, and windy; and it is partly cloudy year round. Over the course of the year, the temperature typically varies from −11° C. to 27° C. and is rarely below −26° C. or above 33° C. The warm season lasts for 3.1 months, from June 11 to September 14, with an average daily high temperature above 22° C. The hottest day of the year is August 5, with an average high of 27° C. and low of 12° C. The cold season lasts for 3.6 months, from November 18 to March 4, with an average daily high temperature below 5° C. The coldest day of the year is January 1, with an average low of −11° C. and high of −1° C. The rainy period of the year lasts for 6.2 months, from April 7 to October 15. Taber, AB receives a yearly average of 260 mm of rain and 107 cm of snow. The growing season around Lethbridge typically lasts for 4.4 months (136 days), from around May 12 to around September 26, rarely starting before April 24 or after May 29, and rarely ending before September 9 or after October 13. According to Health Canada directive DIR2010-05, Taber, AB is located in agro-ecological zone 14.
Weather during 2016 growing season: Between seeding May 11, 2016 and harvest Sep. 2, 2016 there was accumulative 260 mm of precipitation, with frost just prior to emergence. Trial was irrigated on June 7, one week post-herbicide treatment. Rain and hail occurred at bolting/flower stage.
Seeding date: May 11, 2016, harvest date: Sep. 2, 2016.
Comments: No differences in damage were observed between the varieties due to frost, rain, or hail.
Results: No differences were observed for Stand, Vigour, DTF 10 or 100, and DTM at Taber in 2016. 14CS0851-01-14 flowered approximately one day earlier than the checks at 50% flower (DTF 50). HGT was not determined. 14CS0851-01-14 yielded more than SRS 934 but was similar in yield to MIDAS™ (Tables 53 and 61).

TABLE 61
Life history traits in Taber, Alberta, 2016.

| | |
|---|---|
| Seed | Grain |

| Taber, Alberta 2016 | | plant | plant | DTF | DTF | DTF | | yield | moisture | Yield | comments |
|---|---|---|---|---|---|---|---|---|---|---|---|
| entry | Plot | stand | vigor | 10 | 50 | 100 | | (g/plat) | (%) | Kg/ha | regarding |
| 14CS0851-01-14 | 104 | 95 | 5 | 41 | 45 | 55 | 78 | 1700 | 6.8 | 2724 | * Bolt/Flower Stage - |
| | | | | | | | | | | | Rain and Hail (2) |
| 14CS0851-01-14 | 208 | 95 | 5 | 41 | 45 | 55 | 78 | 1800 | 7.1 | 2943 | * Bolt/Flower Stage - |
| | | | | | | | | | | | Rain and Hail (2) |
| 14CS0851-01-14 | 303 | 95 | 5 | 41 | 45 | 55 | 78 | 2000 | 7.3 | 3505 | * Bolt/Flower Stage - |
| | | | | | | | | | | | Rain and Hall (4) |
| 14CS0851-01-14 | 407 | 95 | 5 | 41 | 45 | 55 | 78 | 2100 | 5.8 | 3312 | * Bolt/Flower Stage - |
| | | | | | | | | | | | Rain and Hail (2) |
| Midas | 103 | 95 | 5 | 42 | 45 | 56 | 81 | 2000 | 6.9 | 3208 | * Bolt/Flower Stage - |
| | | | | | | | | | | | Rain and Hail (2) |
| Midas | 201 | 95 | 5 | 42 | 46 | 56 | 79 | 2100 | 7.1 | 3431 | * Bolt/Flower Stage - |
| | | | | | | | | | | | Rain and Hail (2) |
| Midas | 304 | 95 | 5 | 42 | 46 | 56 | 78 | 2000 | 7.2 | 3441 | * Bolt/Flower Stage - |
| | | | | | | | | | | | Rain and Hall (4) |
| Midas | 405 | 95 | 5 | 42 | 47 | 58 | 79 | 1700 | 7 | 2730 | * Bolt/Flower Stage - |
| | | | | | | | | | | | Rain and Hail (2) |
| SRS 934 | 107 | 95 | 5 | 42 | 45 | 56 | 79 | 1500 | 6.9 | 2406 | * Bolt/Flower Stage - |
| | | | | | | | | | | | Rain and Hail (2) |
| SRS 934 | 205 | 95 | 5 | 42 | 46 | 56 | 79 | 1600 | 7 | 2611 | * Bolt/Flower Stage - |
| | | | | | | | | | | | Rain and Hail (2) |
| SRS 934 | 309 | 95 | 5 | 42 | 47 | 56 | 79 | 1100 | 6.7 | 1882 | * Bolt/Flower Stage - |
| | | | | | | | | | | | Rain and Hail (2) |
| SRS 934 | 403 | 95 | 5 | 42 | 45 | 56 | 80 | 1400 | 7.6 | 2338 | * Bolt/Flower Stage - |
| | | | | | | | | | | | Rain and Hail (2) |

### Huntley, Montana (2017)

Location: Montana State University Research Farm, Southern Ag Research Center, Huntley, Montana.
Soil: Clay loam, pH 7.8. Site under no-tillage, wheat-summer fallow-camelina-summerfallow.
Climate: See above.
Weather during 2017 growing season: Over the growing season, rainfall 90.2 mm cumulative, low −0.3° C., high 38.8° C., average low 12° C., average high 29° C.
Seeding date: Apr. 13, 2017; harvest date: Jul. 26, 2017
Results: No differences were observed for Vigour, DTF 10, 50 or 100, and HGT at Huntley in 2017. 14CS0851-01-14's Stand was 11% lower than that of MIDAS™ but similar to that of SRS 934. 14CS0851-01-14 matured two and three days earlier than SRS 934 and MIDAS™, respectively (Tables 53 and 62).

TABLE 62
Life history traits in Huntley, Montana, 2017.

| | | | | | | | DTM | | | |
|---|---|---|---|---|---|---|---|---|---|---|
| | | plant stand | | DTF 10 | DTF 50 | | (50% | Plant | | |
| | | (% plot | plant vigor | (10% of | (50% of | DTF 100 | of plot | height | | biotic or |
| Huntley, Montana, 2017 | | fill, 3-4 | (1-5, 3-4 | plot | plot | (end of | changed | (cm, at | Vield | abiotic |
| entry name | Plot | leaf stage) | leaf stage) | flowering) | flowering) | flowering) | color) | maturity) | (kg/ha) | stress |
| 14CS0851-01 | 106 | 85 | 4 | 53 | 60 | 78 | 89 | 59 | 246.9 | 0 |
| 14CS0851-01 | 211 | 85 | 4 | 53 | 60 | 78 | 88 | 62.2 | 289.2 | 0 |
| 14CS0851-01 | 304 | 80 | 4 | 53 | 60 | 78 | 88 | 61.8 | 284.2 | 0 |
| 14CS0851-01 | 405 | 70 | 4 | 55 | 68 | 85 | 86 | 45.2 | 274.7 | 0 |
| SRS 934 | 112 | 80 | 3 | 53 | 62 | 80 | 88 | 59 | 301.4 | 0 |
| SRS 934 | 203 | 80 | 2 | 53 | 62 | 80 | 91 | 58.4 | 320.0 | 0 |
| SRS 934 | 307 | 70 | 3 | 53 | 64 | 82 | 91 | 60.4 | 341.6 | 0 |
| SRS 934 | 408 | 80 | 4 | 53 | 63 | 80 | 90 | 60.5 | 310.5 | |
| Midas | 107 | 90 | 4 | 53 | 60 | 78 | 91 | 63.2 | 376.4 | 0 |
| Midas | 209 | 90 | 5 | 53 | 62 | 80 | 91 | 63.6 | 431.6 | 0 |
| Midas | 310 | 90 | 5 | 53 | 62 | 80 | 90 | 72.6 | 358.5 | 0 |
| Midas | 401 | 95 | 3 | 53 | 64 | 82 | 91 | 62.6 | 379.9 | 0 |

### Morris, Montana (2017)

Location: Swan Lake Research Farm, Swan Lake Township, Stevens County (Approximately 6 miles north and 4 miles east of Morris, MN) 45° 41′ N Latitude and 95° 48′ W Longitude. Elevation 1211 feet.
Soil: Barnes loam soil (fine-loamy, mixed, superactive, frigid calcic hapludoll).
Climate: See above.
Weather during 2017 growing season: Humid and above average rainfall 391 mm cumulative, low 0° C., high 34° C., average high 24° C., average low 13° C.
Results: No differences were observed for Vigour, DTF 10, 50 or 100, and DTM at Morris in 2017. 14CS0851-01-14's Stand was nine percent lower than that of MIDAS™ and six percent higher than that of SRS 934._14CS0851-01-14 was three cm taller than SRS 934 and of similar height to MIDAS™. 14CS0851-01-14 yielded more than SRS 934 but was similar in yield to MIDAS™ (Tables 53 and 63).

TABLE 63
Life history traits in Morris, Montana, 2017.

| | | plant | | | | | | | | | | |
|---|---|---|---|---|---|---|---|---|---|---|---|---|
| | | stand | plant | | | | | | | | Yield | |
| | | (% plot | vigor | | | | DTM | | | | at 8% | |
| | | fill, | (3-5, | DTF 10 | DTF 50 | | (50% | Plant | | | moisture | comments |
| | | 3-4 | 3-4 | (10% | (50% | DTF 100 | of plot | height | Seed | Grain | adjusted | regarding |
| | | leaf | leaf | of plot | of plot | (end of | changed | form, at | yield | moisture | for | biotic or |
| entry name | Plot | stage) | stage) | flowering) | flowering) | flowering) | color) | maturity) | (g/plot) | (%) | moisture) | abiotic stress |
| 14CS0851-01-14 | 110 | 28 | 5 | 42 | 41 | 47 | 74 | 78 | 1387.8 | 6.99 | 1052.9 | No biotic or |
| | | | | | | | | | | | | abiotic stress |
| | | | | | | | | | | | | observed |
| 14CS0851-01-14 | 201 | 18 | 4 | 43 | 46 | 49 | 79 | 79 | 1195.1 | 10.34 | 906.1 | No biotic or |
| | | | | | | | | | | | | abiotic stress |
| | | | | | | | | | | | | observed |
| 14CS0851-01-14 | 308 | 25 | 5 | 41 | 43 | 47 | 76 | 79 | 1375.9 | 9.41 | 1035.5 | No biotic or |
| | | | | | | | | | | | | abiotic stress |
| | | | | | | | | | | | | observed |
| 14CS0851-01-14 | 413 | 18 | 4 | 42 | 45 | 48 | 78 | 79 | 1797.8 | 8.45 | 1333.1 | No biotic or |
| | | | | | | | | | | | | abiotic stress |
| | | | | | | | | | | | | observed |
| SRS 934 | 103 | 15 | 4 | 43 | 46 | 49 | 78 | 73 | 1277.5 | 8.95 | 959.4 | No biotic or |
| | | | | | | | | | | | | abiotic stress |
| | | | | | | | | | | | | observed |
| SRS 934 | 205 | 15 | 4 | 42 | 44 | 49 | 76 | 74 | 725.7 | 10.10 | 554.2 | Generally, |
| | | | | | | | | | | | | emergence |
| | | | | | | | | | | | | was poor |
| SRS 934 | 312 | 15 | 5 | 42 | 44 | 48 | 74 | 75 | 1000.7 | 6.64 | 752.8 | Generally, |
| | | | | | | | | | | | | emergence |
| | | | | | | | | | | | | was poor |
| SRS 934 | 403 | 20 | 5 | 42 | 44 | 47 | 76 | 79 | 1068.5 | 8.64 | 787.0 | Generally, |
| | | | | | | | | | | | | emergence |
| | | | | | | | | | | | | was poor |
| Midas | 115 | 30 | 5 | 42 | 44 | 47 | 78 | 75 | 1598.5 | 9.95 | 1188.7 | No biotic or |
| | | | | | | | | | | | | abiotic stress |
| | | | | | | | | | | | | observed |
| Midas | 215 | 25 | 5 | 43 | 45 | 48 | 76 | 76 | 1579.9 | 7.90 | 1205.9 | No biotic or |
| | | | | | | | | | | | | abiotic stress |
| | | | | | | | | | | | | observed |
| Midas | 304 | 35 | 5 | 39 | 42 | 47 | 77 | 80 | 2197.4 | 7.92 | 1642.7 | No biotic or |
| | | | | | | | | | | | | abiotic stress |
| | | | | | | | | | | | | observed |
| Midas | 405 | 32 | 5 | 41 | 42 | 47 | 75 | 82 | 1862.2 | 7.62 | 1383.2 | No biotic or |
| | | | | | | | | | | | | abiotic stress |
| | | | | | | | | | | | | observed |

### Saskatoon, Saskatchewan (2017) (AAFR Research Farm)

Location: Legal land location SE1/2 Sec 12-37-5-W3
Soil: Moist Dark Brown Loam, 40% sand, 40% silt, and 20% clay; good soil drainage. Climate: See above.
Weather during 2017 growing season: Between May 1 and August 31 there was 40 days of rain, 162 mm cumulative. Temperature low −2° C., high 34° C., average high 23° C., average low 9° C.
Seeding date: Jun. 1, 2017; harvest date: Sep. 7, 2017.
Results: No differences were observed for Stand, Vigour, DTF 10, 50 or 100, DTM and HGT at Saskatoon in 2017. 14CS0851-01-14 yielded less than the checks (Tables 53 and 64)

TABLE 64
Life history traits in Saskatoon, Saskatchewan, 2017.

| | | | | | | | | | | | biotic or |
|---|---|---|---|---|---|---|---|---|---|---|---|
| | | | | | | | | | | | abiotic |
| | | | plant | | | | DTM | | | | stress |
| | | plant stand | vigor | | | | (50% of | Plant | | | Downy |
| | | (% plot fill, | (1-5, 3-4 | DTF 10 | DTF 50 | DTF 100 | plot | height | Seed | | mildew |
| Saskatoon, SK., AAFC, 2017 | | 3-4 leaf | leaf | (10% of plot | (50% of plot | (end of | changed | (cm, at | yield | Yield | (1-5, July |
| Entry | Plot | stage) | stage) | flowering) | flowering) | flowering) | color) | maturity) | (g/plot) | (kg/ha) | 31st) |
| 14CS0851-01-14 | 114 | 70 | 4 | 37 | 45 | 56 | 77 | 72 | 1347.5 | 1811.1 | 4 |
| 14CS0851-01-14 | 201 | 80 | 4 | 37 | 45 | 58 | 81 | 64 | 1645.6 | 2211.8 | 3 |
| 14CS0851-01-14 | 321 | 100 | 4 | 37 | 45 | 55 | 79 | 79 | 1474.1 | 1981.3 | 4 |
| 14CS0851-01-14 | 419 | 100 | 4 | 37 | 44 | 55 | 78 | 82 | 1428.0 | 1919.4 | 3 |
| SRS 934 | 102 | 60 | 4 | 37 | 45 | 56 | 78 | 66 | 2065.6 | 2776.4 | 2 |
| SRS 934 | 208 | 70 | 4 | 37 | 45 | 56 | 79 | 70 | 2276.7 | 3060.1 | 2 |
| SRS 934 | 311 | 90 | 4 | 37 | 45 | 55 | 80 | 76 | 1870.8 | 2514.6 | 2 |
| SRS 934 | 415 | 90 | 4 | 37 | 45 | 56 | 81 | 78 | 1873.6 | 2518.3 | 1 |
| Midas | 119 | 70 | 4 | 37 | 45 | 55 | 78 | 86 | 2036.8 | 2737.7 | 2 |
| Midas | 206 | 80 | 4 | 37 | 45 | 56 | 80 | 69 | 2804.4 | 3769.3 | 2 |
| Midas | 302 | 100 | 4 | 37 | 45 | 56 | 81 | 68 | 2137.8 | 2873,4 | 2 |
| Midas | 411 | 100 | 4 | 37 | 45 | 56 | 79 | 81 | 1895.9 | 2548.3 | 1 |

### Example 18: Modified MIDAS™ Lines

In order to introduce the herbicide tolerance trait (2 genes) into an elite camelina cultivar (MIDAS™), introgression was performed using 13CS0583 (MIDAS™) and 13CS0780-02 (F3 seed, derived from cross: 12CS0364×12CS0365, each obtained as in Example 2). The following crosses and backcrosses (BC) were performed:
(i) F1: 13CS0583×13CS0780−02=14CS0903
(ii) BC1: 14CS0903 and 13CS0583=15CS0909
(iii) BC2: 15CS0909 and 13CS0583=15CS0985
(iv) BC3: 15CS0985 and 13CS0583=15CS1007
(v) BC4F1: 15CS1007 and 13CS0583=15CS1018
(vi) BC4F2: 15CS1018 selfed=16CS1054
(vii) BC4F3: 16CS1054 selfed=16CS1068
(viii) BC4F4: 16CS1068 bulked and selfed: 17CS1115

17CS1115 was planted and sprayed with Pinnacle™ SG at a 0, 1× and 2× field rate in replicated trials at one or more of 3 sites—Saskatoon AAFC, Montana State University in Huntley, Montana (Dr. Prashant Jha), and USDA in Morris, Minnesota (Dr. Russ Gesch). The protocol employed is as described in Example 7. The data for 17CS1115 at a site in Saskatoon is provided below in Table 65.

TABLE 65
Field Trial Data at Saskatoon, SK in 2017.

| | | | | Plant | Herbicide injury | | | | | | | |
|---|---|---|---|---|---|---|---|---|---|---|---|---|
| | | | | height | (% stunting) | | | Seed | | | | |
| | | | | (cm, at | 7 | 21 | 42 | yield | Yield | Oil | Protein | |
| Plot | Rep | Entry | Rate | maturity) | daa | daa | daa | (g/plot) | (kg/ha) | (%) | (%) | TKW |
| 101 | 1 | SRS 934 | 2x | 59 | 100 | 98 | 98 | 1389.27 | 1867.30 | 38.1 | 30.65 | 0.96 |
| 102 | 1 | SRS 934 | 0 | 66 | n/a. | n/a. | n/a. | 2065.64 | 2776.40 | 38.48 | 30.75 | 1.15 |
| 103 | 1 | SRS 934 | 1x | 61 | 98 | 95 | 95 | 1188.02 | 1596.80 | 36.66 | 30.22 | 0.83 |
| 110 | 1 | 17CS1115 | 0 | 71 | n/a. | n/a. | n/a. | 2585.62 | 3475.30 | 42.35 | 27.93 | 1.31 |
| 111 | 1 | 17CS1115 | 2x | 69 | 10 | 5 | 5 | 1987.45 | 2671.30 | 41.88 | 28.39 | 1.27 |
| 112 | 1 | 17CS1115 | 1x | 71 | 2 | 2 | 0 | 2542.99 | 3418.00 | 41.22 | 28.53 | 1.25 |
| 113 | 1 | 14CS0851- | 1x | 73 | 2 | 2 | 0 | 1629.96 | 2190.80 | 40.04 | 29.98 | 1.07 |
| | | 01-14 | | | | | | | | | | |
| 114 | 1 | 14CS0851- | 0 | 72 | n/a. | n/a. | n/a. | 1347.46 | 1811.10 | 39.39 | 30.44 | 1.07 |
| | | 01-14 | | | | | | | | | | |
| 115 | 1 | 14CS0851- | 2x | 78 | 2 | 5 | 5 | 953.81 | 1282.00 | 37.96 | 30.45 | 0.99 |
| | | 01-14 | | | | | | | | | | |
| 119 | 1 | Midas | 0 | 86 | n/a. | n/a. | n/a. | 2036.85 | 2737.70 | 40.8 | 29.42 | 1.16 |
| 120 | 1 | Midas | 2x | 61 | 90 | 95 | 95 | 656.65 | 882.60 | 37 | 29.01 | 0.84 |
| 121 | 1 | Midas | 1x | 58 | 85 | 90 | 90 | 557.93 | 749.90 | 38.49 | 30.41 | 0.91 |
| 201 | 2 | 14CS0851- | 0 | 64 | n/a. | n/a. | n/a. | 1645.58 | 2211.80 | 40.15 | 30.24 | 1.12 |
| | | 01-14 | | | | | | | | | | |
| 202 | 2 | 14CS0851- | 1x | 60 | 0 | 0 | 0 | 1904.94 | 2560.40 | 39.65 | 30 | 1.07 |
| | | 01-14 | | | | | | | | | | |
| 203 | 2 | 14CS0851- | 2x | 66 | 2 | 2 | 2 | 1370.15 | 1841.60 | 38.47 | 30.9 | 1.12 |
| | | 01-14 | | | | | | | | | | |
| 204 | 2 | Midas | 1x | 58 | 85 | 95 | 95 | 1275.66 | 1714.60 | 38.87 | 29.66 | 0.86 |
| 205 | 2 | Midas | 2x | 54 | 95 | 98 | 98 | 1011.77 | 1359.90 | 36.36 | 29.77 | 0.71 |
| 206 | 2 | Midas | 0 | 69 | n/a. | n/a. | n/a. | 2804.36 | 3769.30 | 40.29 | 29.64 | 1.2 |
| 207 | 2 | SRS 934 | 2x | 56 | 100 | 98 | 98 | 1092.64 | 1468.60 | 36.12 | 30.81 | 0.8 |
| 208 | 2 | SRS 934 | 0 | 70 | n/a. | n/a. | n/a. | 2276.71 | 3060.10 | 38.45 | 30.91 | 1.11 |
| 209 | 2 | SRS 934 | 1x | 70 | 100 | 95 | 95 | 1224.70 | 1646.10 | 37.13 | 30.92 | 0.85 |
| 219 | 2 | 17CS1115 | 2x | 73 | 5 | 5 | 5 | 1972.72 | 2651.50 | 42.94 | 26.85 | 1.26 |
| 220 | 2 | 17CS1115 | 0 | 80 | n/a. | n/a. | /a. | 2756.15 | 3704.50 | 42.99 | 26.81 | 1.25 |
| 221 | 2 | 17CS1115 | 1x | 77 | 2 | 2 | 0 | 2248.00 | 3021.50 | 42.87 | 27.06 | 1.23 |
| 301 | 3 | Midas | 2x | 58 | 90 | 98 | 98 | 856.64 | 1151.40 | 38.44 | 29.86 | 0.75 |
| 302 | 3 | Midas | 0 | 68 | n/a. | n/a. | n/a. | 2137.80 | 2873.39 | 40.35 | 29.4 | 1.2 |
| 303 | 3 | Midas | 1x | 54 | 80 | 85 | 85 | 934.77 | 1256.41 | 38.98 | 30.21 | 0.89 |
| 310 | 3 | SRS 934 | 1x | 59 | 100 | 95 | 95 | 813.61 | 1093.56 | 37.26 | 31.64 | 0.93 |
| 311 | 3 | SRS 934 | 0 | 76 | n/a. | n/a. | n/a. | 1870.83 | 2514.56 | 39.37 | 30.16 | 1.07 |
| 312 | 3 | SRS 934 | 2x | 59 | 100 | 98 | 98 | 611.74 | 822.23 | 36.13 | 31.17 | 0.69 |
| 316 | 3 | 17CS1115 | 1× | 82 | 2 | 0 | 0 | 2192.17 | 2946.47 | 43.12 | 27.03 | 1.23 |
| 317 | 3 | 17CS1115 | 0 | 82 | n/a. | n/a. | n/a. | 2204.01 | 2962.38 | 42.89 | 27.03 | 1.28 |
| 318 | 3 | 17CS1115 | 2x | 70 | 10 | 5 | 5 | 1929.15 | 2592.94 | 42.61 | 27.38 | 1.1 |
| 319 | 3 | 14CS0851- | 1x | 84 | 2 | 2 | 0 | 1205.29 | 1620.01 | 39.8 | 29.57 | 1.01 |
| | | 01-14 | | | | | | | | | | |
| 320 | 3 | 14CS0851- | 2x | 74 | 5 | 5 | 5 | 1099.19 | 1477.41 | 38.3 | 30.11 | 0.94 |
| | | 01-14 | | | | | | | | | | |
| 321 | 3 | 14CS0851- | 0 | 79 | n/a. | n/a. | n/a. | 1474.10 | 1981.32 | 39.55 | 29.98 | 1 |
| | | 01-14 | | | | | | | | | | |
| 407 | 4 | 17CS1115 | 2x | 70 | 5 | 5 | 2 | 2012.39 | 2704.83 | 41.94 | 28.43 | 1.24 |
| 408 | 4 | 17CS1115 | 0 | 74 | n/a. | n/a. | n/a. | 2144.09 | 2881.84 | 42.16 | 28.21 | 1.25 |
| 409 | 4 | 17CS1115 | 1x | 77 | 0 | 0 | 0 | 2048.54 | 2753.41 | 41.62 | 28.39 | 1.19 |
| 410 | 4 | Midas | 1x | 52 | 80 | 85 | 85 | 845.56 | 1136.51 | 39.82 | 30.1 | 0.98 |
| 411 | 4 | Midas | 0 | 81 | n/a. | n/a. | n/a. | 1895.91 | 2548.27 | 41.05 | 29.17 | 1.13 |
| 412 | 4 | Midas | 2x | 43 | 90 | 95 | 95 | 631.18 | 848.36 | 38.24 | 30.06 | 0.72 |
| 413 | 4 | SRS 934 | 1x | 55 | 95 | 95 | 95 | 701.34 | 942.66 | 36.48 | 31.52 | 0.79 |
| 414 | 4 | SRS 934 | 2x | 56 | 100 | 98 | 98 | 372.35 | 500.47 | 33.93 | 29.65 | 0.64 |
| 415 | 4 | SRS 934 | 0 | 78 | n/a. | n/a. | n/a. | 1873.64 | 2518.33 | 38.18 | 31.09 | 1.07 |
| 419 | 4 | 14CS0851- | 0 | 82 | n/a. | n/a. | n/a. | 1428.04 | 1919.41 | 39.85 | 30.17 | 1.12 |
| | | 01-14 | | | | | | | | | | |
| 420 | 4 | 14CS0851- | 1x | 79 | 0 | 0 | 0 | 1412.32 | 1898.28 | 39.62 | 30.15 | 1 |
| | | 01-14 | | | | | | | | | | |
| 421 | 4 | 14CS0851- | 2x | 83 | 2 | 0 | 0 | 1411.68 | 1897.42 | 39.68 | 30.36 | 1.05 |
| | | 01-14 | | | | | | | | | | |

The data demonstrated that the BC4F4 plant of the present disclosure (17CS1115) exhibited significantly increased tolerance or resistance to Group 2 herbicides, similar to the modified Camelina line 14CS0851-01-14. 17CS1115 showed good tolerance to the herbicide and a 1% increase in seed oil content compared to generic AAC 10CS0048/MIDAS™. Additional phenotypic characteristics as described in the protocol as set forth in Example 7 were measured and recorded (data not shown). Other modified MIDAS™ lines have been obtained as above using different introgressions of herbicide resistant plants of the present disclosure, such as 13CS0786, 14CS0814, 13CS0777-02, 13CS0778-02 and 13CS0779-02, crossed with 13CS0583 and subsequently backcrossed (data not shown).

### Example 19: Modified Cypress Lines

In order to introduce the herbicide tolerance trait (2 genes) into an elite camelina cultivar (CYPRESS™), introgression was performed using 13CS0786 (Example 3) and 13CS0787-08 (PBR SES0787LS, a.k.a. CYPRESS™). The following crosses and backcrosses (BC) were performed:
(i) F1: 13CS0786×13CS0787−08=15CS0999
(ii) BC1: 15CS0999×13CS0787−08=15CS1020
(iii) BC2: 15CS1020×13CS0787−08=16CS1056
(iv) BC3: 16CS1056×13CS0787−08=16CS1070
(v) BC4F1: 16CS1070×13CS0787−08=17CS1088
(vi) BC4F2: 17CS1088 selfed=17CS1112
(vii) BC4F3: 17CS1112 selfed=17CS1131
(viii) BC4F4: 17CS1131 (1-5) bulked and selfed: 18CS1152, 18CS1153, 18CS1154, 18CS1155, 18CS1156

One plant from each BC4F4 family was selfed separately and the progeny (BC4F5) was sprayed with a ¼ rate of thifensulfurom-methyl (2 trays of each). Bulk seed from each of the BC4F4 families was also sprayed with a ¼ rate of thifensulfurom-methyl and it was confirmed that all families do not segregate anymore.

The 5 different thifensulfuron-methyl resistant CYPRESS™ lines (18CS1152, 18CS1153, 18CS1154, 18CS1155 and 18CS1156) as well as the MIDAS™ introgressed line 17CS1115 (Example 18) are being evaluated in a replicated field trial at AAFC-Saskatoon. In addition, efficacy field trials are in progress at North Dakota State University in Fargo, ND (Dr. Kirk Howatt and Dr. Marisol Berti) and Montana State University in Huntley, Montana (Dr. Prashant Jha). The field trials are ongoing and results are not yet available.

### Example 20: Modified Pearl Lines

Introgression of the resistance trait into camelina variety SES0887IOR/Pearl is in progress (BC2 completed).

Although the foregoing has been described in some detail by way of illustration and example for purposes of clarity of understanding, it is readily apparent to those of ordinary skill in the art in light of the teachings of the present disclosure that certain changes and modifications may be made thereto without departing from the spirit or scope of the appended claims.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present subject matter is not entitled to antedate such publication by virtue of prior invention.

It must be noted that as used in this specification and the appended claims, the singular forms “a”, “an”, and “the” include plural reference unless the context clearly dictates otherwise. Unless defined otherwise all technical terms used herein have the same meaning as commonly understood to one of ordinary skill in the art to which the present disclosure belongs.

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

As used herein, whether in the specification or the appended claims, the transitional terms “comprising”, “including”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiments herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the subject matter disclosed and/or claimed herein.

### REFERENCES

1) Altschul, S. F., Madden, T. L., Schäffer, A. A., Zhang, J., Zhang, Z., Miller, W., & Lipman, D. J. (1997) “Gapped BLAST and PSI-BLAST: a new generation of protein database search programs”, Nucleic Acids Research, 25(17):3389-3402.
2) Amarowicz, R., Estrella, I., Hernández, T., Robredo, S., Troszyńska, A., Kosińska, A., & Pegg, R. B. (2010) “Free radical-scavenging capacity, antioxidant activity, and phenolic composition of green lentil (Lens culinaris)”, Food chemistry, 121(3):705-711.
3) Berhow, M. A, Vermillion, K., Jham, G. N, Tisserat, B., & Vaughn, S. F. (2010) “Purification of a Sinapine-Glucoraphanin Salt from Broccoli Seeds”, American journal of plant sciences, 1(2):113-118.
4) Berhow, M., Polat, U., Glinski, J., Glensk, M., Vaugh, S., Isbell, T., Ayala-Diaz, I., Marek, L., Gardner, C. (2013) “Optimized analysis and quantification of glucosinolates from Camelina sativa seeds by reverse-phase liquid chromatography”, Industrial Crops and Products, 43:119-125.
5) Bischoff, K. L. (2006). “Glucosinolates”, In: Gupta, R. C. (ed.) Neutraceuticals—efficacy, safety and toxicity, 551-553.
6) Bradford, M. M. (1976) “A rapid and sensitive method for the quantitation of microgram quantities of protein utilizing the principle of protein-dye binding”, Analytical Biochemistry, 72(1-2):248-254.
7) Budin, J. T., Breene, W. M., & Putnam, D. H. (1995) “Some compositional properties of camelina (Camelina sativa L. Crantz) seeds and oils”, Journal of the American Oil Chemists' Society, 72(3):309-315.
8) Butler, E. J., Pearson, A. W., & Fenwick, G. R. (1982) “Problems which limit the use of rapeseed meal as a protein source in poultry diets”, Journal of the Science of Food and Agriculture, 33(9):866-875.
9) Carpenter, C. D., & Simon, A. E. (1998) “Preparation of RNA”, In: Methods in Molecular Biology, Arabidopsis Protocols, Volume 82, Edited by: Martinez-Zapater J. M. and Salinas, J., Totowa: Humana Press, 85-89.
10) Daxenbichler, M. E., Spencer, G. F., Carlson, D. G., Rose, G. B., Brinker, A. M., Powell, R. G. (1991) “Glucosinolate composition of seeds from 297 species of wild plants”, Phytochemistry, 30:2623-2638.
11) De Vos, M., Kim, J. H., and Jander, G. (2007) “Biochemistry and molecular biology of Arabidopsis-aphid interactions”, BioEssays 29:871-883.
12) Dellaporta S. (1994) “Plant DNA Miniprep and Microprep: Versions 2.1-2.3”, In: Freeling M., Walbot V. (eds) The Maize Handbook. Springer Lab Manuals. Springer, New York, NY.
13) Dyer W E, Chee P W, Fay P K. (1993) “Rapid germination of sulfonylurea-resistant Kochia scoparia L. accessions is associated with elevated seed levels of branched-chain amino acids”, Weed Sci., 41:18-22.
14) Eberlein, C. V., M. J. Guttieri, P. H. Berger, J. K. Fellman, C. A. Mallory-Smith, D. C. Thill, R. J. Baerg, and W. R. Belknap. (1999) “Physio-logical consequences of mutation for ALS-inhibitor resistance”, Weed Sci., 47:383-392.
15) Farnham, M. W., Wilson, P. E., Stephenson, K. K., & Fahey, J. W. (2004) “Genetic and environmental effects on glucosinolate content and chemoprotective potency of broccoli”, Plant Breeding, 123(1):60-65.
16) Fahey, J. W., Wade, K. L., Stephenson, K. K., Chou, F. E. (2003) “Separation and purification of glucosinolates from crude plant homogenates by high-speed counter-current chromatography”, J. Chromatogr. A 996:85-93.
17) Ferland, G. (2012) “Vitamin K. Present Knowledge in Nutrition”, Tenth Edition, 230-247.
18) Kagale, S., Koh, C., Nixon, J., Bollina, V. et al. (2014). “The emerging biofuel crop Camelina sativa retains a highly undifferentiated hexaploid genome structure”, Nat. Commun. 5:3706.
19) Kim, Y., Schumaker, K. S., & Zhu, J. K. (2006) “EMS mutagenesis of Arabidopsis”, Methods in Molecular Biology, 323:101-103.
20) Kunst, L., Taylor, D. C., & Underhill, E. W. (1992) “Fatty acid elongation in developing seeds of Arabidopsis thaliana”, Plant Physiol Biochem, 30(4):425-434.
21) Landerouin, A., Quinsac, A., & Ribaillier, D. (1987) “Optimization of silylation reaction of desulphoglucosinolates before gas chromatography”, World Crops: Production and Utilization Descriptions, 13:26-37.
22) Lange, R., Schumann, W., Petrzika, M., Busch, H., Marquard, R., (1995) “Glucosinolates in linseed dodder”, Fett 97:146-152.
23) Löbe, W. (1845) Om Olievaerterne, deres Dyrkning og Behandling samt om Oliepresning og Olierensning (Danish), Translation: Löbe, W., 1845. About the Olievaerterne, their Cultivation and Treatment, and on Oil Pressure and Oil Dilution (Danish).
24) Niciforovic, N. and Abramovi, H. (2014) “Sinapic Acid and Its Derivatives: Natural Sources and Bioactivity”, Comprehensive Reviews in Food Science and Food Safety, 13:34-51.
25) Pearson, W. R., & Lipman, D. J. (1988) “Search for DNA homologies was performed with the FASTA program”, Proc. Natl. Acad. Sci. USA, 85:2444-2448.
26) Price, M. L., Van Scoyoc, S., & Butler, L. G. (1978) “A critical evaluation of the vanillin reaction as an assay for tannin in sorghum grain”, Journal of Agricultural and Food Chemistry, 26(5):1214-1218.
27) Puttick, D., Dauk, M., Lozinsky, S., & Smith, M. A. (2009) “Overexpression of a FAD3 desaturase increases synthesis of a polymethylene-interrupted dienoic fatty acid in seeds of Arabidopsis thaliana L.”, Lipids, 44(8):753-757.
28) Raney, J. P., & Serblowski, G. (2007) “Inserts for FOSS NIRS 6500 spinning ring cups”, In: Quality, Nutrition, Processing and Trade, Eds. Tingdong, F. U. and Chunyun, 39-42.
29) Rode, J. (2002) “Study of autochthon Camelina sativa (L.) Crantz in Slovenia”, Journal of herbs, spices & medicinal plants, 9(4):313-318.
30) Rustgi, S., Matanguihan, J., Mejías, J. H., Gemini, R., Brew-Appiah, R. A., Wen, N., & von Wettstein, D. (2014) “Assessment of genetic diversity among barley cultivars and breeding lines adapted to the US Pacific Northwest, and its implications in breeding barley for imidazolinone-resistance”, PloS one, 9(6), e100998.
31) Schlemmer, U., Frølich, W., Prieto, R. M., & Grases, F. (2009) “Phytate in foods and significance for humans: food sources, intake, processing, bioavailability, protective role and analysis”, Molecular nutrition & food research, 53 (S2).
32) Schumann, W., Stolken, B. (1996) “Glucosinolate content and type of Camelina sativa seed”, VDLUFA-Schriftenr. Kongressband Trier 44:233-236.
33) Schuster, A., Friedt, W. (1998) “Glucosinolate content and composition as parameters of quality of Camelina seed”, Ind. Crops Prod. 7:297-302.
34) Shapiro, T. A., Fahey, J. W., Wade, K. L., Stephenson, K. K., Talalay, P. (2001) “Chemoprotective glucosinolates and isothiocyanates of broccoli sprouts: metabolism and excretion in humans”, Cancer Epidemiol. Biomarkers Prev. 10:501-508.
35) Singh, B. K., Stidham, M. A., & Shaner, D. L. (1988) “Assay of acetohydroxyacid synthase”, Analytical Biochemistry, 171(1):173-179.
36) Sosulski, F. W., & Dabrowski, K. J. (1984) “Determination of glucosinolates in canola meal and protein products by desulfation and capillary gas-liquid chromatography”, Journal of Agricultural and Food Chemistry, 32(5):1172-1175.
37) Talalay, P., Fahey, J. W. (2001) “Phytochemicals from cruciferous plants protect against cancer by modulating carcinogen metabolism”, J. Nutr. 131: 3027S-3033S.
38) Thellin, O. et al. (1999) “Housekeeping genes as internal standards: use and limits”, J. Biotechnol., 75:291.
39) Thies, W. (1980 May) “Analysis of glucosinolates via ‘on-column’ desulfation”, In: Proc Symp Analytical Chemistry of Rapeseed and its Products, Winnipeg, 66-71.
40) Thompson, C. R., D. C. Thill, and B. Shafii. (1994a) “Germination characteristics of sulfonylurea-resistant and -susceptible kochia (Kochia scoparia)”, Weed Sci., 42:50-56.
41) Tranel, P. J., & Wright, T. R. (2002) “Resistance of weeds to ALS-inhibiting herbicides: what have we learned?”, Weed Science, 50(6):700-712.
42) Vandesompele, J., De Preter, K., Pattyn, F., Poppe, B., Van Roy, N., De Paepe, A., & Speleman, F. (2002) “Accurate normalization of real-time quantitative RT-PCR data by geometric averaging of multiple internal control genes”, Genome Biology, 3(7): research0034.1-research0034.11.
43) Vollmann, J., & Eynck, C. (2015) “Camelina as a sustainable oilseed crop: Contributions of plant breeding and genetic engineering”, Biotechnology Journal, 10(4):525-535.
44) Wacker, J. (1934) “Die Glfriichte Anbau, Pflege und Verwertung”, Verlagsbuchhandlung Paul Parey, Berlin, 31-33 (German).
45) Wishart, D., Arndt, D., Pon, A., Sajed, 1., Guo, A. C., Djoumbou, Y., & Liu, Y. (2014) “13 DB: the toxic exposome database”, Nucleic Acids Research, 43 (D1):D928-D934.
46) Yu, Q., Han, H., Vila-Aiub, M. M., & Powles, S. B. (2010) “AHAS herbicide resistance endowing mutations: effect on AHAS functionality and plant growth”, Journal of Experimental Botany, 61(14):3925-3934.
47) Zubr, J. (2009) “Unique dietary oil from Camelina sativa seed”, Agrafood Ind. Hi-tech, 20(2):42-46.
48) Zubr, J., & Matthäus, B. (2002) “Effects of growth conditions on fatty acids and tocopherols in Camelina sativa oil”, Industrial crops and products, 15(2).

What is claimed:

1. A Camelina sativa acetohydroxyacid synthase (CsAHAS) polypeptide variant comprising a substitution of proline with serine at amino acid position P194, wherein the amino acid position is determined by alignment with a wildtype CsAHAS polypeptide of SEQ ID NO: 1 or 2 and the CsAHAS polypeptide variant shares at least 95% sequence identity with the wildtype CsAHAS polypeptide of SEQ ID NO: 1 or 2.

2. The CsAHAS polypeptide variant of claim 1, which is a CsAHAS1 polypeptide variant comprising SEQ ID NO: 1 with a P194S substitution.

3. The CsAHAS polypeptide variant of claim 1, wherein the polypeptide variant consists of the amino acid sequence of SEQ ID NO: 7.

4. The CsAHAS polypeptide variant of claim 1, wherein the polypeptide variant consists of the amino acid sequence of SEQ ID NO: 8.

5. A polynucleotide encoding the CsAHAS polypeptide variant of claim 1.

6. The polynucleotide of claim 5, comprising a nucleotide substitution of cytosine to thymine at nucleotide position 580, wherein the nucleotide position is determined by alignment with a wildtype CsAHAS nucleotide sequence of SEQ ID NO: 4 or 5.

7. The polynucleotide of claim 5, which comprises the nucleotide sequence of SEQ ID NO: 9.

8. The polynucleotide of claim 5, which comprises the nucleotide sequence of SEQ ID NO: 10.

9. An isolated plant cell that expresses the CsAHAS polypeptide variant of claim 1.

10. An isolated plant cell comprising the polynucleotide of claim 5.

11. An isolated plant cell comprising one or more polynucleotides comprising the nucleotide sequence of SEQ ID NO: 9, the nucleotide sequence of SEQ ID NO: 10, or the nucleotide sequence of SEQ ID NOs: 9 and 10.

12. The isolated plant cell of claim 9, which is a Camelina sativa plant cell.

13. The CsAHAS polypeptide variant of claim 1, which is a CsAHAS2 polypeptide variant comprising SEQ ID NO: 2 with a P194S substitution.

14. The CsAHAS polypeptide variant of claim 1, which is a CsAHAS3 polypeptide variant comprising SEQ ID NO: 3 with a P193S substitution.
